(19) United States
(12) Patent Application Publication
(10) Pub. No.: US 2006/0018911 A1
(43) Pub. Date: Jan. 26, 2006

(54) Design of therapeutics and therapeutics

(76) Inventors: Dana Ault-Riche, Los Gatos, CA (US); Ronald Levy, Stanford, CA (US)

Correspondence Address: FISH & RICHARDSON, PC, P.O. BOX 1022, MINNEAPOLIS, MN, 55440-1022, US

(21) Appl. No.: 11/032,773

(22) Filed: Jan. 10, 2005

Related U.S. Application Data
(60) Provisional application No. 60/557,591, filed on Mar. 29, 2004.
(60) Provisional application No. 60/536,184, filed on Jan. 12, 2004.

(51) Int. Cl. A61K 39/395; C07K 16/46

(52) U.S. Cl. 424/178.1; 435/188.5; 530/391.1

(57) ABSTRACT

Therapeutic complexes and components of therapeutic complexes are provided herein. Also provided are methods of preparing therapeutic complexes and methods of administering therapeutic complexes.

Benefit of priority under 35 U.S.C. § 119(e) to U.S. provisional application Ser. No. 60/557,591, filed Mar. 29, 2004, to Dana Ault-Riche and Ronald Levy, entitled “DESIGN OF THERAPEUTICS AND THERAPEUTICS” is claimed. Benefit of priority under 35 U.S.C. § 119(e) to U.S. provisional application Ser. No. 60/536,184, to Dana Ault-Riche, entitled “DESIGN OF THERAPEUTICS AND THERAPEUTICS,” filed Jan. 12, 2004, also is claimed.

This application also is related to U.S. application Ser. No. 10/699,114 and International Application No. WO 2004/042019, each entitled “SYSTEMS FOR CAPTURE AND ANALYSIS OF BIOLOGICAL PARTICLES AND METHODS USING THE SYSTEMS,” and to U.S. Application Ser. No. 10/699,113, published as U.S. Application Serial No. 2004-0241748-A1 and International PCT Application No. WO 2004/071641, each entitled, “SELF-ASSEMBLING ARRAYS AND USES THEREOF,” and U.S. application Ser. No. 10/699,088, published as U.S. Application Serial No. 2004-0209282-A1 and International PCT application No. WO 2004/039962, each entitled “METHODS FOR PRODUCING POLYPEPTIDE-TAGGED COLLECTIONS AND CAPTURE SYSTEMS CONTAINING THE TAGGED POLYPEPTIDES,” each filed Oct. 30, 2003. This application also is related to U.S. application Ser. No. 10/806,924, to H. Mario Geysen and Dana Ault-Riche entitled “METHODS FOR DESIGNING LINEAR EPITOPES AND ALGORITHM THEREFOR AND POLYPEPTIDE EPITOPES,” filed Mar. 22, 2004.

The subject matter of each of the above noted applications and provisional applications is incorporated in its entirety by reference thereto. Also incorporated by reference is International PCT application No. (attorney docket number 17102-013WO1/1762), filed the same day herewith, to Pointillite, Inc., Dana Ault-Riche and Ronald Levy, entitled “DESIGN OF THERAPEUTICS AND THERAPEUTICS.”

### FIELD OF THE INVENTION

Therapeutic complexes, components of therapeutic complexes, methods for the design and construction of therapeutic complexes and components, and methods of use thereof are provided.

### BACKGROUND

There is a continuous need to develop new technologies to discover new and better pharmaceutical products. Genomics and proteomics have delivered massive amounts of information about life's molecular components. In addition, a multitude of technologies are available to gather such information on a faster and faster scale. For example, robotics and miniaturization technologies lead to advances in the rate at which information on complex samples is generated. High-throughput screening technologies permit routine analysis of tens of thousands of samples; microfluidics and DNA array technologies permit information from a single sample to be gathered in a massively parallel manner. DNA array chips can simultaneously measure the quantity of more than 10,000 different RNA molecules in a sample in a single experiment. Thus, technology has provided large numbers of genes and proteins that serve as targets for development of pharmaceuticals.

The conversion of protein and gene information into effective therapeutic treatments is a challenge to the pharmaceutical industry. Often an agent that binds to a target protein or gene product can be identified, but further study reveals that the agent has little or no therapeutic effect. In other examples, an agent that has a therapeutic effect is found, but it is not specific enough and produces unwanted side effects. In addition, some diseases are technically difficult to address. For example, non Hodgkin's Lymphoma (NHL) is the sixth most common cancer in the United States. NHL is considered a disease of B cell “clonality” since the cancer cells originate from a single initial malignant B cell. B cells are responsible for the production of antibodies in the immune response. Each B cell produces a unique antibody and thus every cancerous B cell carries a unique marker, which is the cancer's idiotype (Id) marker. Since each NHL patient has a unique and different idiotype marker, it is not possible to produce a single drug for treatment of the cancers of all NHL patients via the idiotype marker. There are no methods for large-scale development of such anti-idiotype treatments. Patients with aggressively growing lymphoma can be successfully treated with aggressive chemotherapy. Patients with the slower growing form of the disease eventually succumb. The most effective therapy to date for the treatment of this slower growing, yet fatal form of NHL is the drug rituximab (Rituxitan). rituximab is a human mouse chimeric monoclonal antibody that specifically binds to cell surface CD20 on human B cells; rituximab does not cure the disease.

To implement larger scale approaches to subject-specific therapeutics and to develop pharmaceuticals directed to smaller subject populations, there remains a need for new methods and technologies to develop therapeutics and pharmaceuticals in a cost and time efficient manner. Therefore, among the objects herein, it is an object to provide methods, products and technologies to achieve these goals.

### SUMMARY

Provided herein are therapeutic complexes, components of therapeutic complexes and methods of making therapeutic complexes and components of therapeutic complexes. Pharmaceutical compositions containing the therapeutic complexes and methods of screening for therapeutic complexes and components thereof also are provided.

The therapeutic complexes have a targeting domain and an effector. The targeting domain specifically binds to a target and the effector renders the resulting therapeutic complex biologically effective. The targeting domain and effector are linked via the specific interaction of a binding partner and a capture agent. The binding partner is conjugated to the targeting domain. The capture agent is conjugated to the effector molecule.

Therapeutic complexes provided herein are represented by the formula (TR)ᵣ-(L1)ₛ-(B1)ₜ-(B2)ₓ-(L2)_{y}-(E)_{z}. TR is a targeting domain, E is an effector molecule. The number of TR and E moieties present in a complex is r and z, respectively. B1 and B2 are binding partners and capture agents, respectively. “-” represents an interaction between each component, such as an ionic, covalent, hydrophobic or other interaction such that the resulting complex is sufficiently stable upon formation to achieve a desired effect, such as an in vivo therapeutic effect. A binding partner and capture agent specifically interact, such as, but not limited to, the interaction between a ligand and its receptor, an antibody and an antigen, to form the therapeutic complex. The number of B1 and B2 moieties present in a complex is t and x, respectively. The number of each moiety represented by r, t, x, and z are selected independently and each is an integer from 1 to n, where n is any number of moieties that permit the complex to form and carry out its intended effect, and 1 to n is any number such that the resulting complex has an intended therapeutic activity. “n” can be 2, 3, 4, 5, 6, 7, 8, 9, 10 or more, and is typically 1 or 2.

Each complex also contains at least one capture agent and one binding partner. Binding partners and capture agents are joined to the targeting domains and effectors. L1 and L2 are optional linkers that indirectly link the binding partners and capture agents to the targeting domains and effectors. s and y are the number of L1 and L2 moieties in a complex, respectively. s and y are independently chosen and can be zero or any integer between 1 and n, where n is any number of moieties that permit the complex to form and carry out its intended effect, and I to n includes 1-10, 1-6, 1-5, 1-3. s and/or y can each be zero, such that a targeting domain is directly linked to a binding partner, and/or an effector is directly linked to a capture agent.

Targeting domains can be any molecule that specifically binds to a target. In one example, the targeting domain specifically binds to a subject-specific target. In one example, a targeting domain is a polypeptide containing a sufficient number of amino acids to result in a specific interaction with the target. In another example, the targeting domain is a polypeptide, for example, an antibody or fragment thereof, such as a single chain antibody (scFv), a humanized antibody or fragment thereof. In another example a targeting domain contains at least one variable region of an antibody or one or more CDRs of an antibody. Molecules such as a cell surface receptor, a ligand for a receptor, a cell surface antigen, and an adhesion molecule also can be targeting domains. A targeting domain specifically binds to a target, for example, a cell, such as a B cell, a T cell, a tumor cell, an antibody-secreting cell, an antigen presenting cell, a lymphoma cell and a cytokine-secreting cell. In another example, a targeting domain binds to a cell surface molecule, such as a receptor, an antibody, an antigen, a ligand for a receptor and an adhesion molecule. In yet another example a targeting domain binds to a secreted molecule, such as an antibody or a cytokine. Among the targeting domains provided herein also are targeting domains that bind to an antibody, such as an auto-antibody or an anti-idiotype antibody. Also provided are targeting domains that bind to a pathogen, a virus or a parasite.

Therapeutic complexes can contain a plurality of targeting domains and effector molecules. For example, the complex can contain a plurality of targeting domains that bind to the same or different sites (e.g. epitopes) on a target molecule, cell or other surface. Thus, a plurality of targeting domains in the same complex can cross-react with a common site or epitope on a target molecule or with the same receptor or surface protein or other target on a cell or tissue. Each targeting domain in a complex can recognize different targets, such as different target molecules on a cell surface or different sites in a single molecule.

Effectors include any molecules that confer a biological effect to or on the complex, thereby rendering the resulting therapeutic complex biologically effective. Effectors provided herein include polypeptides. In one example, the effector molecule is a polypeptide containing a sufficient number of amino acids to confer the biological effect on the resulting complex. In one example, an effector is an enzyme, a receptor, a ligand for a receptor, and an inhibitor for a receptor. In another example, an effector is an antibody or fragment thereof, or a humanized antibody. Among the effectors provided herein are effectors that interact with an Fc receptor, such as an Fc domain, for example an Fc domain containing a sequence from a murine IgG2a, a human IgG1 or a human IgG3 antibody. Also provided herein are cytokines as effectors, including cytokines such as IL-2, IL-3, IL-4, IL-5, IL-6, IL-7, IL-9, IL-10, IL-11, IL-12, IL-13, IL-15, IL-16, IL-17, IL-18, IL-1α, IL-1β, and IL-1 RA, granulocyte colony stimulating factor (G-CSF), granulocyte-macrophage colony stimulating factor (GM-CSF), oncostatin M, erythropoietin, leukemia inhibitory factor (LIF), interferons, B7.1, B7.2, TNF-α, TNF-β, LT-β, CD40 ligand, Fas ligand, CD27 ligand, CD30 ligand, 4-1BBL, Trail, and MIF.

Among the effectors provided herein are effectors that confer a biological effect such as receptor binding, receptor inhibition, enzymatic modification, and enzymatic degradation. In one example, the effector confers an immunomodulatory effect or an apoptotic effect. Immunomodulatory effects conferred by an effector include neutralization, immunosuppression, clearance, modulation of cytokine expression or secretion, modulation of T cell activation, modulation of immune cell proliferation, complement activation, antibody-dependent cellular cytotoxicity (ADCC), and opsonization. Among the effectors provided herein, are effectors that confer a direct toxic effect such as a radiolabel or toxin. In one example, an effector in a therapeutic complex does not confer a direct toxic effect and is not a radiolabel.

A capture agent and binding partner are pairs of molecules that specifically bind each other. The affinity of the capture agent is such that it preferentially interacts with the binding partner. Hence, the affinity of the capture agent for the binding partner is greater than the affinity of the capture agent is for itself and the affinity of binding partner is for itself. Typically, the affinity of the capture agent for the binding partner is at least about \0.5-fold,1-fold, 2-fold, 5-fold, 10-fold, 50-fold, 100-fold greater than the affinity of the capture agent for itself and the affinity of the binding partner for itself. In one example, the binding of a capture agent and binding partner is a hydrophobic interaction. In preparing the complexes, the interaction also can be covalent or stabilized by cross-linking following or simulataneously with interaction.

A binding partner is any molecule that specifically binds to a capture agent. Among the binding partners provided herein are polypeptide binding partners, including binding partners between 5 and 100 amino acids in length. Polypeptide binding partners can be any length sufficient to bind or be bound by a capture agent, and include polypeptides of about 5 to 8, 5 to 12, 5 to 20, 5 to 50 and 5 to 100 amino acids in length. In one example, a polypeptide binding partner is an antibody, an antibody fragment, an antigen, an epitope for an antibody, a receptor ligand or a receptor. In another example, the binding partner is an antigen or a receptor ligand. In another example, a binding partner and aits capture agent are not constant domains of an antibody.

A binding partner is conjugated to a targeting domain. Binding partners and targeting domains can be conjugated covalently or non-covalently. In one example, a polypeptide binding partner is conjugated to a targeting domain as a fusion protein. In another example, a linker joins the targeting domain and the binding partner.

A capture agent is any molecule that specifically binds to a binding partner. A capture agent is conjugated to an effector. Capture agents and effectors can be conjugated directly or indirectly through a linker. Capture agents and effectors can also be conjugated through one or more non-covalent linkages. A capture agent and effector can be contained in a single molecule, such as in a polypeptide. In one example, a capture agent and effector are contained in an antibody. In another example, a capture agent and effector are contained in a fusion protein.

Therapeutic complexes assemble through the interaction of a capture agent and binding partner, assembling a targeting domain and effector in a complex. Provided herein are complexes where the specific interaction of the binding partner and capture agent is via a non-covalent linkage. Also provided are complexes where the targeting domain and effector molecule are linked via covalent or non-covalent linkages and combinations thereof. In one example, non-covalent linkages are selected from among hydrogen bonding, hydrophobic bonds, Van der Waals interactions and combinations thereof.

Therapeutic complexes assemble through the interaction of a capture agent and binding partner, assembling a targeting domain and effector in a complex. Such complexes are assembled in vitro or in vivo. In one example, a complex is cross-linked or chemically conjugated after assembly, for example, the binding partner and capture agent in the complex are cross-linked after complex assembly. In another example, a therapeutic complex is isolated after assembly. Among the therapeutic complexes provided herein are those where a binding and effector are expressed in a cell.

Also provided herein are pharmaceutical compositions containing a therapeutic complex described herein. In one example, a pharmaceutical composition contains a therapeutic complex where the biological effect of the complex is a therapeutic effect. Among the pharmaceutical compositions provided herein are compositions prepared by mixing a targeting domain and an effector, where the targeting domain specifically binds to a target, the effector molecule confers a biological effect to the complex, the targeting domain and effector molecule are linked via the specific interaction of a binding partner and a capture agent, the binding partner is conjugated to the targeting domain, and the capture agent is conjugated to the effector molecule, such that a therapeutic complex is formed. In one example, the pharmaceutical composition prepared is subject-specific.

Provided herein also are methods of treating a disease or condition by administering a pharmaceutical composition containing a therapeutic complex as provided herein. Also included are methods of treatment for personalized medicine (personalized treatment) by administering a pharmaceutical composition containing a subject-specific complex. The methods are for treatment of diseases or conditions such as B cell-mediated diseases, an autoimmune disease, T cell-mediated diseases, cancers, breast cancer, colorectal cancer, inflammatory diseases, autoimmune diseases, infectious diseases, neurodegenerative diseases and include non-Hodgkin's lymphoma, rheumatoid arthritis, lupus, multiple sclerosis, melanoma, and posterior intraocular inflammation and pathogen and virus infections.

The complexes administered in the methods of treatment include therapeutic complexes where the targeting domain and the effector are administered as a complex, the targeting domain and the effector are administered sequentially, intermittently or separately. The methods provided herein also include administration of one or more doses of the targeting domain prior to administration of the therapeutic complex.

Also provided herein are methods of preparing a therapeutic complex by contacting a targeting domain and an effector molecule under conditions whereby a complex forms, where the targeting domain specifically binds to a target, the effector molecule confers a biological effect to the complex, the targeting domain and effector molecule are linked via the specific interaction of a binding partner and a capture agent, the binding partner is conjugated to the targeting domain and the capture agent is conjugated to the effector molecule; whereby a therapeutic complex is formed. The methods include contacting the targeting domain and effector together in vitro or in vivo. For example, a targeting domain and effector are contacted in a subject after administration of each separately to the subject. The methods also include the optional step of cross-linking or chemically conjugating the complex after formation, such as by cross-linking the binding partner and capture agent after complex formation. Also provided are methods that include the step of isolating the complex after formation. In one example of the methods, a targeting domain and an effector are expressed in a cell. The methods provided herein for preparing a therapeutic complex include preparation of any of the therapeutic complexes described herein, for example, a therapeutic complex containing a subject-specific targeting domain, a therapeutic complex containing a polypeptide effector and a therapeutic complex where the effector confers an immunomodulatory effect. Methods for preparing therapeutic complexes also include complexes where the affinity of the capture agent for the binding partner is greater than the affinity of the capture agent for itself and the affinity of binding partner for itself.

Also provided herein are methods of imparting a therapeutic effect to an antibody or antibody fragment or target-specific polypeptide by generating a therapeutic complex, where the complex contains a targeting domain containing an antibody or antibody fragment that binds to a target, an effector molecule, where the effector molecule confers a therapeutic effect to the complex, the targeting domain and effector molecule are linked via the specific interaction of a binding partner and a capture agent, the binding partner is conjugated to the targeting domain, the capture agent is conjugated to the effector molecule; and assembling the complex to impart the therapeutic effect. The methods include imparting a therapeutic effect to a single chain antibody (scFv), an anti-idiotype antibody, a variable region, a fragment of a variable region sufficient to bind to another molecule, a CDR, a Fab, a F(ab)₂, or an Fv. Also included are antibodies and antibody fragments that bind to a cell-surface molecule or to a subject-specific target. Such therapeutic complexes include those where the interaction between the capture agent and binding partner is non-covalent. In such therapeutic complexes, the affinity of the capture agent for the binding partner is greater than the affinity of the capture agent for itself and the affinity of binding partner for itself, for example where the affinity of the capture agent for the binding partner is at least 2-fold, 5-fold, 10-fold, 50-fold, 100-fold greater than the affinity of the capture agent for itself and the affinity of binding partner for itself.

Also provided herein are methods for screening tests to identify molecules as candidate components of therapeutic complexes. In one example, a method of screening for test molecules for effectors is provided. The method includes a step of generating a complex containing a targeting domain that is an antibody or antibody fragment that specifically binds to a target, and a candidate effector molecule. The targeting domain and candidate effector molecule are linked via the specific interaction of a binding partner and a capture agent, the binding partner is conjugated to the targeting domain, and the capture agent is conjugated to the candidate effector molecule. The complex is administered to a subject and an effect on the subject is detected, thereby identifying an effector that renders the complex biologically effective.

In another example, a method of screening for test molecules for targeting domains is provided. This method includes generating a complex, where the complex contains a candidate targeting domain that is an antibody or antibody fragment and an effector molecule, where the effector molecule renders the resulting complex biologically effective. The candidate targeting domain and effector molecule are linked via the specific interaction of a binding partner and a capture agent, the binding partner is conjugated to the candidate targeting domain, and the capture agent is conjugated to the effector molecule. The complex is administered to a subject; a therapeutic effect of the complex on the subject is detected to thereby identify a targeting domain.

Also provided herein are combinations of a targeting domain, an effector molecule, a binding partner, a capture agent to which the binding partner specifically binds. The combination also contains optionally cross-linking reagents, and optionally, linkers for linking a binding partner and/or capture agent to a targeting domain or an effector. Among the combinations provided are combinations where the binding partner and capture agent are polypeptides. Also provided are kits containing the combinations and instructions for preparation of therapeutic complexes from the targeting domain, effector molecules, binding partners, and capture agents.

Methods are provided herein for imparting a therapeutic effect to an antibody or antibody fragment by generating a therapeutic complex such as any of the therapeutic complexes described herein and assembling the complex to impart the therapeutic effect, where the complex contains a targeting domain that is an antibody or antibody fragment that binds to a target and an effector molecule that confers a therapeutic effect to the complex. The targeting domain and effector molecule are linked via the specific interaction of a binding partner and a capture agent. The binding partner is conjugated to the targeting domain and the capture agent is conjugated to the effector molecule. In one example of the methods, an antibody or antibody fragment is selected from a single chain antibody (scFv), an anti-idiotype antibody, a variable region, a fragment of a variable region sufficient to bind to another molecule, a CDR, a Fab, a F(ab)₂, and an Fv. In one example, the antibody or antibody fragment binds to a cell-surface molecule. In another example of the methods, the antibody or antibody fragment binds to a subject-specific target.

Also provided is a method of imparting a therapeutic effect to target-specific polypeptide by generating a therapeutic complex such as any of the therapeutic complexes described herein and and assembling the complex to impart the therapeutic effect where the complex contains a targeting domain that is a polypeptide, where the polypeptide specifically binds to a target, and an effector molecule that renders the resulting complex therapeutically effective. The targeting domain and effector molecule are linked via the specific interaction of a binding partner and a capture agent. The binding partner is conjugated to the targeting domain. The binding partner is a polypeptide of sufficient length to specifically interact with a capture agent. In one example, the binding partner is less than about 100 amino acids. In another example, the binding partner is a polypeptide between 5 and 50 amino acids in length and the capture agent is conjugated to the effector molecule. In another example, the binding partner contains 5 to 30, 5 to 20, 5 to 12 or 5 to 8 amino acids.

In one example of the method of imparting a therapeutic effect, the interaction between the capture agent and binding partner is non-covalent. In the methods herein, the affinity of the capture agent for the binding partner is greater than the affinity of the capture agent for itself and the affinity of binding partner for itself. For example, the affinity of the capture agent for the binding partner is at least 2-fold, 5-fold, 10-fold, 50-fold, 100-fold or 1000-fold greater than the affinity of the capture agent for itself and the affinity of binding partner for itself.

Also provided are methods of screening test molecules. Among the methods provided is a method of screening test molecules to identify effectors by generating a complex, such as any of the complexes described herein, administering the complex to a subject and assessing the subject to identify a complex with a therapeutic effect and thereby identifying an effector. The complex contains a targeting domain that is an antibody or antibody fragment that specifically binds to a target and a candidate effector molecule. The targeting molecule and candidate effector molecule are linked via the specific interaction of a binding partner and a capture agent, the binding partner is conjugated to the targeting domain and the capture agent is conjugated to the candidate effector molecule.

Also among the methods provided is a method of screening test molecules to identify targeting domains by generating a complex, such as any of the complexes described herein, administering the complex to a subject, assessing the subject to identify a complex with a therapeutic effect and thereby identify a targeting domain. The complex contains a candidate targeting domain that is an antibody or antibody fragment and an effector molecule, wherein the effector molecule renders the resulting complex biologically effective. The candidate targeting domain and effector molecule are linked via the specific interaction of a binding partner and a capture agent, the binding partner is conjugated to the candidate targeting domain and the capture agent is conjugated to the effector molecule.

In one example, the methods of identifying test molecules as targeting domains and effectors further include the step of assembling the identified targeting domain or identified effector into a therapeutic molecule. Also provided are therapeutic molecules containing a targeting domain or effector or fragment thereof identified by the methods.

Also provided herein are methods of imparting a therapeutic effect to a target-specific polypeptide by generating a therapeutic complex by combining a candidate targeting domain with a first effector molecule via the specific interaction of a binding partner and capture agent, where the complex contains a candidate targeting domain that is a polypeptide, where the polypeptide specifically binds to a target, and a first effector molecule, where the first effector molecule renders the resulting complex therapeutically effective. The candidate targeting domain and first effector molecule are linked via the specific interaction of the binding partner and the capture agent, the binding partner is conjugated to the candidate targeting domain and the capture agent is conjugated to the first effector molecule. The complex is assembled to impart the therapeutic effect, administered to a subject and a therapeutic effect of the complex on the subject is detected to thereby identify a targeting domain. The method also includes generating a therapeutic molecule comprising the identified targeting domain or fragment thereof and a second effector molecule in a polypeptide scaffold or fusion protein, where the targeting domain or fragment thereof is a polypeptide containing a sufficient number of amino acids to result in a specific interaction with the target. In one example, the second effector contains the first effector or a portion of the first effector sufficient to render the therapeutic molecule biologically effective.

Also provided herein are therapeutic complexes containing a targeting domain and an effector molecule where the targeting domain specifically binds to a target and the effector renders the resulting therapeutic complex biologically effective. The targeting domain and effector molecule are linked via the specific interaction of a binding partner and a capture agent. The binding partner is conjugated to the targeting domain and the capture agent is conjugated to the effector molecule. The capture agent comprises at least one variable domain of an antibody or a portion thereof sufficient to specifically bind to the binding partner. In one example, the effector and capture agent comprise an antibody or antibody fragment or antibody complex. For example, the antibody, antibody fragment or antibody in the complex is selected from the group consisting of rituximab, trastuzumab, tositumomab, Ibritumomab, Alemtuzumab, infliximab, CDP-571, edrecolomab, muromab-CD3, daclizumab, omalizumab, cetuximab and bevacizumab and antibody fragments thereof. In another example, the targeting domain specifically binds to a subject-specific target.

Therapeutic complexes provided herein also include complexes where the effector molecule binds to a first target that is the same or different from the target of the targeting domain in the therapeutic complex. Such complexes include complexes with effectors such as antibodies, immunotoxins and antibody conjugates. In one example, the first target and the target of the therapeutic complex are different. In another example, the first target is the same as the target of the targeting domain of the therapeutic complex. In yet another example, the effector binds to the first target in the absence of the complex, and binding of the effector to the first target is altered or reduced when the effector is part of the therapeutic complex. In another example, the first target is different from the target of the therapeutic complex, whereby the therapeutic complex binds to either or both targets.

The therapeutic complexes provided herein include therapeutic complexes with 2 or more capture agents and/or 2 or more targeting domains. In one example, therapeutic complexes herein contain 2 capture agents and 2 targeting domains. In another example, therapeutic complexes have 2 or more targeting domains in which each specifically binds to different targets. For example, the different targets occur on the same cell, tissue or molecule.

### DETAILED DESCRIPTION

A. DEFINITIONS
B. Therapeutic Molecules and Components of Therapeutic Complexes

| | |
|---|---|
| 1. | Targeting domain |

| | |
|---|---|
| a. | Exemplary types of targets |

| | |
|---|---|
| i. | Cell-specific antigens |
| ii. | Secreted and circulating molecules |
| iii. | Pathogen targets |

| | |
|---|---|
| b. | Exemplary types of targeting domains |

| | |
|---|---|
| i. | Proteins as targeting domains |

| | |
|---|---|
| (a) | Antibodies |
| (b) | Receptors and ligands |
| (c) | Protein multimers and |
| | multimerization domains |
| (d) | Lectins and cell-surface |
| | adhesion molecules |

| | |
|---|---|
| ii. | Small molecules as targeting domains |

| | |
|---|---|
| 2. | Effectors and capture agents |

| | |
|---|---|
| a. | Capture agents |
| b. | Effectors |

| | |
|---|---|
| i. | Biological effect |

| | |
|---|---|
| (a) | Destruction |
| (b) | Direct cytotoxicity |
| (c) | Immunostimulation |
| (d) | Immunosuppression |
| (e) | Enzymatic modification |

| | |
|---|---|
| c. | Capture Agent-Effector Associations |

| | |
|---|---|
| 3. | Binding partners |

C. Exemplary therapeutic complexes

| | |
|---|---|
| 1. | Subject-specific complexes |
| 2. | Complexes with polypeptide effectors |
| 3. | Complexes with immunomodulatory effectors |
| 4. | Complexes with a plurality of domains |
| 5. | Retargeted Therapeutic Complexes |

D. Methods of Making Therapeutic Complexes

| | |
|---|---|
| 1. | Identifying and Isolating targeting domains |

| | |
|---|---|
| a. | Phage display |
| b. | Two-hybrid methods |
| c. | Small molecule screening |
| d. | Use of known molecules to construct |
| | targeting domains |
| e. | Assays for characterizing targeting domains |

| | |
|---|---|
| 2. | Identification and Generation of Effectors |

| | |
|---|---|
| a. | Constructing effectors from immunomodulators |

| | |
|---|---|
| i. | Known immune modulators |
| ii. | Immunomodulatory screens |

| | |
|---|---|
| b. | Effectors designed from known molecules |
| c. | Assays for characterizing effectors |

| | |
|---|---|
| 3. | Use of Arrays and other addressable systems to identify |
| | targeting and effector domains |

| | |
|---|---|
| a. | Targeting domain identification |
| b. | Effector Identification |
| c. | Interchange of components |

| | |
|---|---|
| 4. | Design, Generation and Selection of binding partners |
| | and capture agents |

| | |
|---|---|
| a. | Phage display |
| b. | Two-hybrid analysis |
| c. | Sequence analysis and molecular modeling |
| d. | Use of known molecules to design binding |
| | partner-capture agent pairs |
| e. | De novo generation |
| f. | Small molecule binding partners |

E. Assembling and producing therapeutic complexes

| | |
|---|---|
| 1. | Conjugating binding partners and capture agents to |
| | targeting domains and effectors |

| | |
|---|---|
| a. | Fusion proteins |
| b. | Chemical conjugation |

| | |
|---|---|
| 2. | Assembling therapeutic complexes |
| 3. | Assays for function of components and assembled |
| | complexes |
| 4. | Optimization of components and complexes |

| | |
|---|---|
| a. | Humanization |
| b. | Optimization of function |

| | |
|---|---|
| 5. | Use of therapeutic complexes as a screening tool |
| 6. | Expression of therapeutic complexes |

| | |
|---|---|
| a. | Hosts and expression systems |

| | |
|---|---|
| i. | Prokaryotic expression |
| ii. | Yeast |
| iii. | Insect cells |
| iv. | Mammalian cells |
| v. | Plants |

| | |
|---|---|
| b. | Purification of therapeutic complexes |

F. Therapies and Treatments with Therapeutic Complexes

| | |
|---|---|
| 1. | Animal models |
| 2. | Human therapies |
| | Adjuvants and other combination therapies |

G. Exemplary molecules and therapies

| | |
|---|---|
| 1. | B-cell lymphoma |
| 2. | T-cell related ocular diseases and conditions |
| 3. | Lupus |
| 4. | Rheumatoid arthritis |
| 5. | Multiple sclerosis |
| 6. | Retargeting therapeutic agents |

| | |
|---|---|
| a. | Retargeting Antibodies |
| b. | Subject-specific retargeting |
| c. | Retargeting with a plurality of domains |

H. EXAMPLES

### A. DEFINITIONS

Unless defined otherwise, all technical and scientific terms used herein have the same meaning as is commonly understood by one of skill in the art to which the invention(s) belong. All patents, patent applications, published applications and publications, GenBank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, a therapeutic complex refers to a complex that contains a targeting domain (TR) and an effector (E). The targeting domain is conjugated to a binding partner; and the effector is conjugated to a capture agent. The interaction of a capture agent with a binding partner associates an effector and a targeting domain to create a therapeutic complex. A plurality of either or both of a TR and an E can be linked to form a therapeutic complex. The interaction of a capture agent with a binding partner can be one or more bonds or a combination thereof such that the resulting complex is sufficiently stable upon administration to remain associated and exhibit a therapeutic effect. The interaction includes hydrophobic, Van der Waals, ionic, and other such interactions and can include covalent linkages, such as those formed by further treatment with cross-linking agents.

As used herein, a targeting domain refers to a molecule that specifically binds to a target molecule or biological particle with a greater affinity than for non-target molecules or particles. Typically, a targeting domain binds to a target with at least 10-fold, 100-fold or greater affinity over its affinity for non-target molecules or particles. Thus, targeting domains can distinguish and specifically bind to a target in a complex mixture such as in an extract, cells, tissues or fluids of a subject. Targeting domains include naturally occurring molecules, synthetic molecules and derivatives of either, and include, but are not limited to, any molecule, including nucleic acids, small organics, proteins and complexes that specifically bind to other molecules or to specific sequences of amino acids. Targeting domains can be used in their unaltered state or as aggregates with other molecules. They can be attached or in physical contact with, covalently or noncovalently or otherwise associated with, a binding partner, either directly by virtue of the interaction of a targeting domain with a capture agent, or indirectly via a specific binding substance or linker. Examples of targeting domains, include, but are not limited to: antibodies, cell membrane receptors, surface receptors and internalizing receptors, monoclonal antibodies and antisera reactive or isolated components thereof with specific antigenic determinants (such as on viruses, cells, or other materials), drugs, polynucleotides, nucleic acids, peptides, cofactors, lectins, lipids, sugars, and polysaccharides.

As used herein, a target is a molecule or biological particle with which a targeting domain interacts, and is typically, that to which a biological and/or therapeutic effect is directed. Examples of targets include proteins, such as receptors, enzymes, antigens, antibodies, carbohydrates, lipids, nucleic acids, small organic molecules, cells, organelles, pathogens, and viruses. Targets can be multi-unit molecules such as complexes and multimerized polypeptides.

As used herein, subject-specific targets are those targets that exhibit variation from subject to subject that result, for example, from genetic or somatic mutations, stochastic events, such as cell-specific gene rearrangements and amplifications, and environmental conditions. Subject-specific components, such as subject-specific targeting domains, and subject-specific therapeutic complexes refer to components and/or complexes that are specific for a subject-specific target.

As used herein, the a “subject” refers to animals, including mammals, such as human beings.

As used herein, patient-specific refers to subject-specific when the subject is a human with a disease or disorder.

As used herein, personalized medicine or personalized treatment refers to treatment tailored to a specific subject or patient, for example, treatment of a patient with a therapeutic complex that contains a subject-specific targeting domain.

As used herein, targeting refers to the ability of a molecule or complex to specifically bind to a particular molecule or locus or site. Hence, a molecule that specifically binds to a receptor is said to be targeted to that receptor. A targeted molecule is one that binds to a particular site or locus or a plurality of sites or loci with greater affinity than to a non-targeted site or locus. The target of a particular molecule can change if the molecule changes conformation or its site of interaction is otherwise altered, such as upon formation of a therapeutic complex.

As used herein, retargeting refers to a change in the target-specificity of a molecule, complex of molecules or a biological particle. A change in target-specificity can include binding to an additional target, binding to a new target, including new loci or sites in molecules or on cells that were originally bound by the molecule, complex of molecules or biological particle, and/or not binding a target originally bound by the molecule, complex of molecules or biological particle.

As used herein, a component of a therapeutic complex, refers to effectors, binding partners, capture agents and targeting domains that are used to construct therapeutic complexes.

As used herein, an effector is a molecule that confers a biological effect on the therapeutic complex and can be conjugated or linked, directly or indirectly, with a capture agent. Effectors and capture agents can be linked by covalent or noncovalent interactions as long as the interaction is stable upon therapeutic complex formation. An effector and capture agent can be joined in one moiety, such as in a single polypeptide or by association of polypeptide chains. For example, a polypeptide can contain an effector domain that confers the biological effect and a capture agent domain that binds to a binding partner. One exemplary effector is an antibody. One or more variable domains of an antibody function as a capture agent for association with a binding partner. An Fc domain of the antibody (effector domain) confers a biological effect, such as an immunomodulatory effect. Antibodies, such as rituximab (Rituxitan) exemplify such effectors.

As used herein, a biological effect refers to an activity or function of a molecule, complex or composition that results upon combination of the molecule and a target. Biological effects encompass therapeutic effects and pharmaceutical activity of such molecules, complexes or compositions. Biological effects can be observed in in vitro and in vivo systems designed to test such effects. For example, capture systems, such as described in U.S. patent application Ser. No. 10/699,114 and International PCT Publication No. WO 2004/042019, can be used to screen for and test biological effects. Biological effects include, but are not limited to, immunomodulatory activities, ability to form complexes with other molecules, catalytic or enzymatic activity, the ability to specifically bind to a receptor or ligand, and activation, modulation of receptor dimerization, inhibition or modulation of target function, toxicity, apoptosis, induction of apoptosis, stimulation or inhibition of signal transduction and/or cellular responses, removal, destruction and degradation.

As used herein, biologically effective refers to a molecule or complex of molecules that perform or are capable of performing a biological effect or activity. For example, an effector molecule confers a biological effect to the therapeutic complex (also referred to herein as conferring a biological effect on the complex), such that when the complex is assembled, the effector renders the resulting complex biologically effective. A therapeutic complex that is biologically effective directs a biological effect to a target.

As used herein, a pharmaceutical effect (or therapeutic effect) refers to an effect observed upon administration of an agent intended for treatment of a disease or disorder or for amelioration of the symptoms thereof.

As used herein, a biological particle refers to any portion of a living organism or a virus or other such agent and includes, but is not limited to, a virus, such as a viral vector or viral capsid with or without packaged nucleic acid, phage, including a phage vector or phage capsid, with or without encapsulated nucleic acid, a single cell, including eukaryotic and prokaryotic cells or fragments thereof, a liposome or micellar agent or other packaging particle, a prion and other such biological materials.

As used herein, treatment means any manner in which the symptoms of a condition, disorder or disease or other indication are ameliorated or otherwise beneficially altered. Treatment also encompasses any pharmaceutical use of the therapeutic complexes, components of therapeutic complexes and compositions provided herein.

As used herein, a capture agent refers to a molecule that has a specificity for another molecule or biological particle, referred to herein as a binding partner. A capture agent is a molecule that has an affinity for another molecule or for a biological particle. Capture agents can specifically bind, for example, a small molecule, a defined sequence of amino acids, a biopolymer or a three-dimensional or other structure. Capture agents include naturally occurring and synthetic molecules and derivatives of either, and include, for example, any molecule, including nucleic acids, small organics, polypeptides and complexes that specifically bind to other molecules or specific sequences of amino acids or specific three dimensional structures. Capture agents can be used in their unaltered state or as aggregates with other species. They can be attached or in physical contact with, covalently or noncovalently, an effector, either directly or indirectly via a linker. One example of a capture agent attached to an effector is an antibody. One or more variable domain is a capture agent and the Fc domain is an effector domain.

Examples of capture agents, include, but are not limited to: antibodies and fragments thereof, cell membrane receptors, surface receptors and internalizing receptors, monoclonal antibodies and antisera reactive or isolated components thereof with specific antigenic determinants (such as on viruses, cells, or other materials), enzymes, including modified enzymes, such as enzymes that lack catalytic activity but retain binding affinity for a substrate, drugs, polynucleotides, nucleic acids, polypeptides, cofactors, lectins, sugars, polysaccharides, cells, cellular membranes, and organelles. For example, the capture agents can specifically bind to DNA binding proteins, such as zinc fingers, leucine zippers and modified restriction enzymes.

As used herein, binding partner and binding partner tag (bp-tag) are used interchangeably and refer to a molecule or biological particle to which a capture agent binds. Molecules useful as binding partners are interchangeable with molecules useful as capture agents and that unless specifically stated otherwise, the arrangement of chosen molecules for capture agents and binding partners in any complex can be interchanged so long as a chosen binding partner binds specifically to a chosen capture agent to associate a targeting domain and an effector in a complex. As further described herein, capture agents and binding partners are pairs of molecules that specifically interact. A capture agent-binding partner pair is composed of two different molecules that bind to each other. Generally, a capture agent and a binding partner have an affinity for each other that is greater than the affinity of the capture agent for itself and the affinity of the binding partner for itself. Typically, the affinity is at least 2-fold, 5-fold, 10-fold, 50-fold, 100-fold greater, such that homodimerization of the capture agent and homodimerization of the binding partner are disfavored.

As described in more detail below, the binding partners can be linked to targeting domains directly or indirectly, for example via one or more linkers. A polypeptide binding partner generally refers to a binding partner that contains a sequence of amino acids (or three-dimensional structure of amino acids) to which a capture agent specifically binds.

As used herein, a three-dimensional structure refers to the physical structure of a molecule or biological particle.

As used herein therapeutic effect means an effect resulting from treatment of a subject that alters, typically improves or ameliorates the symptoms of a disease or condition or that cures a disease or condition.

As used herein, immunomodulation, immunomodulatory effect and immune modulation are used interchangeably to refer to an effect on the immune system of a subject or changes in an immune response including changes to immune cells and the response of immune cells, to receptors on immune cells, subsets and sub-populations of immune cells to their environment. Immunomodulatory effects include, but are not limited to, neutralization, immunosuppression, clearance, modulation of cytokine expression or secretion, modulation of T cell activation, receptor cross-linking or dimerization, modulation of immune cell proliferation, complement activation, antibody-dependent cellular cytotoxicity (ADCC), and opsonization.

As used herein, immune cells include cells of the immune system and cells that perform functions in an immune response, such as, but not limited to, T-cells, B-cells, macrophages, dendritic cells, neutrophils, eosinophils, basophils, mast cells, plasma cells, antigen presenting cells and natural killer cells.

As used herein, the term “polypeptide” is used interchangeably with the term “protein” and includes peptides containing two or more amino acids. A polypeptide can be a single polypeptide chain, or two or more polypeptide chains that are held together by non covalent forces, by disulfide cross-links, or by other linkers (e.g. peptide linkers). Thus, an immunoglobulin, a single heavy or light chain of an antibody, or an antibody fragment containing all or part of the heavy and light chains of an antibody, no matter how the chains are associated or joined, are exemplary molecules that are included within the term “a polypeptide.” A polypeptide can contain non proteinaceous components, such as sugars, lipids, detectable labels or therapeutic moieties. A polypeptide can be derivatized by chemical or enzymatic modifications (e.g. by replacement of hydrogen by an alkyl, acyl, or amino group; esterification of a carboxyl group with a suitable alkyl or aryl moiety; alkylation of a hydroxyl group to form an ether derivative; phosphorylation or dephosphorylation of a serine, threonine or tyrosine residue; or N— or O-linked glycosylation) or can contain substitutions of an L-configuration amino acid with a D-configuration counterpart.

As used herein, antibody refers to an immunoglobulin, whether natural or partially or wholly synthetically, such as recombinantly, produced, including any derivative thereof that retains the specific binding ability of the antibody. Hence, antibody includes any protein having a binding domain that is homologous or substantially homologous to an immunoglobulin binding domain. For purposes herein, antibody includes antibody fragments, such as Fab fragments, which are composed of a light chain and the variable region of a heavy chain. Antibodies include members of any immunoglobulin class, including IgG, IgM, IgA, IgD and IgE.

As used herein, a monoclonal antibody refers to an antibody produced or derived from a single clone, such as an antibody secreted by a hybridoma clone. Because each such clone is derived from a clone, such as single B cell, all of the antibody molecules are identical. Monoclonal antibodies can be prepared using standard methods known to those with skill in the art (see, e.g., Kohler et al. Nature 256:495 (1975) and Kohler et al. Eur. J. Immunol. 6: 511 (1976)). For example, an animal is immunized by standard methods to produce antibody secreting somatic cells. These cells are then removed from the immunized animal for fusion to myeloma cells.

Somatic cells with the potential to produce antibodies, particularly B cells, are suitable for fusion with a myeloma cell line. These somatic cells can be derived from the lymph nodes, spleens and peripheral blood of primed animals. Specialized myeloma cell lines have been developed from lymphocytic tumors for their efficiency and suitability in hybridoma producing fusion procedures (Kohler and Milstein, Eur. J. Immunol. 6:511 (1976); Shulman et al. Nature 276: 269 (1978); Volk et al. J. Virol. 42: 220 (1982)). Such cell lines facilitate the selection of fused hybridomas from unfused and similarly indefinitely self propagating myeloma cells and enable the selection of fused hybrid cell lines with unlimited life spans that produce the desired single antibody under the genetic control of the somatic cell component of the hybridoma. Monoclonal antibodies also can be produced using recombinant means. For example, a population of nucleic acids can be isolated that encode regions of antibodies. PCR using primers to conserved regions can be used to amplify antibody regions from the population and then reconstruct antibodies or fragments thereof, such as variable domains, from the amplified sequences. Such amplified sequences also can be fused to other proteins, for example a bacteriophage coat, for expression and display on phage. Amplified sequences can then be expressed and further selected or isolated based, for example, on the affinity of the expressed antibody or fragment thereof for an antigen or epitope thereof. Other methods for producing hybridomas and monoclonal antibodies are well known to those of skill in the art.

As used herein, antibody fragment refers to any derivative of an antibody that is less than full length, retaining at least a portion of the full-length antibody's specific binding ability. Examples of antibody fragments include, but are not limited to, Fab, Fab′, F(ab)₂, single-chain Fvs (scFv), Fv, dsFv, diabody, bispecific antibodies, and Fd fragments. The fragment can include multiple chains linked together, such as by disulfide bridges.

As used herein, an Fv antibody fragment is composed of one variable heavy domain (V_{H}) and one variable light (V_{L}) domain linked by noncovalent interactions.

As used herein, a dsFv refers to an Fv with an engineered intermolecular disulfide bond, which stabilizes the V_{H}-V_{L} pair.

As used herein, a F(ab)₂ fragment is an antibody fragment that results from digestion of an immunoglobulin with pepsin at pH 4.0-4.5; it can be recombinantly produced.

As used herein, a Fab fragment is an antibody fragment that results from digestion of an immunoglobulin with papain; it can be recombinantly produced.

As used herein, scFvs refer to antibody fragments that contain a variable light chain (V_{L}) and variable heavy chain (V_{H}) covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

As used herein, hsFv refers to antibody fragments in which the constant domains normally present in an Fab fragment have been substituted with a heterodimeric coiled-coil domain (see, e.g., Arndt et al. (2001) J Mol Biol. 7:312:221-228).

As used herein, diabodies are dimeric scFv; diabodies typically have shorter peptide linkers than scFvs, and they preferentially dimerize.

As used herein bispecific antibodies are antibodies constructed to have two antigen binding sites, each for a different antigen or each composed of a different antigen binding site. Bispecific antibodies can be made by fusing hybridoma lines expressing two different antibodies or they can be made through in vitro and recombinant methods to conjugate two antibody fragments containing different antigen binding sites.

As used herein, humanized proteins, such as antibodies (or fragments thereof) refer to proteins from non-human source, such as antibodies (or fragments thereof), that are modified to include “human” sequences of amino acids so that administration to a human does not provoke an immune response. Methods for preparation of such antibodies are known. For example, the hybridoma that expresses the monoclonal antibody is altered by recombinant DNA techniques to express an antibody in which the amino acid composition of the non-variable regions is based on human antibodies. Computer programs have been designed to identify such regions. Hence humanized proteins are those from other non-human sources modified so that they do not elicit an immune response upon administration to humans in whom the unmodified protein elicited such response.

As used herein, idiotype refers to a set of one or more antigenic determinants specific to the variable region of an immunoglobulin molecule.

As used herein, anti-idiotype antibody refers to an antibody directed against the antigen-specific part of the sequence of an antibody or T cell receptor.

As used herein, an auto-antibody refers to an antibody specific for a self-antigen, i.e. an antigen found in the subject who produces the auto-antibody. Autoimmune diseases are diseases in which auto-antibodies are produced and can contribute to the pathology of the disease.

As used herein, the term “antibody scaffold” refers to a scaffold of an antibody or of an antibody fragment that contains all or part of an immunoglobulin. Exemplary antibody scaffolds include whole antibodies, and fragments thereof, such as Fv fragments (that do or do not contain an introduced disulfide bond), Fab fragments, Fab′ fragments, F(ab′)₂ fragments, single-chain scFv fragments, and Fc fragments.

As used herein, a protein scaffold or polypeptide scaffold refers to any polypeptide or portion thereof that is sufficient to form a conformationally stable structural support, or framework, which is able to display one or more sequences of amino acids that bind to an antigen (e.g. CDRs, a variable region) in a localized surface region. A scaffold can be a naturally occurring polypeptide or polypeptide “fold” (a structural motif), or can have one or more modifications, such as additions, deletions or substitutions of amino acids, relative to a naturally occurring polypeptide or fold. A scaffold can be derived from a polypeptide of any species (or of more than one species), such as a human, other mammal, other vertebrate, invertebrate, plant, bacteria or virus.

As used herein, conjugation refers to the formation of a linkage or association between two molecules, such as between a binding partner and a targeting domain. The linkage can be any interaction, including noncovalent bonding, such as ionic, or covalent bonding such as by preparing fusion proteins or by chemically conjugating two or more molecules, such as conjugating a binding partner and targeting domain. Conjugation is effected through an interaction with sufficient affinity (Kₐ typically of at least about 10⁶ l/mol, 10⁷ l/mol, 10⁸ l/mol, 10⁹ l/mol, 10¹⁰ l/mol or greater (generally 10⁸ or greater) such that interaction is stable. For example, conjugation of a binding partner and targeting domain is stable upon binding of a capture agent to the binding partner and stable to the interaction of the targeting domain with a target. Further, the conjugates are such that a binding partner conjugated to a targeting domain retains the specificity for the interaction between the binding partner and capture agent. Unless stated otherwise, the terms “conjugated” and “linked” are used interchangeable herein.

As used herein, a fusion protein refers to a polypeptide that contains at least two components, such as a fusion of a polypeptide binding partner and a targeting domain. A fusion protein can be produced for example, by expression of nucleic acid in a host cell or in vitro or produced by chemical synthesis.

As used herein, to “bind” a molecule or biological particle means to interact with the molecule or biological particle, bringing it in close proximity. For purposes herein, it is an interaction that permits molecules and biological particles to be complexed together. Typically, such interactions are non-covalent interactions and can include hydrophobic and electrostatic interactions, Van der Waals forces and hydrogen bonds. Generally, protein-protein interactions involve hydrophobic interactions and hydrogen bonds. Binding can be influenced by environmental conditions such as temperature, pH, ionic strength and pressure.

As used herein, affinity refers to the strength of interaction between two molecules such as between a binding partner and a capture agent or between a targeting domain and a target. The binding affinity between molecules described herein such as between a capture agent and a binding partner, and between a targeting domain and a target, typically has a binding affinity (Kₐ) of at least about 10⁶ l/mol, 10⁷ l/mol, 10⁸ l/mol, 10⁹ l/mol, 10¹⁰ l/mol or greater (generally 10⁸ or greater).

As used herein, specificity (also referred to herein as selectively) with respect to two molecules, such as a targeting domain and a target or a binding partner and a capture agent, refers to the greater affinity the two molecules exhibit for each other compared to other molecules. Thus, the two molecules are said to specifically bind to each other. For example, targeting domains generally specifically bind to a target with greater affinity (typically at least 1-, 2-, 5-, 10-fold, generally 100-fold) than other non-targeted molecules or biological particles. Specific binding between binding partners and capture agents refers to the greater affinity a binding partner and a capture agent exhibit for each other compared to their affinities for other molecules and biological particles, such as for other binding partners and other capture agents. Specific binding typically results in selective binding.

As used herein, cross-linking refers to a method of chemical conjugation for linking molecules. Cross-linking can be effected by interaction between moieties in two molecules, such as disulfide bonding and/or by use of cross-linking reagents. Cross-linking reagents include, but are not limited to, heterobifunctional, homobifunctional and trifunctional reagents, and can be used to introduce, produce or utilize reactive groups, such as thiols, amines, hydroxyls and carboxyls, on one or both of the molecules, which can then be contacted with the other, containing a second reactive group, such as a thiol, amine, hydroxyl and carboxyl, to form a chemical linkage between the two molecules. These reagents can be used to directly or indirectly, such as through a linker, conjugate two or more molecules. Cross-linking can be used, for example, to stabilize binding interactions between two molecules such as between a binding partner and a targeting domain, between an effector and a capture agent and between a binding partner and a capture agent.

As used herein, a molecule refers to any compound, including any found in nature and derivatives thereof, including but not limited to, for example, biopolymers, biomolecules, macromolecules and components and precursors thereof, such as peptides, proteins, organic compounds, oligonucleotides or monomeric units of the peptides, organics, nucleic acids and other macromolecules.

As used herein, the term “biopolymer” is a biological molecule, including macromolecules, composed of two or more monomeric subunits, or derivatives thereof, which are linked by a bond or a macromolecule. A biopolymer can be, for example, a polynucleotide, a polypeptide, a carbohydrate, or a lipid, or derivatives or combinations thereof, for example, a nucleic acid molecule containing a peptide nucleic acid portion or a glycoprotein, respectively. Biopolymers include, but are not limited to, nucleic acid, proteins, polysaccharides, lipids and other macromolecules. Nucleic acids include DNA, RNA, and fragments thereof. Nucleic acids can be derived from genomic DNA, RNA, mitochondrial nucleic acid, chloroplast nucleic acid and other organelles with separate genetic material.

A monomeric unit refers to one of the constituents from which a resulting biopolymer or other polymer is built. Thus, monomeric units include, but are not limited to, nucleotides, amino acids, and pharmacophores from which small organic molecules are synthesized.

As used herein, domain refers to a portion of a molecule, e.g., proteins or nucleic acids, that is structurally and/or functionally distinct from other portions of the molecule.

As used herein, a composition refers to any mixture. It can be a solution, a suspension, liquid, powder, a paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof.

As used herein, a kit refers to a packaged combination. A packaged combination can optionally include a label or labels, instructions and/or reagents for use with the combination.

As used herein, receptor refers to a biological molecule that specifically binds to (or with) other molecules and receives, transmits and/or propagates a signal. The term “receptor protein” can be used to more specifically indicate the proteinaceous nature of a specific receptor. Typically, receptors participate in signal transduction pathways such as sensing the extracellular or intracellular environment. Generally, a receptor specifically binds to a ligand, which then triggers a signaling pathway within the cell. Ligands can be peptides, polypeptides, carbohydrates, small organic and inorganic molecules. Receptors can be localized for example, extracellularly, membrane bound, transmembrane, intracellularly and nuclear. The relocalization of some receptors is triggered upon ligand binding.

Cytokines are small soluble molecules secreted by cells that can alter the behavior or properties of the secreting cell or another cell. Cytokine receptors bind to cytokines and trigger a behavior or property within the cell, for example cell proliferation, death and differentiation. Exemplary cytokines include, but are not limited to, interleukins (e.g., IL-2, IL-3, IL-4, IL-5, IL-6, IL-7, IL-9, IL-10, IL-11, IL-12, IL-13, IL-15, IL-16, IL-17, IL-18, IL-1α, IL-1β, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), and MIF.

As used herein, a B cell refers to a lymphocyte that develops from hemopoietic stem cells in the bone marrow of adults and the liver of fetuses and is responsible for the production of circulating antibodies.

As used herein, a T cell refers to a lymphocyte that develops in the thymus from precursor cells that migrate there from the hemopoietic tissues via the blood. T cells fall into two main classes, cytotoxic T cells and helper T cells. Cytotoxic T cells kill infected cells, whereas helper T cells help to activate macrophages, B cells and cytotoxic T cells.

As used herein, an antigen presenting cell (APC) refers to a cell that can process antigens and display their peptide fragments on the cell surface together with molecules required for T cell activation. Antigen presenting cells include B cells, macrophages and dendritic cells.

As used herein, a candidate in the context of a candidate molecule such as a candidate effector and a candidate targeting domain, refers to a molecule to be assessed for a particular property, function or activity.

As used herein, phage display refers to the expression of proteins or peptides on the surface of filamentous bacteriophage.

As used herein, panning refers to an affinity-based selection procedure for the isolation of phage displaying a molecule with a specificity for a capture molecule or sequence of amino acids or epitope or locus therein.

As used herein, profiling refers to detection and/or identification of a plurality of components, generally 3 or more, such as 4, 5, 6, 7, 8, 10, 50, 100, 500, 1000, 10⁴, 10⁵, 10⁶, 10⁷ or more, in a sample. A profile refers to the identified loci to which components of a sample detectably bind. The profile can be detected as a pattern on a solid surface, such as in embodiments when the addressable collection includes an array of capture agents on a solid support, in which case the profile can be presented as a visual image. In embodiments, such as those in which the capture agents and bound tagged molecules are on color-coded beads or are otherwise detectably labeled, a profile refers to the identified polypeptide binding partner tags and/or capture agents to which component(s) is(are) detectably bound, which can be in the form of a list or database or other such compendium.

As used herein, a label is a detectable marker that can be attached or linked directly or indirectly or associated with a molecule and/or biological particle. The detection method can be any method known in the art.

As used herein, a fluorescent protein refers to a protein that possesses the ability to fluoresce (i.e., to absorb energy at one wavelength and emit it at another wavelength). These proteins can be used as a fluorescent label or marker and in any applications in which such labels are used, such as immunoassays, CRET, FRET, and FET assays. For example, a green fluorescent protein (GFP) refers to a polypeptide that has a peak in the emission spectrum at about 510 nm. Green, blue and red fluorescent proteins are well known and readily available (Stratagene, see, U.S. Pat. Nos. 6,247,995 and 6,232,107).

As used herein, screening refers to a process for analyzing molecules and/or biological particles, such as sets of molecules and library compounds, by methods that include, but are not limited to, chemilumenescence, ultraviolet-visible (UV-VIS) spectroscopy, infra-Red (IR) spectroscopy, fluorescence spectroscopy, fluorescence resonance energy transfer (FRET), NMR spectroscopy, circular dichroism (CD), mass spectrometry, other analytical methods, high throughput screening, combinatorial screening, enzymatic assays, antibody assays and other biological and/or chemical screening methods or any combination thereof.

As used herein, a secondary agent is a molecule that influences activity of another molecule either directly or indirectly. Effects of secondary molecules can be in vitro or in vivo. Secondary agent effects include, but are not limited to, stimulation, co-stimulation, inhibition, co-inhibition and competitive effects. Secondary agents include, but are not limited to, an organic compound, inorganic compound, metal complex, receptor, enzyme, protein complex, antibody, protein, nucleic acid, peptide nucleic acid, DNA, RNA, polynucleotide, oligonucleotide, oligosaccharide, lipid, lipoprotein, amino acid, peptide, polypeptide, peptidomimetic, carbohydrate, cofactor, drug, prodrug, lectin, sugar, glycoprotein, biomolecule, macromolecule, an antibody or fragment thereof, antibody conjugate, biopolymer, polymer or any combination, portion, salt, or derivative thereof. Some exemplary molecules that can serve as secondary agents include, but are not limited to, adhesion molecules (e.g., ALCAM, BCAM, CADs, EpCAM, ICAMs, Cadherins, Selectins, MCAM, NCAM, PECAM and VCAM); angiogenic factors (e.g., Angiogenin, Angiopoietins, Endothelins, Flk-1, Tie-2 and VEGFs); binding proteins (e.g., IGF binding proteins); cell surface proteins (e.g., B7s, CD14, CD21, CD28, CD34, CD38, CD4, CD6, CD8a, CD64, CTLA-4, decorin, LAMP, SLAM, ST2 and TOSO); chemokines (e.g., 6Ckine, BLC/BCA-1, ENA-78, eotaxins, fractalkine, GROs, HCCs, MCPs, MDC, MIG, MIPs, MPIF-1, PARC, RANTES, TARK, TECK and SDF-1); chemokine receptors (e.g., CCRs, CX3CR-1 and CXCRs); cytokines and their receptors (e.g., Epo, Flt-3 ligand, G-CSF, GM-CSF, interferons, IGFs, IK, leptin, LIF, M-CSF, MIF, MSP, oncostatin M, osteopontin, prolactin, SARPs, PD-ECGF, PDGF A and B chains, Tpo, TIGF and PREF-1, AXL, interferon receptors, c-kit, c-met, Epo R, Flt-s/Flk-2 R, G-CSF R, GM-CSF R, etc.); ephrin and ephrin receptors; epidermal growth factors (e.g., amphiregulin, betacellulin, cripto, erbB1, erbB3, erbB4, HB-EGF and TGF-α); fibroblast growth factors (FGFs) and receptors (FGFRs); platelet-derived growth factors (PDGFs) and receptors (PDGFRs); transforming growth factors beta (TGFs-β, e.g., activins, bone morphogenic proteins (BMPs) and receptors (BMPRs), endometrial bleeding associated factor (EBAF), inhibin A and MIC-1); transforming growth factors alpha (TGFs-α); insulin-like growth factors (IGFs); integrins (alphas and betas); interleukins and interleukin receptors; neutrophic factors (e.g., BDNF, b-NGF, CNTF, CNTF Rα, GDNF, GRFαs, midkine, MUSK, neuritin, neuropilins, NGF R, NT-3, semaphorins, TrkA, TrkB and TrkC); interferons and their receptors; orphan receptors (e.g., Bob, ChemR23, CKRLs, GRPs, RDC-1 and STRL33/Bonzo); proteases and release factors (e.g., matrix metalloproteinases (MMPs), caspases, furin, plasminogen, SPC4, TACE, TIMPs and urokinase R); T cell receptors; MHC peptides; MHC peptide complexes; B cell receptors; intracellular adhesion molecules (ICAMs); Toll-like receptors (TLRs; recognize extracellular pathogens, such as pattern recognitions receptors (PRR receptors) and PPAR ligands (peroxisome proliferative-activated receptors); ion channel receptors; neurotransmitters and their receptors (e.g., nicotinic acetylcholine, acetylcholine, serotonin, γ-aminobutyrate (GABA), glutamate, aspartate, glycine, histamine, epinephrine, norepinephrin, dopamine, adenosine, ATP and nitric oxide); muscarinic receptors; small molecule receptors (e.g., NO and C0₂ receptors); steroid hormones and their receptors (e.g., progesterone, aldosterone, testosterone, estradiol, cortisol, retinoic acid receptors (RARs), retinoid X receptors (RXRs) and PPARs); peptide hormones and their receptors (e.g., human placental lactogen, prolactin, gonadotropins, corticotropins, calcitonin, insulin, glucagon, somatostatin, gastrin and vasopressin); tumor necrosis factors (TNFs, e.g., April, CD27, CD27L, CD30, CD30L, Cd40, CD40L, DR-3, Fas, FasL, HVEM, lymphotoxin β, osteroprotegerin, RANK, TRAILs, TRANCE and TWEAK) and their receptors; nuclear factors; and G proteins and G protein coupled-receptors (GPCRs).

As used herein, In silico refers to research and experiments performed using a computer. In silico methods include, but are not limited to, molecular modeling studies, biomolecular docking experiments, and virtual representations of molecular structures and/or processes, such as molecular interactions.

As used herein, an address refers to a unique identifier whereby an addressed entity can be identified. An addressed entity is one that can be identified by virtue of its address. Addressing can be effected by position on a surface, such as the locus or loci, or by other identifier, such as one encoded with a bar code or other symbology, a chemical tag, an electronic tag, such as an RF tag, a color-coded tag or other such identifier.

A self-assembling array is an addressable collection of capture agents, where the capture agents specifically bind to predetermined binding partners.

As used herein, a self-assembled array is an array that results when a self-assembling array is combined with molecules or biological particles that are conjugated to binding partners specific for the capture agents in a self-assembling array.

As used herein, the components of a self-assembled array include a self-assembling array, and binding partners specific therefor or nucleic acids encoding the binding partners or sequence information for synthesis of the binding partners or nucleic acids encoded thereby, and optionally conjugation reagents. As used herein, a capture system refers to an addressable collection of capture agents and polypeptide binding partner-tagged molecules bound thereto, where each different binding partner specifically binds to a different capture agent.

As used herein, an addressable collection of capture agents is a collection of reagents, such as antibodies, enzymes and other such molecules and biological particles, that specifically bind to pre-selected binding partners that contain sequences of amino acids, such as epitopes in antigens, in which each member of the collection is labeled and/or is positionally located to permit identification of the capture agent, such as the antibody, and binding partner. The addressable collection is typically an array or other encoded collection in which each locus contains capture agents, such as antibodies, of a single specificity and is identifiable. The collection can be in the liquid phase if other discrete identifiers, such as chemical, electronic, colored, fluorescent or other tags are included. Any moiety, such as a protein, nucleic acid or other such moiety, that specifically binds to a pre-determined sequence of amino acids, such as an epitope, is contemplated for use as a capture agent.

As used herein, an addressable collection of binding sites refers to the resulting sites produced upon binding of the capture agents provided herein to binding partner-tagged reagents. Each capture agent sorts reagents (such as molecules and biological particles) by virtue of their binding partner, each binding partner is linked to a plurality of different molecules, generally polypeptides. As a result, upon sorting, the capture agent and binding partner-tagged-reagent form a complex and the resulting complex can bind to further molecules. Since the tagged reagents specific for each capture agent can contain a plurality of different molecules that share the same binding partner, when bound to a plurality of different capture agents the resulting collection presents a highly diverse collection of binding sites. The collection is addressable because the identity of the binding partners is known or can be ascertained.

As used herein, a capture system refers to an addressable collection of capture agents and binding partner-tagged molecules bound thereto, where each different binding partner specifically binds to a different capture agent.

As used herein, array library refers to the collections of molecules created by physical separation of the mixed library into q number of discrete collections. The array libraries serve as the genetic source for the tagged molecules to be expressed and purified and contacted with arrays of capture agents. Nucleic acid molecules from these libraries also serve as the source of template DNA used in the amplification protocols to recover the desired tagged molecules once identified using the arrays.

As used herein, printing refers to immobilization of capture agents onto a solid support, such as, but not limited to, a microarray.

As used herein, staining refers to the visualization of molecules, such as molecules bound to a capture system. Staining can be non-specific, semi-specific or specific depending on what is labeled in a sample and when it is detected. Non-specific staining refers to the labeling of non-fractionated or all components in a particular sample generally, although not necessarily, prior to exposure to the capture system. Semi-specific staining as used herein refers to labeling of a portion of a sample, such as, but not limited to, the proteins located on the cell surface or on cellular membranes, either before, during or after exposure to the capture system. Specific staining as used herein refers to the labeling of a specific component of a sample, typically after the exposure of the sample to the capture system. The stain can be any molecule that associates with and that permits visualization or detection of bound molecules.

As used herein, an array refers to a collection of elements, such as antibodies, containing three or more members. An addressable array is one in which the members of the array are identifiable, typically by position on a solid phase support or by virtue of an identifiable or detectable label, such as by color, fluorescence, electronic signal (i.e. RF, microwave or other frequency that does not substantially alter the interaction of the molecules or biological particles), bar code or other symbology, chemical or other such label. Hence, in general the members of the array are immobilized to discrete identifiable loci on the surface of a solid phase or directly or indirectly linked to or otherwise associated with the identifiable label, such as affixed to a microsphere or other particulate support (herein referred to as beads) and suspended in solution or spread out on a surface. Thus, for example, positionally addressable arrays can be arrayed on a substrate, such as glass, including microscope slides, paper, nylon or any other type of membrane, filter, chip, glass slide, or any other suitable solid support. If needed the substrate surface is functionalized, derivatized or otherwise rendered capable of binding to a binding partner. In some instances, those of skill in the art refer to microarrays. A microarray is a positionally addressable array, such as an array on a solid support, in which the loci of the array are at high density. For example, a typical array formed on a surface the size of a standard 96-well microtiter plate with 96 loci, 384, or 1536 are not microarrays. Arrays at higher densities, such as greater than 2000, 3000, 4000 and more loci per plate are considered microarrays.

As used herein, a support (also referred to as a matrix support, a matrix, an insoluble support or solid support) refers to any solid or semisolid or insoluble support to which a capture agent, typically a molecule, biological particle or biospecific ligand is linked or contacted. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications. A support can be of any geometry, including particulate or can be in the form of a continuous surface, such as a microtiter dish or well, a glass slide, a silicon chip, a nitrocellulose sheet, nylon mesh, or other such materials. When particulate, typically the particles have at least one dimension in the 5 10 mm range or smaller. Such particles, referred collectively herein as “beads,” are often, but not necessarily, spherical. Such reference, however, does not constrain the geometry of the matrix, which can be any shape, including random shapes, needles, fibers, and elongated. Roughly spherical “beads,” particularly microspheres that can be used in the liquid phase, also are contemplated. The “beads” can include additional components, such as magnetic or paramagnetic particles (see, e.g., DynaBeads® (Dynal Inc., Oslo, Norway)) for separation using magnets, as long as the additional components do not interfere with the methods and analyses herein.

As used herein, matrix or support particles refers to matrix materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 mm³ or less, 100 μm or less and can be order of cubic microns. Such particles are collectively called “beads.”

As used herein, biological sample refers to any sample obtained from a living or viral source and includes any cell type or tissue of a subject from which nucleic acid or protein or other macromolecule can be obtained. The biological sample can be a sample obtained directly from a biological source or processed For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants. Also included are soil and water samples and other environmental samples, viruses, bacteria, fungi algae, protozoa and components thereof.

As used herein, macromolecule refers to any molecule having a molecular weight from the hundreds up to the millions. Macromolecules include peptides, proteins, nucleotides, nucleic acids, and other such molecules that generally are synthesized by biological organisms, but can be prepared synthetically or using recombinant molecular biology methods.

As used herein, a biomolecule is any compound found in nature, or derivatives thereof. Biomolecules include but are not limited to: oligonucleotides, oligonucleosides, proteins, peptides, amino acids, peptide nucleic acids (PNAs), oligosaccharides and monosaccharides.

As used herein, a subcellular compartment or an organelle is a membrane-enclosed compartment in a eukaryotic cell that has a distinct structure, macromolecular composition, and function. Organelles include, but are not limited to, the nucleus, mitochondrion, chloroplast, and Golgi apparatus.

As used herein, cell capture refers to the immobilization of a cell by a capture system provided herein.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, and other such analogs and derivatives or combinations thereof. Nucleic acid can refer to polynucleotides such as deoxyribonucleic acid (DNA) and ribonucleic acid (RNA). The term also includes, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine.

As used herein, the term “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a phophorothioate bond, a thioester bond, or a peptide bond (peptide nucleic acid). The term “oligonucleotide” also is used herein essentially synonymously with “polynucleotide,” although those in the art recognize that oligonucleotides, for example, PCR primers, generally are less than about fifty to one hundred nucleotides in length.

Nucleotide analogs contained in a polynucleotide can be, for example, mass modified nucleotides, which allows for mass differentiation of polynucleotides; nucleotides containing a detectable label such as a fluorescent, radioactive, luminescent or chemiluminescent label, which allows for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically. For example, a polynucleotide can include one or more deoxyribonucleotides, followed by one or more ribonucleotides, which can be followed by one or more deoxyribonucleotides, such as a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well known methods (see, for example, Weiler et al. Nucleic acids Res. 25: 2792-2799 (1997)).

As used herein, oligonucleotides refer to polymers that include DNA, RNA, nucleic acid analogues, such as PNA, and combinations thereof. For purposes herein, primers and probes are single-stranded oligonucleotides or are partially single-stranded oligonucleotides.

As used herein, “production by recombinant means by using recombinant DNA methods” means the use of the well known methods of molecular biology for expressing proteins encoded by cloned DNA.

As used herein, the term “vector” refers to a nucleic acid molecule capable of transporting another nucleic acid to which it has been linked. One type of preferred vector is an episome, i.e., a nucleic acid capable of extra chromosomal replication. Preferred vectors are those capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids” that refer to circular double-stranded DNA loops that, in their vector form, are not bound to the chromosome. “Plasmid” and “vector” are used interchangeably as the plasmid is the most commonly used form of vector. Other such other forms of expression vectors that serve equivalent functions and that become known in the art subsequently hereto.

As used herein, “transgenic animal” refers to any animal, preferably a non-human animal, e.g., a mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. This molecule can be stably integrated within a chromosome, i.e., replicate as part of the chromosome, or it can be extrachromosomally replicating DNA. In the typical transgenic animals, the transgene causes cells to express a recombinant form of a protein.

As used herein, a reporter gene construct is a nucleic acid molecule that includes a nucleic acid encoding a reporter operatively linked to a transcriptional control sequence. Transcription of the reporter gene is controlled by these sequences. The activity of at least one or more of these control sequences is directly or indirectly regulated by another molecule such as a cell surface protein, a protein or small molecule involved in signal transduction within the cell. The transcriptional control sequences include the promoter and other regulatory regions, such as enhancer sequences, that modulate the activity of the promoter, or control sequences that modulate the activity or efficiency of the RNA polymerase. Such sequences are herein collectively referred to as transcriptional control elements or sequences. In addition, the construct can include sequences of nucleotides that alter translation of the resulting mRNA, thereby altering the amount of reporter gene product.

As used herein, “reporter” or “reporter moiety” refers to any moiety that allows for the detection of a molecule of interest, such as a protein expressed by a cell, or a biological particle. Typical reporter moieties include, for example, fluorescent proteins, such as red, blue and green fluorescent proteins (see, e.g., U.S. Pat. No. 6,232,107, that provides GFPs from Renilla species and other species), the lacZ gene from E. coli, alkaline phosphatase, chloramphenicol acetyl transferase (CAT) and other such well-known genes. For expression in cells, nucleic acid encoding the reporter moiety, referred to herein as a “reporter gene,” can be expressed as a fusion protein with a protein of interest or under the control of a promoter of interest.

As used herein, the phrase “operatively linked” generally means the sequences or segments have been covalently joined into one piece of DNA, whether in single or double-stranded form, whereby control or regulatory sequences on one segment control or permit expression or replication or other such control of other segments. The two segments are not necessarily contiguous, rather two or more components are juxtaposed so that the components are in a relationship permitting them to function in their intended manner. Thus, in the case of a regulatory region operatively linked to a reporter or any other polynucleotide, or a reporter or any polynucleotide operatively linked to a regulatory region, expression of the polynucleotide/reporter is influenced or controlled (e.g., modulated or altered, such as increased or decreased) by the regulatory region. For gene expression, a sequence of nucleotides and a regulatory sequence(s) are connected in such a way as to control or permit gene expression when the appropriate molecular signal, such as transcriptional activator proteins, are bound to the regulatory sequence(s). Operative linkage of heterologous nucleic acid, such as DNA, to regulatory and effector sequences of nucleotides, such as promoters, enhancers, transcriptional and translational stop sites, and other signal sequences, refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA in reading frame.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences can be cis acting or can be responsive to trans acting factors. Promoters, depending upon the nature of the regulation, can be constitutive or regulated.

As used herein, the term “regulatory region” means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present, or at increased concentration, gene expression increases. Regulatory regions also include sequences that confer repression of gene expression (i.e., a substance or stimulus decreases transcription). When a repressor is present or at increased concentration, gene expression decreases. Regulatory regions are known to influence, modulate or control many in vivo biological activities including cell proliferation, cell growth and death, cell differentiation and immune modulation. Regulatory regions typically bind to one or more trans-acting proteins that results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding sites (IRES) elements for the creation of multigene, or polycistronic, messages, polyadenylation signals to provide proper polyadenylation of the transcript of a gene of interest and stop codons and can be optionally included in an expression vector.

As used herein, substantially identical to a product means sufficiently similar so that the property of interest is sufficiently unchanged so that the substantially identical product can be used in place of the product.

As used herein, equivalent, when referring to two sequences of nucleic acids, means that the two sequences in question encode the same sequence of amino acids or equivalent proteins. When “equivalent” is used in referring to two proteins or peptides, it means that the two proteins or peptides have substantially the same amino acid sequence with only conservative amino acid substitutions (see, e.g., Table 1, below) that do not substantially alter the activity or function of the protein or peptide. When “equivalent” refers to a property, the property does not need to be present to the same extent but the activities generally are substantially the same. “Complementary,” when referring to two nucleotide sequences, means that the two sequences of nucleotides are capable of hybridizing, generally with less than 25%, with less than 15%, and even with less than 5% or with no mismatches between opposed nucleotides. Generally to be considered complementary herein the two molecules hybridize under conditions of high stringency.

The term “substantially” identical or homologous or similar varies with the context as understood by those skilled in the relevant art and generally means at least 70%, at least 80%, at least 90%, and at least 95% identity.

As used herein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224).

Such substitutions can be made in accordance with those set forth in TABLE 1 as follows:

TABLE 1

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art.

As used herein, a highly antigenic, highly specific polypeptide (also referred to herein as HAHS polypeptides) is a polypeptide that specifically binds to a unique member of a collection of capture agents (i.e. binds with at least 1-, 2-, 5- 10-fold or greater affinity to one unique member compared to all other members in a collection of at least 3, 5, 10, 50, 100 or more unique members). Collections of HAHS polypeptides are collections of polypeptides that specifically bind capture agents such that each HAHS polypeptide in the collection will bind to a unique member of a collection of capture agents with greater affinity (typically at least 1, 2, 5, 10-fold or more) than to any other member of the collection of capture agents. The collections of capture agents include at least 3, 5, 10, 50, 100 or more unique capture agents.

The HAHS polypeptides are antigenic in that capture agents that specifically bind HAHS polypeptides are readily designed or prepared. Hence, antigenic refers to the ability of the HAHS polypeptides to bind to capture agents with high affinity and specificity. For example, an HAHS polypeptide specifically binds to a capture agent such as an antibody or any fragment of an antibody of sufficient length to bind to an epitope. The HAHS polypeptides that result from the methods such as described herein can be used to generate capture agents, such as by immunization of animals, particularly rodents and birds, or by in vitro screening methods, such as phage display or other such methods (see, U.S. application Ser. No. 10/806,924, to H. Mario Geysen and Dana Ault-Riche entitled “METHODS FOR DESIGNING LINEAR EPITOPES AND ALGORITHM THEREFOR AND POLYPEPTIDE EPITOPES” filed Mar. 22, 2004. Thus, for example, HAHS polypeptides can be prepared to generate collections of polypeptides that specifically bind capture agents such as antibodies, antibody fragments and engineered molecules that contain binding regions of antibodies and antibody fragments. HAHS polypeptides also can be generated and/or selected to be antigenic in one host and less antigenic in another host. For example, HAHS polypeptides can be highly antigenic in mice but less antigenic or non-antigenic in humans.

As used herein, an epitope refers to a site to which a capture agent specifically binds. Epitopes include antigenic epitopes that are amino acids (contiguous or non-contiguous in the primary sequence) in a polypeptide to which an antibody specifically binds, and also includes other loci, including polypeptides, to which capture agents specifically bind.

As used herein, antigenic when used in the context of highly antigenic highly specific polypeptides refers to polypeptides that induce, upon administration to a host, antibodies that are specific for the HAHS polypeptides or upon screening, or select for (such as in display or panning methods) capture agents, such as antibodies or antibody fragments, with specific and selective binding to the HAHS polypeptides.

As used herein, antigenic ranking refers to a statistical probability that an amino acid or set thereof occurs in an antigenic polypeptide, including epitopes in naturally occurring polypeptides.

As used herein, a similarity ranking refers to a comparison among amino acids and is represented or determined as a probability or fraction that two amino acids are structurally and/or functionally similar. For example, two identical amino acids have a similarity ranking of 100; two very dissimilar amino acids, such as proline and tyrosine have a ranking of 0.

As used herein, a subset of a set contains at least one less member than the set.

As used herein, a critical residue or amino acid in an HAHS polypeptide is one that influences the affinity or specificity of binding to the binding protein (capture agent). Critical residues taken from the set of naturally occurring amino acids can only be replaced by a subset of amino acids (usually 1 or 2 amino acids) or in some cases, can not be replaced by any other amino acid from this set.

As used herein, a non-critical residue or amino acid in an HAHS polypeptide is one that does not influence the affinity or specificity of binding to the binding protein (capture agent). Noncritical residues can be replaced by a larger subset of amino acids (for example, when taken from the set of naturally occurring amino acids, they can be replaced usually 10 or more amino acids or in some cases, by any other amino acid from this set) without affecting the affinity or specificity of binding. In some cases, non-critical residues are used to confer additional functionalities or properties on polypeptides. In this case, they can typically only be replaced by a limited number of amino acids to retain the functionality or property.

As used herein, an amino acid is an organic compound containing an amino group and a carboxylic acid group. A polypeptide comprises two or more amino acids. For purposes herein, amino acids include the twenty naturally-occurring amino acids, non-natural amino acids, and amino acid analogs. These include amino acids wherein a-carbon has a side chain.

As used herein, naturally occurring amino acids refers to the 20 L-amino acids that occur in polypeptides.

As used herein, the term “non-natural amino acid” refers to an organic compound that has a structure similar to a natural amino acid but has been modified structurally to mimic the structure and reactivity of a natural amino acid. Non-naturally occurring amino acids thus include amino acids or analogs of amino acids other than the 20 naturally occurring amino acids and include, but are not limited to, the D-isostereomers of amino acids. Exemplary non-natural amino acids are described herein and are known to those of skill in the art.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726). Each naturally occurring L-amino acid is identified by the standard three letter code (or single letter code) or the standard three letter code (or single letter code) with the prefix “L−;” the prefix “D−” indicates that the stereoisomeric form of the amino acid is D.

### B. THERAPEUTIC MOLECULES AND COMPONENTS OF THERAPEUTIC COMPLEXES

Provided herein are therapeutic complexes, pharmaceutical compositions containing the complexes, methods of making the complexes and methods of using the complexes and pharmaceutical compositions. The therapeutic complexes are designed to specifically recognize a target and provide a biological effect in a target-specific manner. Also provided are methods of providing subject-specific therapeutic complexes and components and methods of making and using the therapeutic complexes.

The therapeutic complexes contain a target recognition function, which can be subject-specific, herein referred to as a targeting domain (TR), and a domain that confers a biological effect, referred to as an effector (E). The effector and targeting domain are brought together by the interaction of two moieties, a binding partner (also referred to herein as a tag and/or binding partner tag) and a capture agent. The binding partner (B1) is conjugated to a targeting domain and the capture agent (B2) is conjugated to an effector. In some embodiments, an effector and capture agent are contained within one molecule.

Thus, the complexes have the formula (TR)ᵣ-(L1)ₛ-(B1)ₜ-(B2)ₓ-(L2)_{y}-(E)_{z}. As noted above, TR is a targeting domain, E is an effector molecule. The number of TR and E moieties present in a complex is r and z, respectively. B1 and B2 are binding partners and capture agents, respectively. The number of B1 and B2 moieties present in a complex is t and x, respectively. The number of each moiety represented by r, t, x, and z are selected independently and each is an integer from 1 to n, where n is any number of moieties that permit the complex to form and carry out its intended effect, and 1 to n includes 1- 10, 1-6, 1-5, 1-3. Thus, each complex has one or more targeting domains and one or more effectors. Each complex also contains one or more pairs of binding partners and capture agents.

Binding partners and capture agents are joined to the targeting domains and effectors. L1 and L2 are linkers that can indirectly link the binding partners and capture agents to the targeting domains and effectors. s and y are the number of L1 and L2 moieties in a complex, respectively. s and y are independently chosen and can be zero or any integer between 1 and n, where n is any number of moieties that permit the complex to form and carry out its intended effect, and 1 to n includes 1-10, 1-6, 1-5, 1-3. In one embodiment, s and/or y are equal to zero, such that a targeting domain is directly conjugated to a binding partner, and/or an effector is directly conjugated to a capture agent. When a targeting domain is directly conjugated to a binding partner and an effector is directly conjugated to a capture agent, the complex also can be represented simply as (TR)ᵣ-(B1 )ₜ-(B2)ₓ-(E)_{z}.

The components of a complex are joined by any stable interaction, including covalent bonds, ionic bonds, hydrophobic, Van der Waals, hydrogen bonds and other such bonds and interactions, such that the resulting complex is stable upon administration to a subject, such that it performs its intended effect. Typically such linkages have a binding affinity (Kₐ) of at least about 10⁶ l/mol, 10⁷ l/mol, 10⁸ l/mol, 10⁹ l/mol, 10¹⁰ l/mol or greater (generally 10⁸ or greater).

The interaction between a binding partner and a capture agent is specific, such that a particular capture agent binds to a particular binding partner with a greater affinity, generally 2-fold, 5-fold, 10-fold, 100-fold or greater, than to other molecules, including other capture agents and other binding partners. Generally, a capture agent and a binding partner have an affinity for each other that is greater than the affinity of the capture agent for itself and the affinity of the binding partner for itself. Typically, the affinity is at least 2-fold, 5-fold, 10-fold, 50-fold, 100-fold greater, such that homodimerization of the capture agent and homodimerization of the binding partner are disfavored. This added specificity promotes efficient complex formation.

Generally, the interaction between binding partner and capture agent (B1 and B2) is non-covalent. It can be stabilized by cross-linking, such as by treating with a compound or condition after forming the complex. Therapeutic complexes can be formulated for administration to a subject (as described below or by other methods known to those of skill in the art).

The therapeutic complexes produced by the association of a targeting domain and an effector have a variety of uses and applications. Because a targeting domain confers target-specificity, therapeutic complexes can be designed for any target by providing a molecule that recognizes or binds to a target of interest. Secondly, because the targeting molecule may or may not need to provide a biological effect directly, target recognition molecules can be converted easily and quickly to pharmaceutically effective molecules. Further, the compositions and methods provided herein permit rapid design, discovery and validation of molecules with therapeutic effectiveness.

Also provided are therapeutic complexes that bind more than one target molecule and/or that have a plurality of effector functions. For example, the complexes can contain a plurality of targeting domains that bind to the same or different targets and/or a plurality of effectors. Therapeutic complexes containing a plurality of effectors can confer a plurality of biological effects; the plurality of effectors can be the same or different.

Molecules particularly targeted therapeutics, can be retargeted and/or given additional biological effects by assembling such molecules into a therapeutic complex that contains the molecule plus one or more of a targeting domain and/or effector. Retargeting of a molecule includes altering or eliminating binding to a first target and providing binding to one or more new or additional targets. Retargeting includes increasing binding to a first target, such as by increasing the affinity or avidity of interaction with a target.

One application of the therapeutic complexes and methods is for the design and preparation of subject-specific therapeutics. In such applications, a targeting domain (TR) is included that is directed to a subject-specific molecule or biological particle, such as a tumor antigen, tumor cell, or auto-antibody. For example, a targeting domain is linked to a binding partner and an effector is linked to a capture agent. The effector and targeting domain are associated to create a therapeutic complex by the specific interaction of the capture agent with the binding partner, thereby resulting in a biological effect on a subject-specific target molecule or biological particle upon administration of the complex.

The design of therapeutic complexes herein is modular. The components, targeting domains, binding partners, capture agents and effectors can be designed together or independently and then assembled into effective complexes. Further, the complexes can be multivalent, composed of one or more effectors (E), one or more binding partners, one or more capture agents and one or more targeting domains (TR). It is to be understood and is further detailed herein, that molecules useful as binding partners and capture agents are interchangeable and that unless specifically stated otherwise, the arrangement of chosen molecules for capture agents and binding partners in any complex can be interchanged so long as a chosen binding partner binds specifically to a chosen capture agent to associate a targeting domain and an effector in a complex.

A detailed description of the exemplary components used in the combinations and methods is set forth below. It is understood that the scope of the disclosure is not limited to the exemplified embodiments, that any of the possible targeting domains, binding partners, capture agents and effector combinations can be used to construct therapeutic complexes.

1. Targeting Domain

A targeting domain confers target recognition, i.e. specificity for a target, to a therapeutic complex. The type of molecule selected as a targeting domain will be influenced by the target that is of interest. Targets include, but are not limited to, cell surface antigens, cell surface receptors, proteins, lipids and carbohydrate moieties on the cell surface or within the cell membrane, molecules processed on the cell surface, secreted and other extracellular molecules and molecules and cells circulating within the body of a subject. Targets include any type of cell such as prokaryotic and eukaryotic cells. Targets also include viruses, including virus particles, virus proteins and naked viral genomes.

Targeting domains are constructed by any method and from any suitable starting materials. For example, they can be constructed from any molecule that specifically binds to a target and that can be linked to a binding partner without destroying the specific binding to the target. Any molecule that specifically binds to a target or that can be engineered or modified to bind to a target is a targeting domain as defined herein. Molecules useful as targeting domains include, but are not limited to, an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; and other such biological materials. Exemplary molecules useful as targeting domains include ligands for receptors, such as proteinaceous and small molecule ligands, and antibodies and binding proteins, such as antigen-binding proteins. The choice of targeting domain will depend on the target to which the therapeutic complex is directed.

a. Exemplary Types of Targets

Cells provide numerous targets on their cell surface and within the cellular membrane that can be effective therapeutic targets. The cell membrane in eukaryotic and prokaryotic cells is a fluid phospholipid bilayer embedded with proteins, lipids, glycolipids and glycoproteins. The proteins and glycoproteins in the cytoplasmic membrane are quite diverse and include, but are not limited to, channel proteins to form pores for the free transport of small molecules and ions across the membrane; carrier proteins for facilitated diffusion and active transport of molecules and ions across the membrane; cell recognition proteins that identify a particular cell; receptor proteins that bind specific molecules such as hormones, cytokines, and antibodies; and enzymatic proteins that catalyze specific chemical reactions.

Cell types differ in the types and number of biomolecules present on the surface of the cell. This variation can be correlated to their function within the larger organism. For example, B cells function as a source of antibodies for the immune system. T cells help to eliminate pathogens that reside inside host cells. For this function, T cells display surface molecules, such epitope receptors called T-cell receptors (TCRs). Such cell-specific differences can be exploited to generate cell-specific targets for therapeutic complexes.

i. Cell-Specific Antigens

Many cancers and auto-immune diseases exhibit specific cell surface markers. For example, when B cells initially develop, an IgM immunoglobulin is displayed on the surface of the cell. IgM is a member of the immunoglobulin superfamily, where all members possess similar structure by virtue of a constant domain; the variable domain provides the diversity between IgM molecules and therefore between different B-cells. When B-cells become cancerous, such as in B cell Non-Hodgkin's lymphoma, a clonal B-cell is expanded. The cancerous cells display a specific IgM molecule that differs from the IgMs displayed by healthy B cells. The specific IgM marker in the cancerous B cell (lymphoma cells) can be used as a cell-specific target for the design of therapeutic complexes. An additional feature of B cell lymphoma is the subject-specific nature of the B cell lymphoma. In each subject, a unique B cell, expressing a unique IgM molecule, is clonally expanded in the lymphoma. Thus, each subject provides a unique target.

Many additional cancers exhibit cell-specific targets. Human epithelial cancers are characterized by overexpression of one or more members of the ErbB/EGFR related family of receptor tyrosine kinases. This family includes ErbB/EGFR, ErbB2/HER-2/neu, ErbB3 and ErbB4. For example, ductal carcinomas express elevated levels of ErbB2. EGFR and ErbB2 have been shown to directly contribute to malignancy. Ovarian cancers express a unique carbohydrate antigen on their surface, CA125. Colorectal cancers also exhibit cell specific markers such as carcino-embryonic antigen (CEA) and glycoproteins 17-1A and gp72. Carbohydrate molecules such as galactosamine and fucosylamine can act as targets for hepatoma and leukemia cells. Epithelial mucin can act as a target for small-cell lung carcinomas. Each of these cell-specific molecules can be a target for a therapeutic complex described herein. Additionally, any cell-specific molecule known in the art can be a target for a therapeutic complex described herein.

ii. Secreted and Circulating Molecules

In some cases, diseases trigger the release of specific circulating molecules. These circulating molecules can have detrimental effects and in some cases can be directly causative of one or more disease phenotypes. Circulating molecules also can be targeted by therapeutic complexes described herein. For example, in many autoimmune diseases specific antibodies are produced that recognize self-antigens. These self-directed antibodies (auto-antibodies) contribute to the severity of the disease symptoms. Examples of such diseases include systemic lupus erythematosus (lupus), rheumatoid arthritis, multiple sclerosis and posterior intraocular inflammation, including uveitic disorders and ocular surface inflammatory disorders. In many cases, disease-specific autoantibodies are not the same from subject to subject. Thus, each subject represents a unique target or set of targets for the design of therapeutic complexes provided herein.

iii. Pathogen Targets

Pathogens often exhibit cell-specific differences from the host simply by nature of being a different organism. These cell-specific differences can be exploited to design pathogen specific targeted therapies. In some cases, pathogens evolve either from subject to subject as they are transferred through the population or they can evolve within a single subject while trying to “outsmart” the host immune system. Examples of such pathogens include malaria, and viruses such as HIV, HPV and influenza. Surface proteins can act as targets for such pathogens. For example, in malaria, the antigens merozoite surface protein (MSP-1), circumsporozoite protein (CSP-1), and apical merozoite protein (AMA-1) can act as surface antigens for recognition and in HIV, gp120, is a surface target.

b. Types of Targeting Domains

Targeting domains are molecules that specifically bind target molecules or biological particles with a greater affinity than for non-target molecules or particles. Targeting domains can specifically bind to a target in a complex mixture such as in an extract, cells, tissues or fluids of a subject. For example, a targeting domain can specifically bind to a specific cell type, a specific cell surface molecule. Targeting domains include any molecule that specifically binds with sufficient affinity to the target. Once a target is identified, such as a cell-specific and/or disease-specific antigen, a targeting domain can be designed, selected or generated to bind to the chosen target.

Any molecule that specifically binds to a target or can be engineered to bind to a target is a targeting domain as defined herein, including, but not limited to, an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; and other such biological materials. Examples of targeting domains are described throughout the disclosure herein. Targeting domains can be naturally occurring or synthetic molecules, and include any molecule, such as nucleic acids, small organics, proteins and complexes that specifically bind to cellular targets. Examples of molecules useful as targeting domains include, but are not limited to: antibodies and binding fragments thereof, cell membrane receptors, surface receptors and internalizing receptors, monoclonal antibodies and antisera reactive or isolated components thereof with specific antigenic determinants (such as on viruses, cells, or other materials), drugs, polynucleotides, nucleic acids, peptides, cofactors, lectins, sugars, polysaccharides, and organic compounds.

Targeting domains provided herein can be obtained by any method known to those of skill in the art. Such methods include, but are not limited to, screens including cellular screens, antibody generation, selection such as with phage display, two-hybrid experiments, and addressable collections, such as arrays, and knowledge of molecules interacting with targets. A particular targeting domain can be selected based on numerous factors including, but not limited to, the ease by which it can be obtained, the ease of its experimental manipulation, and the breadth of experimental data previously known or unknown in the literature or through personal experience.

i. Proteins as Targeting Domains

Any protein or portion thereof that binds specifically and with sufficient affinity to the target can be used as a targeting domain. Proteins that bind targets are known in the art and/or can be isolated by rationale design and screening against targets or cells expressing the targets.

Once a protein or protein region that binds to a target is isolated, it can be further improved with mutagenesis. For example, a nucleic acid sequence encoding the protein or protein region is determined. Mutations are made by random or targeted mutagenesis to generate a pool of variants. Proteins translated from the pool of nucleic acid mutations are screened for either improved specificity for a target (relative to binding a control) and increased affinity for a target (relative to the starting, unmutagenized protein or protein region). The improved proteins and/or protein regions can be used as a targeting domain.

Examples of proteins useful as targeting domains include, but are not limited to, receptors, antibodies, antibody fragments such as Fabs, F(ab′)₂, scFvs, Fc domains, and CDRs, ligands such as small peptide ligands and hormones, multimerization domains, enzymes, proteins that are found as members of a protein complex or multimer, lectins, and cell-surface adhesion molecules.

(a) Antibodies

Targeting domains can be constructed from antibodies that recognize a target, for example antibodies generated against cell surface antigens, cell surface receptors, and membrane proteins. Antibodies useful for targeting domains include, but are not limited to, immunoglobulins of any subtype (IgG, IgM, IgA, IgE, IgE) or those of any species, such as, for example, IgY of avian species (Romito et al. (2001) Biotechniques 31:670, 672, 674-670, 672, 675.; Lemamy et al. (1999) Int. J. Cancer 80:896-902; Gassmann et al. (1990) FASEB J. 4:2528-2532), or the camelid antibodies lacking a light chain (Sheriff et al. (1996) Nat. Struct. Biol. 3:733-736; Hamers-Casterman et al. (1993) Nature 363:446-448). The targeting domain can contain more than one polypeptide chain. For example, polyclonal and monoclonal immunoglobulins that have 2 polypeptide chains joined by disulfide bridges, can be used as targeting domains. Additionally fragments of immunoglobulins derived by enzymatic digestion (Fv, Fab) or produced by recombinant methods (scFv, diabody, Fab, dsFv, single domain Ig) (Arbabi et al. (1997) FEBS Lett. 414:521-526; Martin et al. (1997) Protein Eng 10:607-614; Holt et al. (2000) Curr. Opin. Biotechnol. 11:445-449) are suitable targeting domains. Further, engineered antibodies such as single-chain antibody (scFv), where the variable regions of heavy and light chain are joined covalently without constant domains, and entirely new synthetic proteins and peptide mimetics and analogs thereof can be designed for use as targeting domains (Pessi et al. (1993) Nature 362:367-369).

The binding regions of immunoglobulins and antibodies, such as Fab and scFvs can be used to construct targeting domains. In one embodiment, the variable regions of the heavy and the light chains are used as a targeting domain. The variable regions can be linked by disulfide bridges or they can be joined covalently, such as in a protein fusion. In one such embodiment, an scFv is used as a targeting domain. Camelid antibodies are naturally occurring immunoglobulins that are only a single heavy chain. Camelid antibodies or regions thereof, such as the antigen recognition region, variable domain, can be used as targeting domains.

The binding region of antibodies also can be mapped to complementarity determining regions (CDRs) and the CDRs used as targeting domains. Each variable domain is made up of three CDRs that are groups of amino acids that are involved in contacting and binding the antigen. In some cases, CDR peptides can mimic the activity of an antibody molecule (Williams et al. Proc. Natl. Acad. Sci. U.S.A. 86: 5537 (1989)). CDRs can be identified for example, by comparison of antibody variable domains to the database of protein sequences compiled in “Sequences of Proteins of Immunological Interest,” Fifth Edition, Volume 1, Editors: Kabat et al. (1991) (see, e.g., table on page xvi). Other techniques such as surface plasmon resonance detection can be used to identify CDRs or other antibody regions for use as targeting domains. For example, Pharmacia's BIAcore® system can be used to determine binding constants and dissociation constants of antibody antigen interactions of a plurality of antibodies and antibody fragments to identify and map binding regions. To construct a targeting region with isolated CDRs, the CDR regions can be mapped and placed into an antibody scaffold or they can be placed into a heterologous context. For example, one or more CDRs can be isolated from known antibodies such as, but not limited to, Rituxan® antibody (rituximab), Herceptin® antibody (trastuzumab), Avastin® antibody (bevacizumab) and placed into an antibody or other polypeptide scaffold.

(b) Receptors and Ligands

Receptors and ligands can be used to design and construct targeting domains. Many receptors are found at the cell surface as membrane or transmembrane proteins. Often, a particular domain is associated with the extracellular environment that binds to a ligand and then triggers a cellular response, typically using a cascade of proteins such as kinases, phosphatases and transcription factors.

Ligands for cell surface receptors can be proteins or small peptides. In some cases, peptides are natural ligands, such as growth hormone and insulin. In other cases, peptide ligands can be designed to mimic small organic ligands. Examples of receptors that bind peptide ligands include the erbB2 receptor tyrosine kinase, the bradykinin receptor, insulin-like growth factor receptor, G protein coupled receptors, and the APJ receptor. Targeting domains can be constructed from peptide ligands of such receptors, such that the targeting domain or a portion thereof is recognized by a cell surface receptor, for example, by an interaction with a ligand binding domain of the receptor.

Receptors also can be used as targeting domains. In many cases, ligands are made by cells as secreted molecules that are part of a larger cell surface-bound form that is then released from a cell. For example, tumor necrosis factor (TNF) is produced as a membrane-bound form, which is released after cleavage into the extracellular environment. Receptors or fragments thereof can be used to construct targeting domains that bind to their peptide ligands in the free peptide form and/or the cellular bound pro-form.

(c) Protein Multimers and Multimerization Domains

Proteins that are known to multimerize or complex with cell surface molecules also can be used to construct targeting domains. Such proteins can be known from the literature, available commercially or identified through screening or other experimental means known in the art. One such example is cell surface receptors. Many receptors are multimers and contain domains that facilitate this association. For example, the growth hormone receptor dimerizes at the cell surface in response to ligand. The dimerization is facilitated by the extracellular domain of the receptor. This domain can be used as a targeting domain to design molecules that interact with the growth hormone receptor target at the cell surface.

Another example of multimerizing proteins that can be used to construct targeting domains is GPCRs, which are often dimers. For example, CXCR2 is a constitutive dimer at the cell surface that responds to chemokines such as IL-8. Amino acids in the central domain of the receptor participate in receptor dimerization. Using this region of CXCR2, targeting domains can be designed that bind to this cell-surface receptor. In a similar manner, dimerization domains from other GPCRs can be used to construct targeting domains.

(d) Lectins and Cell-Surface Adhesion Molecules

Another example of proteins interacting with the cell surface is lectins that recognize cell surface carbohydrates. Lectins have a carbohydrate recognition domain (CRD) that mediates the binding to specific carbohydrate structures. Mammalian lectins can be divided into classes based on their CRD conservation and the subtypes of carbohydrates that each binds. Targeting domains can be designed using CRDs specific for the target cell surface.

Cell-surface adhesion molecules mediate cell-cell recognition and interactions. Often, these molecules are cell type specific and their interactions with ligands also are specific. Some examples of cell surface adhesion molecules include selecting, such as P-selectin and E-selectin, integrins such as LFA-a, MAC-1, CR4, and VLA-5, and adhesion molecules in the immunoglobulin super family, such as ICAM-1, ICAM-2, VCAM-1 and PECAM. Targeting domains can be constructed from cell surface adhesion molecules by using a protein or a portion thereof that binds to a specific ligand.

ii. Small Molecules as Targeting Domains

Many small molecules interact with specific targets at the cell surface. These small molecules can be known in the art, discovered through cell-based or target-based screens, including high-throughput based screening technologies with natural and/or synthetic libraries, designed In silico based on knowledge of the target or any other means known to one skilled in the art.

One such class of small molecules is ligands for cell surface receptors. The ligands bind to the receptors present on specific cell surfaces and initiate a signaling cascade that eventually regulates gene expression and cellular responses. The interaction of a ligand and receptor is very specific. The ligand binds only to a particular receptor, or class of related receptors, through interactions with specific region(s) of the receptor. A small molecule ligand can be used as a targeting domain to target a cell surface receptor.

Carbohydrates can be used as targeting domains for cell-specific carbohydrate binding proteins. For example, Beta-glucan can interact with MAC-1 on the surface of macrophages. Carbohydrate molecules can be designed and synthesized for use as targeting domains that interact with specific cell surface lectins. Synthetic small molecules also can be designed that mimic natural ligands, for example, small organics can interact with receptors in place of natural ligands. Another class of small molecules useful as targeting domains is substrates for cell surface enzymes. For example, proteases such as endoproteases and metalloproteases are found on the surface of some cell types. These enzymes are involved for example, in the processing and secretion of cell signaling molecules and in the activation of receptors. Targeting domains can be constructed from small molecules that bind to these enzymes, for example as substrates, agonists or antagonists.

Once a small molecule targeting domain is identified, it can be improved through optimization screens or rational design. Variants of the small molecule can be synthesized and then binding can be tested against a target. Small molecules are identified with either improved specificity for a target (as compared to their binding to a control, non-target molecule), or with increased binding affinity to a target (relative to the starting small molecule).

2. Effectors and Capture Agents

An effector provides a biological effect to a therapeutic complex. An effector molecule confers a biological effect to the therapeutic complex, such that when the complex is assembled, the effector provides a biological effect to the complex, and the biological effect is directed to the target. Additionally, an effector is associated with a capture agent that specifically binds to a preselected binding partner. A binding partner can be linked to a targeting domain, such that the binding of a capture agent to that binding partner associates the effector with the targeting domain. A capture agent and an effector can be contained within a single molecule, such that the molecule binds specifically to a binding partner and confers a biological effect. In other embodiments, the functions of a capture agent and effector are contained in two or more molecules that are associated or conjugated.

a. Capture Agents

Capture agents include any agent that specifically binds with sufficient affinity to a binding partner for further use, including in therapeutic molecules described herein. Capture agents also can be used in capture systems as further described herein to identify and test therapeutic complexes and components thereof.

Examples of capture agents, include, but are not limited to: antibodies and binding fragments thereof, cell membrane receptors, surface receptors and internalizing receptors, monoclonal antibodies and antisera reactive or isolated components thereof with specific antigenic determinants (such as on viruses, cells, or other materials), enzymes and other catalytic polypeptides, including, but are not limited to, portions thereof to which substrates specifically bind, enzymes modified to retain binding activity lack catalytic activity, small chemical entities such as biotin, biotin analogs, digoxin, carbohydrates and drugs, polynucleotides, nucleic acids, peptides, cofactors, lectins, sugars, polysaccharides, cells, cellular membranes, and organelles. In one exemplary embodiment, a capture agent is a polypeptide that has been selected or generated to bind to a preselected binding partner. Such polypeptides can be isolated by methods further described herein and known in the art such as capture systems, phage display, 2-hybrid methods and array and panning technologies.

For purposes herein binding between capture agents and binding partners results from interactions that are specific between the components. Molecules that do not discriminate between or among partners generally are not specific binding for use as capture agents and binding partners. Additionally, a capture agent-binding partner pair is composed of two different molecules that bind to each other. Homodimerization of molecules do not constitute a capture agent-binding partner pair. For example, two immunoglobulin constant domains, which can homodimerize as well as interact with other molecules, are not considered for purposes herein to be a capture agent-bining partner pair. In contrast, a variable domain of an antibody interacts specifically with an antigen and can discriminate that antigen from another. Thus, variable domain interactions with antigens are an example of a specific interaction useful for the design and construction of capture agents and binding partners.

b. Effectors

Effectors can be naturally occurring or synthetic molecules. Effectors are typically polypeptides but also can be small chemicals, radiolabeled molecules and nucleic acids. Exemplary effectors include, but are not limited to, receptors, antibodies, enzymes, peptides, peptidomimetics, nucleic acids, carbohydrates, lipids, drugs, prodrugs, liposomes, micellular agents, metal complexes, nucleotides, inorganic compounds, viral proteins and biopolymers. The choice of effector will depend on the choice of biological effect.

i. Biological Effect

An effector provides a biological effect to a therapeutic complex. The biological effect can be direct or indirect. An effector can trigger a therapeutic response on its own or as a result of its proximity to a target, for example, by contacting molecules on the surface of a targeted cell. Of particular interest are effectors that have immunomodulatory effects, direct or indirect, on the immune system of a subject. Also of interest are effectors that can target molecules for neutralization, removal or destruction. Biological effects conferred by an effector include, but are not limited to, immunomodulation, immunostimulation, immuonsuppressive mechanisms, neutralization, toxicity, enzymatic modification, inhibition of signal transduction and cellular responses, removal, destruction and degradation.

(a) Destruction

Destruction of cells and/or molecules can be used to remove harmful, overstimulated or otherwise unwanted cells and molecules from a subject. Destruction can be direct, such as by cytotoxicity, or it can be mediated indirectly such as by activating the immune system to destroy unwanted cells and/or molecules. Destruction also can be triggered through apoptosis and apoptotic pathways and signals. For example, depleting monoclonal antibodies have been used to direct a target to macrophages and NK cells for destruction. The macrophages and NK cells express Fc receptors that recognize the Fc domain of the monoclonal antibody complexed with a target. Recognition triggers phagocytosis and antibody-dependent cytotoxicity. Another pathway to destruction of unwanted cells or molecules involves activation of cytotoxic T cells such as through interactions with the CD3 antigen on the cell surface. Activation of cytotoxic T cells triggers lysis of a target. Molecules that interact with the CD3-T cell receptor complex, CD2 and CD28 have been shown to activate T cells and generate anti-tumor activity. Receptor dimerization, such as dimerization of a cell-surface receptor can induce apoptosis and thereby induce cell destruction.

Effectors can be designed from molecules that trigger destruction by the immune system. For example, antibody Fc domains can be utilized as effectors to mediate biological effects such as target depletion. Particular forms of Fc domains are more active for such biological effects. For example the Fc domain of human IgG1 and the Fc domain of mouse IgG2a are known to be more effective than other Fc domains for their depletion effects, based on their higher affinity for the Fcy receptor. Site-directed mutation has also been used to enhance activities of Fc domains, for example IgG has been mutated to enhance its plasma clearance rate (Hornick et al., (2000) J. Nucl. Med. 41:355-62; Kim et al., (1994) Eur. J. Immunol. 24:542-8). Other protein regions also are known to trigger phagocytosis effects similar to the Fc domain such as the C-terminal tail of the LDL Receptor-related protein (LRP: Patel et al. (2003) JBC 278:44799-807).

Other proteins that trigger cytotoxicity can be used as effectors. Lymphotoxin can be used to confer cytotoxicity and cellular destruction. Lymphotoxin induces apoptosis. Fas and Fas receptor also are involved in triggering apoptosis. Fas-L, a member of the TNF family, binds to the Fas receptor on T-cells activates a cascade of caspases that then cleave DNA and result in cell death. The cells are packaged into apoptotic blebs and inflammatory reactions are minimized. Effectors can be constructed from apoptosis-inducing molecules such as lymphotoxin and FasL or molecules that mimic such effects.

(b) Direct Cytotoxicity

Direct mechanisms for triggering target destruction also are possible. For example, cytotoxic molecules can be used as the effector. Examples of cytotoxic molecules include but are not limited to toxins, radiolabels and prodrugs that can then be activated by targeted cells. Examples of toxins are ricin, doxorubicin, diphtheria toxin, methotrexate and azathioprine. Examples of cytotoxic radiolabels include ¹¹¹In, ¹³¹I, ¹²⁵I, ⁹⁰Y, ⁹⁹Tcm, ¹⁸⁸Re and ²¹³Bi.

(c) Immunostimulation

In some cases, it is desired that a biological effect stimulate the immune system. In some cancer therapies, it has been found that stimulation of the immune system by adjuvants increases the effectiveness of therapies. Such molecules stimulate cells of the immune system such as lymphocytes, natural killer cells and T cells. Examples of molecules with immunostimulative effects include, but are not limited to, interferon and stimulatory cytokines such as interleukin 2, interleukin 12 and TNFα. These molecules exert anti-tumor effects through a variety of mechanisms, including anti-angiogenic effects, increased permeability of tumor endothelium, and stimulation of fibrin deposition and thrombosis in tumor vasculature accompanied by destruction of endothelial cells. In some cases it has been shown that combinations of these molecules lead to synergistic effects in anti-tumor responses. Effector molecules can be designed to provide IL-2, IL-12 and TNFα immunostimulatory effects. Granulocyte-macrophage colony-stimulating factor (GM-CSF) and IL-4 have also been shown to be anti-tumor immunostimulants. GM-CSF has been shown to augment monocyte mediated antibody dependent cell-mediated cytotoxicity (ADCC). Effectors can be designed to provide GM-CSF and IL-4 effects.

Another example of immune stimulation is the use of CD86 and CD80 to activate a T-cell response. CD86 and CD80 (also referred to as B7-1 and B7-2, respectively) are normally expressed on the surface of antigen presenting cells and interact with CD28 on T-cells as a costimulatory signal to activate T-cell expansion. Many tumor cells do not express members of the B7 family of ligands and can use this mechanism to evade immune surveillance. Effector molecules can be designed for example to provide B7 ligands to tumor cells and activate the T cell response.

(d) Immunosuppression

In some cases, it is desirable to provide an immunosuppressive effect for therapy. For example, many diseases result in unwanted inflammation in particular tissues, for example diseases such as rheumatoid arthritis, multiple sclerosis and ocular disorders such as non-infectious posterior intraocular inflammation, uveitic disorders and scleritis and peripheral ulcerative keratinitis. Effectors can be chosen to provide a suppressive effect to such inflammation. A number of molecules are known to play a role in inflammation. For example, cytokines such as IL-2, IL-6 and TNFα participate in proinflammatory responses. Effector molecules can be designed that prevent molecules such as cytokines from triggering inflammation. For example, effectors can bind cytokines and prevent them from reaching their cellular targets. For example, antibodies or binding proteins can be generated to bind to one or more proinflammatory cytokines. Effectors also can be designed to block cellular receptors for pro-inflammatory molecules.

Another example of immunosuppressive effects are those that block signaling within the immune system. One such example of an immunosuppressive effect is the use of antibodies for inhibition of allograft rejection that bind to a target without killing the displaying cells. In this case, a monoclonal antibody can block the costimulatory signals needed to activate T-cells that would recognize a donor antigen. For example, an antibody against CD40 ligand can block the stimulation of cytokine secretion in dendritic cells and thereby block T-cell recognition of the graft. Effectors can be designed to mediate immunosuppressive effects by using antibodies or regions thereof that recognize signaling molecules such as CD40. Small molecules that act as antagonists for T-cell receptors also can be used as effectors.

Immunosuppression also can be mediated through molecules that prevent the migration of immune cells. For example, integrins are known to play a role in immune cell migration. Effectors can be designed to bind integrins and prevent cellular migration, for example by blocking their interactions with ICAMs or other ligands.

(e) Enzymatic Modification

Effectors can be constructed to effect modifications, for example modifications of surface proteins. Such modifications can modulate interactions of a target and the immune system. One such example is the glycosylation state of the HIV exterior envelope protein gp120. Glycoprotein gp120 is the primary target for neutralizing antibodies made by the host. Primary isolates of HIV can be resistant to such neutralizing antibodies. Removal of a single N-linked glycosylation site at the base of the gp120 renders it more sensitive to antibody neutralization. Effectors can be constructed, for example, using enzymatic deglycosylation of gp120, and thus increase the sensitivity of a target to the immune system.

c. Capture Agent-Effector Associations

An effector molecule (component) is associated with capture agent that binds to a preselected binding partner to associate the effector with a targeting domain, thereby producing a targeted therapeutic complex. A binding partner can be any molecule that can be covalently joined to a targeting domain and that is recognized by a capture agent. In one embodiment, an effector and capture agent are contained within the same molecule. An effector is chosen or selected as a single molecule that has the functions of an effector, conferring a biological effect, and of a capture agent that binds to a preselected binding partner.

In one aspect, a capture agent is a polypeptide such as an antibody or a portion of an antibody that specifically binds to a binding partner, such as a preselected peptide. An effector, such as an Fc domain, also is contained within the antibody molecule. For example, the antibody can be an IgG immunoglobulin containing a variable domain that specifically binds to a binding partner and to an Fc domain that confers a biological effect.

As another example, an effector can be joined or associated with a capture agent to create a moiety that binds to a preselected binding partner and confers a biological effect. In one aspect of the embodiment, a capture agent is a single chain antibody that binds to a preselected binding partner. The capture agent is associated or joined to a molecule such as a cytokine that confers a biological effect. A capture agent can be linked to an effector by any known means in the art, including but not limited to covalent linkage such as a disulfide bond, by protein fusion, by cross-linking or by non-covalent interaction such as charge interactions and Van der Waals interactions. In one embodiment, a capture agent and an effector portion are linked as a fusion protein. In another embodiment, a capture agent and an effector portion are linked by non-covalent linkage.

3. Binding Partners

A binding partner includes any molecule that specifically binds with sufficient affinity to a particular capture agent and for purposes of constructing therapeutic complexes, can be conjugated to a targeting domain. Molecules that specifically bind to other molecules can be used to design binding partners, including, but not limited to: an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; or any combination, portion, salt, or derivative thereof. Binding partner:capture agent interactions can include, but are not limited to, protein:protein, protein:nucleic acid, nucleic acid:nucleic acid, protein:lipid, lipid:lipid, protein:carbohydrate, protein:small molecule, receptor:signal, antibody:antigen, peptide nucleic acid:nucleic acid, and small molecule:nucleic acid pairs. Selection of binding partner-capture agent pairs can be empirically determined by those with skill in the art, such as with binding assays, or can include pairs with known high specificity and affinity.

Binding of a binding partner to a capture agent includes, but is not limited to, covalent, ionic, hydrophobic, van der Waals and other such interactions, that results in the complex of an effector with a binding partner-targeting domain. The interaction must be of sufficient affinity to produce a stable complex. Generally the binding affinity (Kₐ) of a binding partner and effector is at least about 10⁶ l/mol, 10⁷ l/mol, 10⁸ l/mol, 10⁹l/mol, 10¹⁰ l/mol or greater (generally 10⁸ or greater).

In one embodiment, a binding partner is a polypeptide binding partner that includes the sequence of amino acids to which a capture agent, such as an antibody, specifically binds. Exemplary polypeptides for use as binding partners can be, for example, short polypeptide molecules, such as molecules with at least 4, 5, 6, 8, 10, 15, 20 or more amino acid residues, or can be a full length protein or fragment thereof capable of binding to a capture agent. Generally, short polypeptides for use as binding partners are between 4-100 amino acids, 4-50 amino acids, 4-20 amino acids and 4-12 amino acids. Antigens for antibodies can serve as binding partners. An antibody binds to a small portion of its cognate antigen, known as its epitope, which contains as few as 3 6 amino acid residues (Pellequer et al. (1991) Methods in Enzymology 208:176). The amino acid residues can be contiguous, or they can be discontinuous within the antigen sequence. When the amino acid residues of the antigen sequence are discontinuous, they are presented in close proximity for recognition by the cognate antibody through three dimensional folding of the antigen. Antigens and epitopes can used to construct binding partners for use in the compositions and methods herein. Some exemplary binding partners provided herein include E-tag polypeptide (SEQ ID NO: 912), a FLAG polypeptide (SEQ ID NO: 913), a Glu-Glu polypeptide (SEQ ID NO: 914), a HA.11 polypeptide (SEQ ID NO: 915), a HSV-tag polypeptide (SEQ ID NO: 916), a c-myc polypeptide (SEQ ID NO: 917), a T7 tag polypeptide (SEQ ID NO: 918), a VSV-G polypeptide (SEQ ID NO: 919), a V5 polypeptide (SEQ ID NO: 920), an AB2 polypeptide (SEQ ID NO: 921), an AB4 polypeptide (SEQ ID NO: 922), a B34 polypeptide (SEQ ID NO: 923), a P5D4-A polypeptide (SEQ ID NO: 924), a P5D4-B polypeptide (SEQ ID NO: 925), a 4C10 polypeptide (SEQ ID NO: 926), an AB3 polypeptide (SEQ ID NO: 927), an AB6 polypeptide (SEQ ID NO: 928), a KT3 -A polypeptide (SEQ ID NO: 929), a KT3 -B polypeptide (SEQ ID NO: 930), a KT3-C polypeptide (SEQ ID NO: 931), a 7.23 polypeptide (SEQ ID NO: 932), a HOPC1 polypeptide (SEQ ID NO: 933), a S1 polypeptide (SEQ ID NO: 934), an E2 polypeptide (SEQ ID NO: 935), a His tag polypeptide (SEQ ID NO: 936), an AUI polypeptide (SEQ ID NO: 937), an AU5 polypeptide (SEQ ID NO: 938), an IRS polypeptide (SEQ ID NO: 939), a KT3 polypeptide (SEQ ID NO: 945), a S-tag polypeptide (SEQ ID NO: 944), NusA (SEQ ID NO: 940), Maltose binding protein (SEQ ID NO: 941), TATA-box binding protein (SEQ ID NO: 942), thioredoxin (SEQ ID NO: 943) and highly specific highly antigenic polypeptides, described further herein, such as SEQ ID NOS: 1-911.

Binding partners also can be small molecules such as ligands for receptors and signaling molecules, antagonists and agonists for receptors, enzymes and signaling molecules. For example, binding partners can be peptides or peptide mimetics for receptor molecules. Examples of receptor:peptide pairs that can be used to construct capture agent:binding partner pairs include TNF receptor:TNF, bradykinin receptor:bradykinin, GPCRs: GPCR peptide ligand (e.g., neurotensins, G_{βγ}); LHRH:cetrorelix, and APJ receptor:apelin. Intracellular receptor:ligand pairs also can used to design capture agent:binding partner pairs. Examples of such receptor:ligands include but are not limited to, steroid hormone receptor:hormone such as estrogen receptor: estrogen and estrogen agonists and antagonists, glucocorticoid receptor: glucocorticoids, progesterone:receptor: progesterones, and ecdysone receptor:ecdysones and ecdysone antagonists.

### C. EXEMPLARY THERAPEUTIC COMPLEXES

An exemplary feature of therapeutic complexes is their modular nature, thus providing flexibility in combining components for design, testing and administration. The complexes, although modular, remain specific by the nature of the interaction between a capture agent associated with an effector and a binding partner that is conjugated to a targeting domain. The interaction between a binding partner and a capture agent is specific, typically a capture agent and a binding partner bind each other with greater affinity (typically at least 10-fold, generally 100-fold) than other molecules or biological particles. This specific binding provides specificity to the complex, associating an effector and a targeting domain together in a complex.

The complexes, (TR)ᵣ-(L1)ₛ-(B1)ₜ-(B2)ₓ-(L2)_{y}-(E)_{z}, composed of targeting domains, binding partners, capture agents, effectors and optionally one or more linkers can be designed together or independently and then assembled into an effective complex. Such modularity allows design of the complexes for a wide number of applications, including the tailoring of such complexes to subject-specific therapies. Additionally, such modularity permits the assembly of complexes with components suited to particular modes of administration based on their stability, ease of production and purification and choice of biological effect.

1. Subject-Specific Complexes

One application of therapeutic complexes is the ability to direct a biological effect such as a therapeutic effect, to a subject-specific target, such as a subject-specific molecule or biological particle. Subject-specific targets are those targets that exhibit variation from subject to subject, due to genetic or somatic mutations, stochastic events, such as cell-specific gene rearrangements and amplifications, and environmental conditions. For example, although all human subjects have clonal populations of B cells, the populations of cells differ between different subjects. Additional examples of subject-specific targets include, but are not limited to, immune cells, such as clonal populations of B cells and T-cells, antibodies such as anti-idiotype antibodies and autoantibodies, tumor-specific antigens and tumor-specific cells, and molecules produced through subject-specific genetic variation, such as variants of expressed proteins.

Using the methods herein, therapeutic complexes can be designed for subject-specific targets. A targeting domain is chosen, generated or selected that specifically binds to the subject-specific target. The targeting domain can be any molecule that binds to the subject-specific target and includes, but is not limited to, an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; and other such biological materials. Examples of proteins useful as subject-specific targeting domains include, but are not limited to, receptors, antibodies, antibody fragments such as Fabs, F(ab′)₂, scFvs, Fc domains, and CDRs, ligands such as small peptide ligands and hormones, multimerization domains, enzymes, proteins that are found as members of a protein complex or multimer, lectins, and cell-surface adhesion molecules.

An effector is chosen for a subject-specific therapeutic molecule to confer a desired biological effect on the target. An effector can be selected, generated or constructed from naturally occurring or synthetic molecules. Effectors useful for subject-specific therapeutic complexes include, but are not limited to peptides and polypeptides such as receptors, antibodies, enzymes, viral proteins and peptidomimetics, small molecules such as radiolabeled molecules, metal complexes, nucleotides, drugs, prodrugs and inorganic compounds, polymers and biomolecules such as nucleic acids, carbohydrates, lipids, liposomes, micellular agents, and biopolymers.

Biological effects conferred by an effector of a subject-specific therapeutic complex include, but are not limited to, immunomodulation, immunostimulation, immunosuppressive mechanisms, neutralization, toxicity, enzymatic modification, inhibition of signal transduction and cellular responses, removal, destruction and degradation. In one embodiment, a subject-specific therapeutic molecule has an effector that has an immunomodulatory effect, direct or indirect, on the immune system of a subject. In another embodiment, the therapeutic molecule has an effector that has a direct cytotoxic effect, for example, an effector containing a radiolabel.

A subject-specific therapeutic complex can have an effector and capture agent contained together in one molecule or alternatively, two or more molecules can be associated to form a moiety that binds to a binding partner and confers a biological effect on the resulting subject-specific complex. In one embodiment, an effector for a subject-specific therapeutic complex has an effector and capture agent contained together in one molecule. In one aspect of the embodiment, a subject-specific therapeutic complex contains an antibody, which binds to a selected binding partner and that confers a biological effect.

An effector chosen for a subject-specific therapeutic complex associates with a chosen targeting domain through interaction of the binding partner joined to the targeting domain with a capture agent associated with the chosen effector. Any binding partner that specifically binds with sufficient affinity to a particular capture agent and that can be conjugated to a targeting domain can be used. Molecules useful as binding partners include, but are not limited to, an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; or any combination, portion, or derivative thereof. Exemplary binding partners useful for subject-specific therapeutic molecules include binding partners generated using methods to design highly specific, highly antigenic polypeptides (for example, SEQ ID NOS: 1-911) and binding partners with identified capture agents such as SEQ ID NOS: 912-945.

2. Complexes with Polypeptide Effectors

Many biological effects are mediated by polypeptides and thus this class of biomolecules offers a large variety of candidates for effector components. Additionally, because polypeptides can be easily manipulated and produced through recombinant means, they offer flexibility in the design of effectors that can mediate a biological effect and in conjugation to a capture agent.

In one embodiment, therapeutic complexes are constructed with a polypeptide effector. Exemplary polypeptide effectors include but are not limited to, receptors, antibodies, antibody fragments, enzymes, viral proteins and peptides. Exemplary biological effects conferred by a polypeptide effector include, but are not limited to, immunomodulation, immunostimulation, immunosuppressive mechanisms, neutralization, toxicity, enzymatic modification, inhibition of signal transduction and cellular responses, removal, destruction and degradation. In one embodiment, the effector is a molecule that confers an immunomodulatory effect.

An effector can be a single polypeptide, a multidomain polypeptide, fusion protein or multichain polypeptide. In one aspect of the embodiment, a capture agent conjugated to a polypeptide effector also is a polypeptide. Polypeptide capture agents and effectors can be linked by covalent or non-covalent interactions. A polypeptide effector also can contain a capture agent function within the same polypeptide. For example, a polypeptide can be composed of a capture agent domain and/or polypeptide chain and a domain or polypeptide chain that confers a biological effect. The domains and/or polypeptide chains can be joined covalently, such as by protein fusion, or by chemical linkage, such as by cross-linking. Alternatively, domains and or polypeptide chains can be associated by non-covalent interactions.

A binding partner recognized by a capture agent joined to a polypeptide effector can be any molecule to which the capture agent specifically binds. Molecules useful as binding partners include, but are not limited to, an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; or any combination, portion, or derivative thereof. In one embodiment, a binding partner is a polypeptide binding partner that specifically binds to a capture agent joined to an effector polypeptide.

Exemplary polypeptides for use as binding partners can be, for example, short polypeptide molecules, such as molecules with at least 4, 5, 6, 8, 10, 15, 20 or more amino acid residues, or can be a full length protein or fragment thereof capable of binding to a capture agent. Generally, short polypeptides for use as binding partners are between 4-100 amino acids, 4-50 amino acids, 4-20 amino acids and 4-12 amino acids. In one embodiment, a binding partner is a polypeptide binding partner that includes the sequence of amino acids to which a capture agent, such as an antibody or variable domain of an antibody, specifically binds. In another embodiment, is an HAHS polypeptide (described further herein). Antigens for antibodies can serve as binding partners.

A targeting domain is selected for a target of interest. Targeting domains can be constructed from any molecule that binds to a chosen target and can be conjugated to a binding partner. Such targeting domains can include, but are not limited to: an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; and other such biological materials. Examples of molecules useful as targeting domains include, but are not limited to: antibodies and binding fragments thereof, cell membrane receptors, surface receptors and internalizing receptors, monoclonal antibodies and antisera reactive or isolated components thereof with specific antigenic determinants (such as on viruses, cells, or other materials), drugs, polynucleotides, nucleic acids, peptides, cofactors, lectins, sugars, polysaccharides, and organic compounds.

3. Complexes with Immunomodulatory Effectors

Therapeutic complexes can be designed that confer a biological effect by targeting the immune system or immune cells of a subject. Such effects can include but are not limited to, neutralization, immunosuppression, clearance, modulation of cytokine expression or secretion, modulation of T cell activation, modulation of immune cell proliferation, complement activation, antibody-dependent cellular cytotoxicity (ADCC), and opsonization. Direct cytotoxicity such as induced by toxins and radiolabels generally are not considered an immunomodulatory effect.

In one embodiment, a therapeutic complex confers an immunomodulatory effect. Effectors, which confer the immune modulation can include, but are not limited to, small molecules and polypeptides. An effector, conferring an immunomodulatory effect is conjugated with a capture agent. The capture agent can be any molecule capable of specifically binding a binding partner. In one example, the capture agent is a polypeptide, such as an antibody and the binding partner is an epitope for the antibody, such as a small polypeptide (e.g. between 5 and 20 amino acids in length).

In one embodiment, the ability to specifically bind to a binding partner is provided by a polypeptide molecule that also confers an immunomodulatory effect (i.e. a capture agent and effector are contained within one molecule). In one aspect of the embodiment, a polypeptide containing an effector and capture agent is an antibody, antibody fragment, cytokine, hormone, or enzyme. In another embodiment, capture agent and effector functions are provided by two or more molecules associated to form a moiety with the ability to specifically bind to a binding partner and confer an immunomodulatory effect. In one aspect of the embodiment, a capture agent and effector are joined by cross-linking.

A targeting domain is selected for a target of interest. Targeting domains can be constructed from any molecule that binds to a chosen target and can be conjugated to a binding partner. Such targeting domains can include, but are not limited to: an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; and other such biological materials. Examples of molecules useful as targeting domains include, but are not limited to: antibodies and binding fragments thereof, cell membrane receptors, surface receptors and internalizing receptors, monoclonal antibodies and antisera reactive or isolated components thereof with specific antigenic determinants (such as on viruses, cells, or other materials), drugs, polynucleotides, nucleic acids, peptides, cofactors, lectins, sugars, polysaccharides, and organic compounds.

4.Complexes with a Plurality of Targeting Domains and/or Effectors

The modular nature of therapeutic complexes permits flexible design and allows the inclusion of a plurality of domains such as a plurality of targeting domains, capture agents, binding partners and effectors. For example, target molecules, such as cell surface receptors and antigens, can be polymorphic among a population of subjects, or within a population of target cells in an individual subject. For example, polymorphic differences in a target molecule result in changes in targeted loci, such as epitopes, thereby accounting, for example for differences in efficacy of a drug or other therapeutic among treated subjects. As a result, targeting domains that specifically bind to a target molecule present in some subjects, or in populations of target cells in a subject, may not bind with the same specificity or affinity to the same locus, if it exists in other subjects or to all cells in a subject. Therapeutic complexes containing a plurality of targeting domains can be designed to bind to a plurality of different alleles or loci or epitopes on targeted molecules.

Hence, provided are therapeutic complexes that contain a plurality of capture agents or capture agents to which a plurality binding partners binds. Each binding partner is conjugated to a targeting domain, thereby producing a therapeutic complex that contains a plurality of targeting domains through capture agent-binding partner interactions. The plurality of targeting domains can be the same or different targeting domains. Different targeting domains can bind to the same or a different targeted molecule or locus.

Targeting domains can be selected to bind to any target, including subject-specific and non-subject-specific targets. In one embodiment, a plurality of targeting domains target subject-specific targets. In another embodiment, at least one targeting domain targets a subject-specific target and at least one targeting domain targets a non-subject-specific target. In another embodiment, a plurality of targeting domains bind to targets on the same cell type or tissue or target molecule.

Therapeutic complexes provided herein can contain a plurality of capture agents. One or more of the capture agents can be an antibody or antibody fragment. For example, one or more of the capture agents is a variable domain of an antibody or contains a portion of a variable domain sufficient to specifically bind to an epitope. In one example, an antibody contains two variable domains that are each capture agents and a therapeutic complex is assembled using the specific binding of the variable domain capture agents to binding partner-targeting domain conjugates.

Targeting domains are conjugated to binding partners that specifically bind to capture agents. The interaction of the binding partners and capture agents associates the targeting domains with an effector. In one embodiment, a plurality of capture agents in a therapeutic complex bind to the same binding partner. The common binding partner can be separately conjugated to each of the targeting domains such that each common binding partner molecule is associated with only one targeting domain. The different binding partner-targeting domains can be mixed in varying ratios to create therapeutic complexes with specificity for multiple targets. For example, the targeting domains can bind to a common epitope, or different epitopes, on the same target molecule. Ratios of mixing include, but are not limited to, 1:1, 1:2, 1:3, 1:4, 1:5, 1:6,1:10, 1:20 and 1:50.

5. Retargeted Therapeutic Complexes

The methods herein can be used to retarget a molecule (or complex of molecules) by assembling a molecule into a therapeutic complex. Such methods can be used to alter, extend or enhance target specificity of a molecule that binds to a first target T1 by providing additional or alternate targeting domains. Such domains can increase the specificity or avidity of binding to a target T1 and/or provide specific binding to one or more additional targets.

Association of new or additional targeting domains is effected through assembly of therapeutic complexes, also referred to herein as retargeting, through the interaction of capture agents and binding partners. For example, a molecule or complex that exhibits a biological effect and binds to a first target T1 can be retargeted to a new or additional target T2 through the association of a targeting domain, such as any of the targeting domains described herein. To effect association of a targeting domain and a molecule or complex to be retargeted, as described herein, one or more capture agents can be conjugated to the molecule or complex and a binding partner conjugated to the targeting domain. For example, a molecule to be retargeted, M1, contains an effector domain and binds to an original target T1. A capture agent is conjugated to M1. A new targeting domain that specifically binds target T2 is conjugated to a binding partner. Assembly of the therapeutic complex associates the new targeting domain with M1, through the interaction of the capture agent and binding partner, retargeting it to target T2.

In another embodiment of the methods, the ability of a molecule to bind target T1 can be harnessed to create a capture agent-binding partner interaction for use in assembling a therapeutic complex. For example, a molecule to be retargeted, M1, binds target T1 and confers a biological effect. A therapeutic complex can be constructed using M1 as an effector-capture agent and T1 or a portion thereof sufficient to specifically bind M1 as a binding partner. T1 or a portion thereof is conjugated to a targeting domain that specifically binds target T2. The interaction of M1 and T1 associates the targeting domain with M1 and thus retargets the molecule to target T2.

In retargeting methods herein, a retargeted therapeutic complex that specifically target a new or additional target T2 can retain the ability to specifically bind target T1. Alternatively, a therapeutic complex can target T2 and the complex does not retain the ability to bind target T1. Retargeting methods also include assembling therapeutic complexes that retain the ability to bind targets T1 and T2 where T1 and T2 are binding sites on a same cell, tissue or target molecule. For example, T1 and T2 can each be cell surface molecules on a B cell. In another example, T1 and T2 are different binding sites on a molecule, such as different epitopes within a polypeptide such as an idiotype receptor or autoantibody. Although a therapeutic complex can retain the ability to bind both T1 and T2 targets, it is not necessary that the therapeutic complex bind both targets simultaneously. In some cases, only T1 or only T2 may be present, for example if T1 and/or T2 is a subject-specific target, only T1 or T2 may be found in particular subjects.

Molecules for retargeting include molecules that confer a biological effect, e.g. effectors and effector domains as described herein. Molecules for retargeting include, but are not limited to, antibodies, antibody fragments, antibody conjugates and immunotoxins. In one exemplary embodiment, an antibody or a portion of an antibody, such as one or more variable domains conjugated to an effector domain is retargeted by assembly into a therapeutic complex. One or more binding partners are designed or identified that bind to one or more of the variable domains; the binding partners are conjugated to one or more targeting domains. Assembly of the complexes associates the targeting domain(s) with the antibody or portion thereof, retargeting it to new and/or additional targets.

### D. METHODS OF MAKING THERAPEUTIC COMPLEXES

Therapeutic complexes are assembled from targeting domain, binding partner and effector components. An advantage of this modular assembly is that different methods can be used to identify and optimize each component and then assemble the complex. Further, once optimized, the components can be mixed and matched to generate additional therapeutic molecules. For example, once an effector has been identified, it can be used with any targeting domain where the biological effect of the effector is desired. Identified targeting domains also can be matched with new effectors to enable new mechanisms of therapy to be assessed quickly and in parallel on the same target. Similarly, once a binding partner and a capture agent are identified or generated that bind to each other, the pair can be used with any targeting domain or effector of choice.

Any methods known in the art can be used to isolate therapeutic complex components, including but not limited to, use of known molecules from the literature or research community, use of commercially available molecules, de novo design and synthesis, cellular screens, in vitro screens, in vivo screens, array technology, phage display and panning, 2-hybrid methods, immunizations, mutagenesis and chemical synthesis. One such method for identifying components of therapeutic complexes is screening with capture systems and addressable collections and arrays, such as those disclosed in U.S. application Ser. Nos. 10/351,891, 10/699,114, 10/699,113 and 10/699,088 and International PCT Publication Nos. WO 2004/042019, WO 2004/071641, WO 2004/039962. The method chosen will depend on the component to be identified.

1. Identifying and Isolating Targeting Domains

Targeting domains are identified by their ability to recognize and bind to a target of interest. Thus, one of the initial steps in identifying a targeting domain is the selection of a target. A target can be an isolated molecule such as an antigen, a polypeptide, a lipid, carbohydrate, a small molecule. A target also can be a cell type, membrane, extract, virus particle or other biological material. Targeting domains that bind to a selected target can be already known, for example many targets and molecules that interact with them are known in the art. Alternatively, screens can be performed to identify molecules that interact with a target. For example, library screening methods can be used to isolate targeting domains. Libraries can be generated from collections of molecules, such as collections of proteins, small molecules and nucleic acids encoding proteins. Libraries can be screened for targeting domains by any means known in the art to identify molecules that interact with a target, including but not limited to, cell-based screens, arrays, phage display and 2-hybrid assays.

a. Phage Display

In one example, a phage display library is used to display potential targeting domains. The library is contacted with the target, such as cells or molecules. Phage that display a polypeptide that interacts with the target are identified and isolated. The bacteriophage that display peptides that interact with the target cells and/or molecules can be isolated through washing and then enriched through multiple panning steps, resulting in a high population of phage displaying a peptide that can be used as a targeting domain. The isolated phage then contain nucleic acid encoding targeting domains that can be recloned into suitable expression vectors for further analysis and also for therapeutic complex construction. Phage libraries can be constructed to express random peptide libraries or collections of polypeptides such as collections of known molecules, collections of related molecules (such as families of receptors), and collections of antibodies, such as single chain antibodies and CDRs, which can be screened for targeting domains. In one embodiment, a phage display library is constructed to display a library of single chain antibodies. For example, hybridoma cells can be used as starting material for the generation of such a library. The hybridoma cells can be generated from non-immunized mice or from mice immunized with the target. PCR can then be used to amplify a library of nucleic acid sequences encoding the heavy and light chains of the antibodies expressed in the hybridoma cells. The PCR is designed such that the amplification method generates a fusion of the heavy and light chain variable domains in a single chain molecule for expression on the phage surface. In other methods, one or more single chain antibodies can be mutated by directed and/or random mutagenesis to create a library of antibody molecules with different binding specificities that can be used to screen against the target for targeting domains.

b. Two-Hybrid Methods

Two-hybrid methods also can be used to isolate targeting domains. In two hybrid assays, the target polypeptide is expressed fused to a molecule such as a DNA binding domain. A library of potential targeting domains is then constructed fused to a transcriptional activation domain. The fusion library is expressed in host cells with the target-DNA binding fusion and a reporter construct. When a targeting domain is expressed that interacts with the target, a functional transcription factor is formed and the reporter is activated. When there is no interaction, the reporter construct is not activated and no reporter molecule is produced. The reporter is a visible or otherwise detectable molecule and cells that express the reporter can be identified. Targeting domains can then be isolated from the identified cells and used for the construction of therapeutic complexes. Two hybrid systems have also been used that are more amenable to expression of molecules in a membrane or cell surface environment and can be useful for isolating targeting domains for membrane bound and secreted targets.

c. Small Molecule Screening

Small molecule targeting domains can be identified through screening of libraries. Such libraries can be constructed from natural and/or synthetic molecules. For example, natural products can be isolated and screened. Synthetic chemical libraries, such as combi-chem libraries, can be constructed or obtained (for example obtained from commercial sources) and screened for targeting domains. Small molecule libraries are often compatible with high-throughput based screening methods known in the art. Small molecule libraries can be screened in cell-based, subject-based and in vitro assays.

d. Use of Known Molecules to Construct Targeting Domains

In some cases, potential targeting domains can be designed and constructed from molecules known to interact with the target. For example, a cell surface receptor can have a known ligand, antagonist or agonist as an interacting molecule. Similarly, a monoclonal antibody that is known to interact with a cell surface antigen can be identified . These molecules can then be used as starting material for generating targeting domains. For example, small molecules can be designed from a known ligand that can be used as targeting domains and can be conjugated to a binding partner. Monoclonal antibodies can be cloned by nature of the conserved domains in antibodies, for example by using collections of PCR primers, and a single chain antibody or other binding domain can be constructed for use as a targeting domain.

e. Assays for Characterizing Targeting Domains

Targeting domains can be characterized by any methods known in the art. Of particular usefulness are assays that characterize the binding of the targeting domain to the target, including the assessment of the affinity and specificity of the targeting domain for the target. Examples of such assays include, but are not limited to, affinity chromatography, western blots, immunoprecipitations, ELISAs, BIAcore® interaction assays, circular dichroism, and cell reporter assays.

Assays can be performed with isolated target molecules such as purified proteins and small molecules. Assays also can be performed on whole cells to assess the binding to the target as it is found in a cellular environment. Such assays can include the use of expression or production of targets in heterologous cells and the isolation of cells that express and/or produce the target molecules. Whole tissues, organs and animals also can be used to assess the targeting domain interaction with the target in a more complex environment. Such assays can include labeling the targeting molecule with a visible or otherwise detectable molecule to assess interaction with the target. Examples of labels include radiolabeling, reporter fusions, enzyme fusions, fluorescent molecules and bioluminescent molecules. Immunohistochemistry also can be used for detection, for example, by using an antibody that recognizes some portion of the targeting domain or molecule fused to the targeting domain.

In one example of such assays, a candidate targeting domain can be tested in vitro for interaction with a selected target. As shown in Example 2, an anti-idiotype monoclonal antibody (S1C5 anti-IgM antibody) is tested for binding to a B cell antibody target, 38C13 antibody. Binding assays can include interaction of the targeting domain with a purified target and a target in cellular extract. Cell-based assays can be also used to assess interactions of a targeting domain and cells expressing the target. For example, as shown in Example 2, a candidate targeting domain S1C5 anti-IgM antibody can be assessed for its binding to 38C13 B cells.

2. Identification and Generation of Effectors

An effector confers a biological effect on a therapeutic complex. Effectors also are associated with capture agents such that when a capture agent binds to a binding partner-targeting domain moiety, the effector is then associated with the targeting domain in a therapeutic complex. In some embodiments, capture agents and effectors are contained in a single molecule. For example, molecules can be selected that possess both functions. Alternatively, candidate effectors can be engineered to contain a capture agent function. For example, a capture agent can be joined covalently or non -covalently to an effector. Alternatively, effectors can be screened and/or selected and/or engineered to identify those that can bind to a binding partner and fulfill a capture agent function. Effectors can be identified from molecules known to have a biological effect such as immunomodulatory molecules and/or screening for biological effects using in vitro and in vivo based screening methods.

a. Constructing Effectors from Immunomodulators

Molecules that can act as immunomodulators are candidate molecules from which effectors can be constructed. Such molecules can be identified from molecules known to have an immunomodulatory effect, by screening molecules for their immunomodulatory effect, by mutating and optimizing molecules with potential to have an immunomodulatory effect or by any other means known in the art.

i. Known Immune Modulators

Molecules of several classes are known to have immunomodulatory activities. Such molecules include antibodies and domains of antibodies such as the Fc domain and other fragments of antibodies, and proteins such as LRP that bind to the Fc receptor. Cytokines also are examples of immunomodulatory molecules. Exemplary cytokines include, but are not limited to, interleukins (e.g., IL-2, IL-3, IL-4, IL-5, IL-6, IL-7, IL-9, IL-10, IL-11, IL-12, IL-13, IL-15, IL-16, IL-17, IL-18, IL-11α, IL-1β, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), and MIF.

Cytokines are known to be immunostimulatory to particular cell types. For example, interleukins such as IL-4 and IL-5 stimulate B cells, IL-2 stimulates T cells, IL-2, IL-3, IL-4, IL-5 and IL-10 have stimulatory roles with classes of hematopoietic cells. Interferon-γ, lymphotoxin and tumor necrosis factor stimulate macrophages. Granulocyte macrophage colony stimulating factor and transforming growth factor-β stimulate hematopoietic cells. Cytokines also have inhibitory effects on particular cell types. For example, lymphotoxin is cytotoxic to tumor cells, IL-4 inhibits macrophage activation, and IL-10 can inhibit T_{H}1 T-cells. Effectors can be constructed from immunomodulatory molecules, in particular proteins such as antibodies, cytokines and fragments thereof. Nucleic acids encoding cytokines, antibodies, in particular Fc regions of IgG, human IgG1, mouse IgG2a, and cytokines including but not limited to IL-2,3,4,5,10 and 12, interferons, TGF-β, TNF, lymphotoxin, and GM-CSF are known in the art. Such nucleic acids can be used, such as described herein, to construct effectors conferring biological effects. For example, a nucleic acid encoding an effector can be joined with a nucleic acid encoding a capture agent to construct a nucleic acid for the expression of an effector containing an immunomodulatory molecule linked to a capture agent.

In another embodiment, immunomodulatory molecules can be generated that also bind to binding partners and thus the effector and capture agent are contained within the same molecule. For example, IgG molecules containing an Fc domain can be generated that are capable of binding partner binding using the antigen binding sites (variable domains) of the immunoglobulin. In one example, binding partners such as small polypeptide binding partners are used to immunize animals such as mice. Antibodies are generated that bind to the peptides and that are immunoglobulins of the IgG class, such as IgG2a immunoglobulins. The immunoglobulin molecules are cloned and the nucleic acid molecules can be used to construct effectors; for example, a single chain antibody can be constructed.

In another embodiment, immunomodulatory molecules can be selected that bind binding partners. For example, chosen immunomodulatory molecules are used to generate a library of molecules, for example by mutagenesis, gene shuffling, PCR or other techniques known to those of skill in the art. The libraries are then screened with sets of binding partners for immunomodulatory molecules with the ability to specifically bind to a binding partner. Such molecules that bind to a binding partner and retain the ability to confer a biological effect can then be isolated from the library. Assays such as described herein or known in the art can be used to confirm effector function in the presence of binding a binding partner. Binding partner binding also can be identified by screening an unmutagenized and/or mutagenized library of immunomodulatory molecules with a random library of polypeptides and identifying from the immunomodulatory molecule library molecules capable of binding partner binding and from the random polypeptide library polypeptides specifically bound by an immunomodulatory molecules.

ii. Immunomodulatory Screens

Immunomodulators can be identified and isolated using a variety of in vitro and cell-based screens. Such screens include but are not limited to, screens that identify cytotoxic, immunostimulatory, immunosuppressive and neutralization effects. Examples of such screens include, but are not limited to, binding assays, such as binding to receptors such as the T cell receptor complex and Fc receptors, proliferation assays, for example T cell proliferation assays for the detection of cytokines, cytotoxicity assays such as a ¹²⁵ITdR release assay and assays for detecting of intracellular esterase activity and plasma membrane integrity, apoptosis assays such as TUNEL staining, assays for activated T cells, such as assays to detect ⁵¹Cr-release from cells targeted by activated CD8 T cells, and assays that detect cytokine secretion from cells such as ELISA and ELISPOT assays. Such assays and screens can be used in cell-based and in vitro screening methods, including high-throughput screens. Assays and screens can include the use of secondary agents. Secondary agents can include molecules that have a co-stimulatory or co-inhibitory effect with an effector. Assays also can include the use of animal models, such as administration to rodents, to determine biological effect.

b. Effectors Designed from Known Molecules

Effectors can be constructed from molecules known to confer a specific biological effect. For example, receptors, ligands, agonists, antagonists, enzymes and other known molecules can be selected. Molecules can be selected, for example, on the basis of their ability to form complexes with other molecules, catalytic or enzymatic activity, the ability to specifically bind to a receptor or ligand, and activation, inhibition or modulation of target function, toxicity, inhibition of signal transduction and cellular responses, destruction and degradation activities. The choice of the effect will depend on the chosen therapeutic target and suitable biological activity applicable to such therapy.

In one exemplary embodiment, an effector is constructed from an antibody such as a known antibody. For example, the antibody can be one that is registered for therapeutic use such as monoclonal antibodies registered with the FDA (Food and Drug Administration) registration. Antibodies for use as effectors include, but are not limited to, the anti-Her-2 monoclonal antibody trastuzumab (Herceptin®), anti-CD20 monoclonal antibodies tositumomab (Bexxar®), rituximab (Rituxan®) and Ibritumomab (Zevalin®), the anti-CD52 monoclonal antibody Alemtuzumab (Campath®), the anti-TNFα antibodies infliximab (Remicade®) and CDP-571 (Humicade®), the monoclonal antibody edrecolomab (Panorex®), the anti-CD3 antibody muromab-CD3 (Orthoclone®), the anti-IL-2R antibody daclizumab (Zenapax®), the omalizumab antibody against IgE (Xolair®), the monoclonal antibody bevacizumab (Avastin®), and the monoclonal antibody against EGFR cetuximab (Erbitux®).

c. Assays for Characterizing Effectors

Effectors can be assayed for biological effect using any assays known in the art for assessing such effects. Biological effect can be assayed in vitro or in vivo including cell-based and subject-based assays. Such assays include those that assess immunomodulatory activities, ability to form complexes with other molecules, catalytic or enzymatic activity, the ability to specifically bind to a receptor or ligand, and activation, inhibition or modulation of target function, toxicity, stimulation or inhibition of signal transduction and cellular responses, removal, destruction and degradation.

Assays for biological effect also can include assessment of therapeutic effects and pharmaceutical activity in subject-based assays. For example, as described in Example 4, effector molecules can be delivered to an animal, such as a mouse injected with tumor cells, and assayed for survival as well as additional biological effects.

Biological effect also can be assessed by other methods, for example, the use of capture systems and cell capture assays, such as assays to assess apoptosis and other biological effects, as disclosed in U.S. patent application Ser. No. 10/699,114 and International PCT Publication No. WO 2004/042019.

3. Use of Arrays and Other Addressable Systems to Identify Targeting and Effector Domains

Arrays and other addressable systems can be used to identify targeting domains and effectors. Such technologies allow molecules to be displayed and/or sorted based on position or other identifiers and assayed for a function, phenotype or biological effect. Any array or addressable system suitable for screening, binding between molecules or for screening biological effect can be utilized to identify targeting domains and effectors. For example, arrays and addressable collections of molecules can be screened against a selected target to identify targeting domains. Arrays and addressable collections of molecules can be screened with selected targets and biological effect can be monitored to identify effectors.

An exemplary method for identifying components of therapeutic complexes is screening with capture systems and addressable collections and arrays, such as those disclosed in U.S. application Ser. Nos. 10/351,891, 10/699,114, 10/699,113 and 10/699,088 and International PCT Publication Nos. WO 2004/042019, WO 2004/071641, WO 2004/039962. The capture systems use collections of capture agents and binding partners such as described herein where each binding partner specifically binds to a capture agent. The collection of capture agents are addressed, such as by positional information or other identifiers. The addressable capture agent collections contain a collection of different capture agents, each of which bind to a unique binding partner. Each locus or address contains a single type of capture agent that binds to a single specific binding partner. Capture agents can be positionally addressed. Alternatively, each can be addressed by associating them with unique identifiers, such as by linkage to optically encoded identifiers, including colored beads or bar coded beads or supports, or linked to electronic identifiers, such as by providing microreactors with electronic tags or bar coded supports or colored identifiers or other such addressing methods that can be used in place of physically addressable arrays.

In screening for components of therapeutic complexes, such as targeting domains and effectors, the collections of capture agents and binding partners can be used to display and test candidate molecules. The same capture agents and binding partners can be used with the tested and identified components to construct therapeutic complexes. Alternatively, different capture agents and binding partners can be used for screening and for constructing therapeutic complexes.

Candidate components, for example candidate targeting domains and candidate effectors are tagged with binding partners. Tagged molecules are contacted with the collection of capture agents in an array, under conditions suitable for complexation with the capture agent via the binding partner. As a result, molecules are sorted according to the binding partner tag each possesses and displayed. The specificity of each capture agent for a particular binding partner is known or can be readily ascertained, such as by arraying the capture agent so that all of the capture agents at a locus have the same specificity. Therefore, candidates binding to each locus based on their binding partner can be identified.

a. Targeting Domain Identification

Capture systems can be used to identify targeting domains. Candidate targeting domains can be tagged with binding partners and displayed to screen against targets. Alternatively, candidate target molecules can be tagged and displayed and targeting domains screened. For exemplification purposes, display of targeting domains is described. Candidate targeting domains are conjugated to binding partners specific for a capture agent that is addressable such as within an array. Generally, although not necessarily, each candidate is conjugated to a different binding partner. Molecules are conjugated such that the aspect that makes them of interest, such as their 3-D structure or binding activity, is not altered. Optionally, the molecule of interest can be labeled with a detectable label, such as a luminescent label, to permit or provide for detection of the displayed molecule particle within an addressable array.

Conjugated molecules are then contacted with the addressable arrayed capture agents. Particular conditions for capture depend upon the type of capture agents, binding partners and molecules conjugated thereto. Such conditions are standard, such as those for forming complexes between antibodies and antigens, and/or can be empirically determined. Once the conjugated molecule is sorted onto the array, one or more targets are added and interactions with the displayed molecules are assessed. Displayed molecules are identified that bind to a target (positive loci). Such assessment can include one or more washing steps to remove non-specific, or low-affinity interactions, before target-binding displayed molecules are identified. Identification can be assessed by direct or indirect means, by any method known to those skilled in the art, including, but not limited to, detection of a secondary antibody; a conformational change; a binding interaction; complexation; hybridization; transfection; hydrophobic interaction; signal transduction; membrane translocation; electron transfer; conversion of a reactant to a product via a catalytic mechanism; chaperoning of compounds inter- and intracellularly; fusion of liposomes to membranes; infection of a foreign pathogen into a host cell or organism, such as a virus (HIV, influenza virus, polio virus, adenovirus, etc.) or bacteria (Escherichia coli, Pseudomonas aeruginosa, Salmonella enteritidis, etc.); initiation of a regulatory cascade, detoxification of cells and organisms; and cell replication and division.

The identity of displayed molecules that bind to a target is determined from their position on the addressable array of capture agents, (or by an identifier if other addressable systems are used) thereby identifying the binding partner and thus displayed molecule at the positive loci. Identified displayed molecules from each positive of the loci are then available to be used as targeting domains.

b. Effector Identification

Capture systems and other addressable collections also can be used to screen for effector molecules. For example, a capture system is generated containing a collection of binding sites with capture agents preselected to bind to a binding partner, and a plurality of binding partner tagged candidate effectors. The binding sites of the capture system also can contain one or more anchor molecules that bind to a biological particle or molecule and anchor it to each site. The capture system is contacted with a sample of biological particles or molecules under conditions whereby the biological particles or molecules bind to one or more sites of the addressable collection. An interaction between the candidate effectors and the biological particles or molecules is detected and thus, effectors are identified as tagged reagent(s) at site(s) that interact with a biological particle or molecule.

Biological particles and molecules used in screens for effectors can be potential targets or they can be cells involved in mediating an effect, such as an immune cell. Interactions between candidate effectors and the biological particles are monitored to identify effectors that mediate an effect. Examples of interactions between candidate effectors and biological particles and molecules include, but are not limited to, specific binding, modulation of signal transduction, activation of apoptosis and cell death pathways, enzymatic modification, degradation, receptor activation, endocytosis or inhibition, activation or inhibition of cell migration, modulation of cell proliferation, modulation of response to secondary agents, modulation of transcription and translation, modulation of replication, stimulation of phagocytosis and modulation of secretion.

Interactions between candidate effectors and biological particles and molecules can be detected by direct or indirect detection methods. For example, staining can be used to monitor for the presence or absence of molecules. Staining can be specific staining for a molecule or class of molecules such as staining for the presence of an antibody, a protein or a carbohydrate. For example, antibodies can be used to detect the presence of a secreted antigen, the production of a cell surface antibody, and endocytosis and recycling of cell surface receptors. Reporter systems can be used to monitor signal transduction, and functions such as transcription and translation. For example, cells can be used that contain a reporter that is activated by a specific pathway, such as stimulation by cytokines. In the presence of candidate effectors with cytokine activity, a reporter is activated and can be detected by direct or indirect means. Exemplary reporter include fluorescent and bioluminescent molecules, enzymes, such as enzymes that act on chromogenic and fluorogenic substrates that are detected by visible or spectrophotometric detection. Radiolabeled molecules can be used to detect cell proliferation, such as ³H-thymidine assays, and cell intactness, such as ⁵¹Cr-release assays.

Secondary agents can be added to effector assays. Such secondary agents can be molecules with co-stimulatory or co-inhibitory effects or for example, agents that modulate the state of the biological particle or its response to candidate effectors. Secondary agents can be added prior to detecting the interaction between the candidate effectors and the biological particles or molecules. Interaction of candidate effectors and biological particles or molecules also can be compared in the presence and absence of a secondary agent.

c. Interchange of Components

Binding partners and capture agents used in capture systems can be used as components for constructing therapeutic complexes. For example, binding partners for the capture systems also can be used as binding partners in the construction of therapeutic complexes. Once targeting domains are identified from the array that bind to the target, a binding partner-targeting domain conjugate can then be used as the binding domain-binding partner conjugate in the therapeutic complex. Capture agents used in capture systems also can be used as capture agents associated with effectors in therapeutic molecules. In some examples, capture agents used in capture systems also can contain an effector function. For example, a capture agent array can be an array of antibodies with Fc domains, for example an array of IgG antibodies, which interact with binding partners. Such capture agents can be used as components in a therapeutic complex with capture agent and effector function. For example, an Fc domain can be used to provide an immunomodulatory effect to a therapeutic complex and a variable domain can bind to a preselected binding partner.

4. Design, Generation and Selection of Binding Partners and Capture Agents

Capture agents and binding partners are pairs of molecules that specifically bind to each other. Each can be used in therapeutic molecules as described herein to associate targeting domains and effectors. Further, binding partners and capture agents also can be used in capture systems useful for screening therapeutic components of therapeutic molecules. Any method known in the art for finding pairs of molecules that bind can be used to generate binding partners and capture agents. In most cases, capture agents and binding partners are generated by methods that generate one set of molecules (binding partners or capture agents) and then use subsequent design and/or selection to generate the remaining set of the pair. Exemplary methods include phage display of a random polypeptide library of candidate capture agents or binding partners followed by biopanning with preselected binding partners or capture agents; 2-hybrid analysis of an expression library of candidate polypeptides with either preselected capture agents or binding partners; theoretical molecular modeling of the sequence and three dimensional structure of a polypeptide, for example a capture agent, to design a binding partner; de novo design of binding partners and generation and/or selection of capture agents that bind to binding partners; and use of known binding pairs as capture agent-binding partners.

a. Phage Display

One method for identifying binding partners and capture agents employs panning phage displayed polypeptide libraries, such as random polypeptide libraries, for molecules that interact with chosen candidates. For example, molecules for use as capture agents can be chosen and then phage display used to identify binding partners. Alternatively, for example, binding partners can be selected or chosen, and a phage display library is panned to select capture agents that bind to the binding partners. For exemplification, phage display of binding partners is described. Polypeptides that interact with a specific capture agent can be identified by displaying random libraries of polypeptides on the surface of a phage molecule and monitoring their interactions with a capture agent. Capture agents can be displayed for example, on a solid support or an addressable array and the bacteriophage that display polypeptides that interact with capture agents can be isolated through washing and then enriched through multiple panning steps, resulting in a high population of phage displaying a polypeptide that can be used as a binding partner. Panning of phage displayed peptide libraries also can be used to map the binding site of a capture agent, thereby identifying the exact amino acid residues required for interaction with a binding partner. Such information can be used to construct additional capture agents or transfer the binding partner interaction function to another molecule such as by joining it with an effector. Once a polypeptide that reacts with a capture agent is identified, the polypeptide for use as a binding partner can be synthesized and conjugated to a targeting domain as described below. This conjugate can then be tested to determine whether the binding partner portion when conjugated to the targeting domain retains the ability to interact with high affinity and specificity with the capture agent.

b. Two-Hybrid Analysis

Another method for identifying binding partners and capture agents employs a two-hybrid screen for molecules, such as polypeptides, that interact. One set (such as potential binding partners) is expressed in a host such as yeast, E. coli, insect and mammalian cells as a fusion protein with a DNA binding domain. Examples of DNA binding domains include but are not limited to, Ga14, GCN4, lambda repressor, Sp1, and TATA-binding protein (TBP). An expression library is constructed with candidate polypeptides (such as candidate capture agents) fused to a transcriptional activation domain. Examples of activation domains include VP16, relA and p65. The expression library is transformed into and expressed in host cells also expressing candidate binding partner-DNA binding domain fusions. The assay is designed such that if a candidate capture agent binds to a binding partner, the complex activates a reporter gene such as GFP or B-galactosidase. Positive cells are identified and the binding partner-capture agent pairs are thereby identified. Further rounds of screening or other binding assays known in the art can be used to confirm and further characterize the interaction of the binding partner-capture agent pairs.

c. Sequence Analysis and Molecular Modeling

In silico methods can used to identify candidate binding partners and capture agents. For example, if a chosen capture agent is an antibody or fragments thereof, structural information (such as by NMR and X-ray known for numerous immunoglobulins) can be manipulated In silico to identify potential molecules that can interact with the variable region of the antibody. The energy of interaction between the antibody and potential binding partner can be determined using a molecular docking program such as DOCK, which is commercially available; see, also, e.g., (online at cmpharm.ucsf.edu/kuntz/dock.html), AutoDock (online at scripps.edu/pub/olson-web/doc/autodock/), IDock (on line at archive.ncsa.uiuc.edu/Vis/Projects/Docker/) or SPIDeR (on line at simbiosys.ca/sprout/eccc/spider.html). Once identified and the binding energy is determined In silico, polypeptides that constitute the binding partners can be synthesized or purchased commercially and tested in vitro for their specificity and affinity for a chosen capture agent.

For polypeptide capture agents, sequence alignments with related molecules also can be used to identify binding partners that bind to a capture agent. For example, if a chosen capture agent is an antibody or fragment thereof, binding partners can be identified by analyzing complementarity determining regions (CDRs) in the capture agent antibody. Translation of available cDNA sequences of the variable light and variable heavy chains of a particular antibody permit the delineation of the CDRs by comparison to the database of protein sequences compiled in “Sequences of Proteins of Immunological Interest,” Fifth Edition, Volume 1, Editors: Kabat et al. (1991) (see, e.g., table on page xvi).

d. Use of Known Molecules to Design Binding Partner-Capture Agent Pairs

Binding partners and capture agents also can be generated from known binding pairs. For example, a capture agent can be chosen that is known to bind to another molecule, and that molecule or a portion thereof sufficient to bind to the capture agent can be chosen as a binding partner. Examples of known binding pairs include but are not limited to antibody-antigen, receptor ligand, heterodimerization partners such as leucine zipper proteins and basic-helix-loop-helix domain proteins and DNA binding domain-nucleic acid pairs. Exemplary binding partners also are polypeptides that are recognized by antibodies. These polypeptides can be used as binding partners to bind capture agents containing the corresponding antibody or portion thereof sufficient to bind to the binding partner. Some exemplary binding partners provided herein include E-tag polypeptide (SEQ ID NO: 912), a FLAG polypeptide (SEQ ID NO: 913), a Glu-Glu polypeptide (SEQ ID NO: 914), a HA.11 polypeptide (SEQ ID NO: 915), a HSV-tag polypeptide (SEQ ID NO: 916), a c-myc polypeptide (SEQ ID NO: 917), a T7 tag polypeptide (SEQ ID NO: 918), a VSV-G polypeptide (SEQ ID NO: 919), a V5 polypeptide (SEQ ID NO: 920), an AB2 polypeptide (SEQ ID NO: 921), an AB4 polypeptide (SEQ ID NO: 922), a B34 polypeptide (SEQ ID NO: 923), a P5D4-A polypeptide (SEQ ID NO: 924), a P5D4-B polypeptide (SEQ ID NO: 925), a 4C10 polypeptide (SEQ ID NO: 926), an AB3 polypeptide (SEQ ID NO: 927), an AB6 polypeptide (SEQ ID NO: 928), a KT3-A polypeptide (SEQ ID NO: 929), a KT3-B polypeptide (SEQ ID NO: 930), a KT3-C polypeptide (SEQ ID NO: 931), a 7.23 polypeptide (SEQ ID NO: 932), a HOPC1 polypeptide (SEQ ID NO: 933), a S1 polypeptide (SEQ ID NO: 934), an E2 polypeptide (SEQ ID NO: 935), a His tag polypeptide (SEQ ID NO: 936), an AU1 polypeptide (SEQ ID NO: 937), an AU5 polypeptide (SEQ ID NO: 938), an IRS polypeptide (SEQ ID NO: 939), a KT3 polypeptide (SEQ ID NO: 945), a S-tag polypeptide (SEQ ID NO: 944), NusA (SEQ ID NO: 940), Maltose binding protein (SEQ ID NO: 941), TATA-box binding protein (SEQ ID NO: 942) and thioredoxin (SEQ ID NO: 943).

Another example of known molecules that can be used to design binding partner-capture agent pairs are antibodies. One or more variable domains or portions thereof that specifically bind to an epitope can be used as a binding partner or capture agent, and the epitope or a molecule containing the epitope can be used as the corresponding capture agent or binding partner, respectively. For example, an antibody or portion thereof can be used as a capture agent; the antibody can be one that is registered for therapeutic use such as monoclonal antibodies registered with the FDA (Food and Drug Administration). Epitopes specifically bound by the antibody can be used as binding partners. Such binding partners can then be conjugated to targeting domains, such as described herein. Examples of antibodies for use as capture agents (or binding partners) include, but are not limited to the anti-Her-2 monoclonal antibody trastuzumab (Herceptin®), anti-CD20 monoclonal antibodies rituximab, (Rituxan®), tositumomab (Bexxar®) and Ibritumomab (Zevalin®), the anti-CD52 monoclonal antibody Alemtuzumab (Campath®), the anti-TNFα antibodies infliximab (Remicade®) and CDP-571 (Humicade®), the monoclonal antibody edrecolomab (Panorex®), the anti-CD3 antibody muromab-CD3 (Orthoclone®), the anti-IL-2R antibody daclizumab (Zenapax®), the omalizumab antibody against IgE (Xolair®), the monoclonal antibody bevacizumab (Avastin®) and the monoclonal antibody against EGFR cetuximab (Erbitux®).

e. De novo Generation

Binding partners and capture agents can be generated by de novo design. For example, as described further herein and in International PCT Publication No. WO 2004/039962 and U.S. application Ser. No. 10/699,088, published as US 2004-0209282-A1, and U.S. application Ser. No. 10/806,924, entitled “METHODS FOR DESIGNING LINEAR EPITOPES AND ALGORITHM THEREFOR AND POLYPEPTIDE EPITOPES” filed Mar. 22, 2004, polypeptide binding partners can be designed and constructed that are highly antigenic and that can induce, upon administration to a host, antibodies that are specific for the polypeptide binding partners that can be used as capture agents. The highly antigenic highly specific (HAHS) polypeptides also can be used for screening antibody and other libraries, including single chain antibody libraries to select capture agents.

HAHS polypeptides can be used as binding partners in therapeutic molecules described herein. Capture agents for use in therapeutic molecules can be generated from antibodies or selected single chains or other binding agents identified that specifically bind HAHS polypeptides.

In one example, methods for designing and generating HAHS polypeptides use statistical probabilities that a particular amino acid appears in an antigenic polypeptide. These statistical probabilities can be calculated or generated empirically. Statistical probabilities for naturally occurring amino acids are exemplified herein. The same or similar methods can be applied to any sets of amino acids including non-naturally occurring amino acids and analogs thereof. For example, sequences of antigenic polypeptides can be obtained by empirical methods, such as by injecting mice with polypeptides representing all the possibilities of a set length of polypeptides. The polypeptides are injected into mice and antisera is collected. The antisera then is tested on collections of polypeptides and the antigenic polypeptides are identified based on their reactivity with the antisera. Non-antigenic polypeptides are identified by their lack of reactivity with the antisera. The frequency of an amino acid appearing in a polypeptide that is antigenic is used to determine that amino acids are more likely to be found in an antigenic polypeptide.

Statistical prediction can be made based on the frequency of an amino acid appearing in a polypeptide that is antigenic. The likelihood that an amino acid appears in a polypeptide that is antigenic can be determined based on a representative set of data, for example, based on immunizing animals with a representative subset of all the possibilities of that polypeptide length. Based on the subset of polypeptides injected that are antigenic and non-antigenic, amino acids are identified that either are more likely to be present in antigenic polypeptides or are more likely to be present on non-antigenic polypeptides. The likelihood of an amino acid's presence in an antigenic polypeptide gives an observed antigenic ranking. Using polypeptides of the 20 naturally occurring amino acids, a ranking of antigenicity for each amino acid can be obtained. Similarly, an antigenic ranking of amino acids also can be obtained by mapping epitopes in known proteins. Antibodies to known proteins are used to determine the sequence of amino acids to which they bind, for example by deletion or replacement mutagenesis or by synthesizing subsets of amino acid sequences found within the protein sequence. The antibodies are tested for reactivity with the mutants or with subsets of peptide sequences from the protein. The shortest sequence of amino acids from the protein that retains binding to the antibody defines a linear epitope. Epitope mapping can be performed with a representative number of proteins and antibodies and the statistical occurrence of each of the 20 amino acids found in the epitopes is determined to generate the antigenic ranking of the amino acids (see, e.g., Geysen et al., (1988). J. Molecular Recognition 1:32 41; Getzoff et al., (1988) “The Chemistry and Mechanism of Antibody Binding to Protein Antigens” in Advances in Immunology. Vol 43:1 98, Academic Press). For example, a propensity factor can be calculated by comparing the ratio of the observed frequency of a chosen amino acid appearing in an antigenic polypeptide to the frequency that would be expected if it appeared by chance alone (Geysen et al., (1988). J. Molecular Recognition 1:32 41). Epitope mapping and antigenic ranking such as with known proteins or by injecting collections of random polypeptides can be done in any species of interest that raises an immune response, for example mice, rabbit, rat, human, monkey, dog, chicken, and goat. For example, using data obtained from epitope mapping (Geysen et al., (1988). J. Molecular Recognition 1:32 41), the amino acids were assigned the following antigenic rankings, with 1 being the highest and 20 the lowest probability (Table 2).

TABLE 2
Antigenicity Ranking

| Ranking | amino acid |
|---|---|
| 1 | E |
| 2 | P |
| 3 | Q |
| 4 | N |
| 5 | F |
| 6 | H |
| 7 | T |
| 8 | K |
| 9 | L |
| 10 | D |
| 11 | V |
| 12 | I |
| 13 | G |
| 14 | Y |
| 15 | S |
| 16 | C |
| 17 | A |
| 18 | M |
| 19 | R |
| 20 | W |

Antigenic ranking can be obtained using data from a single species or a plurality of species. Antigenic ranking also can compare antigenicity between hosts such that HAHS polypeptides can be generated that are antigenic in one species but less antigenic or non-antigenic in another species. For example, antigenicity rankings can reflect high antigenicity in mice but lower antigenicity in humans.

Epitope mapping and antigenic ranking also can be performed using recombinant means, by screening libraries of antibodies or antibody fragments with polypeptides containing sequences of epitopes, such as collections of sequences of critical amino acids. The polypeptides that are bound by the antibodies can be identified and the frequency of the amino acids appearing in polypeptides bound by the antibodies can be determined. Experimental conditions such as washing conditions in a phage library panning assay can be used to control the affinity of the interaction between the antibodies and the peptides.

For a given length of polypeptides, amino acids are selected from the antigenic ranking list. Polypeptides can be any length sufficient for an antibody epitope, generally less than 20 amino acids. For example, the polypeptide's length is between 2 and 20 amino acids, such as 2, 3, 4, 5, 6, 7, 8, 9, 10, 11, 12, 13, 14, 15, 16, 17, 18, 19 and 20 amino acids in length. In one exemplary embodiment, 4-mers are selected using the antigenic ranking list of amino acids.

A threshold ranking of antigenicity can be chosen to limit the possible number of polypeptides in the subset (subset A) and to bias the subset to more antigenic sequences. For example, if the polypeptide length is 20 amino acids, each of the 20 positions can be selected from the top 19 antigenic ranking amino acids, limiting the subset from the total possibilities of all 20 amino acids at each position. The threshold can be set according to the number of polypeptides desired in the subset and the level of dissimilarity chosen for the subset. In one embodiment, the amino acids are chosen from the top n-1 antigenic ranking amino acids, where n is the total number of ranked amino acids. In one aspect of the embodiment, the top 19, 18, 17, 16, 15, 14, 13, 12, 11, 10, 9, 8, 7, 6, or 5 antigenic ranking amino acids are used to design and construct the polypeptide sequences. In one exemplary embodiment, the top 10 antigenic ranking amino acids are used to design and construct polypeptide sequences. In another exemplary embodiment, the amino acids E, P, Q, N, F, H, T, K, L, and D are used to design and construct polypeptide sequences.

In a given length of polypeptides, to further bias the specificity of the polypeptides and reduce potential cross reactivity between binding proteins and polypeptides outside the partner pairs, the amino acids within each polypeptide are different from each other such that there are no duplicates. This further reduces the number of polypeptides in the subset (subset B). For example, if the polypeptide is a 4-mer and 10 amino acids are chosen from the antigenic ranking list, the number of possibilities in 10×9×8×7, where each amino acid is unique within a 4-mer (i.e., there is no duplication or any multiples of a chosen amino acid within the polypeptide length). Thus, for a 4-mer there are 5040 possibilities in this subset B.

Subset B represents the list of antigenic polypeptide possibilities for the chosen length of polypeptide. Optionally, these polypeptides can be incorporated in larger polypeptides, such that the polypeptides derived from subset B are designated the critical residues in the polypeptide, composed of antigenic amino acids and the remaining positions in the polypeptide length are noncritical positions (subset C). The length of such polypeptides can be generally less than 50 amino acids, typically less than 20 amino acids. For example, the polypeptides length can be between 2 and 20 amino acids, such as 2, 3, 4, 5, 6, 7, 8, 9, 10, 11, 12, 13, 14, 15, 16, 17, 18, 19 and 20 amino acids in length. The number of critical residues is larger than the number of non-critical residues. Generally, for peptides of 9 or less amino acids, the number of critical residues is approximately 55%, 60%, 70%, 80%, 85%, 90% or 95% of the total number of amino acids in the polypeptide.

A non-critical position does not determine the affinity or specificity of binding to a capture agent for a HAHS polypeptide such that noncritical residues can be replaced by another amino acid without substantially affecting the affinity or specificity of binding of the HAHS polypeptide and capture agent. Generally, non-critical positions can be replaced with a larger set of amino acids. For example, when taken from the set of naturally occurring amino acids, non-critical positions can be replaced usually 10 or more amino acids or in some cases, by any other amino acid from the set of naturally occurring amino acids.

Non-critical positions also can be utilized to introduce added functionalities into the polypeptide, such as enhancing solubility and folding. In one exemplary embodiment, amino acids that increase solubility and permit flexibility and folding are used at the non-critical positions. For example, the amino acids S, G and Y are utilized at the non-critical positions.

The non-critical positions can be designated at specific sites within the polypeptide length to construct subset D. For example, it can be designated that the N and C terminal residues of the polypeptide are critical residues. In another example, it can be designated that the non-critical residues are found in pairs. In one exemplary embodiment 6-mer polypeptides are designed whereby the first and last (N and C terminal) positions are critical residues and 2 additional positions of the remaining 4 residues of the 6-mer also are critical residues chosen from a set of antigenic amino acids. The remaining 2 positions are non-critical residues and are designated to be in adjacent positions in the 6-mer.

In the above example, with 6-mers, the following possible polypeptides are generated for subset D:
X N N X X X
X X N N X X
X X X N N X
   where X's are critical residues and N's are non-critical residues and the 3 polypeptides show the possible arrangement to generate adjacent non-critical residues and polypeptides with critical residues at the ends.

Subset D can then be further restricted to generate a new subset of polypeptides, subset E, that are dissimilar from each other. To extract a subset E, a single polypeptide is chosen at random from subset D as the first, reference polypeptide. A similarity ranking is calculated for all of the polypeptides in subset D using a replaceability matrix (also referred to herein as a similarity matrix) that compares the similarity of the amino acids at the critical positions to each other (see e.g., Geysen et al. (1988) J. Mol. Recog. 1(1): 32-41).

A similarity (replaceability) matrix can be constructed empirically. For example, a collection of protein antigens and antisera and/or antibodies that bind to the antigens is generated. The binding sites within the antigens for the antibodies, epitopes, are identified. Such epitopes can be identified by methods such as deletion analysis where amino acids are deleted until the smallest epitope(s) are identified. Epitopes also can be identified by scanning analysis where overlapping sets of polypeptides composed of the possible amino acid oligomers, e.g. 5-mers, 6-mers, 7-mers, or 8-mers etc., of the full-length polypeptide are generated and the antigenic oligomers identify epitopes. Once identified, each epitope is then further analyzed by synthesizing the epitope along with a set of peptide analogs that replaces each residue with other amino acids. For example, a set can be constructed that replaces each residue, one at a time, with the other 19 naturally occurring amino acids. Such replacement sets also can be constructed with non-naturally occurring amino acids or a combination of naturally occurring and non-naturally occurring amino acids. Such sets can be constructed for example, using combinatorial peptide libraries (Pinilla et al. (1999) Curr. Opin. Immunol. 11:193-202), and multipin synthesis (Geysen et al., (1987) J. Immunol. Methods 102:259-274, Rodda et al. (1996) Methods: A companion to Methods Enz. 9: 473-481). Alternatively, mutagenesis can be used to introduce amino acid changes in the protein containing the epitope, and the effect of the changes assessed to determine replaceability (Alexander et al., (1992) Proc. Natl. Acad. Sci. USA 89:3352-3356). Using the replacement sets, the variants are each tested against antibodies for the epitope and binding is assessed as compared to the unaltered epitope, for example by using an ELISA assay. The comparison of the variants and unaltered epitopes generates scores (for example, scores based on comparison of antigenicity) that can then be integrated with scores from other antigen replacement sets and antibodies to generate a database of replaceability in epitopes and produce a replaceability (similarity) matrix (Geysen et al. (1988) J. Mol. Recog. 1(1): 32-41). Replaceability scores can be based, for example, on the frequency that an amino acid when used to replace another maintains or decreases antigenicity of an epitope.

Non-naturally occurring amino acids also can be assigned a similarity ranking for use with the methods. For example, a similarity matrix can be constructed based on their structural and functional similarity to each other and to naturally occurring amino acids. A similarity matrix also can be constructed by replacing naturally occurring amino acids in epitopes with non-natural amino acids and assessing the binding of antibodies to the replacement epitopes such as by ELISA.

An example of a similarity (replaceability) matrix is given in Table 3 (Geysen et al. (1988) J. Mol. Recog. 1(1): 32-41):

TABLE 3
Similarity Matrix

| | E | P | Q | N | F | H | T | K | L | D | G | S | Y |
|---|---|---|---|---|---|---|---|---|---|---|---|---|---|
| E | 100 | 13 | 33 | 13 | 2 | 8 | 10 | 6 | 8 | 42 | 13 | 15 | 6 |
| P | 5 | 100 | 16 | 11 | 8 | 11 | 11 | 16 | 3 | 3 | 14 | 14 | 0 |
| Q | 15 | 10 | 100 | 25 | 5 | 10 | 10 | 5 | 5 | 5 | 20 | 15 | 10 |
| N | 4 | 0 | 13 | 100 | 4 | 9 | 4 | 9 | 4 | 4 | 4 | 9 | 0 |
| F | 11 | 11 | 11 | 11 | 100 | 5 | 26 | 5 | 37 | 16 | 0 | 32 | 21 |
| H | 8 | 23 | 23 | 15 | 0 | 100 | 15 | 15 | 0 | 0 | 23 | 8 | 8 |
| T | 15 | 6 | 12 | 12 | 6 | 9 | 100 | 12 | 9 | 6 | 3 | 44 | 6 |
| K | 0 | 3 | 26 | 23 | 10 | 26 | 23 | 100 | 10 | 10 | 10 | 29 | 0 |
| L | 2 | 4 | 12 | 6 | 22 | 8 | 4 | 18 | 100 | 8 | 2 | 4 | 10 |
| D | 50 | 4 | 12 | 42 | 4 | 23 | 15 | 0 | 4 | 100 | 0 | 27 | 0 |
| G | 3 | 0 | 9 | 3 | 6 | 12 | 3 | 12 | 6 | 6 | 100 | 24 | 3 |
| S | 17 | 6 | 0 | 0 | 11 | 39 | 22 | 11 | 6 | 0 | 6 | 100 | 6 |
| Y | 0 | 0 | 0 | 0 | 29 | 0 | 0 | 14 | 14 | 0 | 0 | 0 | 100 |

A similarity score is determined for each polypeptide in subset D as compared with the first reference polypeptide chosen for subset E. The similarity score can be determined for example, by combining the similarity probabilities (represented in Table 3 above as 0-100%) to determine an overall score for the polypeptide. For example, if subset D is a collection of 6-mer polypeptides and the first polypeptide chosen is EPNGYF (SEQ ID NO:1), each polypeptide in subset D is compared with the reference first polypeptide, EPNGYF (SEQ ID NO:1), using the similarity matrix to calculate a similarity score by combining the similarity value at each of the 4 critical positions to the corresponding positions in the reference polypeptide. The maximum score is 100% (identical polypeptide) and the minimum score is zero.

The number of members for subset E is set at a desired number of polypeptides, for example 10, 20, 30, 40, 50, 100, 200 or 1000 polypeptides. A threshold value is determined that will generate the desired number of polypeptides for subset E. For example, if the threshold is set very low, and therefore the degree of similarity is very low and a smaller subset E of polypeptides will be generated. Conversely, if the threshold of similarity is set high, the subset E will be a larger number of polypeptides. The number of polypeptides can be determined by one skilled in the art based on the intended subsequent use of the polypeptides. For example, if a library of polypeptides of several thousand polypeptides is desired, the threshold can be set higher. If fewer, such as ten, polypeptides that are dissimilar from each other are desired, the threshold can be set lower.

From subset E, amino acids are added into the non-critical positions to create subset F. Non-critical positions can be any amino acid, including naturally occurring and non-natural amino acids. Non-critical positions also can be utilized to introduce added functionalities into the polypeptide, such as enhancing solubility and folding. In one exemplary embodiment, amino acids that increase solubility and permit flexibility and folding are used at the non-critical positions. For example, the amino acids S, G and Y are utilized at the non-critical positions. The non-critical positions can be further restricted by designating each as unique, i.e., there is no duplication or any multiples of a chosen amino acid within the polypeptide length. For example, in a given set, such as the exemplary subset of 6-mers described herein, the two non-critical positions are designated as S and G. Non-critical positions also can include additional amino acids at either the N or C terminus. For example, one or more amino acids can be added at either or both termini.

The methods for generating highly antigenic highly specific polypeptides can include the use of natural and non-natural amino acids. The use of non-naturally occurring amino acids increases the diversity and thus uniqueness of the polypeptides that can be generated. For example, there are several hundred non-naturally occurring amino acids that are commercially available and an even larger number that can be synthesized by standard chemistry methods known in the art. Non-naturally occurring amino acids can be used at either critical or non-critical residues or at critical and non-critical residues. The ability to incorporate non-naturally occurring amino acids also permits linear, cyclic and branched polypeptide structures to be designed and constructed.

Non-natural amino acids include, but are not limited to, non-natural β-amino acids; amino acids having alkyl, cycloalkyl, heterocyclyl, aromatic, heteroaromatic, electroactive, conjugated, azido, carbonyl and unsaturated side chain functionalities; isomeric N-substituted glycine, wherein the side chain of an α-amino acid is attached to the amino nitrogen instead of to the α-carbon of that molecule. The following are representative examples of non-natural amino acids.

Non-natural amino acids that are modifications of natural amino acids such that the amino group is attached to β-carbon atom of the natural amino acid (e.g. β-tyrosine). Non-natural amino acids that are modifications of natural amino acids in the side chain functionality, such that the imino groups or divalent non-carbon atoms such as oxygen or sulfur of the side chain of the natural amino acids have been substituted by methylene groups, or, alternatively, amino groups, hydroxyl groups or thiol groups have been substituted by methyl groups, olefin, or azido groups, so as to eliminate their ability to form hydrogen bonds, or to enhance their hydrophobic properties (e.g. methionine to norleucine).

Non-natural amino acids that are modifications of natural amino acids in the side chain functionality, such that the methylene groups of the side chain of the natural amino acids have been substituted by imino groups or divalent non-carbon atoms or, alternatively, methyl groups have been substituted by amino groups, hydroxyl groups or thiol groups, so as to add ability to form hydrogen bonds or to reduce their hydrophobic properties (e.g. leucine to 2-aminoethylcysteine, or isoleucine to o-methylthreonine).

Non-natural amino acids that are modifications of natural amino acids in the side chain functionality, such that a methylene group or methyl groups have been added to the side chain of the natural amino acids to enhance their hydrophobic properties (e.g. Leucine to gamma-Methylleucine, Valine to beta-Methylvaline (t-Leucine)).

Non-natural amino acids that are modifications of natural amino acids in the side chain functionality, such that a methylene group or methyl groups of the side chain of the natural amino acids have been removed to reduce their hydrophobic properties (e.g. Isoleucine to Norvaline).

Non-natural amino acids that are modifications of natural amino acids in the side chain functionality, such that the amino groups, hydroxyl groups or thiol groups of the side chain of the natural amino acids have been removed or methylated to eliminate their ability to form hydrogen bonds (e.g. Threonine to o-methylthreonine or Lysine to Norleucine). Non-natural amino acids that are optical isomers of the side chains of natural amino acids (e.g. Isoleucine to Alloisoleucine).

Non-natural amino acids that are modifications of natural amino acids in the side chain functionality, such that the substituent groups have been introduced as side chains to the natural amino acids (e.g. Asparagine to beta-fluoroasparagine). Non-natural amino acids that are modifications of natural amino acids where the atoms of aromatic side chains of the natural amino acids have been replaced to change the hydrophobic properties, electrical charge, fluorescent spectrum or reactivity (e.g. Phenylalanine to Pyridylalanine, Tyrosine to p-Aminophenylalanine).

Non-natural amino acids that are modifications of natural amino acids where the rings of aromatic side chains of the natural amino acids have been expanded or opened so as to change hydrophobic properties, electrical charge, fluorescent spectrum or reactivity (e.g. Phenylalanine to Naphthylalanine, Phenylalanine to Pyrenylalanine). Non-natural amino acids that are modifications of the natural amino acids in which the side chains of the natural amino acids have been oxidized or reduced so as to add or remove double bonds (e.g. Alanine to Dehydroalanine, Isoleucine to Beta-methylenenorvaline).

Non-natural amino acids that are modifications of proline in which the five-membered ring of proline has been opened or, additionally, substituent groups have been introduced (e.g. Proline to N-methylalanine). Non-natural amino acids that are modifications of natural amino acids in the side chain functionality, in which the second substituent group has been introduced at the alpha-position (e.g. Lysine to alpha-difluoromethyllysine).

Non-natural amino acids that are combinations of one or more alterations, as described supra (e.g. Tyrosine to p-Methoxy-m-hydroxyphenylalanine). Non-natural amino acids that are isomeric N-substituted glycines, wherein the side chain of an a-amino acid is attached to the amino nitrogen instead of to the a-carbon of that molecule (e.g. N-methyl glycine, N-isopropyl glycine). Non-natural amino acids that differ in chemical structures from natural amino acids but are compatible, in protected or unprotected form, with a hybrid synthesis of peptide chemistry.

Non-natural amino acids are readily available and widely known. Exemplary non-natural amino acids (with their abbreviations) include, but are not limited to, for example: Aib for 2-amino-2-methylpropionic acid, β-Ala for β-alanine, α-Aba for L α aminobutanoic acid; D-α-Aba for D-α aminobutanoic acid; Ac₃c for 1 aminocyclopropane-carboxylic acid; Ac₄c for 1 aminocyclobutanecarboxylic acid; Ac₅c for 1-aminocyclopentanecarboxylic acid; Ac₆c for 1-aminocyclohexanecar-boxylic acid; Ac₇c for 1-aminocycloheptanecarboxylic acid; D-Asp(ONa) for sodium D-aspartate; D-Bta for D-3-(3-benzo[b]thienyl)alanine; C₃al for L-3-cyclopropylalanine; C₄al for L-3-cyclobutylalanine; C₅al for L-3-cyclopentylalanine; C₆al for L-3-cyclohexylalanine; D-Chg for D-2-cyclohexylglycine; CmGly for N-(carboxymethyl)glycine; D-Cpg for D-2-cyclopentylglycine; CpGly for N-cyclopentylglycine; Cys(O₃Na) for sodium L-cysteate; D-Cys(O₃H) for D-cysteic acid; D-Cys(O₃Na) for sodium D-cysteate; D-Cys(O₃BU₄N) for tetrabutylammonium D-cysteate; D-Dpg for D-2-(1,4 cyclohexadienyl)-glycine; D-Etg for (2S)-2 ethyl-2-(2 thienyl)glycine; D-Fug for D-2-(2 furyl)glycine; Hyp for 4-hydroxy-L-proline; leGly for [2-(4-imidazolyl)ethyl]glycine; alle for L-L-alloisoleucine; D-alle for D-alloisoleucine; D-ltg for D-2-(isothiazolyl)glycine; D-tertLeu for D-2-amino-3,3-dimethylbutanoic acid; Lys(CHO) for N⁶-formyl-L-lysine; MeAla for N-methyl-L-alanine; MeLeu for N-methyl-L-leucine; MeMet for N-methyl-L-methionine; Met(O) for L-methionine sulfoxide; Met(O₂) for L-methionine sulfone; D-Nal for D-3-(1-naphthyl)alanine; Nle for L-norleucine; D-Nle for D-nor-leucine; Nva for L-norvaline; D-Nva for D-norvaline; Orn for L-ornithine; Orn(CHO) for N⁵-formyl-L-omithine; D-Pen for D-penicillamine; D-Phg for D-phenylglycine; Pip for L-pipecolinic acid; ⁱPrGly for N-isopropylglycine; Sar for sarcosine; Tha for L-3-(2-thienyl)alanine; D-Tha for D-3(2-thienyl)-alanine; D-Thg for D-2-(2-thienyl)glycine; Thz for L-thiazolidine-4-carboxylic acid; D-Trp(CHO) for Nⁱⁿ-formyl-D-hytophan; D-trp(O) for D-3-(2,3-di-hydro-2-oxoindol-3-yl)alanine; D-trp((CH₂)ₘCOR¹) for D-tryptophan substituted by a (CH₂)ₘCOR¹ group at the 1-position of the indole ring; Tza for L-3-(2-thiazolyl)alanine; D-Tza for D-3-(2-thiazolyl)alanine; D-Tzg for D-2-(thiazolyl)glycine.

Non-naturally occurring amino acids can be ranked for antigenicity using methods applied to the naturally occurring amino acids, for example by testing sequences against antisera or libraries of antibodies (described herein) and can be ranked along-side naturally occurring amino acids. For example, a representative set of polypeptides composed of non-naturally occurring amino acids and/or a combination of non-naturally occurring and naturally occurring amino acids of a chosen polypeptide length can be used to immunize animals. Based on the subset of polypeptides injected that are antigenic and non-antigenic, amino acids are identified that either are more likely to be present in antigenic polypeptides or are more likely to be present on non-antigenic polypeptides. The likelihood of an amino acid's presence in antigenic polypeptide gives an observed antigenic ranking. Some non-natural amino acids are very structurally similar to naturally occurring amino acids and to other non-naturally occurring amino acids. This similarity can be factored in to provide antigenicity rankings based on these similarities. Non-naturally occurring amino acids also can be assigned a similarity ranking for use with the methods as described, based on their structural and functional similarity to each other and to naturally occurring amino acids. For example, a collection of polypeptides can be generated containing non-natural amino acids and tested for antigenicity. Polypeptides that are antigenic can be used to create further sets of polypeptides (replacement sets) by systematically replacing some or all of the amino acids systematically to determine that amino acids are critical. The data can then be analyzed for the replacement sets to determine a factor for each non-natural amino acid, where the factor represents the frequency of finding the particular non-natural amino acid in a critical position within an antigenic polypeptide.

Once designed, highly specific highly antigenic polypeptides can be synthesized by chemical synthesis or by expression systems known in the art. Highly specific highly antigenic polypeptides can be used as binding partners in the therapeutic molecules described herein. Also, as described further herein, these highly antigenic highly specific polypeptides can be used to generate and select binding molecules, such as antibodies, which are then used to construct capture agents for use in therapeutic complexes. For example, a set of highly specific, highly antigenic polypeptides are used to immunize mice and antibodies are isolated that bind to the polypeptides. Antibodies that bind to specific polypeptide are used to construct capture agents for association with effectors. The highly specific, highly antigenic polypeptides can be used as binding partners for conjugating to targeting domains and constructing therapeutic complexes.

f. Small Molecule Binding Partners

Small molecule binding partners can be designed de novo or based on known structures, for example, based on ligands for a receptor. For example, a collection of binding partner:capture agent pairs can be designed from a receptor:ligand pair. Receptor diversity can be generated by targeted or random mutagenesis in the ligand binding domain. A library of small molecules based on the ligand structure can be designed through rationale design, combinatorial chemistry or other methods known in the art. Interactions can be tested between the receptor molecules and the ligand-based library to identify receptor:ligand pairs that can then be used to construct binding partner:capture agent pairs for use in therapeutic complexes as described herein.

### E. ASSEMBLING AND PRODUCING THERAPEUTIC COMPLEXES

As described, the design of the therapeutic complexes herein is modular. The complexes, (TR)ᵣ-(L1)ₛ-(B1)ₜ-(B2)ₓ-(L2)_{y}-(E)_{z}, composed of targeting domains, binding partners, capture agents, effectors and optionally one or more linkers can be designed together or independently and then assembled into an effective complex.

The components of a complex are joined by any stable interaction, including covalent bonds, ionic bonds, hydrophobic, Van der Waals, hydrogen bonds and other such bonds and interactions, such that the resulting complex is stable upon administration to a subject, such that it performs its intended effect. Typically such linkages have a binding affinity (Kₐ) of at least about 10⁶ l/mol, 10⁷ l/mol, 10⁸ l/mol, 10⁹ l/mol, 10¹⁰ l/mol or greater (generally 10⁸ or greater).

1. Conjugating Binding Partners and Capture Agents to Targeting Domains and Effectors

To construct therapeutic complexes, generally, binding partners are linked to targeting domains either directly or optionally with one or more linkers. Similarly, capture agents and effectors can be linked either directly or optionally with one or more linkers. Linkage can be effected by any means of covalent bonding such as by preparing fusion proteins or by chemically conjugating two or more components, such as chemically conjugating an effector and a capture agent. The linkage can be direct or a linker can be used to act as an intermediate molecule to join the components. Conjugation by recombinant methods results in a fusion protein, where typically one component, such as a binding partner, is linked to either the N-terminus or C-terminus of another component, for example, a targeting domain, but can be inserted elsewhere. In chemical conjugates, the components can be linked directly or indirectly via a linker anywhere that conjugation can be effected.

a. Fusion Proteins

Fusion proteins can be produced by recombinant expression of nucleic acids that encode the fusion protein. The formation of a fusion protein involves the placement of two separate coding sequences, such as genes or nucleotide sequences, for example one encoding a targeting domain and the second encoding a binding partner, in sequential order. The nucleic acid encoding the components are joined in-frame, such that when translated a single protein is produced. In cases where one of the components has more than one polypeptide chain, such as an immunoglobulin, a fusion can be constructed with one of the polypeptide chains.

A linker can be used between components, for example, to facilitate cloning, add a spacer sequence or add additional functionality to the molecule. Linkers can be nucleic acid encoding 1 or more amino acids joined in-frame with the components. Additional sequences also can be joined to the fusion protein such as a tag for purification or detection. For example, a myc epitope or a His₆ tag can be joined to facilitate purification.

Nucleic acids encoding the components can be obtained by a variety of means known in the art. They can be generated synthetically, isolated from known sources such as cell lines, gene banks, tissues, subject samples and recombinant organisms using standard molecular biology techniques known to those of skill in the art. For example, PCR can be used to amplify the nucleic acid from biological material and/or libraries can be screened by hybridization techniques. Methods for the formation of a nucleic acid encoding a protein fusion include, but are not limited to ligation of nucleic acid sequences, primer extension, PCR including overlap PCR methods, insertion by gene shuffling, recombination strategies, incorporation by transposases; and incorporation by splicing. Nucleic acids encoding components for fusion proteins are cloned into an appropriate cloning vector. Methods for creating an expression vector are well known to those of skill in the art (see, e.g., Sambrook et al. (1989) Molecular Cloning: A Laboratory Manual, Cold Spring Harbor Laboratories, Cold Spring Harbor, N.Y.).

b. Chemical Conjugation

To effect chemical conjugation described herein, components such as an effector and capture agent are linked directly or indirectly, such as through a linker. Chemical conjugation can be used with any of the components herein, particularly when one or more of the chosen components is other than a polypeptide, such as a nucleic acid or small molecule, or when production of a fusion protein is not required. Any methods known to those of skill in the art for chemically conjugating selected moieties can be used.

Cross-linking is an exemplary method of chemical conjugation for linking binding partners and targeting domains. Cross-linking can be effected between the covalent interaction of moieties in the partner and targeting domain and/or by use of cross-linking reagents. Such reagents include, but are not limited to, heterobifunctional, homobifunctional and trifunctional reagents, and can be used to introduce, produce or utilize reactive groups, such as thiols, amines, hydroxyls and carboxyls, on one or both of the molecules, which can then be contacted with the other, containing a second reactive group, such as a thiol, amine, hydroxyl and carboxyl, to form a chemical linkage between two or more components. These reagents can be used to directly or indirectly, such as through a linker, to conjugate a binding partner and a targeting domain and/or conjugate an effector and capture agent. Generally, cross-linking reagents have two reactive groups connected by a flexible spacer arm. The reagents differ in their spacer arm length, cleavability, solubility and reactive groups, and can be selected to alter a characteristic of the conjugate complex, such as the solubility, steric hindrance and permeability. Some cross-linking reagents (i.e., homobifunctional cross-linkers) have the same reactive groups at both ends, others (i.e., hetero-bifunctional cross-linkers) have different reactive groups at the ends and some cross-linkers contain additional functional groups to allow the cross-linker molecule to be labeled. Additionally, some cross-linking reagents (i.e., trifunctional cross-linkers) have three reactive groups to make trimeric complexes.

Cross-linking reactions involving molecules such as proteins, generally are reactive group reactions, such as side chain reactions, and are nucleophilic, resulting in a portion of the end of the cross-linker being displaced in the reaction (the leaving group). Nucleophilic attack is dependent on the pH, temperature and ionic strength of the cross-linking buffer. For example, when the buffer is one to two pH units below the pKₐ of the reactive group, such as a side chain, the species is highly protonated and is most reactive. One to two pH units above the pKₐ, the species is not protonated and not reactive. The majority of proteins have reactive groups, such as primary amines and free sulfhydryls, available at the surface or terminus of the molecule. These are the two most commonly used groups in molecular cross-linking strategies. Cross-linking strategies also can use carbohydrates, carboxyls or other reactive functional groups.

Many factors are considered to obtain optimal cross-linking for a particular application. Factors that affect molecular folding, such as protein folding, (e.g., pH, salt, additives and temperature) can alter conjugation results. Other factors such as molecule or binding partner concentration, cross-linker concentration, number of reactive functional groups available, cross-linker spacer arm length, and conjugation buffer composition should also be considered.

Exemplary cross-linking strategies include thiol-thiol linkages, amine-amine linkages, disulfide bonds, amine-carboxylic acid and thiol-carboxylic acid crosslinking. Among the reagents for crosslinking binding partners and targeting domains are described for cross-linking the conjugates below. Linkers that are suitable for chemically linking the complexes include disulfide bonds, thioether bonds, hindered disulfide bonds, esters, and covalent bonds between free reactive groups, such as amine and thiol groups. These bonds are produced using heterobifunctional reagents to produce reactive thiol groups on one or both of the polypeptides and then reacting the thiol groups on one polypeptide with reactive thiol groups or amine groups on the other. Other linkers include, acid cleavable linkers, such as bismaleimidoethoxy propane, acid labile-transferrin conjugates and adipic acid dihydrazide, that would be cleaved in more acidic environments; photocleavable cross-linkers that are cleaved by visible or UV light.

Numerous heterobifunctional cross-linking reagents that are used to form covalent bonds between amino groups and thiol groups and to introduce thiol groups into proteins, are known to those of skill in this art (see, e.g., the PIERCE CATALOG, ImmunoTechnology Catalog & Handbook, 1992-1993, which describes the preparation of and use of such reagents and provides a commercial source for such reagents; see, also, e.g., Cumber et al. (1992) Bioconjugate Chem. 3:397-401; Thorpe et al. (1987) Cancer Res. 47:5924-5931; Gordon et al. (1987) Proc. Natl. Acad Sci. 84:308-312; Walden et al. (1986) J. Mol. Cell Immunol. 2:191-197; Carlsson et al. (1978) Biochem. J. 173: 723-737; Mahan et al. (1987) Anal. Biochem. 162:163-170; Wawryznaczak et al. (1992) Br. J. Cancer 66:361-366; Fattom et al. (1992) Infection & Immun. 60:584-589). These reagents may be used to form covalent bonds between the TA and targeted agent. These reagents include, but are not limited to: N-succinimidyl-3-(2-pyridyldithio)propionate (SPDP; disulfide linker); sulfosuccinimidyl 6-[3-(2-pyridyldithio)propionamido]hexanoate (sulfo-LC-SPDP); succinimidyloxycarbonyl-a-methyl benzyl thiosulfate (SMBT, hindered disulfate linker); succinimidyl 6-[3-(2-pyridyldithio) propionamido]hexanoate (LC-SPDP); sulfosuccinimidyl 4-(N-maleimidomethyl)cyclohexane-1-carboxylate (sulfo-SMCC); succinimidyl 3-(2-pyridyldithio)butyrate (SPDB; hindered disulfide bond linker); sulfosuccinimidyl 2-(7-azido-4-methylcoumarin-3-acetamide) ethyl-1,3′-dithiopropionate (SAED); sulfo-succinimidyl 7-azido4-methylcoumarin-3-acetate (SAMCA); sulfosuccinimidyl 6-[alpha-methyl-alpha-(2-pyridyldithio)toluamido]hexanoate (sulfo-LC-SMPT); 1,4-di-[3′-(2′-pyridyldithio)propionamido]butane (DPDPB); 4-succinimidyloxycarbonyl-α-methyl-α-(2-pyridylthio)toluene (SMPT, hindered disulfate linker);sulfosuccinimidyl6[α-methyl-α-(2-pyridyldithio)toluamido]hexanoate (sulfo-LC-SMPT); m-maleimidobenzoyl-N-hydroxysuccinimide ester (MBS); m-maleimidobenzoyl-N-hydroxysulfosuccinimide ester (sulfo-MBS); N-succinimidyl(4-iodoacetyl)aminobenzoate (SIAB; thioether linker); sulfosuccinimidyl(4-iodoacetyl)amino benzoate (sulfo-SIAB); succinimidyl4(p-maleimidophenyl)butyrate (SMPB); sulfosuccinimidyl4-p-maleimidophenyl)butyrate (sulfo-SMPB); azidobenzoyl hydrazide (ABH).

Acid cleavable linkers, photocleavable and heat sensitive linkers may also be used, particularly where it may be necessary to cleave the targeted agent to permit it to be more readily accessible to reaction. Acid cleavable linkers include, but are not limited to, bismaleimideothoxy propane; and adipic acid dihydrazide linkers (see, e.g., Fattom et al. (1992) Infection & Immun. 60:584-589) and acid labile transferrin conjugates that contain a sufficient portion of transferrin to permit entry into the intracellular transferrin cycling pathway (see, e.g., Welhbner et al. (1991) J. Biol. Chem. 266:4309-4314).

Photocleavable linkers are linkers that are cleaved upon exposure to light (see, e.g., Goldmacher et al. (1992) Bioconj. Chem. 3:104-107, which linkers are herein incorporated by reference), thereby releasing the targeted agent upon exposure to light. Photocleavable linkers that are cleaved upon exposure to light are known (see, e.g., Hazum et al. (1981) in Pept., Proc. Eur. Pept. Symp., 16th, Brunfeldt, K (Ed), pp. 105-110, which describes the use of a nitrobenzyl group as a photocleavable protective group for cysteine; Yen et al. (1989) Makromol. Chem 190:69-82, which describes water soluble photocleavable copolymers, including hydroxypropylmethacrylamide copolymer, glycine copolymer, fluorescein copolymer and methylrhodamine copolymer; Goldmacher et al. (1992) Bioconj. Chem. 3:104-107, which describes a cross-linker and reagent that undergoes photolytic degradation upon exposure to near UV light (350 nm); and Senter et al. (1985) Photochem. Photobiol 42:231-237, which describes nitrobenzyloxycarbonyl chloride cross-linking reagents that produce photocleavable linkages), thereby releasing the targeted agent upon exposure to light. Such linkers would have particular use in treating dermatological or ophthalmic conditions that can be exposed to light using fiber optics. After administration of the conjugate, the eye or skin or other body part can be exposed to light, resulting in release of the targeted moiety from the conjugate. Such photocleavable linkers are useful in connection with diagnostic protocols in which it is desirable to remove the targeting agent to permit rapid clearance from the body of the animal.

2. Assembling Therapeutic Complexes

Binding partner-targeting domain conjugates are assembled with capture agent-effector conjugates to form a therapeutic complex. Assembly can be performed in vitro or in vivo, with purified components, or in a mixture, extract, partially purified extract, cell or in an animal, subject or patient. Generally, the interaction between binding partner and capture agent to associate the complex is non-covalent. The affinity of the capture agent for the binding partner in a complex should be sufficient such that the complex is stable to routine manipulations for the preparation of compositions for administration (as described below or known to those of skill in the art). Capture agent:binding partner interactions can be fixed by cross-linking, such as by treating with a compound or condition after forming the complex (such as the use of conjugating agents described herein). Conjugated (e.g. cross-linked) therapeutic complexes can then be formulated for administration to a subject.

In one embodiment, complexes are assembled prior to administration. Binding partner-targeting domain and capture agent-effector conjugates are mixed together to initiate complex formation. The ratio of binding partner-targeting domain to effector can be modulated based on the availability of the components and their affinity of interaction. Such ratios will typically be on the order of 10:1, 4:1, 2:1, 1:1,1:2, 1:4, and 1:10. Once the complex is formed the binding partner-target domain-capture agent-effector can be in a ratio dependent on the number of interacting regions of a capture agent for a binding partner. Typically, a capture agent can bind to one or two binding partners. For example, if a capture agent is an immunoglobulin, it can bind 1 or 2 binding partners. Capture agents are not limited to binding 1 or 2 binding partners, capture agents can be constructed that are multivalent and bind to a plurality of binding partners.

In one embodiment, therapeutic complexes are provided that contain a plurality of capture agents and/or a plurality of targeting domains and/or effectors. In one aspect of the embodiment, the multiple targeting domains are different from each other and each targeting domain is conjugated to a common binding partner that specifically binds to a capture agent. A common binding partner conjugated to each targeting domain can create a competitive reaction between targeting domain-binding partner conjugates for binding to a corresponding capture agent. Thus, by mixing different proportions of conjugates with a capture agent-effector containing multiple capture agents, sets of therapeutic complexes can be formed that contain different targeting domains and in different proportions. Ratios between different binding partner-targeting domain conjugates for mixing are dependent on the desired proportions of therapeutic complexes with each targeting domain and includes, but is not limited to ratios of 1:1, 1:2, 1:3, 1:4, 1:5, 1:6, 1:8, 1:10, 1:20, and 1:50. Sets of therapeutic complexes also can be formed by mixing different ratios of targeting domains with one or more common binding partners in a conjugation reaction, for example under conditions where only a desired number of targeting domains are linked to each binding partner, for example one targeting domain linked to one binding partner. Any number of targeting domains, effectors, capture agents and binding partners can be used to create therapeutic complexes so long as the therapeutic complexes can assemble and carry out their intended effects.

For example, two different targeting domains (TR₁ and TR₂) are each conjugated to a common binding partner B1₁ to create conjugates B1-TR₁ and B1-TR₂. The binding partner B1 specifically binds to a capture agent B2 and there are two similar capture agents B2 conjugated to an effector E₁, generating (B2)₂-E1. B1-TR₁ and B1-TR₂ are mixed together in varying ratios and then assembled with (B2)₂-E1. Therapeutic complexes are formed that include (B1-TR₁)₂-(B2)₂-E₁,(B1-TR₂)₂-(B2)₂-E₁) and (B1-TR₁)-(B2)-E₁-(B2)-(B1-TR₂). Altering the ratios of B1-TR₁ and B1-TR₂ mixed with (B2)₂-E₁ or altering the ratios of TR₁ and TR₂ mixed with Bi, will change the proportion of the resulting complexes in the assembled set of therapeutic complexes.

Once formed, therapeutic complexes can then be used for formulation, functional assays and administration. In some instances, it can be beneficial to separate assembled complexes from remaining unassembled components. Any method known in the art to effect such separation can be used. For example, gel filtration and size separation methods can be used to separate the larger complex from unassembled components. Affinity chromatography methods also can be used to enrich for complexes that contain the targeting and the effector functions.

In another embodiment, therapeutic complexes are assembled in vivo. For example, polypeptide binding partner-targeting domains fusion proteins and capture agent-effector fusion proteins can be expressed in the same host cell or separately in host cells that are then fused to form a hybrid cell. The affinity of the capture agent for the binding partner forms a complex between the binding partner-targeting polypeptides and the capture agent-effector polypeptides. Such complexes can then be used for assays and administration directly, or first purified or partially purified. In one example, a therapeutic complex is formed in a subject. A binding partner-targeting domain is administered to a subject, and following, an effector-capture agent is administered. A complex of the components is formed in the subject based on the affinity of the capture agent for the binding partner.

In another embodiment, binding partner-targeting domains and capture agent-effectors are assembled prior to administration and the complex is then cross-linked to add further stability. For example, a binding partner-targeting domain and a capture agent-effector can be mixed and complexes are separated from unassembled components. Conjugation methods, such as chemical conjugation methods described herein and others known in the art, can be used to conjugate the binding partner-targeting domain complexed to the capture agent-effector. For example, functional groups on the binding partner and capture agent brought in close proximity by their interaction can then be cross-linked for further complex stability. Such cross-links can include chemical cross-linking and photo-activatable cross-linking.

3. Assays for Function of Components and Assembled Complexes

Assays can be used to monitor functional and structural properties of the therapeutic complexes and their components. Such assays include concentrations of components and their stability, formation of binding partner-targeting domain conjugates, complex formation, interaction of binding partners and capture agents, interaction of binding partner-targeting domains and capture agent-effectors, targeting domain interaction with the target, therapeutic complex interaction with the target, effector function, and biological effect of the therapeutic complex.

Effectors function as an effector component, as a capture agent-effector conjugate and within the therapeutic complex can be assessed for example, using cell proliferation, cytotoxicity, cell signaling assays, immunoassays, enzyme assays and binding assays. Such assays include, but are not limited to, ³H thymidine incorporation assays, trypan blue cell counts, competitive and non competitive immunoassays, western blots, immunohistochemistry radioimmunoassays, ELISA (enzyme linked immunosorbent assay) and other “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays, FACS analysis, ⁵¹Cr release assay, kinase assays, receptor binding assays, and electromobility shift assays (EMSAs).

Cellular-based assays can be used to monitor effector and therapeutic complex function and to demonstrate biological effect or biological activity. For example, the components and complexes can be tested for their effect on a cell line, for example a specific type, such a model cell line for a disease or condition or a subject tissue sample. Such assays are known to those of skill in the art and include, but are not limited to, rosette formation assays, cell lysis assays and immune cell modulation. For example, the effectors and therapeutic complexes can be tested for their ability to augment activated immune cells by contacting them with activated immune cells and determining the ability of the effector or therapeutic complex to modulate the biological activity of the activated immune cells. The ability to modulate the biological activity of activated immune cells can be assessed for example, by detecting the expression of cytokines or antigens, detecting the proliferation of immune cells and detecting the activation of signaling molecules. One of skill in the art is capable of choosing the most appropriate assays based on the type of effector.

The binding of a binding partner and binding partner-targeting domain to a capture agent and/or capture agent-effector can be assayed using a variety of interaction assays known in the art. Exemplary assays include but are not limited to, electromobility shift assays (EMSAs), immunoprecipitation, western blot, BIAcore® analysis, ELISAs, analysis with addressable arrays such as described herein and in WO 03/062402 (see also U.S. Patent Applications 60/422,923 and 60/423,018), chromatography and affinity chromatography. Interactions between binding partners and capture agents also can be monitored using surface plasmon resonance detection such as the Pharmacia BIAcore® system. This technology provides the ability to determine binding constants and dissociation constants of protein-protein interactions. The use of the BIAcore® system requires purified components and a source of soluble target molecules.

Therapeutic complexes and components also can be assayed using in vivo models. Such assessments of therapeutic complexes and components of therapeutic complexes can include assessment of biological effect, therapeutic effects and pharmaceutical activity in subject-based assays. For example, as described in Example 4, components such as effector molecules and therapeutic complexes can be delivered to an animal, such as a mouse injected with tumor cells, and assayed for survival as well as additional biological effects.

4. Optimization of Components and Complexes

Once the components are isolated, each can be optimized if required. For example, optimization can include improvements to expression, stability, purification, activity (such as effector function, binding, target recognition). Such improvements can be made by any methods known in the art.

a. Humanization

The modification of proteins can be necessary to improve the therapeutic effectiveness and safety of such proteins when used for treatment of humans. One such example is the humanization of proteins in which the amino acid sequences are altered to resemble proteins endogenous to humans. One such example is the humanization of antibodies (Hurle and Gross (1994) Current Opinion in Biotechnology 5:428-433). Murine antibodies are often isolated in the course of identifying antibody derived therapeutics. The most common method for humanizing antibodies is to retain the antigen binding regions (the CDR regions) from the murine (or other non-human) antibody and to replace the remaining structure with a human antibody structure. This replaces greater than 90% of the amino acid sequences of the antibody with human amino acid sequences. Another humanization method known as veneering reduces the immunogenicity by targeting surface-exposed residues and altering only those residues to human sequences. The non-surface exposed residues are not altered and in some cases this can result in better stability and activity of the molecule.

b. Optimization of Function

In some cases, it can be desirable to improve a particular function of one or more of the components of a therapeutic complex. For example, improvements can be made in activities such as effector function, the affinity and specificity of a capture agent to bind to a binding partner and target recognition by a targeting domain. For small molecule components such improvements can be made by rationale design, the generation and screening of libraries generated around a selected structure (based on the original isolated component) or any other methods known in the art for optimization. For polypeptide components such improvements can be made by similar rationale design and library screening methods. For example, to improve the affinity of a targeting domain for a target, a phage display library can be constructed from variants of a targeting domain. Such variants can be generated through targeted mutagenesis of specific residues within the targeting domain and/or by random mutagenesis of regions including all the regions of a targeting domain. The library is panned against the target and washing conditions are chosen to enrich for high affinity targeting domain-target interactions. Phage are selected that bind to the target with a higher affinity than the starting targeting domain. Multiple rounds of selection can be used to further enrich for high affinity clones. Targeting domains are then isolated from the selected phage and their higher affinity is reconfirmed in target interaction assays such as described herein or known in the art. Effector domains also can be improved using rounds of mutagenesis and screens for improved function, such as by the functional screens described herein or known in the art. Such screens include in vitro and cell-based screening as well as assays based on the administration to subjects, such as mice and animal disease models. Capture agents and binding partners can be improved in a similar manner, for example to alter their specificity and or affinity of interaction.

5. Use of Therapeutic Complexes as a Screening Tool

Therapeutic complexes are designed to specifically recognize a target and confer a biological effect in a target-specific manner. As such, they offer a valuable screening tool for screening candidate molecules for the ability to specifically bind to a target and/or for the ability to provide a biological effect. Therapeutic complexes can be assembled for example with candidate molecules as targeting domains. An effector is chosen that confers a biological effect. Assembled complexes are screened for biological effect on a target to identify targeting domains. In one example, the assembled complexes are screened for a therapeutic effect on a target. For example, single chain antibodies are screened as candidate molecules for targeting domains by testing therapeutic complexes assembled with single chain antibodies as candidate targeting domains such as in an in vivo mouse assay. In another example, molecules identified to bind to a target, can then be used as candidate molecules for targeting domains in therapeutic complexes. Such assays allow efficient testing of molecules that bind to a target for their ability to be converted into therapeutic molecules and uses. Molecules to be used as candidates can be identified by any methods known in the art that identify molecules that bind to a target.

Candidate molecules also can be screened to identify effectors. Therapeutic complexes can be assembled with a targeting domain and candidate molecules as effectors. Assembled complexes with the candidate components are screened for biological effect on a target to identify effectors.

Components identified through such screening assays can be used in the therapeutic complexes described herein. Identified targeting domains and effectors also can be used to engineer other therapeutic molecules such as single molecule therapeutics. In one example, an identified targeting domain and effector are used to validate a selected combination of target and targeting domain for therapeutic effect. For example, single chain antibodies are screened as candidate targeting domains. Particular single chain antibodies are identified as targeting domains, which when complexed with an effector, such as an Fc domain, in a therapeutic complex confer a biological effect, such as a therapeutic effect, on a target. Such single chain antibodies and Fc domains can then be used to design a therapeutic molecule, such as an antibody, which encompasses the target binding and the effector function. For example, a therapeutic molecule can be constructed that includes a variable domains from the single chain antibody and an Fc domain of the effector in an antibody or other suitable scaffold.

6. Expression of Therapeutic Complexes and Components Thereof

Therapeutic complexes can be produced by any means known in the art including in vivo and in vitro methods. In cases where one or more of the components contains a polypeptide, expression of the molecule in a suitable host allows the production of large amounts of the molecule. Hence, typically components of the complexes can be so-expressed and then combined to produce the therapeutic complexes.

a. Hosts and Expression Systems

Components of therapeutic complexes (and the complexes) can be expressed in any organism suitable to produce the required amounts of therapeutic complexes needed. Expression hosts include E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification. Components of a therapeutic complex need not all be expressed in the same host.

Expression in eukaryotic hosts can include expression in yeasts such as Saccharomyces cerevisae and Picchia Pastoria, insect cells such as Drosophila cells and lepidopteran cells, plants and plant cells such as tobacco, corn, rice, algae and lemna. Eukaryotic cells for expression also include mammalian cell lines such as Chinese hamster ovary (CHO) cells, hybridoma and heterohybridoma cell lines, Balb/3T3 cells, and myeloma cells.

Many expression vectors are available for the expression of therapeutic complexes and components of therapeutic complexes. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker that allows selection and maintenance of the transformed cells. In some cases, an origin of replication can be used to amplify the copy number of the vector.

i. Prokaryotic Expression

Prokaryotes, especially E. coli, provide a system for producing large amounts of therapeutic complexes and components of therapeutic complexes. Transformation of E. coli is a simple and rapid technique well known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated XPL promoter.

Therapeutic components and complexes can be expressed in the cytoplasmic environment of E. coli. The cytoplasm is a reducing environment and for some molecules, this can result in the formation of insoluble inclusion bodies. Reducing agents such as dithiolthreotol and β-mercaptoethanol and denaturants, such as guanidine-HCl and urea can be used to resolubilize the proteins. An alternative approach is the expression of therapeutic components and complexes in the periplasmic space of bacteria that provides an oxidizing environment and chaperonin-like and disulfide isomerases can lead to the production of soluble protein. Typically, a leader sequence is fused to the protein to be expressed that directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Mutations also can be used to increase solubility of expressed proteins. For example, point mutations in the heavy chain of antibodies have been used to increase solubility of the expressed protein. Typically, bacteria produce aglycosylated proteins. Thus, if proteins require glycosylation for function, glycosylation can be added in vitro after purification from host cells.

ii. Yeast

Yeasts such as Saccharomyces cerevisae, Schizosaccharomyces pombe, Yarrowia lipolytica, Kluyveromyces lactis and Pichia pastoris are useful expression hosts for components of therapeutic complexes. Yeast can be transformed with episomal replicating vectors or by stable chromosomal integration by homologous recombination. Typically, inducible promoters are used to regulate gene expression. Examples of such promoters include GAL1, GAL7 and GAL5 and metallothionein promoters such as CUP1. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improved expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed therapeutic components and complexes as they exit the secretion pathway. Yeast also is capable of glycosylation at Asn-X-Ser/Thr motifs.

iii. Insect Cells

Insect cells, particularly using baculovirus expression, are useful for expressing components of therapeutic complexes and the complexes. Insect cells express high levels of protein and are capable of most of the post-translational modifications used by higher eukaryotes. Baculovirus have a restrictive host range that improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the bombyx mori nuclear polyhedrosis virus (BmNPV) and an insect cell line such as Sf9 derived from Spodoptera frugiperda, Pseudaletia unipuncta (A7S) and Danaus plexippus (DpN1). For high level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schneider 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

iv. Mammalian Cells

Mammalian expression systems can be used to express components of the therapeutic complexes and the complexes. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. Such vectors often include transcriptional promoter-enhancers for high level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NSO (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media that facilitates purification of secreted proteins from the cell culture media. One such example is the serum-free EBNA-1 cell line (Pham et al., (2003) Biotechnol. Bioeng. 84:332-42.)

v. Plants

Transgenic plant cells and plants can be used for the expression of components of therapeutic complexes and complexes. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline syntase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Because plants can be cross-pollinated, therapeutic complexes can be combined by crossing one transformed plant with another expressing a second molecule, generating progeny seed that express both molecules. Transgenic plant cells also can include algae engineered to produce therapeutic complexes and components of therapeutic complexes (see for example, Mayfield et al. (2003) PNAS 100:438-442). Because plants have different glycosylation patterns than mammalian cells, this can influence the choice of therapeutic complexes produced in these hosts.

b. Purification of Therapeutic Complexes

Method for purification of therapeutic complexes and components from host cells will depend on the chosen host cells and expression systems. For example, for secreted molecules, proteins can be purified from the culture media after removing the cells. For intracellular expression, cells can be lysed and the proteins purified from the extract. When transgenic organisms such as transgenic plants and animals are used for expression, tissues or organs can be used as a starting material to make a lysed cell extract.

Therapeutic complexes and components can be purified using standard protein purification techniques known in the art including, but not limited to, SDS-PAGE, size fraction and size exclusion chromatography, ammonium sulfate precipitation and ionic exchange chromatography. Affinity purification techniques also can be utilized to improve the efficiency and purity of the preparations. For example, protein A columns can be used for molecules with Fc domains, ligands and antigens bound to solid supports can be used for purification of receptors and antibodies. Expression constructs also can be engineered to add an affinity tag to a protein such as a myc epitope, GST fusion or His₆ and affinity purified with myc antibody, glutathione resin and Ni-resin, respectively. Purity can be assessed by any method known in the art including gel electrophoresis and staining and spectrophotometric techniques.

### F. THERAPIES AND TREATMENTS WITH THERAPEUTIC COMPLEXES

1. Animal Models

Animal models are useful tools to assess therapeutic complexes and components of therapeutic complexes. For example, animals can be used as models for a disease or condition. Animals can be injected with disease and/or phenotype-inducing substances and then therapeutic complexes administered to monitor the effects on disease progression. Genetic models also are useful. Animals such as mice can be generated that mimic a disease or condition by the overexpression, underexpression or knock-out of one or more genes. Such animals can be generated by transgenic animal production techniques well known in the art or using naturally occurring or induced mutant strains. Examples of animal models include the humanized CD4⁺ T cell mouse model for rheumatoid arthritis, experimental autoimmune encephalomyelitis in rodents and monkeys, NOD/SCID mouse model transplanted with human cord blood B cells as a model for SLE, the Ecker rat model for renal carcinoma and NOD mice for a model of diabetes.

Therapeutic complexes can be assessed in such animal models. For example, tumors such as B-cell tumors can be injected sub-cutaneously into mice. Subsequently, doses of a therapeutic complex are injected on the days following. Tumor size is monitored by visual measurement and mortality is assessed, comparing groups of mice treated and untreated with the therapeutic complex.

Animal models can be used to monitor half-life and clearance of therapeutic complexes. Such assays can be useful for comparing therapeutic complexes and for calculating doses and dose regimens for further animal and human trials. For example, a therapeutic complex can be injected into the tail vein of mice. Blood samples are then taken at time points after injection (such as minutes, hours and days afterwards) and then the level of therapeutic complex is monitored for example by an ELISA assay. Half-life and clearance assays can be performed on individual components such as binding partner-targeting domain conjugates and effectors as well as the assembled complexes.

Animal models also can be used to assess biodistribution of a therapeutic complexes and components, for example to assess targeting of such molecules and complexes. For example, a labeled therapeutic complex can be administered to an animal, and then waiting for a time interval following the administration for permitting the labeled molecule to preferentially concentrate at the targeted site in the animal. The labeled molecules are then detected and measured in the animal, and compared with non-target sites to assess the proportion of the therapeutic complex that reaches the target. Non-targeted molecules (such as a therapeutic complex where the targeting domain has been removed or disabled) can be used for comparison.

2. Human and Other Animal Therapies

Therapeutic complexes can be administered with a variety of techniques that include intramuscular, intravenous, intradermal, intraperitoneal injection, subcutaneous, epidural, nasal, oral, rectal, topical, inhalational, buccal (e.g., sublingual), and transdermal administration or any other route. Therapeutic complexes can be administered by any convenient route, for example by infusion or bolus injection, by absorption through epithelial or mucocutaneous linings (e.g., oral mucosa, rectal and intestinal mucosa, etc.) and can be administered together with other biologically active agents. Administration can be local or systemic. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Administration also can include controlled release systems including controlled release formulations and device controlled release, such as by means of a pump. The most suitable route in any given case will depend on the nature and severity of the disease or condition being treated and on the nature of the particular composition that is used.

Various delivery systems are known and can be used to administer therapeutic complexes, such as but not limited to, encapsulation in liposomes, microparticles, microcapsules, recombinant cells capable of expressing the compound, receptor mediated endocytosis, and delivery of nucleic acid molecules encoding therapeutic complexes such as retrovirus delivery systems.

Pharmaceutical compositions can be prepared. Generally, pharmaceutically acceptable compositions are prepared in view of approvals from a regulatory agency or other prepared in accordance with generally recognized pharmacopeia for use in animals and in humans. Pharmaceutical compositions can include carriers such as a diluent, adjuvant, excipient, or other vehicles with which a therapeutic complex is administered. Such pharmaceutical carriers can be sterile liquids, such as water and oils, including those of petroleum, animal, vegetable or synthetic origin, such as peanut oil, soybean oil, mineral oil, and sesame oil. Water is a preferred carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents. These compositions can take the form of solutions, suspensions, emulsion, tablets, pills, capsules, powders, and sustained release formulations. A composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and other such agents. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

Formulations are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable derivatives thereof. Pharmaceutically therapeutically active compounds and derivatives thereof are typically formulated and administered in unit dosage forms or multiple dosage forms. Unit dose forms as used herein refers to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging.

Dosage forms or compositions containing active ingredient in the range of 0.005% to 100% with the balance made up from non toxic carrier can be prepared. For oral administration, pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Pharmaceutical preparation also can be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

Formulations suitable for rectal administration are preferably presented as unit dose suppositories. These can be prepared by admixing the active compound with one or more conventional solid carriers, for example, cocoa butter, and then shaping the resulting mixture.

Formulations suitable for topical application to the skin or to the eye preferably take the form of an ointment, cream, lotion, paste, gel, spray, aerosol and oil. Carriers that can be used include vaseline, lanoline, polyethylene glycols, alcohols, and combinations of two or more thereof. The topical formulations can further advantageously contain 0.05 to 15 percent by weight of thickeners selected from among hydroxypropyl methyl cellulose, methyl cellulose, polyvinylpyrrolidone, polyvinyl alcohol, poly (alkylene glycols), poly/hydroxyalkyl, (meth)acrylates or poly(meth)acrylamides. A topical formulation is often applied by instillation or as an ointment into the conjunctival sac. It also can be used for irrigation or lubrication of the eye, facial sinuses, and external auditory meatus. It also can be injected into the anterior eye chamber and other places. Topical formulations in the liquid state can be also present in a hydrophilic three-dimensional polymer matrix in the form of a strip or contact lens, from which the active components are released.

For administration by inhalation, the compounds for use herein can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Pharmaceutical compositions of therapeutic complexes and/or components can be formulated for parenteral administration by injection, e.g., by bolus injection or continuous infusion. Formulations for injection can be presented in unit dosage form, e.g., in ampules or in multi-dose containers, with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use.

Formulations suitable for transdermal administration can be presented as discrete patches adapted to remain in intimate contact with the epidermis of the recipient for a prolonged period of time. Such patches suitably contain the active compound as an optionally buffered aqueous solution of, for example, 0.1 to 0.2M concentration with respect to the active compound. Formulations suitable for transdermal administration also can be delivered by iontophoresis (see, e.g., Pharmaceutical Research 3(6), 318 (1986)) and typically take the form of an optionally buffered aqueous solution of the active compound.

Pharmaceutical compositions also can be administered by controlled release means and/or delivery devices (see, e.g., in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566).

Desirable blood levels can be maintained by a continuous infusion of the active agent as ascertained by plasma levels. It should be noted that the attending physician would know how to and when to terminate, interrupt or adjust therapy to lower dosage due to toxicity, or bone marrow, liver or kidney dysfunctions. Conversely, the attending physician would also know how to and when to adjust treatment to higher levels if the clinical response is not adequate (precluding toxic side effects). administered, for example, by oral, pulmonary, parental (intramuscular, intraperitoneal, intravenous (IV) or subcutaneous injection), inhalation (via a fine powder formulation), transdermal, nasal, vaginal, rectal, or sublingual routes of administration and can be formulated in dosage forms appropriate for each route of administration (see, e.g., International PCT application Nos. WO 93/25221 and WO 94/17784; and European Patent Application 613,683).

A therapeutic complex is included in the pharmaceutically acceptable carrier in an amount sufficient to exert a therapeutically useful effect in the absence of undesirable side effects on the subject treated. The therapeutically effective concentration can be determined empirically by testing the compounds in known in vitro and in vivo systems, such as the assays provided herein.

The concentration of therapeutic complex and components in the composition will depend on absorption, inactivation and excretion rates of the complex, the physicochemical characteristics of the complex, the dosage schedule, and amount administered as well as other factors known to those of skill in the art.

The amount of a therapeutic complex to be administered for the treatment of a disease or condition, for example cancer, autoimmune disease and viral infection can be determined by standard clinical techniques. In addition, in vitro assays and animal model can be employed to help identify optimal dosage ranges. The precise dosage will also depend on the route of administration and the seriousness of the disease. Suitable dosage ranges for administration can range from about 0.01 pg/kg body weight to 1 mg/kg body weight and more typically 0.05 mg/kg to 200 mg/kg therapeutic complex: subject weight.

A therapeutic complex can be administered at once, or can be divided into a number of smaller doses to be administered at intervals of time. Therapeutic complexes can be administered in one or more doses over the course of a treatment time for example over several hours, days, weeks, or months. In some cases, continuous administration is useful. It is understood that the precise dosage and duration of treatment is a function of the disease being treated and can be determined empirically using known testing protocols or by extrapolation from in vivo or in vitro test data. It is to be noted that concentrations and dosage values also can vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of the compositions and combinations containing them nor of the methods of use thereof.

Therapeutic complexes herein can be administered as an assembled complex of targeting domain-binding partner-capture agent-effector. In another embodiment, therapeutic complexes are delivered as sequential components. For example, a targeting domain-binding partner component is administered, followed by a capture agent-effector. In one such example, a targeting domain-binding partner component is delivered followed by a subsequent administration of an assembled therapeutic complex. In another example, a capture-agent effector is delivered first followed by a targeting domain-binding partner molecule and/or an assembled therapeutic complex. The administration of the components of a therapeutic complex can be separated by suitable intervals such as minutes, hours or days. Such intervals can be determined empirically using assays, including the use of animal models and labeled complexes and components, such as described herein.

To monitor the course of therapy, blood and tissue samples are collected and assayed for the presence of the administered therapeutic complex as well as molecules associated with the disease such as circulating antibodies, presence of B and T cell populations and cytokine levels. Safety parameters such as toxicity and symptoms of adverse reactions also are monitored. In some cases, biopsies of cells, tissues or organs are used to monitor the progress of treatment. Imaging techniques such as radioimmunoscintigraphy with radiolabeled therapeutic complexes also can be used to monitor biodistribution of the therapeutic complexes and their components.

Adjuvants and Other Combination Therapies

Adjuvants and other immune modulators can be used in combination with the therapeutic complexes described herein. Combination therapy can increase the effectiveness of treatments and in some cases, create synergistic effects such the combination is more effective than the additive effect of the treatments separately. Examples of adjuvants include, but are not limited to, bacterial DNA, nucleic acid fraction of attenuated mycobacterial cells (BCG; Bacillus-Calmette-Guerin), synthetic oligonucleotides from the BCG genome, and synthetic oligonucleotides containing CpG motifs (CpG ODN; Wooldridge et al. (1997) Blood 89:2994-2998), levamisole, aluminum hydroxide (alum), BCG, Incomplete Freud's Adjuvant (IFA), QS-21 (a plant derived immunostimulant), keyhole limpet hemocyanin (KLH), and dinitrophenyl (DNP). Examples of immune modulators useful with therapeutic complexes described herein include but are not limited to cytokines such as interleukins (e.g., IL-2, IL-3, IL-4, IL-5, IL-6, IL-7, IL-9, IL-10, IL-11, IL-12, IL-13, IL-15, IL-16, IL-17, IL-18, IL-1α, IL-1β, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligahd, CD27 ligand, CD30 ligand, 4-1BBL, Trail), and MIF, interferon, cytokines such as IL-2 and IL-12; and chemotherapy agents such as methotrexate and chlorambucil.

Combination therapies also can be administered by using two or more of the therapeutic complexes described herein. In one embodiment, two or more therapeutic complexes are directed to the same target but have different effectors. Such combination therapies can combine immunomodulatory effects, for example neutralization and T-cell induction. Such combinations also can provide costimulatory molecules to the same cell target or receptor. For example, two or more therapeutic complexes can be administered that target the T cell complex and provide effectors with costimulatory molecules such as CD2 and CD28. In another embodiment, two or more therapeutic complexes can be administered to treat a disease or condition directed to different targets. For example, an autoimmune disease can have two or more circulating autoantibodies. Therapeutic complexes can be administered to target each autoantibody. In another example, two or more therapeutic complexes can be administered to target the autoantibodies and others targeted to the autoantibody secreting cells.

### G. EXEMPLARY MOLECULES AND THERAPIES

The therapeutic complexes, components and methods described herein can be used for the treatment of a wide variety of diseases and conditions. Therapeutic complexes are designed to bind to a target associated with a disease or condition by means of a targeting domain. A targeting domain is conjugated to a binding partner which effects binding of a capture agent linked to an effector. An effector provides a biological effect to the therapeutic complex for treatment of a disease or condition. Thus, therapeutic complexes are composed of a targeting domain-binding partner moiety and a capture agent-effector moiety that specifically partner to create a targeted biological effect and/or treatment. Examples of diseases and conditions that can be treated by therapeutic complexes include, but are not limited to cancers, infectious diseases, allergies, microbial diseases, pregnancy-related diseases, bacterial diseases, heart diseases, viral diseases, histological diseases, genetic diseases, blood-related diseases, fungal diseases, adrenal diseases, liver diseases, autoimmune diseases, growth disorders, diabetes, neurodegenerative diseases, including multiple sclerosis, Parkinson's disease and Alzheimer's disease.

The following sections and subsections describe the design and use of therapeutic complexes for exemplary diseases and conditions. It is understood that these are exemplary only and other applications are intended to be included.

1. B-Cell Lymphoma

Some disease targets are more difficult to address from a technical standpoint, because each subject presents a unique target. Such is the case with non Hodgkin's Lymphoma (NHL) and other B-cell lymphomas such as leukemias. B cell lymphomas are characterized by the enhancement of specific populations of B cells, and such clonal populations are often subject-specific.

Each B cell produces a unique antibody and thus every cancerous B cell carries a unique marker. This unique surface-expressed marker is known as the cancer's idiotype marker. Unfortunately, each NHL subject has a unique and different idiotype marker. Therefore, a single drug cannot be made to target the cancers of all NHL subjects via the idiotype marker. For an effective treatment, subjects must be treated with a subject-specific therapy.

Therapeutic complexes directed against B cell diseases such as NHL can be produced using the methods and compositions provided herein. Lymphoma samples from a subject (or subjects) can be obtained and screened with a library of candidate targeting domains such as scFvs. For example, using addressable arrays displaying scFvs and antibodies, targeting domains are identified and isolated that specifically bind to the lymphoma cells. A therapeutic complex is constructed by using the isolated lymphoma-specific scFv as a targeting domain and conjugating it to a binding partner. An effector is chosen, such as an immune modulator, for example, an IgG1 or IgG2a molecule, which provides an Fc domain. A capture agent is selected that specifically binds to the binding partner and can be conjugated to the chosen effector. In one example, an effector and capture agent are chosen as one molecule that encompasses both functions, for example an immunoglobulin that specifically binds to a binding partner and that has an Fc domain to confer an immunomodulatory effect.

In another example, a monoclonal antibody, rituximab, is employed as an effector. A binding partner recognized by a variable domain of the monoclonal antibody is generated. Such binding partners can be synthetically generated from known epitope sequences or for example, by screening for epitopes bound by the monoclonal antibody. The epitope binding partner can be further optimized such that when conjugated to a targeting domain, it retains optimal binding to the monoclonal antibody. A targeting domain is generated by screening for or identifying a single chain antibody that recognizes an anti-idiotype receptor on B-cells, such as subject-specific B cells from a subject. The targeting domain is conjugated to the binding partner; a complex is assembled with the effector and binding partner-targeting domain conjugate.

Therapeutic complexes can be assembled in vitro, formulated as a pharmaceutical composition by methods such as described herein and administered to a subject. In one embodiment, subject-specific or subject-specific binding partner-targeting domain and capture-effector components for treating B-cell diseases such as B cell lymphomas, are formulated and administered separately. For example, a binding partner-targeting domain is administered initially, and then followed by administration of a capture agent-effector. Assays such as described herein, for example blood sampling and ELISAs and tumor imaging are used to monitor biological effects of the complex.

2. T-Cell Related Ocular Diseases and Conditions

Non-infectious posterior intraocular inflammation (PSII) includes uvetic disorders and ocular surface disorders such as scleritis and peripheral ulcerative keratitis are autoimmune disorders that result in visual loss. Many of the diseases are limited to immune disorders within the eye and the remainder is part of larger disorders involving connective tissue and multi-system granulomatous disorders. There is evidence for antigen specific T_{H}1 T cell activations in PSII disorders. Proinflammatory responses in uveitic subjects correspond with T_{H}1 CD4+ T cells.

The T cell receptor complex is composed of multiple transmembrane polypeptide chains on the cell surface of T lymphocytes. T cell populations are clonally expanded. Each clonal population has a unique disulfide linked alpha-beta heterodimer (Ti) that contains a site for recognition of antigen in the context of the major histocompatibility complex (MHC). The CD3 components of T cells are invariant. Thus populations of T cells can be uniquely targeted by using the Ti heterodimer. Animal studies have suggested that brief courses of monoclonal antibodies directed at T cells turn off autoaggressive responses permanently.

Therapeutic complexes directed against ocular inflammatory diseases can be produced using the methods and compositions provided herein. For example, targeting domains can be isolated that target clonal populations of T cells, for example T cells specific for retinal-specific S-antigen or interphotoreceptor retinoid binding protein can be used as targets as whole cells or as extracts. Targeting domains that bind to the target are isolated. The targeting domains then are linked to binding partners as described herein. Capture agent-effectors are provided to specifically bind to chosen binding partners and to mediate a biological effect. For example, effectors can be provided that mediate a neutralization, cytotoxic or immunosuppressive effect.

3. Lupus

Systemic lupus erythematosus (SLE) is an autoimmune disease characterized by undesirable immune responses against a broad range of self-antigens. Each subject generates a polyclonal B cell activation that produces auto-antibodies. Each subject's immune response is unique, although certain self-antigens appear to be targeted in common. SLE is characterized by antibodies against double-stranded DNA (dsDNA) as well as antibodies that recognize ribosomal proteins, neuronal antigens and phospholipids.

Using the methods and compositions herein, therapeutic complexes can be designed for SLE therapy, including subject-specific therapy. For example, samples such as blood samples, can be taken from a subject (and/or subjects) and antibodies against antigens such as DNA are isolated, for example, by affinity selection. These antibodies can then be used as targets for isolating targeting domains methods as described herein or by any methods known in the art. Targeting domains can be selected that bind to one or more of the auto-antibodies. The targeting domain is then conjugated to a binding partner. A capture agent, such as described herein, is provided that binds to a binding partner and is conjugated with an effector. Interaction of the capture agent:binding partner forms a therapeutic complex that targets an auto-antibody (and/or auto-antibodies) and mediates a biological effect. For example, a neutralization effector can be provided that removes the auto-antibodies from active circulation in the body.

In another example, specific B cell populations that secrete anti-dsDNA antibodies can be used as a target. A targeting domain that binds to the specific B cells and a binding partner is isolated and is conjugated as described herein. An effector molecule that mediates the biological effect is provided and conjugated to a capture agent that specifically binds to the binding partner. Examples of effector molecules include blockers of CD28/B7 and CD40/CD40L interactions that mediate costimulation of B cells.

4. Rheumatoid Arthritis

Rheumatoid arthritis (RA) has three main phases: initiation, an inflammatory phase and destruction of the synovium. The inflammatory phase appears to be antigen-specific and can involve auto-antigen recognition. Enhanced B cell activity also is seen in RA subjects, particularly in the inflamed synovium. In some cases, specific subgroups of B cells are amplified.

Therapeutic complexes can be designed for rheumatoid arthritis treatment using the compositions and methods described herein. For example, targeting domains can be isolated that recognize specific B cell populations from subject samples. A binding partner can then be conjugated to the selected targeting domain. A capture agent is then selected or generated that binds to the binding partner and conjugated to an effector that provides a biological effect. For example, an effector can be chosen that results in the destruction of the targeted B-cells.

In another example, targeting domains can be isolated that bind to auto-antibodies found in RA samples. Such targeting domains are conjugated with a binding partner. A capture agent is then selected or generated that binds to the binding partner and conjugated to an effector that provides a biological effect. For example, a neutralizing effector can be chosen such that once the circulating auto-antibodies are bound by the therapeutic complexes, the complex is removed from circulation.

5. Multiple Sclerosis

Evidence suggests autoantigens are involved in the pathogenesis of multiple sclerosis (MS). One such autoantigen is myelin basic protein (MBP). MBP-specific T cells have been found in the cerebrospinal fluid of MS subjects. Further, T cells isolated from MS subjects have specificity for a variety of MBP epitopes. Although subjects share some commonalities of immunodominant epitopes recognized by these T cells, a wide variety of subject-specific-responses have been observed.

Therapeutic complexes for MS can be designed using the methods and compositions described herein. For example, samples such as T cells from an MS subject (or subjects), can be used as the target. T-cells specific for MBP can be isolated using methods such as the “split-well” method that allows rapid isolation of antigen specific CD4+ cells using an antigen-specific proliferation assay (Meinl et al. (1993) J Clin. Invest. 92: 2633-2643). Specific MBP peptides can be used to map the epitopes recognized by the T cell samples. Controls taken from healthy donors can optionally be used to map epitopes recognized also by T-cells from non-diseased samples. Disease-specific T cells (those recognizing MBP epitopes present only in the MS sample(s)) can be chosen as targets. The cells can be used directly to screen for targeting domains or the variable domains of the T cell receptors from such lines can be isolated and used as targets.

Targeting domains are selected that are specific for the chosen T cell target(s) and the targeting domains are then conjugated to a binding partner that binds to a capture agent. An effector molecule is chosen to provide the desired biological effect and additionally, such that it can be conjugated to a capture agent. For example, an effector can be chosen that confers a cytotoxic effect such that the targeted therapeutic complex effects destruction of the targeted clonal T cell population. In another example, an effector is chosen that blocks T cell signaling and thereby preventing T-cells from becoming activated by the MBP epitope.

6. Retargeting Therapeutic Agents

The methods herein can be employed to retarget molecules and complexes of molecules, such as therapeutic molecules and agents by assembling such molecules into therapeutic complexes. Such methods can be used to alter or extend target specificity of a molecule that binds to a target, T1, by providing additional or alternate targeting domains. Such domains can increase the specificity or avidity of binding to target T1 and/or provide specific binding to one or more additional targets different from T1. For example, molecules that bind to non-subject-specific targets and confer a biological effect can be retargeted to subject-specific targets. In another example, molecules can be retargeted such that there are more binding sites available for the retargeted molecules as compared to the starting molecule. Such retargeted molecules, thus, could have an enhanced or otherwise altered therapeutic effect on a target.

Molecules for retargeting include molecules that confer a biological effect, e.g. effectors and effector domains as described herein. Molecules for retargeting include, but are not limited to, antibodies, antibody fragments and immunotoxins. Such molecules for retargeting include molecules that bind to a target (also referred to herein as T1 and/or original target).

Retargeted can be accomplished by assembling a molecule to be retargeted, referred to herein as M1 or molecule M1, into therapeutic complexes, thereby associating M1 with one or more new or additional targeting domains. A new or additional targeting domain can be directed to a new or additional target of interest or can increase or enhance specificity for an original target (T1). The new or additional target, thus, can be the same or different from the original target of molecule M1, and can be in addition to the original target of M1. Retargeting also can be accomplished by adding a targeting domain with specificity for the same target, such as a tumor cell, but by targeting or binding to a different molecule, such as a different cell surface marker. This can result, for example, in more molecules of the therapeutic complex binding to a target cell compared to molecules of M1 alone bound to a target cell, and/or increased specificity and/or conferring additional biological effects (e.g. participate in co-stimulation).

Formation of a therapeutic complex that retargets the molecule M1 occurs via capture agent—binding partner interactions. Generally, a molecule M1 chosen for retargeting confers a biological effect and thus M1 or a portion thereof serves as an effector. In addition, molecule M1 can include or serve as the capture agent for formation of the complex. A binding partner can be selected or produced that specifically binds to the capture agent and can be conjugated to the targeting domain.

These methods have a variety of applications for retargeting molecules, for example, expanding the uses and applications of known molecules with biological effects. Any targeted molecule can be retargeted as described herein or by methods adapted therefrom. Exemplary molecules for retargeting include, but are not limited to, immunomodulatory molecules, immunotoxins, antibodies and other conjugates, such as antibody conjugates and antibody fragment-conjugates.

a. Retargeting Antibodies

In one exemplary embodiment, molecule M1 to be retargeted is an antibody or antibody fragment, such as a monoclonal antibody. The antibody can be any immunoglobulin, such as an IgG molecule, including but not limited to IgG1 and other antibodies, such as a humanized antibody, which confers a biological effect, such as an immunomodulatory effect. The antibody can be an IgG molecule such as a IgG2a that confers a biological effect in mice and that can be used for example, for testing of retargeting in a mouse model. The molecule to be retargeted also includes antibody fragments, such as, but not limited to, a Fab or single chain antibody. Complexes of molecules also can be retargeted for example, complexes of antibodies, such as bispecific antibodies.

In one aspect of the embodiment, a portion of molecule M1, such as one or more variable domains where M1 is an antibody, serves as a capture agent. A binding partner is generated that specifically binds to all or a portion of the variable domain of the antibody. For example, if a variable domain of an antibody acts as a capture agent, an epitope for that variable domain, such as a polypeptide or small molecule to which the variable domain specifically binds can be used as a binding partner. The binding partner is conjugated to a targeting domain to be used to retarget the antibody. A targeting domain can be chosen from any of the targeting domains such as those described herein. For example, a targeting domain is chosen that specifically binds to a subject-specific target; for example, a targeting domain can be an anti-idiotype antibody. Among molecules for use in retargeting are included antibodies, antibody fragments and antibody complexes and any molecule containing a variable domain of an antibody can be retargeted to redirect therapeutic effects, enhance therapeutic effects and/or additionally for use in combination therapies.

b. Subject-Specific Retargeting

Retargeting can be used to render an existing therapeutic subject-specific by adding a subject-specific targeting domain. For example, a monoclonal antibody is retargeted to an idiotypic marker such as an idiotype receptor, converting the specificity of the antibody into a subject-specific therapeutic complex. Thus, for example, a monoclonal antibody can be chosen for example, based on its biological effect, its antigenicity (or lack thereof) in a subject and other desirable properties, such as monoclonal antibodies approved for use in humans by the Food and Drug Administration (FDA), and then modified as described herein.

A monoclonal antibody (or other antibody or binding fragment thereof) generally contains one or more variable domains, which specifically binds to antigens (or an epitope) and for purposes herein can serve as a capture agent(s) (binding partner), and a constant domain, which is as an effector. A binding partner includes an epitope or other locus to which a variable domain of a monoclonal antibody binds. An epitope includes any molecule or locus thereon or in or on a surface to which a variable domain specifically binds. For example, an epitope includes, but is not limited to, a sequence of amino acids, in linear or three dimensional conformation, a carbohydrate or a small molecule. An epitope also can be a portion (locus) of a larger molecule, such as a portion of a receptor or cell-surface molecule.

Interaction of the variable domains of a monoclonal antibody with a binding partner can mask the ability of the variable domains to specifically bind to a target. By virtue of such masking a monoclonal antibody is retargeted to a new target by interaction of the variable domains with a binding partner conjugated to a targeting domain.

Antibodies (or fragments thereof) that can be retargeted include any antibody of fragment that has a target, and particularly includes therapeutic and other commercially available antibodies, such as, for example, rituximab (Rituxan®). rituximab is a genetically engineered chimeric mouse/human IgG1 Kappa monoclonal antibody directed against B-cell lymphoma. rituximab binds to CD20 antigen expressed on the surface of normal and malignant B lymphocytes, including ˜90% of non-Hodgkin's lymphomas. The variable domains (Fabs) of rituximab specifically bind to CD20 or a portion thereof. rituximab contains an Fc domain that causes cells to lyse through ADCC or complement-dependent cytotoxicity, apoptosis and/or other related mechanisms. rituximab can be retargeted by the methods herein to bind to additional molecules on targeted lymphocytes or to have increased specificity for the targeted site or to be directed to other cells.

For example, a therapeutic complex is constructed using rituximab as an effector-capture agent. A binding partner is constructed from CD20 or a portion thereof sufficient to specifically bind to the variable domain of rituximab. The CD20-binding partner is conjugated to a targeting domain. For example, subject-specific targeting domains can be generated, such as described herein, to specifically bind to subject-specific targets, such as subject-specific targets on B-cells, for example anti-idiotype receptors. A therapeutic complex of rituximab and CD20-binding partner-targeting domain is assembled in vitro or in vivo by methods described herein or by methods known to those of skill in the art. The targeting domain retargets rituximab through the interaction of the CD20-binding partner with the variable domain (capture agent) of rituximab. This type of retargeting by preparing therapeutic complexes can be performed on any antibody or specific binding molecule or complex or conjugate.

Thus, other antibodies, such as monoclonal antibodies, can be retargeted in a similar manner. Any antibody or specific binding agent for which an epitope is known or can be identified or can be generated synthetically can be retargeted. Other examples of specific binding molecules that can be assembled into therapeutic complexes and retargeted include, but are not limited to, anti-Her-2 monoclonal antibody trastuzumab (Herceptin®), anti-CD20 monoclonal antibodies tositumomab (Bexxar®) and Ibritumomab (Zevalin®), anti-CD52 monoclonal antibody Alemtuzumab (Campath®), anti-TNFα antibodies infliximab (Remicade®) and CDP-571 (Humicade®), monoclonal antibody edrecolomab (Panorex®), the anti-CD3 antibody muromab-CD3 (Orthoclone®), anti-IL-2R antibody daclizumab (Zenapax®), omalizumab antibody against IgE (Xolair®), monoclonal antibody bevacizumab (Avastin®) monoclonal antibody against EGFR cetuximab (Erbitux®), and any other such antibodies and fragments thereof and specific binding molecules, such as enzymes, including those that are modified to reduce or eliminate catalytic activity.

c. Retargeting of Therapeutic Complexes, and Therapeutic Complexes with a Plurality of Targeting Domains and/or Effector Domains

Thus, therapeutic complexes that contain a plurality of targeting domains and one or more effector domains are provided herein. Such retargeting or targeting to a plurality of sites can be used, for example, to expand the subject population for which a drug or therapeutic is effective by adding a variety of targeting domains, each specific for different alleles. In other embodiments, the target of a therapeutic molecule, particularly an antibody, by adding a targeting domain that supplements or replaces an existing targeting domain. Thus, a therapeutic can be rendered subject specific or its specificity and/or target(s) can be altered or increased. In an exemplary embodiment, a therapeutic molecule for use in treating non-Hodgkin's lymphoma, in which an effector domain and capture agent are contained within a molecule (designated M1) is retargeted. M1, for example, is the monoclonal antibody rituximab (Rituxan®). M1 can be retargeted by forming a therapeutic complex of M1 and a targeting domain to retarget rituximab to render it more subject specific or to render it capable of binding to a larger number of subjects by adding an additional targeting domain(s). Thus, targeting domains for the therapeutic complex are selected that specifically bind to a target molecule, for example, CD20 from a subject, by cloning scFvs from a panel of hybridomas producing different monoclonal antibodies to the target molecule CD20. A common binding partner is selected that specifically binds to rituximab, for example, by designing a polypeptide that specifically binds to variable domains of rituximab. The common binding partner is conjugated to each targeting domain, such that the different scFv-binding partner conjugates contain the common binding partner. Binding partner-targeting domains are mixed in different ratios and then combined with rituximab (effector-capture agents) to form different mixtures of therapeutic complexes. Ratios of mixing include, but are not limited to, 1:1, 1:2, 1:4, 1:10, 1:15 and 1:20 or 1:greater than 20. Such therapeutic complexes can be used to treat subjects that are refractory to treatment with rituximab alone (uncomplexed with the scFvs).

In another exemplary embodiment, a therapeutic complex is constructed for use in treating non-Hodgkin's lymphoma, in which an effector domain and capture agent are contained within a molecule to be retargeted (designated M1). The monoclonal antibody rituximab (Rituxan®) is selected as an exemplary molecule M1. A targeting domain is selected for retargeting that specifically binds to a subject specific idiotype receptor; for example, an anti-idiotype scFv. A binding partner is joined to the targeting domain. A binding partner is employed that specifically binds to rituximab, for example, by designing a polypeptide or small molecule containing a region specifically bound by variable domains of rituximab. The anti-idiotype scFv is conjugated to the selected binding partner. The binding partner-targeting domain conjugate is combined with rituximab to assemble a therapeutic complex in which the rituximab is retargeted to idiotype receptors. Such complexes can be used to treat subjects with non-Hodgkin's lymphoma, such as subjects that are refractory to treatment with rituximab (uncomplexed with the scFv), or as a supplemental to or combination treatment with rituximab.

In another exemplary embodiment, a therapeutic complex is constructed for use in treating non-Hodgkin's lymphoma, in which an effector domain and capture agent are contained within a molecule to be retargeted (designated M1). The monoclonal antibody rituximab (Rituxan®) is selected as molecule M1. A plurality of targeting domains are used for retargeting that specifically bind to a subject specific idiotype receptor. For example, targeting domains are cloned from a panel of hybridomas producing different monoclonal antibodies to a target molecule, such as a subject specific idiotype receptor. A binding partner is selected that specifically binds rituximab, for example, by designing a polypeptide or small molecule containing an epitope specifically bound by variable domains of rituximab. The binding partner is joined to each targeting domain, such that the same binding partner is common to each of the different scFv-binding partner conjugates. The anti-idiotype scFvs are separately conjugated to the selected binding partner. The binding partner-targeting domains are combined in different ratios and then combined with rituximab to form sets of therapeutic complexes, containing different ratios of the different targeting domains. Such complexes can be used to treat non-Hodgkin's lymphoma subjects including subjects that are refractory to treatment with rituximab (uncomplexed with the scFv), or as a supplemental or combination treatment to rituximab.

In yet another example, a therapeutic complex is created to treat colorectal cancer. A molecule such as the antibody bevacizumab (Avastin®) is selected for retargeting. Targeting domains are selected that are different scFvs and bind to vascular endothelial growth factor (VEGF; a target molecule of bevacizumab). For example, the scFvs are cloned from a panel of hybridomas producing different monoclonal antibodies to VEGF. The targeting domains are separately conjugated to a common binding partner such as a polypeptide that is specifically bound by bevacizumab. The binding partner-targeting domain conjugates (binding partner-anti-VEGF scFvs) are combined in different ratios and then combined with bevacizumab to form sets of therapeutic complexes. Such complexes can be used in the treatment of colorectal cancers including treatment of subjects that are refractory to treatment with bevacizumab (uncomplexed with the scFvs).

In another example, a therapeutic complex is produced to treat colorectal cancer in which the starting molecule is the antibody cetuximab (Erbitux®). Targeting domains are selected that specifically bind to the target molecule epidermal growth factor receptor (EGFR); for example, scFvs are cloned from a panel of hybridomas producing different monoclonal antibodies to EGFR. The targeting domains are separately conjugated to a common binding partner that is a polypeptide that is specifically bound by cetuximab. The binding partner-targeting domain conjugates are combined in different ratios and then combined with cetuximab to form sets of therapeutic complexes. Such complexes can be used in the treatment of colorectal cancers such as treatment of subjects that are refractory to treatment with cetuximab (uncomplexed with the scFvs).

In another example, a therapeutic complex is created to treat metastatic breast cancer, in which the molecule to be retargeted, M1, is the molecule trastuzumab (Herceptin®) can be rendered subject specific by adding a targeting domain, which masks or supplements that targeting portion of trastuzumab. Targeting domains are selected that specifically bind to the target molecule HER2 receptor, a growth factor-like receptor and a target molecule of trastuzumab. Such targeting domains can be cloned from a panel of hybridomas producing different monoclonal antibodies to HER2 receptor. Targeting domains are separately conjugated to a common binding partner that is a polypeptide that is specifically bound by trastuzumab. The binding partner-targeting domain conjugates are combined in different ratios and then combined with trastuzumab to form sets of therapeutic complexes. Such complexes can be used in the treatment of metastatic breast cancer including subjects that are refractory to treatment with trastuzumab (uncomplexed with the scFvs).

The following examples are included for illustrative purposes only and are not intended to limit the scope of the invention.

### H. EXAMPLES

### Example 1

Targeting Domain—Target Interactions

A. MicroArray Printing

Stock solutions of the anti-IgM antibody (S1C5; anti-idiotype monoclonal antibody), the goat anti-mouse Fc antibody (this antibody recognizes the constant (Fc) regions of mouse antibodies) and anti-flag antibody were prepared at a concentration of 1 mg/ml or greater in PBS. For printing, the antibodies were brought to 800 μg/ml in 1× Print Buffer (1× PBS, 20% glycerol, 0.001% Tween-20) by adding ¼ volume of 4× Print Buffer (4× PBS, 80% glycerol, 0.004% Tween-20) to ¾ volume of a 1 mg/ml antibody solution in PBS. Two-fold serial dilutions were made of each antibody such that all antibodies were at 9 different concentrations in 1× Print Buffer. Forty μl of antibody solution was transferred to a 96-well PCR plate.

Each of the antibodies were printed on FAST nitrocellulose—coated glass slides (Schleicher and Schuell) using a Telechem pin (CM-2) in a Cartesian printer (MicroSys 5100) as described in U.S. application Ser. No. 10/699,088. Printing was performed at 55 to 60% relative humidity. The slides were subsequently incubated overnight at 4° C. for maximum adsorption to the nitrocellulose.

B. Preparation of 38C13 Cell Extract

B cells (38C13) were grown in culture (Growth medium: RPMI 1640, 10% fetal calf serum, 55 μl 2-mercaptoethanol, penicillin and streptomycin) in 5% CO₂, 90% relative humidity and 37° C. to a density of 0.7×10⁶ cells/ml. A 2.5 ml aliquot (1.75×10⁶ cells total) was spun down at 1200 rpm for 5 minutes at 4° C. The pellet then was washed one time with 4 ml of RPMI-1640 (Gibco), and spun down again at 1200 rpm for 5 minutes at 4° C. The pellet then was resuspended at 4° C. in 175 μl of RPMI-1640 (Gibco), giving a concentration of 10⁶ cells per 100 μl. Resuspension was carried out by gently pipetting up and down 3-4 times.

Small (less than 1 ml) aliquots of tissue culture cells (38C13 and C6VL cells) prepared as described above were stored frozen in liquid nitrogen or at −80° C. in Freezing Medium (frequently 90% fetal calf serum/10% DMSO). The frozen cells were thawed quickly by rolling tube containing the aliquot between the palms. The cells were diluted immediately 10-fold with 4° C. PBS and centrifuged at 1200 rpm for 5 minutes at 4° C. Cells were then washed three times with 4° C. PBS at a density of 10⁶ cells/ml, based on the number of cells that were frozen for storage. The resuspended cells were used immediately for capture.

C. Array Incubations

The printed slides were brought to room temperature and washed three times each for one minute with PBS. Following the wash step, the slides were blocked with 1 ml of Block Buffer (3% NMF/PBS/1% Triton X-100) on an orbital shaker in a humidified chamber for 1 hour at room temperature. The slides were then incubated with 38C13 cell extract and control 38C13 purified antibody. The extract was diluted 1:1 with Block Buffer for the highest concentration, then serially by factors of 10. Fifty μl of each sample was added to the wells and incubated with the array for 1 hour at room temperature on an orbital shaker.

Following the incubation, the wells were then washed three times with 200 μl of PBS/1% Triton X-100 for one minute on an orbital shaker. Fifty microliters of detection antibody (goat anti-mouse IgM HRP 1:5,000 in Block Buffer) were then added to each well and incubated for one hour at room temperature on an orbital shaker. The wells were then washed again three times with 200 μl of PBS/1% Triton X-100 for one minute on an orbital shaker. The slides were then removed from the chamber and rinsed with 500 μl PBS/1% Triton X-100. The arrays were then imaged on Kodak IS 1000 in a petri dish, raised from the surface of the dish with two layers of plastic cover slips, with about 1 ml of luminol.

D. Results

The purified IgM antibody (38C13) gave a strong signal on the SIC5 monoclonal antibody loci, down to a concentration of 25 μg/ml spotted protein and at an IgM concentration of 0.1 μg/ml, the lowest IgM concentration used. The 38C13 IgM in the 38C13 cell extracts were detected at a 1:2000 dilution of the extract, the lowest used, down to a concentration of 50 μg/ml printed S1C5. The 38C13 IgM did not bind to the anti-Flag monoclonal negative control, though non-specific binding of the Goat anti-Mouse IgM—HRP antibody can be seen.

### Example 2

Targeting domain-interactions with Cells Expressing a Target

A. MicroArray Printing

Stock solutions of the anti-M2 capture monoclonal antibody (M2), anti-Myc capture monoclonal antibody (Myc), anti-IgM (S1C5; anti-idiotype monoclonal antibody) and anti-T cell receptor antibody (C6VL) were prepared at concentrations of 1 mg/ml or greater in PBS. For printing, the antibodies were brought to 800 μg/ml in 1× Print Buffer (1× PBS, 20% glycerol, 0.001% Tween-20) by adding ¼ volume of 4× Print Buffer (4× PBS, 80% glycerol, 0.004% Tween-20) to ¾ volume of a 1 mg/ml antibody solution in PBS. Two-fold serial dilutions were made of each antibody such that all antibodies were at 9 different concentrations in 1× Print Buffer. Forty μl of antibody solution was transferred to a 96-well PCR plate.

Each of the antibodies were printed on FAST nitrocellulose - coated glass slides (Schleicher and Schuell) using a Telechem pin (CM4) in a Cartesian printer (MicroSys 5100). Printing was performed at 55 to 60% relative humidity. The slides were subsequently incubated overnight at 4° C. for maximum adsorption to the nitrocellulose.

B. Preparation of Non-Adherent Cells for Capture

B cells (38C13) and T cells (C6VL) were grown in culture (Growth medium: RPMI-1640, 10% fetal calf serum, 55 μl 2-mercaptoethanol, penicillin and streptomycin) in 5% CO2, 90% relative humidity and 37° C. 38C13 B cells were grown to a density of 0.7×10⁶ cells/ml in growth medium. A 2.5 ml aliquot (1.75×10⁶ cells total) was spun down at 1200 rpm for 5 minutes at 4° C. The C6VL T cells were grown to a density of 0.35×10⁶ cells/ml in growth medium. A 5 ml aliquot (1.75×10⁶ cells total) was spun down at 1200 rpm for 5 minutes at 4° C. The two pellets were then washed one time with 4 ml each of RPMI-1640, and spun down again at 1200 rpm for 5 minutes at 4° C. The two pellets were then resuspended at 4° C. in 175 μl of RPMI-1640, giving a concentration of 10⁶ cells per 100 μl. Resuspension was carried out by gently pipetting up and down 3-4 times. The resuspended cells were used immediately for capture.

C. Cell Capture Assay

1. Monoclonal Anti-Cell Surface Antigen Arrays

The printed slides was brought to room temperature and washed three times each for one minute with PBS. Following the wash step, the slides were blocked with I ml of PBS containing 0.5% Bovine Serum Albumin on an orbital shaker in a humidified chamber for 1 hour at room temperature.

Following the blocking, excess Block Buffer was removed by tilting the slide and absorbing liquid from the lower end with a Kimwipe. One Hundred μl (containing 10⁶ cells total in Incubation Buffer) of C6VL cells (T cells) were added to one slide and 100 μl (containing 10⁶ cells total in Incubation Buffer) of 38C13 cells (B cells) were added to the second slide by pipetting cells down the middle of the slides in sequential drops. The slides were then incubated again for 20-30 minutes at room temperature on an orbital shaker. Following the incubation, the slides were viewed immediately in a microscope differential interference contrast (DIC) microscopy (Nikon E800 with Locus CCD Camera). Optionally, the slides were gently washed first in Incubation Buffer at room temperature then viewed as above. In all cases, the printed slide was situated in the microscope such that the printed side with the cells was facing up.

2. Monoclonal Antibody/Binding Partner-scFv Arrays

Printed slides were incubated for 1 hour in Block Buffer as described above. Following the incubation, a mask was placed on the slide to create wells and separates the arrays. Peptide tag—scFv fusion protein (binding partner-scFv fusion), previously purified from bacteria by His-tag metal affinity chromatography, and stored in PBS at about 1 mg/ml, was diluted 10-fold or more into Incubation Buffer. The slides were then incubated for 1 hour at room temperature with the purified peptide tag-scFv (1 ml/slide or if slides are in the 10—well mask, 50 μl/well) on an orbital shaker in either a humidified chamber or with an adhesive seal over the mask. The slides were washed 3 times with 200 μl of Incubation Buffer, I minute each time on an orbital shaker and then incubated with cells at 10⁷ cells/ml in Incubation Buffer for 20-30 minutes. One hundred μl was used for an entire slide. If slides were masked, then 50 μl of a 2×10⁶ cells/ml solution were applied per well. Slides were viewed directly in a microscope, or, optionally, gently washed first in Incubation Buffer then viewed in a microscope. In a mask, slides were washed 3 times with 400 μl Wash Buffer (0.5% BSA with buffered salt solution containing either culture medium with 10 mM HEPES pH 7.4, lacking phenol red, or PBS) one minute each time, on an orbital shaker at room temperature. Excess Wash Buffer was removed after each wash by aspirating all but about 100 μl of Buffer.

D. Chemical Fixation of Cells to Arrays

Following cell capture on the arrays, cells were fixed with a 4% Formaldehyde Solution. The 4% solution was prepared by diluting 37% formaldehyde (Histology Grade, Sigma) 10-fold into the buffered salt solution used for capture. Following capture, excess Wash Solution was removed from the slide by tilting it and absorbing the run-off with a Kimwipe. The slide then was placed horizontally in a humidified chamber and 1 ml of the 4% Formaldehyde Solution was added to the array surface in drops along the length of the slide. The slide then was incubated at room temperature for 10 minutes and washed 3 times for 5 minutes each with 50 ml each time of PBS in either Complin jars or 50 ml conical tubes. Cells were permeabilized with Permeabilization Solution (0.1% TX-100, PBS and 0.02% sodium azide) for 5 minutes at room temperature. The slides were then stored at 4° C. in the Permeabilization Solution.

E. Results

The cells were captured on the arrays in a target domain dependent manner. 38C13 cells were captured by the S1C5 antibody loci (which recognizes IgM on the 38C13 cells) and T cells were captured by the anti-C6V loci. The antibody against human fibronectin was used as a negative control. There was no cross-reactivity between the control antibody and the cells. Cells bound only to the antibodies that were specific for antigens expressed on their cells surface.

### Example 3

Assembling a Targeting Domain-Binding Partner: Capture Agent-Effector Complex

A. Cloning of SiC5: scFv Targeting Domain with a Binding Partner

Hybridoma cells expressing S1C5:IgG (Maloney et al. (1985) Hybridoma 4(3):191-209; see also U.S. Pat. No. 6,099,846) were used for extraction of PolyA+ RNA. First strand cDNA was synthesized from 2 μg polyA+ RNA using random hexamers and Superscript® II reverse transcriptase (Invitrogen; Carlsbad, Calif.) according to standard protocols.

scFv was prepared from the cDNA following the protocol described by Burmester and Pluckthun (pages 19-40 in Antibody Engineering: Construction ofscFv fragments from hybridoma or spleen cells by PCR assembly, Kontermann and Duibel eds. Springer-Verlag (2001)). Briefly, the cDNA was amplified using sets of primers to generate 5 different mixes of heavy chain and light chain fragments. These were mixed in equimolar amounts along with a linker fragment to generate the scFv by PCR amplification. Restriction sites were incorporated at both ends of the scFv (SfiI at the 5′ end and NotI at the 3′ end) to enable cloning into bacterial expression vectors.

The scFv then was cloned into the pBAD:tag:His vector (Invitrogen, Carlsbad, Calif.) containing a binding partner selected from Table 4 below, to generate the targeting domain-binding partner moieties.

TABLE 4
Exemplary Binding Partners for scFv fusions

| | | | |
|---|---|---|---|
| | Amino acid | | |
| Tag | sequence | Length | SEQ ID NO: |

| | | | |
|---|---|---|---|
| E-tag | GAPVPYPDPLEPR | 13 | 912 |
| Flag | DYKDDDDK | 8 | 913 |
| Glu-Glu | EEEEYMPME | 9 | 914 |
| HA.11 | YPYDVPDYA | 9 | 915 |
| HSV | QPELAPEDPED | 11 | 916 |
| Myc | EQKLISEEDL | 10 | 917 |
| T7 | MASMTGGQQMG | 11 | 918 |
| VS | GKPIPNPLLGLDST | 14 | 920 |
| VSV-G | YTDIEMNRLGK | 11 | 919 |
| Ab2 | LTPPMGPVIDQR | 12 | 921 |
| Ab4 | QPQSKGFEPPPP | 12 | 922 |

B. Expression of S1C5:scFv

ScFv-tag clones were used to transform E. coli Top10 Cell (Invitrogen, Carlsbad, Calif.) and the transformed bacteria were screen for expression of the recombinant protein using an anti-(His)₆ antibody-HRP conjugate. The plasmid chosen, positive for scFv-tag expression, was sequenced to ensure lack of cloning and PCR errors. The E. coli cells transformed with the plasmid expressing the scFv-tags were then grown and induced for expression with arabinose according to standard protocols.

The cells were harvested and periplasmic proteins were extracted as described by Lindner et al., (Methods: A Companion to Methods in Enzymology (1992) 4:41-56). ScFv-tag fusions were purified using an Ni—NTA sepharose column and then used in subsequent assays and methods. Initial analysis included (1) measuring the amount of purified protein using a BCA assay (2) assessing homogeneity by SDS-PAGE and (3) determining functionality by measuring the binding of the scFv-tag to its cognate antigen (target) 38C13:IgM.

C. Forming Complexes of the S1C5 scFv-Tag with the Effector

An anti-tag antibody (IgG2a) was used in this experiment as the effector-capture agent. The scFv-tag was incubated with the corresponding anti-tag antibody (i.e. the antibody that binds to the tag selected for the fusion, as described above). Anti-tag antibody was incubated with a 4-fold molar excess of scFv-tag in sterile phosphate-buffered saline (PBS) for 60 minutes at 37° C. The complex then was passed through a 0.2μ filter for sterilization. Complex formation was determined by western blotting an aliquot (1%) of the complex after native PAGE and the blot was developed with an anti-(His)₆ antibody-HRP conjugate (that binds to the scFv in the complex). A band at ˜220 kDa was detected (antibody is 150 kDa and the two scFv-tag molecules are each ˜36 kDa). This assay successfully demonstrated the assembly of a targeting domain-tag:capture agent-effector complex.

D. Assessment of Interaction of the Complex with the Target

The ability of the complex to bind to the target, antigen 38C13:IgM, on the surface of cells was assessed. Briefly, 38C13 cells expressing the 38C13:IgM on the cell membrane) were added to wells of a 96-well round bottom plate (10³-10⁴ cells/well). The cells were washed with PBS containing 1% bovine serum albumin (BSA) at 4° C., followed by incubation on ice for 30 minutes with the scFv-Tag-anti-tag antibody complex diluted 4-fold in washing buffer (PBS+1% BSA). The cells were then washed and incubated on ice for 30 minutes with the detection antibody goat anti-mouse IgG conjugated to Alexa-488. The cells were washed, fixed and visualized using a Nikon E800 fluorescence microscope. The scFv-Tag-anti-tag antibody complex (targeting domain-binding partner-capture agent-effector) was successfully detected using this assay.

### Example 4

Therapy with a Targeting Domain-Binding Partner-Capture Agent-Effector Complex

Six groups of 8 C3H/HeN mice each (10±2 weeks old) were injected with 10³ 38C13 lymphoma cells in PBS either intraperitoneally (i.p.) or sub-cutaneously (s.c.). Three hours after lymphoma injection, groups were injected i.p. with varying doses of the S1C5: scFv:V5 complex, individual components of the complex or with control reagents as shown in Table 5 below. Mice were then monitored over a 90 day period for survival.

TABLE 5

| | | | amount |
|---|---|---|---|
| | Reagent for | | injected per |
| Group | injection | component | mouse |
| 1 | PBS | Negative control | |
| 2 | S1C5:scFv | targeting domain | 25 μg |
| 3 | S1C5:scFv:V5 | complex | 100 μg |
| | complex | | (˜0.67 nmol) |
| 4 | huFN:scFv: V5 | complex with control | 100 μg |
| | complex | targeting domain | |
| 5 | V5:IgG | Positive control | 100 μg |
| 6 | V5 | Effector-capture agent | 100 μg |

Results:

The negative control mice (group 1), injected only with PBS, began exhibiting mortality at day 18, with a sharp decrease in live mice at days 22-23, such that only a single mouse remained alive after day 23, which later died at day 34. The positive control mice had a high survival rate through day 30 (7 out of 8 mice), and then gradually decreased to 50% survival by the end of the experiment on day 42.

The mice injected with the targeting domain (group 2) followed a similar pattern to group 1, although no mice survived after day 23. The mice injected with the complex constructed with a control targeting domain (recognizing human fibronectin) exhibited a similar pattern to groups 1 and 2, with a sharp decrease in survival at day 21-23, with a single mouse remaining between days 23-31. The capture agent-effector alone (group 6) had a gradual decrease in survival from day 17-32. The complex of targeting domain-binding partner:capture agent-effector (group 3) showed a high survival rate through day 25, followed by a sharp decrease on day 26 to 50% survival and gradually decreasing through day 31.

In general, the mice with individual components or control complex reached 50% mortality at days 20-22, and exhibited the greatest increase in mortality at about day 21. Whereas the complex reached 50% mortality at day 26 and exhibited the greatest change in mortality at days 25-27.

### Example 5

Generation of Additional Binding Partner-Capture Agent Pairs

A. Generation of 6-mer Polypeptides

A collection of 6 amino acid polypeptides (6-mers) were designed using the method described herein for designing HAHS polypeptides. The polypeptides were designed for screening suitability and use as binding partners paired with capture agents.

Peptides (6-mers) were synthesized with a C terminal cysteine residue as: cysteine-(amino acid)₆-NH₂. Diphtheria toxoid was activated using MCS to add maleimido groups to lysine side chains (Lee et al. (1985) Mol. Immunol. 17:749 756). A 1.5 molar excess of the activated carrier protein was incubated with the polypeptides. The ratio ensures the lack of free unconjugated polypeptides such that unconjugated polypeptides or carrier proteins are not separated from the conjugated sample.

The 6-mer polypeptides also are synthesized with biotin at the C terminal end with a 4-mer linker polypeptide for use in screening assays: Biotin-SGSG-(amino acid)₆-NH₂.

B. Immunization of Mice with DT-Peptide Conjugates

The DT peptide conjugates were dissolved in PBS. To formulate the mixture of conjugates, 0.5 mg of each of four peptides is added into one tube and the volume made to 2 ml with sterile PBS. The conjugates are mixed well before dispensing so that any particulate is well suspended. Each group of four polypeptide conjugates is designated by a group name, for example, as Grp1, Grp2, Grp3, and so on.

Three mice were immunized with each group of polypeptide conjugates. Mice were immunized with 200 μg protein/mouse for initial immunization (day 0) and boosts of 100 μg protein/mouse at days 21, 35, 49 and 63. Tail bleeds were taken at day 42 and day 70 and analyzed by ELISA assays. Samples of serum were taken from tail bleeds of the mice before day 0 immunizations to serve as pre-immune control serum.

Mice were analyzed by ELISA as follows. Biotinylated polypeptides were dissolved in DMSO at final concentrations of 5 mg/ml. NUNC Maxisorp plates are coated with 5 μg/ml Neutravidin in PBS and incubated at 4° C. until use (up to 30 days). The Neutravidin is aspirated off and the plates incubated with biotinylated polypeptides at 5 μg/ml in PBS for 60 min at 37° C. as indicated in Table 6.

TABLE 6

| | Plate 1 | Plate 2 | Plate 3 | Plate 4 | Plate 5 | Plate 6 |
|---|---|---|---|---|---|---|
| A | Peptide 1 | Peptide 9 | Peptide 17 | Peptide 25 | Peptide 33 | Peptide 41 |
| B | Peptide 2 | Peptide 10 | Peptide 18 | Peptide 26 | Peptide 34 | Peptide 42 |
| C | Peptide 3 | Peptide 11 | Peptide 19 | Peptide 27 | Peptide 35 | Peptide 43 |
| D | Peptide 4 | Peptide 12 | Peptide 20 | Peptide 28 | Peptide 36 | Peptide 44 |
| E | Peptide 5 | Peptide 13 | Peptide 21 | Peptide 29 | Peptide 37 | Peptide 45 |
| F | Peptide 6 | Peptide 14 | Peptide 22 | Peptide 30 | Peptide 38 | Peptide 46 |
| G | Peptide 7 | Peptide 15 | Peptide 23 | Peptide 31 | Peptide 39 | Peptide 47 |
| H | Peptide 8 | Peptide 16 | Peptide 24 | Peptide 32 | Peptide 40 | Peptide 48 |

The plates were blocked with 1× Blocker BSA in PBS T for 60 min at 37° C. One hundred microliters of each tail bleed sample is added to Row A at a 1:100 dilution (2.5 μl of a 1:10 diluted tail-bleed and 22.5 μl Blocker BSA). To each plate, tail bleeds were added as shown in Table 7 (group refers to the groups of polypeptide-conjugates used for immunization, Mu1-Mu9 refer to the individual mice that were immunized with each group of peptides, described above).

TABLE 7

| 1 | 2 | 3 | 4 | 5 | 6 | 7 | 8 | 9 |
|---|---|---|---|---|---|---|---|---|
| Tail | Tail | Tail | Tail | Tail | Tail | Tail | Tail | Tail |
| bleed | bleed | bleed | bleed | bleed | bleed | bleed | bleed | bleed |
| Grp1 | Grp1 | Grp1 | Grp2 | Grp2 | Grp2 | Grp3 | Grp3 | Grp3 |
| Mu1 | Mu2 | Mu3 | Mu4 | Mu5 | Mu6 | Mu7 | Mu8 | Mu9 |

The plates were incubated for 60 min at 37° C. and then washed 3× with 1× TBS-T. They then were incubated with 100 μl of a 1:2000 dilution of goat anti-mouse IgG HRP conjugate for 60 min at 37° C., washed again 3 times with TBS-T and developed with OPD. The absorbance measured at 492 nm.

C. Generation of a Library of Hybridoma Cells

An additional 1.2 mg of conjugate peptide mixtures (0.3 mg of each) was prepared for injection into mice prior to fusion. The mice were boosted with injections of polypeptides for three days prior to fusion. Fusion of spleen cells with mouse myeloma cells was performed on Day 84 and the hybridoma cells were grown in selection medium for 4 weeks. The medium was removed 3 weeks after fusion and fresh medium was added. The medium was harvested on Week 4 after fusion and tested for presence of anti-peptide antibodies by ELISA as described above. The assay was performed only for determination of antibodies to the immunized polypeptides and not for cross reactivity. The cells were harvested, aliquoted and stored (Fusion library) until the results from analysis of supernatants were obtained.

D. Cloning of Hybridomas to Generate Monoclonal Antibodies

A vial of the fusion library was thawed and the cells grown in medium for 2 weeks. Cells then were sorted using a FACS into ten 96-well plates such that each well received a single cell. The cells were grown for 2 weeks and the supernatant from each clone analyzed for presence of anti-peptide antibody as for the fusion library supernatant.

Positive clones were identified and ranked in order of ELISA signal intensities. Twelve clones with the highest signal intensities were scaled up and assayed for polypeptide-specific antibody after 2 weeks. The supernatants then were assayed for antibody titre determination and two clones showing the highest anti-peptide antibody titre were selected for scale-up and storage. The clones were grown to obtain 100 ml of medium and the cells then were frozen at −80° C.

E. Purification and Isotyping of IgG from Hybridoma Lines

The selected clones were grown for 2 weeks and the medium was used for analysis of antibody class and for specificity of binding to polypeptides by performing the assay described above. IgG was isotyped using Isotype mouse isotyping kits (Roche). The antibody from the supernatant was purified using Protein G affinity chromatography and stored in liquid nitrogen.

F. Results

Peptides used for the immunizations were as follows:

TABLE 8

| | | | |
|---|---|---|---|
| SEQ ID NO: | Peptide | SEQ ID NO: | Peptide |

| | | | |
|---|---|---|---|
| 1 | EPNGYF | 287 | QGKEYF |
| 5 | EGYPNF | 344 | NSFEGP |
| 137 | PEQGYN | 346 | NFKSGH |
| 141 | PGYEQN | 350 | NSGFKH |
| 236 | QESGPD | 351 | NGFKYH |
| 251 | QPGYEH | 372 | NTSGHK |
| 329 | NQHGYD | 379 | NKGYHL |
| 341 | NGYFEP | 428 | FPSGNE |
| 8 | ESPNGF | 450 | FNPSGE |
| 10 | EPHSGK | 454 | FSGNPE |
| 14 | ESGPHK | 455 | FGNPYE |
| 15 | EGPHYK | 481 | FTLGYQ |
| 19 | EQGYPN | 485 | FGYTLQ |
| 28 | EQSGFH | 488 | FSTLGQ |
| 144 | PSEQGN | 566 | HSGQEL |
| 146 | PEFSGQ | 570 | HQTSGN |
| 150 | PSGEFQ | 585 | HNDGYT |
| 151 | PGEFYQ | 595 | HFGYTK |
| 155 | PEGYKD | 636 | HDSGTL |
| 172 | PNSGEF | 691 | TLGYNF |
| 261 | QGYNHE | 735 | KGQNYT |
| 264 | QSNHGE | 747 | KNGYDQ |
| 265 | QFEGYK | 773 | KGYHPD |
| 282 | QKESGF | 776 | KSHPGD |

Peptides were injected singly or in groups of 2-4 polypeptides/animal as described above. Antisera were analyzed as described. The injected polypeptides raised antisera with high specificity and affinity.

### Example 6

Constructing a Retargeted Molecule for Assembly into Therapeutic Complexes Cloning of the CD20 Extra Cellular Loop into pBAD S1C5 scFv His

The extracellular domain of the human CD20 molecule, identified as KISHFLKMESLNFIRAHTPYINIYNCEPANPSEKNSPSTQYCY (SEQ ID NO:946), was reverse translated into double-stranded DNA and a digested XbaI site was added to the 5′ end of the double-stranded DNA and a digested XhoI site was added to the 3′ end of the double-stranded DNA. This represented the DNA insert that was to be cloned into the pBAD S1C5 His construct. The DNA insert sequence was separated into forward and reverse strands and each strand was broken into 40 base pair fragments and then these were ordered as oligonucleotides from Biosource International (Camarillo, Calif.). The 5′ ends of the oligonucleotides were phosphorylated so that they could be cloned. Sequence of the ordered oligonucleotides was as follows:

TABLE 9

| Oligo | | | SEQ ID |
|---|---|---|---|
| Name | Sequence | Length | NO |
| JKF660 | ctagaaAAAATTTCTCATTTTCTTAAAATGGAATCTCTTA | 40 | 947 |
| JKF661 | ATTTTATTCGTGCTCATACTCCTTATATTAATATTTATAA | 40 | 948 |
| JKF662 | TTGTGAACCTGCTAATCCTTCTGAAAAAAATTCTCCTTCT | 40 | 949 |
| JKF663 | ACTCAATATTGTTATc | 16 | 950 |
| JKF664 | tcgagATAACAATATTGAGTAGAAGGAGAATTTTTTTCAG | 40 | 951 |
| JKF665 | AAGGATTAGCAGGTTCACAATTATAAATATTAATATAAGG | 40 | 952 |
| JKF666 | AGTATGAGCACGAATAAAATTAAGAGATTCCATTTTAAGA | 40 | 953 |
| JKF667 | AAATGAGAAATTTTtt | 16 | 954 |

Oligonucleotides were resuspended at 100 uM in H₂O and 10 ul of each oligonucleotide was combined in a single 1.5 mL tube and heated to 94° C. for 10 minutes. Reaction was cooled to room temperature and then diluted by the addition of 1 mL H₂O. This generated insert was then ready for cloning.

The pBAD S1C5 V5 His construct was digested with XbaI and XhoI and the treated with CIP to generate a vector for cloning the CD20 insert. Approximately 50 ng of prepared vector was ligated with 1 ng of insert. Reactions were purified with a PCR purification column from Qiagen (Hilden, Germany) and 10% of the eluate was electroporated with electro competent cells from Invitrogen (La Jolla, Calif.) and plated on LB amp plates. Colonies were then screened for successful cloning by PCR and then clones were confirmed as having the CD20 epitope by sequencing.

The XbaI digestion to generate the vector removed a fragment of the S1C5 scFv and this had to be replaced to regenerate the full length S1C5 scFv. A PCR was performed with primers that spanned the entire full length of the S1C5 scFv. The amplicon was digested with XbaI and purified with a Qiagen PCR purification kit to generate insert for cloning.

A construct with the CD20 correctly cloned was digested with XbaI and treated with CIP to generate a vector for cloning. Approximately 50 ng of prepared vector was ligated with Ing of insert. Reactions were purified with a PCR purification column from Qiagen (Hilden, Germany) and 10% of the eluate was electroporated with electro competent cells from Invitrogen (La Jolla, Calif.) and plated on LB amp plates. Colonies were then screened for successful cloning by PCR and then clones were confirmed as having the full length S1C5 scFv by sequencing.

Since modifications will be apparent to those of skill in this art, it is intended that this invention be limited only by the scope of the appended claims.

1. A therapeutic complex, selected from among:
(a) a therapeutic complex, comprising:
a targeting domain and an effector molecule, wherein:
the targeting domain specifically binds to a subject-specific target;
the effector molecule renders the resulting therapeutic complex biologically effective;
the targeting domain and effector molecule are linked via the specific interaction of a binding partner and a capture agent;
the binding partner is conjugated to the targeting domain; and
the capture agent is conjugated to the effector molecule;
(b) therapeutic complex, comprising:
a targeting domain and an effector molecule, wherein:
the targeting domain specifically binds to a target;
the effector molecule is a polypeptide and renders the resulting therapeutic complex biologically effective;
the targeting domain and effector molecule are linked via the specific interaction of a binding partner and a capture agent;
the binding partner is a polypeptide between 5 and 100 amino acids in length;
the binding partner is conjugated to the targeting domain;
the capture agent is conjugated to the effector molecule; and
(c) a therapeutic complex, comprising:
a targeting domain and an effector molecule, wherein:
the targeting domain specifically binds to a target;
the effector molecule renders the resulting therapeutic complex biologically effective, wherein the biological effect of the therapeutic complex is selected from the group consisting of an immunomodulatory effect and an apoptotic effect;
the targeting domain and effector molecule are linked via the specific interaction of a binding partner and a capture agent;
the binding partner is conjugated to the targeting domain; and
the capture agent is conjugated to the effector molecule.

2. A therapeutic complex of claim 1 that is represented by the formula: (TR)ᵣ-(L1)ₛ-(B1)ₜ-(B2)ₓ-(L2)_{y}-(E)_{z}, wherein:
TR is a targeting domain;
E is an effector molecule;
r and z represent the number of TR and E moieties present in a complex, respectively;
B1 and B2 are binding partners and capture agents, respectively;
t and x represent the number of B 1 and B2 moieties present in a complex, respectively;
L1 and L2 are optional linkers;
s and y represent the number of linker moieties L1 and L2 in a complex, respectively, wherein each of s and y is selected independently and each is zero or an integer from 1 to n and n is any number of moieties that permit the complex to form and carry out its intended effect;
“-” represents an interaction between each component such that the resulting therapeutic complex is sufficiently stable upon formation to achieve the therapeutic effect;
each of r, t, x, and z is selected independently and each is an integer from 1 to n, where n is any number of moieties that permit the complex to form such that the resulting complex has an intended therapeutic activity.

3. A therapeutic complex of claim 1, wherein the effector is not a radiolabel.

4. The therapeutic complex of claim 1(b), wherein the polypeptide binding partner is a polypeptide of a length of amino acids selected from the group consisting of 5 to 50 amino acids, 5 to 20 amino acids, 5 to 12 amino acids, and 5 to 8 amino acids.

5. A therapeutic complex of claim 1, wherein the targeting domain is a polypeptide.

6. A therapeutic complex of claim 1, wherein the targeting domain is an antibody or fragment thereof.

7. The therapeutic complex of claim 6, wherein the antibody is a single chain antibody (scFv).

8. The therapeutic complex of claim 7, wherein the antibody or fragment thereof is humanized.

9. The therapeutic complex of claim 1, wherein the targeting domain comprises at least one variable region of an antibody.

10. The therapeutic complex of claim 1, wherein the targeting domain comprises one or more CDRs of an antibody.

11. The therapeutic complex of claim 5, wherein the polypeptide targeting domain is selected from the group consisting of a cell surface receptor, a ligand for a receptor, a cell surface antigen, and an adhesion molecule.

12. The therapeutic complex of claim 1, wherein the targeting domain binds to a cell.

13. The therapeutic complex of claim 12, wherein the cell is a B cell or a T cell.

14. The therapeutic complex of claim 12, wherein the cell is selected from a tumor cell, an antibody-secreting cell, an antigen presenting cell, a lymphoma cell and a cytokine-secreting cell.

15. The therapeutic complex of claim 1, wherein the targeting domain binds to a cell surface molecule.

16. The therapeutic complex of claim 15, wherein the cell surface molecule is selected from the group consisting of a receptor, an antibody, an antigen, a ligand for a receptor and an adhesion molecule.

17. The therapeutic complex of claim 1, wherein the targeting domain binds to a secreted molecule, an antibody, a cytokine, or a pathogen.

18. The therapeutic complex of claim 17, wherein the antibody is an auto-antibody or an anti-idiotype antibody.

19. The therapeutic complex of claim 17, wherein the pathogen is a virus or a parasite.

20. The therapeutic complex of claim 1, wherein the binding partner is a polypeptide binding partner.

21. The therapeutic complex of claim 20, wherein the polypeptide binding partner and targeting domain comprises a fusion protein.

22. The therapeutic complex of claim 20, wherein the polypeptide binding partner is selected from the group consisting of an antibody, an antibody fragment, an antigen, an epitope for an antibody, a receptor ligand and a receptor.

23. The therapeutic complex of claim 1, wherein the targeting domain and binding partner are linked directly or indirectly through a linker via covalent linkage.

24. The therapeutic complex of claim 1, wherein the capture agent and effector are linked directly or indirectly through a linker via non-covalent linkage.

25. The therapeutic complex of claim 1, wherein the capture agent and the binding partner are not constant domains of an antibody.

26. The therapeutic complex of claim 1, wherein the effector comprises a polypeptide.

27. The therapeutic complex of claim 26, wherein:
the effector comprises an antibody or fragment thereof.

28. The therapeutic complex of claim 27, wherein the antibody or fragment thereof is humanized.

29. The therapeutic complex of claim 1, wherein the effector interacts with an Fc receptor.

30. The therapeutic complex of claim 29, wherein the effector comprises an Fc domain.

31. The therapeutic complex of claim 30, wherein the Fc domain amino acid sequence comprises an Fc domain sequence from a murine IgG2a, a human IgG1 or a human IgG3 antibody.

32. The therapeutic complex of claim 1, wherein the effector is a cytokine.

33. The therapeutic complex of claim 1, wherein the effector and the capture agent comprise a fusion protein.

34. The therapeutic complex of claim 1, wherein the effector is selected from the group consisting of an enzyme, a receptor, a ligand for a receptor, and an inhibitor of a receptor.

35. The therapeutic complex of claim 1, wherein the biological effect is selected from among an immunomodulatory effect, receptor binding, receptor inhibition, enzymatic modification, and enzymatic degradation.

36. The therapeutic complex of claims 35, wherein the immunomodulatory effect is selected from the group consisting of neutralization, immunosuppression, clearance, modulation of cytokine expression or secretion, modulation of T cell activation, modulation of immune cell proliferation, complement activation, antibody-dependent cellular cytotoxicity (ADCC), and opsonization.

37. The therapeutic complex of claim 1, wherein binding between the capture agent and the binding partner is effected via hydrophobic interaction.

38. The therapeutic complex of claim 1, wherein components of the complex are cross-linked or chemically conjugated.

39. A pharmaceutical composition, comprising a therapeutic complex of claim 1.

40. The pharmaceutical composition of claim 39, wherein the biological effect comprises a therapeutic effect.

41. A method of treating a disease or condition, comprising: administering a pharmaceutical composition of claim 39.

42. The method of claim 41, wherein the composition comprises a therapeutic complex designed for personalized treatment.

43. The method of claim 41, wherein the disease is selected from B cell-mediated diseases, an autoimmune disease and T cell-mediated diseases.

44. The method of claim 41, wherein the disease or condition is selected from cancers, inflammatory diseases, autoimmune diseases, infectious diseases, neurodegenerative diseases, and ophthalmic diseases.

45. The method of claim 41, wherein the disease or condition is selected from non-Hodgkin's lymphoma, rheumatoid arthritis, lupus, multiple sclerosis, melanoma, a posterior intraocular inflammation, pathogen and virus infection.

46. The method of claim 41, wherein the targeting domain and the effector are administered as a complex, or wherein the targeting domain and the effector are administered sequentially, simultaneously or intermittently.

47. The method of claim 41, wherein:
the targeting domain and effector are administered separately; and either
one or more doses of the targeting domain is(are) administered prior to administration of a therapeutic complex also comprising the targeting domain; or
one or more doses of the effector prior is (are) administered prior to administration of a therapeutic complex comprising the effector.

48. A method of preparing a therapeutic complex of claim 1, comprising:
contacting a targeting domain and an effector molecule under conditions, whereby a complex forms, wherein,
the targeting domain specifically binds to a target;
the effector molecule renders the resulting therapeutic complex biologically effective;
the targeting domain and effector molecule are linked via the specific interaction of a binding partner and a capture agent;
the binding partner is conjugated to the targeting domain; and
the capture agent is conjugated to the effector molecule.

49. The method of claim 48, wherein the targeting domain and effector molecule are contacted in vitro.

50. The method of claim 48, wherein the complex is cross-linked or chemically conjugated after formation.

51. The method of claim 50, further comprising the step of cross-linking the binding partner and capture agent after complex formation.

52. The method of claim 48, further comprising isolating the complex after formation.

53. The method of claim 48, wherein the targeting domain and effector molecule are contacted in vivo in a subject after separate administration of each to the subject.

54. The method of claim 48, wherein the targeting domain and the effector molecule are expressed in a cell, wherein the complex forms.

55. The method of claim 48, wherein the targeting domain is subject-specific.

56. The method of claim 48, wherein the effector molecule is a polypeptide.

57. The method of claim 48, wherein the effector molecule confers an immunomodulatory effect.

58. A method of rendering an antibody or antibody fragment therapeutically effective, comprising:
preparing a therapeutic by combining a targeting domain that comprises an antibody or fragment thereof with an effector molecule via the specific interaction of a binding partner and capture agent to form a therapeutic complex of claim 1, whereby the complex is therapeutically effective, wherein the complex comprises:
a targeting domain comprising an antibody or antibody fragment that specifically binds to a target;
an effector molecule;
the effector molecule renders the resulting complex therapeutically effective;
the targeting domain and effector molecule are linked via the specific interaction of a binding partner and a capture agent;
the binding partner is conjugated to the targeting domain;
the capture agent is conjugated to the effector molecule.

59. A method of rendering a target-specific polypeptide therapeutically effective, comprising:
preparing a therapeutic complex by combining a targeting domain with an effector molecule via the specific interaction of a binding partner and capture agent, to form a therapeutic complex of claim 1, wherein the complex comprises:
a targeting domain comprising a polypeptide, wherein the polypeptide specifically binds to a target;
an effector molecule, wherein:
the effector molecule renders the resulting complex therapeutically effective;
the targeting domain and effector molecule are linked via the specific interaction of a binding partner and a capture agent;
the binding partner is conjugated to the targeting domain;
the binding partner is a polypeptide of length sufficient to specifically interact with a capture agent and is less than about 100 amino acids;
the capture agent is conjugated to the effector molecule.

60. The method of claim 59, wherein the binding partner contains 5 to 50 amino acids, 5 to 30 amino acids, 5 to 20 amino acids, 5 to 12 amino acids or 5 to 8 amino acids.

61. The method of claim 59, wherein the polypeptide targeting domain comprises an antibody or an antibody fragment.

62. The method of claim 61, wherein the antibody or antibody fragment is selected from the group consisting of a single chain antibody (scFv), an anti-idiotype antibody, a variable region, a fragment of a variable region sufficient to bind to another molecule, a CDR, a Fab, a F(ab)₂, and an Fv.

63. The method of claim 61, wherein:
the antibody or antibody fragment binds to a cell-surface molecule; or
the antibody or antibody fragment binds to a subject-specific target.

64. A method of screening test molecules to identify effectors for use in the therapeutic complexes of claim 1, comprising:
a) preparing a complex by combining:
a targeting domain comprising an antibody or antibody fragment, wherein the antibody or antibody fragment specifically binds to a target; and
a candidate effector molecule, wherein:
the targeting molecule and candidate effector molecule are linked via the specific interaction of a binding partner and a capture agent;
the binding partner is conjugated to the targeting domain; and
the capture agent is conjugated to the candidate effector molecule;
b) administering the complex to a subject; and
c) detecting an effect on the subject to thereby identify an effector molecule.

65. A method of screening test molecules to identify targeting domains for use in the therapeutic complexes of claim 1, comprising:
a) generating a complex, by combining:
a candidate targeting domain comprising an antibody or antibody fragment;
an effector molecule, wherein:
the effector molecule renders the resulting complex biologically effective;
the candidate targeting domain and effector molecule are linked via the specific interaction of a binding partner and a capture agent;
the binding partner is conjugated to the candidate targeting domain; and
the capture agent is conjugated to the effector molecule;
b) administering the complex to a subject; and
c) detecting a therapeutic effect of the complex on the subject to thereby identify a targeting domain.

66. A therapeutic complex of claim 1, wherein the specific interaction of the binding partner and capture agent is non-covalent.

67. The therapeutic complex of claim 66, wherein the non-covalent linkages are selected from among hydrogen bonding, hydrophobic bonds, Van der Waals interactions and combinations thereof.

68. A therapeutic complex, comprising:
a targeting domain and an effector molecule, wherein:
the targeting domain specifically binds to a target;
the effector molecule renders the resulting therapeutic complex biologically effective;
the targeting domain and effector molecule are linked via the specific interaction of a binding partner and a capture agent;
the binding partner is conjugated to the targeting domain;
the capture agent is conjugated to the effector molecule; and
the capture agent comprises at least one variable domain of an antibody or a portion thereof sufficient to specifically bind to the binding partner.

69. The therapeutic complex of claim 68, wherein the effector molecule and capture agent comprise an antibody or antibody fragment or antibody complex.

70. The therapeutic complex of claim 69, wherein the antibody, antibody fragment or antibody in the complex is selected from the group consisting of rituximab, trastuzumab, tositumomab, ibritumomab, alemtuzumab, infliximab, CDP-571, edrecolomab, muromab-CD3, daclizumab, omalizumab, cetuximab and bevacizumab and antibody fragments thereof.

71. The therapeutic complex of claim 68, wherein the targeting domain specifically binds to a subject-specific target.

72. The therapeutic complex of claim 68, wherein the effector molecule binds to a first target that is the same as the target of the targeting domain in the therapeutic complex.

73. The therapeutic complex of claim 68, wherein the effector molecule is selected from among antibodies, immunotoxins and antibody conjugates.

74. The therapeutic complex of claim 68, wherein the effector molecule binds to a first target that is different from the target of the targeting domain in the therapeutic complex.

75. The therapeutic complex of claim 68, wherein the effector molecule binds to the first target in the absence of the complex, and binding by the effector molecule to the first target is altered or reduced when the effector molecule is part of the therapeutic complex.

76. The therapeutic complex of claim 68, wherein the first target is different from the target of the therapeutic complex, whereby the therapeutic complex binds to either or both targets.

77. The therapeutic complex of claim 76, wherein the different targets occur on the same cell, tissue or molecule.


SEQUENCE LISTING (958 sequences)

SEQ ID NO: 1 - protein, 6 aa, Artificial Sequence
  synthetic peptide
EPNGYF

SEQ ID NO: 2 - protein, 6 aa, Artificial Sequence
  synthetic peptide
EPNSGF

SEQ ID NO: 3 - protein, 6 aa, Artificial Sequence
  synthetic peptide
EPGYNF

SEQ ID NO: 4 - protein, 6 aa, Artificial Sequence
  synthetic peptide
EPSGNF

SEQ ID NO: 5 - protein, 6 aa, Artificial Sequence
  synthetic peptide
EGYPNF

SEQ ID NO: 6 - protein, 6 aa, Artificial Sequence
  synthetic peptide
ESGPNF

SEQ ID NO: 7 - protein, 6 aa, Artificial Sequence
  synthetic peptide
EGPNYF

SEQ ID NO: 8 - protein, 6 aa, Artificial Sequence
  synthetic peptide
ESPNGF

SEQ ID NO: 9 - protein, 6 aa, Artificial Sequence
  synthetic peptide
EPHGYK

SEQ ID NO: 10 - protein, 6 aa, Artificial Sequence
  synthetic peptide
EPHSGK

SEQ ID NO: 11 - protein, 6 aa, Artificial Sequence
  synthetic peptide
EPGYHK

SEQ ID NO: 12 - protein, 6 aa, Artificial Sequence
  synthetic peptide
EPSGHK

SEQ ID NO: 13 - protein, 6 aa, Artificial Sequence
  synthetic peptide
EGYPHK

SEQ ID NO: 14 - protein, 6 aa, Artificial Sequence
  synthetic peptide
ESGPHK

SEQ ID NO: 15 - protein, 6 aa, Artificial Sequence
  synthetic peptide
EGPHYK

SEQ ID NO: 16 - protein, 6 aa, Artificial Sequence
  synthetic peptide
ESPHGK

SEQ ID NO: 17 - protein, 6 aa, Artificial Sequence
  synthetic peptide
EQPGYN

SEQ ID NO: 18 - protein, 6 aa, Artificial Sequence
  synthetic peptide
EQPSGN

SEQ ID NO: 19 - protein, 6 aa, Artificial Sequence
  synthetic peptide
EQGYPN

SEQ ID NO: 20 - protein, 6 aa, Artificial Sequence
  synthetic peptide
EQSGPN

SEQ ID NO: 21 - protein, 6 aa, Artificial Sequence
  synthetic peptide
EGYQPN

SEQ ID NO: 22 - protein, 6 aa, Artificial Sequence
  synthetic peptide
ESGQPN

SEQ ID NO: 23 - protein, 6 aa, Artificial Sequence
  synthetic peptide
EGQPYN

SEQ ID NO: 24 - protein, 6 aa, Artificial Sequence
  synthetic peptide
ESQPGN

SEQ ID NO: 25 - protein, 6 aa, Artificial Sequence
  synthetic peptide
EQFGYH

SEQ ID NO: 26 - protein, 6 aa, Artificial Sequence
  synthetic peptide
EQFSGH

SEQ ID NO: 27 - protein, 6 aa, Artificial Sequence
  synthetic peptide
EQGYFH

SEQ ID NO: 28 - protein, 6 aa, Artificial Sequence
  synthetic peptide
EQSGFH

SEQ ID NO: 29 - protein, 6 aa, Artificial Sequence
  synthetic peptide
EGYQFH

SEQ ID NO: 30 - protein, 6 aa, Artificial Sequence
  synthetic peptide
ESGQFH

SEQ ID NO: 31 - protein, 6 aa, Artificial Sequence
  synthetic peptide
EGQFYH

SEQ ID NO: 32 - protein, 6 aa, Artificial Sequence
  synthetic peptide
ESQFGH

SEQ ID NO: 33 - protein, 6 aa, Artificial Sequence
  synthetic peptide
ENPGYT

SEQ ID NO: 34 - protein, 6 aa, Artificial Sequence
  synthetic peptide
ENPSGT

SEQ ID NO: 35 - protein, 6 aa, Artificial Sequence
  synthetic peptide
ENGYPT

SEQ ID NO: 36 - protein, 6 aa, Artificial Sequence
  synthetic peptide
ENSGPT

SEQ ID NO: 37 - protein, 6 aa, Artificial Sequence
  synthetic peptide
EGYNPT

SEQ ID NO: 38 - protein, 6 aa, Artificial Sequence
  synthetic peptide
ESGNPT

SEQ ID NO: 39 - protein, 6 aa, Artificial Sequence
  synthetic peptide
EGNPYT

SEQ ID NO: 40 - protein, 6 aa, Artificial Sequence
  synthetic peptide
ESNPGT

SEQ ID NO: 41 - protein, 6 aa, Artificial Sequence
  synthetic peptide
ENFGYD

SEQ ID NO: 42 - protein, 6 aa, Artificial Sequence
  synthetic peptide
ENFSGD

SEQ ID NO: 43 - protein, 6 aa, Artificial Sequence
  synthetic peptide
ENGYFD

SEQ ID NO: 44 - protein, 6 aa, Artificial Sequence
  synthetic peptide
ENSGFD

SEQ ID NO: 45 - protein, 6 aa, Artificial Sequence
  synthetic peptide
EGYNFD

SEQ ID NO: 46 - protein, 6 aa, Artificial Sequence
  synthetic peptide
ESGNFD

SEQ ID NO: 47 - protein, 6 aa, Artificial Sequence
  synthetic peptide
EGNFYD

SEQ ID NO: 48 - protein, 6 aa, Artificial Sequence
  synthetic peptide
ESNFGD

SEQ ID NO: 49 - protein, 6 aa, Artificial Sequence
  synthetic peptide
ENDGYP

SEQ ID NO: 50 - protein, 6 aa, Artificial Sequence
  synthetic peptide
ENDSGP

SEQ ID NO: 51 - protein, 6 aa, Artificial Sequence
  synthetic peptide
ENGYDP

SEQ ID NO: 52 - protein, 6 aa, Artificial Sequence
  synthetic peptide
ENSGDP

SEQ ID NO: 53 - protein, 6 aa, Artificial Sequence
  synthetic peptide
EGYNDP

SEQ ID NO: 54 - protein, 6 aa, Artificial Sequence
  synthetic peptide
ESGNDP

SEQ ID NO: 55 - protein, 6 aa, Artificial Sequence
  synthetic peptide
EGNDYP

SEQ ID NO: 56 - protein, 6 aa, Artificial Sequence
  synthetic peptide
ESNDGP

SEQ ID NO: 57 - protein, 6 aa, Artificial Sequence
  synthetic peptide
EFQGYP

SEQ ID NO: 58 - protein, 6 aa, Artificial Sequence
  synthetic peptide
EFQSGP

SEQ ID NO: 59 - protein, 6 aa, Artificial Sequence
  synthetic peptide
EFGYQP

SEQ ID NO: 60 - protein, 6 aa, Artificial Sequence
  synthetic peptide
EFSGQP

SEQ ID NO: 61 - protein, 6 aa, Artificial Sequence
  synthetic peptide
EGYFQP

SEQ ID NO: 62 - protein, 6 aa, Artificial Sequence
  synthetic peptide
ESGFQP

SEQ ID NO: 63 - protein, 6 aa, Artificial Sequence
  synthetic peptide
EGFQYP

SEQ ID NO: 64 - protein, 6 aa, Artificial Sequence
  synthetic peptide
ESFQGP

SEQ ID NO: 65 - protein, 6 aa, Artificial Sequence
  synthetic peptide
EFKGYT

SEQ ID NO: 66 - protein, 6 aa, Artificial Sequence
  synthetic peptide
EFKSGT

SEQ ID NO: 67 - protein, 6 aa, Artificial Sequence
  synthetic peptide
EFGYKT

SEQ ID NO: 68 - protein, 6 aa, Artificial Sequence
  synthetic peptide
EFSGKT

SEQ ID NO: 69 - protein, 6 aa, Artificial Sequence
  synthetic peptide
EGYFKT

SEQ ID NO: 70 - protein, 6 aa, Artificial Sequence
  synthetic peptide
ESGFKT

SEQ ID NO: 71 - protein, 6 aa, Artificial Sequence
  synthetic peptide
EGFKYT

SEQ ID NO: 72 - protein, 6 aa, Artificial Sequence
  synthetic peptide
ESFKGT

SEQ ID NO: 73 - protein, 6 aa, Artificial Sequence
  synthetic peptide
EFDGYH

SEQ ID NO: 74 - protein, 6 aa, Artificial Sequence
  synthetic peptide
EFDSGH

SEQ ID NO: 75 - protein, 6 aa, Artificial Sequence
  synthetic peptide
EFGYDH

SEQ ID NO: 76 - protein, 6 aa, Artificial Sequence
  synthetic peptide
EFSGDH

SEQ ID NO: 77 - protein, 6 aa, Artificial Sequence
  synthetic peptide
EGYFDH

SEQ ID NO: 78 - protein, 6 aa, Artificial Sequence
  synthetic peptide
ESGFDH

SEQ ID NO: 79 - protein, 6 aa, Artificial Sequence
  synthetic peptide
EGFDYH

SEQ ID NO: 80 - protein, 6 aa, Artificial Sequence
  synthetic peptide
ESFDGH

SEQ ID NO: 81 - protein, 6 aa, Artificial Sequence
  synthetic peptide
EHNGYQ

SEQ ID NO: 82 - protein, 6 aa, Artificial Sequence
  synthetic peptide
EHNSGQ

SEQ ID NO: 83 - protein, 6 aa, Artificial Sequence
  synthetic peptide
EHGYNQ

SEQ ID NO: 84 - protein, 6 aa, Artificial Sequence
  synthetic peptide
EHSGNQ

SEQ ID NO: 85 - protein, 6 aa, Artificial Sequence
  synthetic peptide
EGYHNQ

SEQ ID NO: 86 - protein, 6 aa, Artificial Sequence
  synthetic peptide
ESGHNQ

SEQ ID NO: 87 - protein, 6 aa, Artificial Sequence
  synthetic peptide
EGHNYQ

SEQ ID NO: 88 - protein, 6 aa, Artificial Sequence
  synthetic peptide
ESHNGQ

SEQ ID NO: 89 - protein, 6 aa, Artificial Sequence
  synthetic peptide
EHKGYP

SEQ ID NO: 90 - protein, 6 aa, Artificial Sequence
  synthetic peptide
EHKSGP

SEQ ID NO: 91 - protein, 6 aa, Artificial Sequence
  synthetic peptide
EHGYKP

SEQ ID NO: 92 - protein, 6 aa, Artificial Sequence
  synthetic peptide
EHSGKP

SEQ ID NO: 93 - protein, 6 aa, Artificial Sequence
  synthetic peptide
EGYHKP

SEQ ID NO: 94 - protein, 6 aa, Artificial Sequence
  synthetic peptide
ESGHKP

SEQ ID NO: 95 - protein, 6 aa, Artificial Sequence
  synthetic peptide
EGHKYP

SEQ ID NO: 96 - protein, 6 aa, Artificial Sequence
  synthetic peptide
ESHKGP

SEQ ID NO: 97 - protein, 6 aa, Artificial Sequence
  synthetic peptide
ETNGYK

SEQ ID NO: 98 - protein, 6 aa, Artificial Sequence
  synthetic peptide
ETNSGK

SEQ ID NO: 99 - protein, 6 aa, Artificial Sequence
  synthetic peptide
ETGYNK

SEQ ID NO: 100 - protein, 6 aa, Artificial Sequence
  synthetic peptide
ETSGNK

SEQ ID NO: 101 - protein, 6 aa, Artificial Sequence
  synthetic peptide
EGYTNK

SEQ ID NO: 102 - protein, 6 aa, Artificial Sequence
  synthetic peptide
ESGTNK

SEQ ID NO: 103 - protein, 6 aa, Artificial Sequence
  synthetic peptide
EGTNYK

SEQ ID NO: 104 - protein, 6 aa, Artificial Sequence
  synthetic peptide
ESTNGK

SEQ ID NO: 105 - protein, 6 aa, Artificial Sequence
  synthetic peptide
EKPGYH

SEQ ID NO: 106 - protein, 6 aa, Artificial Sequence
  synthetic peptide
EKPSGH

SEQ ID NO: 107 - protein, 6 aa, Artificial Sequence
  synthetic peptide
EKGYPH

SEQ ID NO: 108 - protein, 6 aa, Artificial Sequence
  synthetic peptide
EKSGPH

SEQ ID NO: 109 - protein, 6 aa, Artificial Sequence
  synthetic peptide
EGYKPH

SEQ ID NO: 110 - protein, 6 aa, Artificial Sequence
  synthetic peptide
ESGKPH

SEQ ID NO: 111 - protein, 6 aa, Artificial Sequence
  synthetic peptide
EGKPYH

SEQ ID NO: 112 - protein, 6 aa, Artificial Sequence
  synthetic peptide
ESKPGH

SEQ ID NO: 113 - protein, 6 aa, Artificial Sequence
  synthetic peptide
ELNGYD

SEQ ID NO: 114 - protein, 6 aa, Artificial Sequence
  synthetic peptide
ELNSGD

SEQ ID NO: 115 - protein, 6 aa, Artificial Sequence
  synthetic peptide
ELGYND

SEQ ID NO: 116 - protein, 6 aa, Artificial Sequence
  synthetic peptide
ELSGND

SEQ ID NO: 117 - protein, 6 aa, Artificial Sequence
  synthetic peptide
EGYLND

SEQ ID NO: 118 - protein, 6 aa, Artificial Sequence
  synthetic peptide
ESGLND

SEQ ID NO: 119 - protein, 6 aa, Artificial Sequence
  synthetic peptide
EGLNYD

SEQ ID NO: 120 - protein, 6 aa, Artificial Sequence
  synthetic peptide
ESLNGD

SEQ ID NO: 121 - protein, 6 aa, Artificial Sequence
  synthetic peptide
EDPGYF

SEQ ID NO: 122 - protein, 6 aa, Artificial Sequence
  synthetic peptide
EDPSGF

SEQ ID NO: 123 - protein, 6 aa, Artificial Sequence
  synthetic peptide
EDGYPF

SEQ ID NO: 124 - protein, 6 aa, Artificial Sequence
  synthetic peptide
EDSGPF

SEQ ID NO: 125 - protein, 6 aa, Artificial Sequence
  synthetic peptide
EGYDPF

SEQ ID NO: 126 - protein, 6 aa, Artificial Sequence
  synthetic peptide
ESGDPF

SEQ ID NO: 127 - protein, 6 aa, Artificial Sequence
  synthetic peptide
EGDPYF

SEQ ID NO: 128 - protein, 6 aa, Artificial Sequence
  synthetic peptide
ESDPGF

SEQ ID NO: 129 - protein, 6 aa, Artificial Sequence
  synthetic peptide
EDFGYP

SEQ ID NO: 130 - protein, 6 aa, Artificial Sequence
  synthetic peptide
EDFSGP

SEQ ID NO: 131 - protein, 6 aa, Artificial Sequence
  synthetic peptide
EDGYFP

SEQ ID NO: 132 - protein, 6 aa, Artificial Sequence
  synthetic peptide
EDSGFP

SEQ ID NO: 133 - protein, 6 aa, Artificial Sequence
  synthetic peptide
EGYDFP

SEQ ID NO: 134 - protein, 6 aa, Artificial Sequence
  synthetic peptide
ESGDFP

SEQ ID NO: 135 - protein, 6 aa, Artificial Sequence
  synthetic peptide
EGDFYP

SEQ ID NO: 136 - protein, 6 aa, Artificial Sequence
  synthetic peptide
ESDFGP

SEQ ID NO: 137 - protein, 6 aa, Artificial Sequence
  synthetic peptide
PEQGYN

SEQ ID NO: 138 - protein, 6 aa, Artificial Sequence
  synthetic peptide
PEQSGN

SEQ ID NO: 139 - protein, 6 aa, Artificial Sequence
  synthetic peptide
PEGYQN

SEQ ID NO: 140 - protein, 6 aa, Artificial Sequence
  synthetic peptide
PESGQN

SEQ ID NO: 141 - protein, 6 aa, Artificial Sequence
  synthetic peptide
PGYEQN

SEQ ID NO: 142 - protein, 6 aa, Artificial Sequence
  synthetic peptide
PSGEQN

SEQ ID NO: 143 - protein, 6 aa, Artificial Sequence
  synthetic peptide
PGEQYN

SEQ ID NO: 144 - protein, 6 aa, Artificial Sequence
  synthetic peptide
PSEQGN

SEQ ID NO: 145 - protein, 6 aa, Artificial Sequence
  synthetic peptide
PEFGYQ

SEQ ID NO: 146 - protein, 6 aa, Artificial Sequence
  synthetic peptide
PEFSGQ

SEQ ID NO: 147 - protein, 6 aa, Artificial Sequence
  synthetic peptide
PEGYFQ

SEQ ID NO: 148 - protein, 6 aa, Artificial Sequence
  synthetic peptide
PESGFQ

SEQ ID NO: 149 - protein, 6 aa, Artificial Sequence
  synthetic peptide
PGYEFQ

SEQ ID NO: 150 - protein, 6 aa, Artificial Sequence
  synthetic peptide
PSGEFQ

SEQ ID NO: 151 - protein, 6 aa, Artificial Sequence
  synthetic peptide
PGEFYQ

SEQ ID NO: 152 - protein, 6 aa, Artificial Sequence
  synthetic peptide
PSEFGQ

SEQ ID NO: 153 - protein, 6 aa, Artificial Sequence
  synthetic peptide
PEKGYD

SEQ ID NO: 154 - protein, 6 aa, Artificial Sequence
  synthetic peptide
PEKSGD

SEQ ID NO: 155 - protein, 6 aa, Artificial Sequence
  synthetic peptide
PEGYKD

SEQ ID NO: 156 - protein, 6 aa, Artificial Sequence
  synthetic peptide
PESGKD

SEQ ID NO: 157 - protein, 6 aa, Artificial Sequence
  synthetic peptide
PGYEKD

SEQ ID NO: 158 - protein, 6 aa, Artificial Sequence
  synthetic peptide
PSGEKD

SEQ ID NO: 159 - protein, 6 aa, Artificial Sequence
  synthetic peptide
PGEKYD

SEQ ID NO: 160 - protein, 6 aa, Artificial Sequence
  synthetic peptide
PSEKGD

SEQ ID NO: 161 - protein, 6 aa, Artificial Sequence
  synthetic peptide
PQTGYE

SEQ ID NO: 162 - protein, 6 aa, Artificial Sequence
  synthetic peptide
PQTSGE

SEQ ID NO: 163 - protein, 6 aa, Artificial Sequence
  synthetic peptide
PQGYTE

SEQ ID NO: 164 - protein, 6 aa, Artificial Sequence
  synthetic peptide
PQSGTE

SEQ ID NO: 165 - protein, 6 aa, Artificial Sequence
  synthetic peptide
PGYQTE

SEQ ID NO: 166 - protein, 6 aa, Artificial Sequence
  synthetic peptide
PSGQTE

SEQ ID NO: 167 - protein, 6 aa, Artificial Sequence
  synthetic peptide
PGQTYE

SEQ ID NO: 168 - protein, 6 aa, Artificial Sequence
  synthetic peptide
PSQTGE

SEQ ID NO: 169 - protein, 6 aa, Artificial Sequence
  synthetic peptide
PNEGYF

SEQ ID NO: 170 - protein, 6 aa, Artificial Sequence
  synthetic peptide
PNESGF

SEQ ID NO: 171 - protein, 6 aa, Artificial Sequence
  synthetic peptide
PNGYEF

SEQ ID NO: 172 - protein, 6 aa, Artificial Sequence
  synthetic peptide
PNSGEF

SEQ ID NO: 173 - protein, 6 aa, Artificial Sequence
  synthetic peptide
PGYNEF

SEQ ID NO: 174 - protein, 6 aa, Artificial Sequence
  synthetic peptide
PSGNEF

SEQ ID NO: 175 - protein, 6 aa, Artificial Sequence
  synthetic peptide
PGNEYF

SEQ ID NO: 176 - protein, 6 aa, Artificial Sequence
  synthetic peptide
PSNEGF

SEQ ID NO: 177 - protein, 6 aa, Artificial Sequence
  synthetic peptide
PFEGYQ

SEQ ID NO: 178 - protein, 6 aa, Artificial Sequence
  synthetic peptide
PFESGQ

SEQ ID NO: 179 - protein, 6 aa, Artificial Sequence
  synthetic peptide
PFGYEQ

SEQ ID NO: 180 - protein, 6 aa, Artificial Sequence
  synthetic peptide
PFSGEQ

SEQ ID NO: 181 - protein, 6 aa, Artificial Sequence
  synthetic peptide
PGYFEQ

SEQ ID NO: 182 - protein, 6 aa, Artificial Sequence
  synthetic peptide
PSGFEQ

SEQ ID NO: 183 - protein, 6 aa, Artificial Sequence
  synthetic peptide
PGFEYQ

SEQ ID NO: 184 - protein, 6 aa, Artificial Sequence
  synthetic peptide
PSFEGQ

SEQ ID NO: 185 - protein, 6 aa, Artificial Sequence
  synthetic peptide
PFHGYL

SEQ ID NO: 186 - protein, 6 aa, Artificial Sequence
  synthetic peptide
PFHSGL

SEQ ID NO: 187 - protein, 6 aa, Artificial Sequence
  synthetic peptide
PFGYHL

SEQ ID NO: 188 - protein, 6 aa, Artificial Sequence
  synthetic peptide
PFSGHL

SEQ ID NO: 189 - protein, 6 aa, Artificial Sequence
  synthetic peptide
PGYFHL

SEQ ID NO: 190 - protein, 6 aa, Artificial Sequence
  synthetic peptide
PSGFHL

SEQ ID NO: 191 - protein, 6 aa, Artificial Sequence
  synthetic peptide
PGFHYL

SEQ ID NO: 192 - protein, 6 aa, Artificial Sequence
  synthetic peptide
PSFHGL

SEQ ID NO: 193 - protein, 6 aa, Artificial Sequence
  synthetic peptide
PHEGYK

SEQ ID NO: 194 - protein, 6 aa, Artificial Sequence
  synthetic peptide
PHESGK

SEQ ID NO: 195 - protein, 6 aa, Artificial Sequence
  synthetic peptide
PHGYEK

SEQ ID NO: 196 - protein, 6 aa, Artificial Sequence
  synthetic peptide
PHSGEK

SEQ ID NO: 197 - protein, 6 aa, Artificial Sequence
  synthetic peptide
PGYHEK

SEQ ID NO: 198 - protein, 6 aa, Artificial Sequence
  synthetic peptide
PSGHEK

SEQ ID NO: 199 - protein, 6 aa, Artificial Sequence
  synthetic peptide
PGHEYK

SEQ ID NO: 200 - protein, 6 aa, Artificial Sequence
  synthetic peptide
PSHEGK

SEQ ID NO: 201 - protein, 6 aa, Artificial Sequence
  synthetic peptide
PHTGYF

SEQ ID NO: 202 - protein, 6 aa, Artificial Sequence
  synthetic peptide
PHTSGF

SEQ ID NO: 203 - protein, 6 aa, Artificial Sequence
  synthetic peptide
PHGYTF

SEQ ID NO: 204 - protein, 6 aa, Artificial Sequence
  synthetic peptide
PHSGTF

SEQ ID NO: 205 - protein, 6 aa, Artificial Sequence
  synthetic peptide
PGYHTF

SEQ ID NO: 206 - protein, 6 aa, Artificial Sequence
  synthetic peptide
PSGHTF

SEQ ID NO: 207 - protein, 6 aa, Artificial Sequence
  synthetic peptide
PGHTYF

SEQ ID NO: 208 - protein, 6 aa, Artificial Sequence
  synthetic peptide
PSHTGF

SEQ ID NO: 209 - protein, 6 aa, Artificial Sequence
  synthetic peptide
PTLGYD

SEQ ID NO: 210 - protein, 6 aa, Artificial Sequence
  synthetic peptide
PTLSGD

SEQ ID NO: 211 - protein, 6 aa, Artificial Sequence
  synthetic peptide
PTGYLD

SEQ ID NO: 212 - protein, 6 aa, Artificial Sequence
  synthetic peptide
PTSGLD

SEQ ID NO: 213 - protein, 6 aa, Artificial Sequence
  synthetic peptide
PGYTLD

SEQ ID NO: 214 - protein, 6 aa, Artificial Sequence
  synthetic peptide
PSGTLD

SEQ ID NO: 215 - protein, 6 aa, Artificial Sequence
  synthetic peptide
PGTLYD

SEQ ID NO: 216 - protein, 6 aa, Artificial Sequence
  synthetic peptide
PSTLGD

SEQ ID NO: 217 - protein, 6 aa, Artificial Sequence
  synthetic peptide
PKHGYT

SEQ ID NO: 218 - protein, 6 aa, Artificial Sequence
  synthetic peptide
PKHSGT

SEQ ID NO: 219 - protein, 6 aa, Artificial Sequence
  synthetic peptide
PKGYHT

SEQ ID NO: 220 - protein, 6 aa, Artificial Sequence
  synthetic peptide
PKSGHT

SEQ ID NO: 221 - protein, 6 aa, Artificial Sequence
  synthetic peptide
PGYKHT

SEQ ID NO: 222 - protein, 6 aa, Artificial Sequence
  synthetic peptide
PSGKHT

SEQ ID NO: 223 - protein, 6 aa, Artificial Sequence
  synthetic peptide
PGKHYT

SEQ ID NO: 224 - protein, 6 aa, Artificial Sequence
  synthetic peptide
PSKHGT

SEQ ID NO: 225 - protein, 6 aa, Artificial Sequence
  synthetic peptide
PLDGYN

SEQ ID NO: 226 - protein, 6 aa, Artificial Sequence
  synthetic peptide
PLDSGN

SEQ ID NO: 227 - protein, 6 aa, Artificial Sequence
  synthetic peptide
PLGYDN

SEQ ID NO: 228 - protein, 6 aa, Artificial Sequence
  synthetic peptide
PLSGDN

SEQ ID NO: 229 - protein, 6 aa, Artificial Sequence
  synthetic peptide
PGYLDN

SEQ ID NO: 230 - protein, 6 aa, Artificial Sequence
  synthetic peptide
PSGLDN

SEQ ID NO: 231 - protein, 6 aa, Artificial Sequence
  synthetic peptide
PGLDYN

SEQ ID NO: 232 - protein, 6 aa, Artificial Sequence
  synthetic peptide
PSLDGN

SEQ ID NO: 233 - protein, 6 aa, Artificial Sequence
  synthetic peptide
QEPGYD

SEQ ID NO: 234 - protein, 6 aa, Artificial Sequence
  synthetic peptide
QEPSGD

SEQ ID NO: 235 - protein, 6 aa, Artificial Sequence
  synthetic peptide
QEGYPD

SEQ ID NO: 236 - protein, 6 aa, Artificial Sequence
  synthetic peptide
QESGPD

SEQ ID NO: 237 - protein, 6 aa, Artificial Sequence
  synthetic peptide
QGYEPD

SEQ ID NO: 238 - protein, 6 aa, Artificial Sequence
  synthetic peptide
QSGEPD

SEQ ID NO: 239 - protein, 6 aa, Artificial Sequence
  synthetic peptide
QGEPYD

SEQ ID NO: 240 - protein, 6 aa, Artificial Sequence
  synthetic peptide
QSEPGD

SEQ ID NO: 241 - protein, 6 aa, Artificial Sequence
  synthetic peptide
QETGYF

SEQ ID NO: 242 - protein, 6 aa, Artificial Sequence
  synthetic peptide
QETSGF

SEQ ID NO: 243 - protein, 6 aa, Artificial Sequence
  synthetic peptide
QEGYTF

SEQ ID NO: 244 - protein, 6 aa, Artificial Sequence
  synthetic peptide
QESGTF

SEQ ID NO: 245 - protein, 6 aa, Artificial Sequence
  synthetic peptide
QGYETF

SEQ ID NO: 246 - protein, 6 aa, Artificial Sequence
  synthetic peptide
QSGETF

SEQ ID NO: 247 - protein, 6 aa, Artificial Sequence
  synthetic peptide
QGETYF

SEQ ID NO: 248 - protein, 6 aa, Artificial Sequence
  synthetic peptide
QSETGF

SEQ ID NO: 249 - protein, 6 aa, Artificial Sequence
  synthetic peptide
QPEGYH

SEQ ID NO: 250 - protein, 6 aa, Artificial Sequence
  synthetic peptide
QPESGH

SEQ ID NO: 251 - protein, 6 aa, Artificial Sequence
  synthetic peptide
QPGYEH

SEQ ID NO: 252 - protein, 6 aa, Artificial Sequence
  synthetic peptide
QPSGEH

SEQ ID NO: 253 - protein, 6 aa, Artificial Sequence
  synthetic peptide
QGYPEH

SEQ ID NO: 254 - protein, 6 aa, Artificial Sequence
  synthetic peptide
QSGPEH

SEQ ID NO: 255 - protein, 6 aa, Artificial Sequence
  synthetic peptide
QGPEYH

SEQ ID NO: 256 - protein, 6 aa, Artificial Sequence
  synthetic peptide
QSPEGH

SEQ ID NO: 257 - protein, 6 aa, Artificial Sequence
  synthetic peptide
QNHGYE

SEQ ID NO: 258 - protein, 6 aa, Artificial Sequence
  synthetic peptide
QNHSGE

SEQ ID NO: 259 - protein, 6 aa, Artificial Sequence
  synthetic peptide
QNGYHE

SEQ ID NO: 260 - protein, 6 aa, Artificial Sequence
  synthetic peptide
QNSGHE

SEQ ID NO: 261 - protein, 6 aa, Artificial Sequence
  synthetic peptide
QGYNHE

SEQ ID NO: 262 - protein, 6 aa, Artificial Sequence
  synthetic peptide
QGYNHE

SEQ ID NO: 263 - protein, 6 aa, Artificial Sequence
  synthetic peptide
QGNHYE

SEQ ID NO: 264 - protein, 6 aa, Artificial Sequence
  synthetic peptide
QSNHGE

SEQ ID NO: 265 - protein, 6 aa, Artificial Sequence
  synthetic peptide
QFEGYK

SEQ ID NO: 266 - protein, 6 aa, Artificial Sequence
  synthetic peptide
QFESGK

SEQ ID NO: 267 - protein, 6 aa, Artificial Sequence
  synthetic peptide
QFGYEK

SEQ ID NO: 268 - protein, 6 aa, Artificial Sequence
  synthetic peptide
QFSGEK

SEQ ID NO: 269 - protein, 6 aa, Artificial Sequence
  synthetic peptide
QGYFEK

SEQ ID NO: 270 - protein, 6 aa, Artificial Sequence
  synthetic peptide
QSGFEK

SEQ ID NO: 271 - protein, 6 aa, Artificial Sequence
  synthetic peptide
QGFEYK

SEQ ID NO: 272 - protein, 6 aa, Artificial Sequence
  synthetic peptide
QSFEGK

SEQ ID NO: 273 - protein, 6 aa, Artificial Sequence
  synthetic peptide
QTFGYN

SEQ ID NO: 274 - protein, 6 aa, Artificial Sequence
  synthetic peptide
QTFSGN

SEQ ID NO: 275 - protein, 6 aa, Artificial Sequence
  synthetic peptide
QTGYFN

SEQ ID NO: 276 - protein, 6 aa, Artificial Sequence
  synthetic peptide
QTSGFN

SEQ ID NO: 277 - protein, 6 aa, Artificial Sequence
  synthetic peptide
QGYTFN

SEQ ID NO: 278 - protein, 6 aa, Artificial Sequence
  synthetic peptide
QSGTFN

SEQ ID NO: 279 - protein, 6 aa, Artificial Sequence
  synthetic peptide
QGTFYN

SEQ ID NO: 280 - protein, 6 aa, Artificial Sequence
  synthetic peptide
QSTFGN

SEQ ID NO: 281 - protein, 6 aa, Artificial Sequence
  synthetic peptide
QKEGYF

SEQ ID NO: 282 - protein, 6 aa, Artificial Sequence
  synthetic peptide
QKESGF

SEQ ID NO: 283 - protein, 6 aa, Artificial Sequence
  synthetic peptide
QKGYEF

SEQ ID NO: 284 - protein, 6 aa, Artificial Sequence
  synthetic peptide
QKSGEF

SEQ ID NO: 285 - protein, 6 aa, Artificial Sequence
  synthetic peptide
QGYKEF

SEQ ID NO: 286 - protein, 6 aa, Artificial Sequence
  synthetic peptide
QSGKEF

SEQ ID NO: 287 - protein, 6 aa, Artificial Sequence
  synthetic peptide
QGKEYF

SEQ ID NO: 288 - protein, 6 aa, Artificial Sequence
  synthetic peptide
QSKEGF

SEQ ID NO: 289 - protein, 6 aa, Artificial Sequence
  synthetic peptide
QLHGYT

SEQ ID NO: 290 - protein, 6 aa, Artificial Sequence
  synthetic peptide
QLHSGT

SEQ ID NO: 291 - protein, 6 aa, Artificial Sequence
  synthetic peptide
QLGYHT

SEQ ID NO: 292 - protein, 6 aa, Artificial Sequence
  synthetic peptide
QLSGHT

SEQ ID NO: 293 - protein, 6 aa, Artificial Sequence
  synthetic peptide
QGYLHT

SEQ ID NO: 294 - protein, 6 aa, Artificial Sequence
  synthetic peptide
QSGLHT

SEQ ID NO: 295 - protein, 6 aa, Artificial Sequence
  synthetic peptide
QGLHYT

SEQ ID NO: 296 - protein, 6 aa, Artificial Sequence
  synthetic peptide
QSLHGT

SEQ ID NO: 297 - protein, 6 aa, Artificial Sequence
  synthetic peptide
QLDGYE

SEQ ID NO: 298 - protein, 6 aa, Artificial Sequence
  synthetic peptide
QLDSGE

SEQ ID NO: 299 - protein, 6 aa, Artificial Sequence
  synthetic peptide
QLGYDE

SEQ ID NO: 300 - protein, 6 aa, Artificial Sequence
  synthetic peptide
QLSGDE

SEQ ID NO: 301 - protein, 6 aa, Artificial Sequence
  synthetic peptide
QGYLDE

SEQ ID NO: 302 - protein, 6 aa, Artificial Sequence
  synthetic peptide
QSGLDE

SEQ ID NO: 303 - protein, 6 aa, Artificial Sequence
  synthetic peptide
QGLDYE

SEQ ID NO: 304 - protein, 6 aa, Artificial Sequence
  synthetic peptide
QSLDGE

SEQ ID NO: 305 - protein, 6 aa, Artificial Sequence
  synthetic peptide
NEPGYL

SEQ ID NO: 306 - protein, 6 aa, Artificial Sequence
  synthetic peptide
NEPSGL

SEQ ID NO: 307 - protein, 6 aa, Artificial Sequence
  synthetic peptide
NEGYPL

SEQ ID NO: 308 - protein, 6 aa, Artificial Sequence
  synthetic peptide
NESGPL

SEQ ID NO: 309 - protein, 6 aa, Artificial Sequence
  synthetic peptide
NGYEPL

SEQ ID NO: 310 - protein, 6 aa, Artificial Sequence
  synthetic peptide
NSGEPL

SEQ ID NO: 311 - protein, 6 aa, Artificial Sequence
  synthetic peptide
NGEPYL

SEQ ID NO: 312 - protein, 6 aa, Artificial Sequence
  synthetic peptide
NSEPGL

SEQ ID NO: 313 - protein, 6 aa, Artificial Sequence
  synthetic peptide
NEFGYH

SEQ ID NO: 314 - protein, 6 aa, Artificial Sequence
  synthetic peptide
NEFSGH

SEQ ID NO: 315 - protein, 6 aa, Artificial Sequence
  synthetic peptide
NEGYFH

SEQ ID NO: 316 - protein, 6 aa, Artificial Sequence
  synthetic peptide
NESGFH

SEQ ID NO: 317 - protein, 6 aa, Artificial Sequence
  synthetic peptide
NGYEFH

SEQ ID NO: 318 - protein, 6 aa, Artificial Sequence
  synthetic peptide
NSGEFH

SEQ ID NO: 319 - protein, 6 aa, Artificial Sequence
  synthetic peptide
NGEFYH

SEQ ID NO: 320 - protein, 6 aa, Artificial Sequence
  synthetic peptide
NSEFGH

SEQ ID NO: 321 - protein, 6 aa, Artificial Sequence
  synthetic peptide
NPEGYF

SEQ ID NO: 322 - protein, 6 aa, Artificial Sequence
  synthetic peptide
NPESGF

SEQ ID NO: 323 - protein, 6 aa, Artificial Sequence
  synthetic peptide
NPGYEF

SEQ ID NO: 324 - protein, 6 aa, Artificial Sequence
  synthetic peptide
NPSGEF

SEQ ID NO: 325 - protein, 6 aa, Artificial Sequence
  synthetic peptide
NGYPEF

SEQ ID NO: 326 - protein, 6 aa, Artificial Sequence
  synthetic peptide
NSGPEF

SEQ ID NO: 327 - protein, 6 aa, Artificial Sequence
  synthetic peptide
NGPEYF

SEQ ID NO: 328 - protein, 6 aa, Artificial Sequence
  synthetic peptide
NSPEGF

SEQ ID NO: 329 - protein, 6 aa, Artificial Sequence
  synthetic peptide
NQHGYD

SEQ ID NO: 330 - protein, 6 aa, Artificial Sequence
  synthetic peptide
NQHSGD

SEQ ID NO: 331 - protein, 6 aa, Artificial Sequence
  synthetic peptide
NQGYHD

SEQ ID NO: 332 - protein, 6 aa, Artificial Sequence
  synthetic peptide
NQSGHD

SEQ ID NO: 333 - protein, 6 aa, Artificial Sequence
  synthetic peptide
NGYQHD

SEQ ID NO: 334 - protein, 6 aa, Artificial Sequence
  synthetic peptide
NSGQHD

SEQ ID NO: 335 - protein, 6 aa, Artificial Sequence
  synthetic peptide
NGQHYD

SEQ ID NO: 336 - protein, 6 aa, Artificial Sequence
  synthetic peptide
NSQHGD

SEQ ID NO: 337 - protein, 6 aa, Artificial Sequence
  synthetic peptide
NFEGYP

SEQ ID NO: 338 - protein, 6 aa, Artificial Sequence
  synthetic peptide
NFESGP

SEQ ID NO: 339 - protein, 6 aa, Artificial Sequence
  synthetic peptide
NFGYEP

SEQ ID NO: 340 - protein, 6 aa, Artificial Sequence
  synthetic peptide
NFSGEP

SEQ ID NO: 341 - protein, 6 aa, Artificial Sequence
  synthetic peptide
NGYFEP

SEQ ID NO: 342 - protein, 6 aa, Artificial Sequence
  synthetic peptide
NSGFEP

SEQ ID NO: 343 - protein, 6 aa, Artificial Sequence
  synthetic peptide
NGFEYP

SEQ ID NO: 344 - protein, 6 aa, Artificial Sequence
  synthetic peptide
NSFEGP

SEQ ID NO: 345 - protein, 6 aa, Artificial Sequence
  synthetic peptide
NFKGYH

SEQ ID NO: 346 - protein, 6 aa, Artificial Sequence
  synthetic peptide
NFKSGH

SEQ ID NO: 347 - protein, 6 aa, Artificial Sequence
  synthetic peptide
NFGYKH

SEQ ID NO: 348 - protein, 6 aa, Artificial Sequence
  synthetic peptide
NFSGKH

SEQ ID NO: 349 - protein, 6 aa, Artificial Sequence
  synthetic peptide
NGYFKH

SEQ ID NO: 350 - protein, 6 aa, Artificial Sequence
  synthetic peptide
NSGFKH

SEQ ID NO: 351 - protein, 6 aa, Artificial Sequence
  synthetic peptide
NGFKYH

SEQ ID NO: 352 - protein, 6 aa, Artificial Sequence
  synthetic peptide
NSFKGH

SEQ ID NO: 353 - protein, 6 aa, Artificial Sequence
  synthetic peptide
NHPGYT

SEQ ID NO: 354 - protein, 6 aa, Artificial Sequence
  synthetic peptide
NHPSGT

SEQ ID NO: 355 - protein, 6 aa, Artificial Sequence
  synthetic peptide
NHGYPT

SEQ ID NO: 356 - protein, 6 aa, Artificial Sequence
  synthetic peptide
NHSGPT

SEQ ID NO: 357 - protein, 6 aa, Artificial Sequence
  synthetic peptide
NGYHPT

SEQ ID NO: 358 - protein, 6 aa, Artificial Sequence
  synthetic peptide
NSGHPT

SEQ ID NO: 359 - protein, 6 aa, Artificial Sequence
  synthetic peptide
NGHPYT

SEQ ID NO: 360 - protein, 6 aa, Artificial Sequence
  synthetic peptide
NSHPGT

SEQ ID NO: 361 - protein, 6 aa, Artificial Sequence
  synthetic peptide
NHTGYD

SEQ ID NO: 362 - protein, 6 aa, Artificial Sequence
  synthetic peptide
NHTSGD

SEQ ID NO: 363 - protein, 6 aa, Artificial Sequence
  synthetic peptide
NHGYTD

SEQ ID NO: 364 - protein, 6 aa, Artificial Sequence
  synthetic peptide
NHSGTD

SEQ ID NO: 365 - protein, 6 aa, Artificial Sequence
  synthetic peptide
NGYHTD

SEQ ID NO: 366 - protein, 6 aa, Artificial Sequence
  synthetic peptide
NSGHTD

SEQ ID NO: 367 - protein, 6 aa, Artificial Sequence
  synthetic peptide
NGHTYD

SEQ ID NO: 368 - protein, 6 aa, Artificial Sequence
  synthetic peptide
NSHTGD

SEQ ID NO: 369 - protein, 6 aa, Artificial Sequence
  synthetic peptide
NTHGYK

SEQ ID NO: 370 - protein, 6 aa, Artificial Sequence
  synthetic peptide
NTHSGK

SEQ ID NO: 371 - protein, 6 aa, Artificial Sequence
  synthetic peptide
NTGYHK

SEQ ID NO: 372 - protein, 6 aa, Artificial Sequence
  synthetic peptide
NTSGHK

SEQ ID NO: 373 - protein, 6 aa, Artificial Sequence
  synthetic peptide
NGYTHK

SEQ ID NO: 374 - protein, 6 aa, Artificial Sequence
  synthetic peptide
NSGTHK

SEQ ID NO: 375 - protein, 6 aa, Artificial Sequence
  synthetic peptide
NGTHYK

SEQ ID NO: 376 - protein, 6 aa, Artificial Sequence
  synthetic peptide
NSTHGK

SEQ ID NO: 377 - protein, 6 aa, Artificial Sequence
  synthetic peptide
NKHGYL

SEQ ID NO: 378 - protein, 6 aa, Artificial Sequence
  synthetic peptide
NKHSGL

SEQ ID NO: 379 - protein, 6 aa, Artificial Sequence
  synthetic peptide
NKGYHL

SEQ ID NO: 380 - protein, 6 aa, Artificial Sequence
  synthetic peptide
NKSGHL

SEQ ID NO: 381 - protein, 6 aa, Artificial Sequence
  synthetic peptide
NGYKHL

SEQ ID NO: 382 - protein, 6 aa, Artificial Sequence
  synthetic peptide
NSGKHL

SEQ ID NO: 383 - protein, 6 aa, Artificial Sequence
  synthetic peptide
NGKHYL

SEQ ID NO: 384 - protein, 6 aa, Artificial Sequence
  synthetic peptide
NSKHGL

SEQ ID NO: 385 - protein, 6 aa, Artificial Sequence
  synthetic peptide
NLFGYD

SEQ ID NO: 386 - protein, 6 aa, Artificial Sequence
  synthetic peptide
NLFSGD

SEQ ID NO: 387 - protein, 6 aa, Artificial Sequence
  synthetic peptide
NLGYFD

SEQ ID NO: 388 - protein, 6 aa, Artificial Sequence
  synthetic peptide
NLSGFD

SEQ ID NO: 389 - protein, 6 aa, Artificial Sequence
  synthetic peptide
NGYLFD

SEQ ID NO: 390 - protein, 6 aa, Artificial Sequence
  synthetic peptide
NSGLFD

SEQ ID NO: 391 - protein, 6 aa, Artificial Sequence
  synthetic peptide
NGLFYD

SEQ ID NO: 392 - protein, 6 aa, Artificial Sequence
  synthetic peptide
NSLFGD

SEQ ID NO: 393 - protein, 6 aa, Artificial Sequence
  synthetic peptide
NDLGYF

SEQ ID NO: 394 - protein, 6 aa, Artificial Sequence
  synthetic peptide
NDLSGF

SEQ ID NO: 395 - protein, 6 aa, Artificial Sequence
  synthetic peptide
NDGYLF

SEQ ID NO: 396 - protein, 6 aa, Artificial Sequence
  synthetic peptide
NDSGLF

SEQ ID NO: 397 - protein, 6 aa, Artificial Sequence
  synthetic peptide
NGYDLF

SEQ ID NO: 398 - protein, 6 aa, Artificial Sequence
  synthetic peptide
NSGDLF

SEQ ID NO: 399 - protein, 6 aa, Artificial Sequence
  synthetic peptide
NGDLYF

SEQ ID NO: 400 - protein, 6 aa, Artificial Sequence
  synthetic peptide
NSDLGF

SEQ ID NO: 401 - protein, 6 aa, Artificial Sequence
  synthetic peptide
FEQGYP

SEQ ID NO: 402 - protein, 6 aa, Artificial Sequence
  synthetic peptide
FEQSGP

SEQ ID NO: 403 - protein, 6 aa, Artificial Sequence
  synthetic peptide
FEGYQP

SEQ ID NO: 404 - protein, 6 aa, Artificial Sequence
  synthetic peptide
FESGQP

SEQ ID NO: 405 - protein, 6 aa, Artificial Sequence
  synthetic peptide
FGYEQP

SEQ ID NO: 406 - protein, 6 aa, Artificial Sequence
  synthetic peptide
FSGEQP

SEQ ID NO: 407 - protein, 6 aa, Artificial Sequence
  synthetic peptide
FGEQYP

SEQ ID NO: 408 - protein, 6 aa, Artificial Sequence
  synthetic peptide
FSEQGP

SEQ ID NO: 409 - protein, 6 aa, Artificial Sequence
  synthetic peptide
FEKGYT

SEQ ID NO: 410 - protein, 6 aa, Artificial Sequence
  synthetic peptide
FEKSGT

SEQ ID NO: 411 - protein, 6 aa, Artificial Sequence
  synthetic peptide
FEGYKT

SEQ ID NO: 412 - protein, 6 aa, Artificial Sequence
  synthetic peptide
FESGKT

SEQ ID NO: 413 - protein, 6 aa, Artificial Sequence
  synthetic peptide
FGYEKT

SEQ ID NO: 414 - protein, 6 aa, Artificial Sequence
  synthetic peptide
FSGEKT

SEQ ID NO: 415 - protein, 6 aa, Artificial Sequence
  synthetic peptide
FGEKYT

SEQ ID NO: 416 - protein, 6 aa, Artificial Sequence
  synthetic peptide
FSEKGT

SEQ ID NO: 417 - protein, 6 aa, Artificial Sequence
  synthetic peptide
FEDGYH

SEQ ID NO: 418 - protein, 6 aa, Artificial Sequence
  synthetic peptide
FEDSGH

SEQ ID NO: 419 - protein, 6 aa, Artificial Sequence
  synthetic peptide
FEGYDH

SEQ ID NO: 420 - protein, 6 aa, Artificial Sequence
  synthetic peptide
FESGDH

SEQ ID NO: 421 - protein, 6 aa, Artificial Sequence
  synthetic peptide
FGYEDH

SEQ ID NO: 422 - protein, 6 aa, Artificial Sequence
  synthetic peptide
FSGEDH

SEQ ID NO: 423 - protein, 6 aa, Artificial Sequence
  synthetic peptide
FGEDYH

SEQ ID NO: 424 - protein, 6 aa, Artificial Sequence
  synthetic peptide
FSEDGH

SEQ ID NO: 425 - protein, 6 aa, Artificial Sequence
  synthetic peptide
FPNGYE

SEQ ID NO: 426 - protein, 6 aa, Artificial Sequence
  synthetic peptide
FPNSGE

SEQ ID NO: 427 - protein, 6 aa, Artificial Sequence
  synthetic peptide
FPGYNE

SEQ ID NO: 428 - protein, 6 aa, Artificial Sequence
  synthetic peptide
FPSGNE

SEQ ID NO: 429 - protein, 6 aa, Artificial Sequence
  synthetic peptide
FGYPNE

SEQ ID NO: 430 - protein, 6 aa, Artificial Sequence
  synthetic peptide
FSGPNE

SEQ ID NO: 431 - protein, 6 aa, Artificial Sequence
  synthetic peptide
FGPNYE

SEQ ID NO: 432 - protein, 6 aa, Artificial Sequence
  synthetic peptide
FSPNGE

SEQ ID NO: 433 - protein, 6 aa, Artificial Sequence
  synthetic peptide
FPKGYL

SEQ ID NO: 434 - protein, 6 aa, Artificial Sequence
  synthetic peptide
FPKSGL

SEQ ID NO: 435 - protein, 6 aa, Artificial Sequence
  synthetic peptide
FPGYKL

SEQ ID NO: 436 - protein, 6 aa, Artificial Sequence
  synthetic peptide
FPSGKL

SEQ ID NO: 437 - protein, 6 aa, Artificial Sequence
  synthetic peptide
FGYPKL

SEQ ID NO: 438 - protein, 6 aa, Artificial Sequence
  synthetic peptide
FSGPKL

SEQ ID NO: 439 - protein, 6 aa, Artificial Sequence
  synthetic peptide
FGPKYL

SEQ ID NO: 440 - protein, 6 aa, Artificial Sequence
  synthetic peptide
FSPKGL

SEQ ID NO: 441 - protein, 6 aa, Artificial Sequence
  synthetic peptide
FQNGYK

SEQ ID NO: 442 - protein, 6 aa, Artificial Sequence
  synthetic peptide
FQNSGK

SEQ ID NO: 443 - protein, 6 aa, Artificial Sequence
  synthetic peptide
FQGYNK

SEQ ID NO: 444 - protein, 6 aa, Artificial Sequence
  synthetic peptide
FQSGNK

SEQ ID NO: 445 - protein, 6 aa, Artificial Sequence
  synthetic peptide
FGYQNK

SEQ ID NO: 446 - protein, 6 aa, Artificial Sequence
  synthetic peptide
FSGQNK

SEQ ID NO: 447 - protein, 6 aa, Artificial Sequence
  synthetic peptide
FGQNYK

SEQ ID NO: 448 - protein, 6 aa, Artificial Sequence
  synthetic peptide
FSQNGK

SEQ ID NO: 449 - protein, 6 aa, Artificial Sequence
  synthetic peptide
FNPGYE

SEQ ID NO: 450 - protein, 6 aa, Artificial Sequence
  synthetic peptide
FNPSGE

SEQ ID NO: 451 - protein, 6 aa, Artificial Sequence
  synthetic peptide
FNGYPE

SEQ ID NO: 452 - protein, 6 aa, Artificial Sequence
  synthetic peptide
FNSGPE

SEQ ID NO: 453 - protein, 6 aa, Artificial Sequence
  synthetic peptide
FGYNPE

SEQ ID NO: 454 - protein, 6 aa, Artificial Sequence
  synthetic peptide
FSGNPE

SEQ ID NO: 455 - protein, 6 aa, Artificial Sequence
  synthetic peptide
FGNPYE

SEQ ID NO: 456 - protein, 6 aa, Artificial Sequence
  synthetic peptide
FSNPGE

SEQ ID NO: 457 - protein, 6 aa, Artificial Sequence
  synthetic peptide
FHEGYP

SEQ ID NO: 458 - protein, 6 aa, Artificial Sequence
  synthetic peptide
FHESGP

SEQ ID NO: 459 - protein, 6 aa, Artificial Sequence
  synthetic peptide
FHGYEP

SEQ ID NO: 460 - protein, 6 aa, Artificial Sequence
  synthetic peptide
FHSGEP

SEQ ID NO: 461 - protein, 6 aa, Artificial Sequence
  synthetic peptide
FGYHEP

SEQ ID NO: 462 - protein, 6 aa, Artificial Sequence
  synthetic peptide
FSGHEP

SEQ ID NO: 463 - protein, 6 aa, Artificial Sequence
  synthetic peptide
FGHEYP

SEQ ID NO: 464 - protein, 6 aa, Artificial Sequence
  synthetic peptide
FSHEGP

SEQ ID NO: 465 - protein, 6 aa, Artificial Sequence
  synthetic peptide
FHKGYE

SEQ ID NO: 466 - protein, 6 aa, Artificial Sequence
  synthetic peptide
FHKSGE

SEQ ID NO: 467 - protein, 6 aa, Artificial Sequence
  synthetic peptide
FHGYKE

SEQ ID NO: 468 - protein, 6 aa, Artificial Sequence
  synthetic peptide
FHSGKE

SEQ ID NO: 469 - protein, 6 aa, Artificial Sequence
  synthetic peptide
FGYHKE

SEQ ID NO: 470 - protein, 6 aa, Artificial Sequence
  synthetic peptide
FSGHKE

SEQ ID NO: 471 - protein, 6 aa, Artificial Sequence
  synthetic peptide
FGHKYE

SEQ ID NO: 472 - protein, 6 aa, Artificial Sequence
  synthetic peptide
FSHKGE

SEQ ID NO: 473 - protein, 6 aa, Artificial Sequence
  synthetic peptide
FTHGYN

SEQ ID NO: 474 - protein, 6 aa, Artificial Sequence
  synthetic peptide
FTHSGN

SEQ ID NO: 475 - protein, 6 aa, Artificial Sequence
  synthetic peptide
FTGYHN

SEQ ID NO: 476 - protein, 6 aa, Artificial Sequence
  synthetic peptide
FTSGHN

SEQ ID NO: 477 - protein, 6 aa, Artificial Sequence
  synthetic peptide
FGYTHN

SEQ ID NO: 478 - protein, 6 aa, Artificial Sequence
  synthetic peptide
FSGTHN

SEQ ID NO: 479 - protein, 6 aa, Artificial Sequence
  synthetic peptide
FGTHYN

SEQ ID NO: 480 - protein, 6 aa, Artificial Sequence
  synthetic peptide
FSTHGN

SEQ ID NO: 481 - protein, 6 aa, Artificial Sequence
  synthetic peptide
FTLGYQ

SEQ ID NO: 482 - protein, 6 aa, Artificial Sequence
  synthetic peptide
FTLSGQ

SEQ ID NO: 483 - protein, 6 aa, Artificial Sequence
  synthetic peptide
FTGYLQ

SEQ ID NO: 484 - protein, 6 aa, Artificial Sequence
  synthetic peptide
FTSGLQ

SEQ ID NO: 485 - protein, 6 aa, Artificial Sequence
  synthetic peptide
FGYTLQ

SEQ ID NO: 486 - protein, 6 aa, Artificial Sequence
  synthetic peptide
FSGTLQ

SEQ ID NO: 487 - protein, 6 aa, Artificial Sequence
  synthetic peptide
FGTLYQ

SEQ ID NO: 488 - protein, 6 aa, Artificial Sequence
  synthetic peptide
FSTLGQ

SEQ ID NO: 489 - protein, 6 aa, Artificial Sequence
  synthetic peptide
FKQGYH

SEQ ID NO: 490 - protein, 6 aa, Artificial Sequence
  synthetic peptide
FKQSGH

SEQ ID NO: 491 - protein, 6 aa, Artificial Sequence
  synthetic peptide
FKGYQH

SEQ ID NO: 492 - protein, 6 aa, Artificial Sequence
  synthetic peptide
FKSGQH

SEQ ID NO: 493 - protein, 6 aa, Artificial Sequence
  synthetic peptide
FGYKQH

SEQ ID NO: 494 - protein, 6 aa, Artificial Sequence
  synthetic peptide
FSGKQH

SEQ ID NO: 495 - protein, 6 aa, Artificial Sequence
  synthetic peptide
FGKQYH

SEQ ID NO: 496 - protein, 6 aa, Artificial Sequence
  synthetic peptide
FSKQGH

SEQ ID NO: 497 - protein, 6 aa, Artificial Sequence
  synthetic peptide
FKLGYP

SEQ ID NO: 498 - protein, 6 aa, Artificial Sequence
  synthetic peptide
FKLSGP

SEQ ID NO: 499 - protein, 6 aa, Artificial Sequence
  synthetic peptide
FKGYLP

SEQ ID NO: 500 - protein, 6 aa, Artificial Sequence
  synthetic peptide
FKSGLP

SEQ ID NO: 501 - protein, 6 aa, Artificial Sequence
  synthetic peptide
FGYKLP

SEQ ID NO: 502 - protein, 6 aa, Artificial Sequence
  synthetic peptide
FSGKLP

SEQ ID NO: 503 - protein, 6 aa, Artificial Sequence
  synthetic peptide
FGKLYP

SEQ ID NO: 504 - protein, 6 aa, Artificial Sequence
  synthetic peptide
FSKLGP

SEQ ID NO: 505 - protein, 6 aa, Artificial Sequence
  synthetic peptide
FLEGYD

SEQ ID NO: 506 - protein, 6 aa, Artificial Sequence
  synthetic peptide
FLESGD

SEQ ID NO: 507 - protein, 6 aa, Artificial Sequence
  synthetic peptide
FLGYED

SEQ ID NO: 508 - protein, 6 aa, Artificial Sequence
  synthetic peptide
FLSGED

SEQ ID NO: 509 - protein, 6 aa, Artificial Sequence
  synthetic peptide
FGYLED

SEQ ID NO: 510 - protein, 6 aa, Artificial Sequence
  synthetic peptide
FSGLED

SEQ ID NO: 511 - protein, 6 aa, Artificial Sequence
  synthetic peptide
FGLEYD

SEQ ID NO: 512 - protein, 6 aa, Artificial Sequence
  synthetic peptide
FSLEGD

SEQ ID NO: 513 - protein, 6 aa, Artificial Sequence
  synthetic peptide
FLHGYQ

SEQ ID NO: 514 - protein, 6 aa, Artificial Sequence
  synthetic peptide
FLHSGQ

SEQ ID NO: 515 - protein, 6 aa, Artificial Sequence
  synthetic peptide
FLGYHQ

SEQ ID NO: 516 - protein, 6 aa, Artificial Sequence
  synthetic peptide
FLSGHQ

SEQ ID NO: 517 - protein, 6 aa, Artificial Sequence
  synthetic peptide
FGYLHQ

SEQ ID NO: 518 - protein, 6 aa, Artificial Sequence
  synthetic peptide
FSGLHQ

SEQ ID NO: 519 - protein, 6 aa, Artificial Sequence
  synthetic peptide
FGLHYQ

SEQ ID NO: 520 - protein, 6 aa, Artificial Sequence
  synthetic peptide
FSLHGQ

SEQ ID NO: 521 - protein, 6 aa, Artificial Sequence
  synthetic peptide
FDTGYE

SEQ ID NO: 522 - protein, 6 aa, Artificial Sequence
  synthetic peptide
FDTSGE

SEQ ID NO: 523 - protein, 6 aa, Artificial Sequence
  synthetic peptide
FDGYTE

SEQ ID NO: 524 - protein, 6 aa, Artificial Sequence
  synthetic peptide
FDSGTE

SEQ ID NO: 525 - protein, 6 aa, Artificial Sequence
  synthetic peptide
FGYDTE

SEQ ID NO: 526 - protein, 6 aa, Artificial Sequence
  synthetic peptide
FSGDTE

SEQ ID NO: 527 - protein, 6 aa, Artificial Sequence
  synthetic peptide
FGDTYE

SEQ ID NO: 528 - protein, 6 aa, Artificial Sequence
  synthetic peptide
FSDTGE

SEQ ID NO: 529 - protein, 6 aa, Artificial Sequence
  synthetic peptide
HEQGYF

SEQ ID NO: 530 - protein, 6 aa, Artificial Sequence
  synthetic peptide
HEQSGF

SEQ ID NO: 531 - protein, 6 aa, Artificial Sequence
  synthetic peptide
HEGYQF

SEQ ID NO: 532 - protein, 6 aa, Artificial Sequence
  synthetic peptide
HESGQF

SEQ ID NO: 533 - protein, 6 aa, Artificial Sequence
  synthetic peptide
HGYEQF

SEQ ID NO: 534 - protein, 6 aa, Artificial Sequence
  synthetic peptide
HSGEQF

SEQ ID NO: 535 - protein, 6 aa, Artificial Sequence
  synthetic peptide
HGEQYF

SEQ ID NO: 536 - protein, 6 aa, Artificial Sequence
  synthetic peptide
HSEQGF

SEQ ID NO: 537 - protein, 6 aa, Artificial Sequence
  synthetic peptide
HEKGYP

SEQ ID NO: 538 - protein, 6 aa, Artificial Sequence
  synthetic peptide
HEKSGP

SEQ ID NO: 539 - protein, 6 aa, Artificial Sequence
  synthetic peptide
HEGYKP

SEQ ID NO: 540 - protein, 6 aa, Artificial Sequence
  synthetic peptide
HESGKP

SEQ ID NO: 541 - protein, 6 aa, Artificial Sequence
  synthetic peptide
HGYEKP

SEQ ID NO: 542 - protein, 6 aa, Artificial Sequence
  synthetic peptide
HSGEKP

SEQ ID NO: 543 - protein, 6 aa, Artificial Sequence
  synthetic peptide
HGEKYP

SEQ ID NO: 544 - protein, 6 aa, Artificial Sequence
  synthetic peptide
HSEKGP

SEQ ID NO: 545 - protein, 6 aa, Artificial Sequence
  synthetic peptide
HPEGYD

SEQ ID NO: 546 - protein, 6 aa, Artificial Sequence
  synthetic peptide
HPESGD

SEQ ID NO: 547 - protein, 6 aa, Artificial Sequence
  synthetic peptide
HPGYED

SEQ ID NO: 548 - protein, 6 aa, Artificial Sequence
  synthetic peptide
HPSGED

SEQ ID NO: 549 - protein, 6 aa, Artificial Sequence
  synthetic peptide
HGYPED

SEQ ID NO: 550 - protein, 6 aa, Artificial Sequence
  synthetic peptide
HSGPED

SEQ ID NO: 551 - protein, 6 aa, Artificial Sequence
  synthetic peptide
HGPEYD

SEQ ID NO: 552 - protein, 6 aa, Artificial Sequence
  synthetic peptide
HSPEGD

SEQ ID NO: 553 - protein, 6 aa, Artificial Sequence
  synthetic peptide
HPFGYL

SEQ ID NO: 554 - protein, 6 aa, Artificial Sequence
  synthetic peptide
HPFSGL

SEQ ID NO: 555 - protein, 6 aa, Artificial Sequence
  synthetic peptide
HPGYFL

SEQ ID NO: 556 - protein, 6 aa, Artificial Sequence
  synthetic peptide
HPSGFL

SEQ ID NO: 557 - protein, 6 aa, Artificial Sequence
  synthetic peptide
HGYPFL

SEQ ID NO: 558 - protein, 6 aa, Artificial Sequence
  synthetic peptide
HSGPFL

SEQ ID NO: 559 - protein, 6 aa, Artificial Sequence
  synthetic peptide
HGPFYL

SEQ ID NO: 560 - protein, 6 aa, Artificial Sequence
  synthetic peptide
HSPFGL

SEQ ID NO: 561 - protein, 6 aa, Artificial Sequence
  synthetic peptide
HQEGYL

SEQ ID NO: 562 - protein, 6 aa, Artificial Sequence
  synthetic peptide
HQESGL

SEQ ID NO: 563 - protein, 6 aa, Artificial Sequence
  synthetic peptide
HQGYEL

SEQ ID NO: 564 - protein, 6 aa, Artificial Sequence
  synthetic peptide
HQSGEL

SEQ ID NO: 565 - protein, 6 aa, Artificial Sequence
  synthetic peptide
HGYQEL

SEQ ID NO: 566 - protein, 6 aa, Artificial Sequence
  synthetic peptide
HSGQEL

SEQ ID NO: 567 - protein, 6 aa, Artificial Sequence
  synthetic peptide
HGQEYL

SEQ ID NO: 568 - protein, 6 aa, Artificial Sequence
  synthetic peptide
HSQEGL

SEQ ID NO: 569 - protein, 6 aa, Artificial Sequence
  synthetic peptide
HQTGYN

SEQ ID NO: 570 - protein, 6 aa, Artificial Sequence
  synthetic peptide
HQTSGN

SEQ ID NO: 571 - protein, 6 aa, Artificial Sequence
  synthetic peptide
HQGYTN

SEQ ID NO: 572 - protein, 6 aa, Artificial Sequence
  synthetic peptide
HQSGTN

SEQ ID NO: 573 - protein, 6 aa, Artificial Sequence
  synthetic peptide
HGYQTN

SEQ ID NO: 574 - protein, 6 aa, Artificial Sequence
  synthetic peptide
HSGQTN

SEQ ID NO: 575 - protein, 6 aa, Artificial Sequence
  synthetic peptide
HGQTYN

SEQ ID NO: 576 - protein, 6 aa, Artificial Sequence
  synthetic peptide
HSQTGN

SEQ ID NO: 577 - protein, 6 aa, Artificial Sequence
  synthetic peptide
HNKGYD

SEQ ID NO: 578 - protein, 6 aa, Artificial Sequence
  synthetic peptide
HNKSGD

SEQ ID NO: 579 - protein, 6 aa, Artificial Sequence
  synthetic peptide
HNGYKD

SEQ ID NO: 580 - protein, 6 aa, Artificial Sequence
  synthetic peptide
HNSGKD

SEQ ID NO: 581 - protein, 6 aa, Artificial Sequence
  synthetic peptide
HGYNKD

SEQ ID NO: 582 - protein, 6 aa, Artificial Sequence
  synthetic peptide
HSGNKD

SEQ ID NO: 583 - protein, 6 aa, Artificial Sequence
  synthetic peptide
HGNKYD

SEQ ID NO: 584 - protein, 6 aa, Artificial Sequence
  synthetic peptide
HSNKGD

SEQ ID NO: 585 - protein, 6 aa, Artificial Sequence
  synthetic peptide
HNDGYT

SEQ ID NO: 586 - protein, 6 aa, Artificial Sequence
  synthetic peptide
HNDSGT

SEQ ID NO: 587 - protein, 6 aa, Artificial Sequence
  synthetic peptide
HNGYDT

SEQ ID NO: 588 - protein, 6 aa, Artificial Sequence
  synthetic peptide
HNSGDT

SEQ ID NO: 589 - protein, 6 aa, Artificial Sequence
  synthetic peptide
HGYNDT

SEQ ID NO: 590 - protein, 6 aa, Artificial Sequence
  synthetic peptide
HSGNDT

SEQ ID NO: 591 - protein, 6 aa, Artificial Sequence
  synthetic peptide
HGNDYT

SEQ ID NO: 592 - protein, 6 aa, Artificial Sequence
  synthetic peptide
HSNDGT

SEQ ID NO: 593 - protein, 6 aa, Artificial Sequence
  synthetic peptide
HFTGYK

SEQ ID NO: 594 - protein, 6 aa, Artificial Sequence
  synthetic peptide
HFTSGK

SEQ ID NO: 595 - protein, 6 aa, Artificial Sequence
  synthetic peptide
HFGYTK

SEQ ID NO: 596 - protein, 6 aa, Artificial Sequence
  synthetic peptide
HFSGTK

SEQ ID NO: 597 - protein, 6 aa, Artificial Sequence
  synthetic peptide
HGYFTK

SEQ ID NO: 598 - protein, 6 aa, Artificial Sequence
  synthetic peptide
HSGFTK

SEQ ID NO: 599 - protein, 6 aa, Artificial Sequence
  synthetic peptide
HGFTYK

SEQ ID NO: 600 - protein, 6 aa, Artificial Sequence
  synthetic peptide
HSFTGK

SEQ ID NO: 601 - protein, 6 aa, Artificial Sequence
  synthetic peptide
HTPGYN

SEQ ID NO: 602 - protein, 6 aa, Artificial Sequence
  synthetic peptide
HTPSGN

SEQ ID NO: 603 - protein, 6 aa, Artificial Sequence
  synthetic peptide
HTGYPN

SEQ ID NO: 604 - protein, 6 aa, Artificial Sequence
  synthetic peptide
HTSGPN

SEQ ID NO: 605 - protein, 6 aa, Artificial Sequence
  synthetic peptide
HGYTPN

SEQ ID NO: 606 - protein, 6 aa, Artificial Sequence
  synthetic peptide
HSGTPN

SEQ ID NO: 607 - protein, 6 aa, Artificial Sequence
  synthetic peptide
HGTPYN

SEQ ID NO: 608 - protein, 6 aa, Artificial Sequence
  synthetic peptide
HSTPGN

SEQ ID NO: 609 - protein, 6 aa, Artificial Sequence
  synthetic peptide
HTFGYQ

SEQ ID NO: 610 - protein, 6 aa, Artificial Sequence
  synthetic peptide
HTFSGQ

SEQ ID NO: 611 - protein, 6 aa, Artificial Sequence
  synthetic peptide
HTGYFQ

SEQ ID NO: 612 - protein, 6 aa, Artificial Sequence
  synthetic peptide
HTSGFQ

SEQ ID NO: 613 - protein, 6 aa, Artificial Sequence
  synthetic peptide
HGYTFQ

SEQ ID NO: 614 - protein, 6 aa, Artificial Sequence
  synthetic peptide
HSGTFQ

SEQ ID NO: 615 - protein, 6 aa, Artificial Sequence
  synthetic peptide
HGTFYQ

SEQ ID NO: 616 - protein, 6 aa, Artificial Sequence
  synthetic peptide
HSTFGQ

SEQ ID NO: 617 - protein, 6 aa, Artificial Sequence
  synthetic peptide
HKPGYE

SEQ ID NO: 618 - protein, 6 aa, Artificial Sequence
  synthetic peptide
HKPSGE

SEQ ID NO: 619 - protein, 6 aa, Artificial Sequence
  synthetic peptide
HKGYPE

SEQ ID NO: 620 - protein, 6 aa, Artificial Sequence
  synthetic peptide
HKSGPE

SEQ ID NO: 621 - protein, 6 aa, Artificial Sequence
  synthetic peptide
HGYKPE

SEQ ID NO: 622 - protein, 6 aa, Artificial Sequence
  synthetic peptide
HSGKPE

SEQ ID NO: 623 - protein, 6 aa, Artificial Sequence
  synthetic peptide
HGKPYE

SEQ ID NO: 624 - protein, 6 aa, Artificial Sequence
  synthetic peptide
HSKPGE

SEQ ID NO: 625 - protein, 6 aa, Artificial Sequence
  synthetic peptide
HLEGYF

SEQ ID NO: 626 - protein, 6 aa, Artificial Sequence
  synthetic peptide
HLESGF

SEQ ID NO: 627 - protein, 6 aa, Artificial Sequence
  synthetic peptide
HLGYEF

SEQ ID NO: 628 - protein, 6 aa, Artificial Sequence
  synthetic peptide
HLSGEF

SEQ ID NO: 629 - protein, 6 aa, Artificial Sequence
  synthetic peptide
HGYLEF

SEQ ID NO: 630 - protein, 6 aa, Artificial Sequence
  synthetic peptide
HSGLEF

SEQ ID NO: 631 - protein, 6 aa, Artificial Sequence
  synthetic peptide
HGLEYF

SEQ ID NO: 632 - protein, 6 aa, Artificial Sequence
  synthetic peptide
HSLEGF

SEQ ID NO: 633 - protein, 6 aa, Artificial Sequence
  synthetic peptide
HDTGYL

SEQ ID NO: 634 - protein, 6 aa, Artificial Sequence
  synthetic peptide
HDTSGL

SEQ ID NO: 635 - protein, 6 aa, Artificial Sequence
  synthetic peptide
HDGYTL

SEQ ID NO: 636 - protein, 6 aa, Artificial Sequence
  synthetic peptide
HDSGTL

SEQ ID NO: 637 - protein, 6 aa, Artificial Sequence
  synthetic peptide
HGYDTL

SEQ ID NO: 638 - protein, 6 aa, Artificial Sequence
  synthetic peptide
HSGDTL

SEQ ID NO: 639 - protein, 6 aa, Artificial Sequence
  synthetic peptide
HGDTYL

SEQ ID NO: 640 - protein, 6 aa, Artificial Sequence
  synthetic peptide
HSDTGL

SEQ ID NO: 641 - protein, 6 aa, Artificial Sequence
  synthetic peptide
TEFGYL

SEQ ID NO: 642 - protein, 6 aa, Artificial Sequence
  synthetic peptide
TEFSGL

SEQ ID NO: 643 - protein, 6 aa, Artificial Sequence
  synthetic peptide
TEGYFL

SEQ ID NO: 644 - protein, 6 aa, Artificial Sequence
  synthetic peptide
TESGFL

SEQ ID NO: 645 - protein, 6 aa, Artificial Sequence
  synthetic peptide
TGYEFL

SEQ ID NO: 646 - protein, 6 aa, Artificial Sequence
  synthetic peptide
TSGEFL

SEQ ID NO: 647 - protein, 6 aa, Artificial Sequence
  synthetic peptide
TGEFYL

SEQ ID NO: 648 - protein, 6 aa, Artificial Sequence
  synthetic peptide
TSEFGL

SEQ ID NO: 649 - protein, 6 aa, Artificial Sequence
  synthetic peptide
TPDGYK

SEQ ID NO: 650 - protein, 6 aa, Artificial Sequence
  synthetic peptide
TPDSGK

SEQ ID NO: 651 - protein, 6 aa, Artificial Sequence
  synthetic peptide
TPGYDK

SEQ ID NO: 652 - protein, 6 aa, Artificial Sequence
  synthetic peptide
TPSGDK

SEQ ID NO: 653 - protein, 6 aa, Artificial Sequence
  synthetic peptide
TGYPDK

SEQ ID NO: 654 - protein, 6 aa, Artificial Sequence
  synthetic peptide
TSGPDK

SEQ ID NO: 655 - protein, 6 aa, Artificial Sequence
  synthetic peptide
TGPDYK

SEQ ID NO: 656 - protein, 6 aa, Artificial Sequence
  synthetic peptide
TSPDGK

SEQ ID NO: 657 - protein, 6 aa, Artificial Sequence
  synthetic peptide
TQLGYE

SEQ ID NO: 658 - protein, 6 aa, Artificial Sequence
  synthetic peptide
TQLSGE

SEQ ID NO: 659 - protein, 6 aa, Artificial Sequence
  synthetic peptide
TQGYLE

SEQ ID NO: 660 - protein, 6 aa, Artificial Sequence
  synthetic peptide
TQSGLE

SEQ ID NO: 661 - protein, 6 aa, Artificial Sequence
  synthetic peptide
TGYQLE

SEQ ID NO: 662 - protein, 6 aa, Artificial Sequence
  synthetic peptide
TSGQLE

SEQ ID NO: 663 - protein, 6 aa, Artificial Sequence
  synthetic peptide
TGQLYE

SEQ ID NO: 664 - protein, 6 aa, Artificial Sequence
  synthetic peptide
TSQLGE

SEQ ID NO: 665 - protein, 6 aa, Artificial Sequence
  synthetic peptide
TNDGYL

SEQ ID NO: 666 - protein, 6 aa, Artificial Sequence
  synthetic peptide
TNDSGL

SEQ ID NO: 667 - protein, 5 aa, Artificial Sequence
  synthetic peptide
TNGYD

SEQ ID NO: 668 - protein, 6 aa, Artificial Sequence
  synthetic peptide
TNSGDL

SEQ ID NO: 669 - protein, 6 aa, Artificial Sequence
  synthetic peptide
TGYNDL

SEQ ID NO: 670 - protein, 6 aa, Artificial Sequence
  synthetic peptide
TSGNDL

SEQ ID NO: 671 - protein, 6 aa, Artificial Sequence
  synthetic peptide
TGNDYL

SEQ ID NO: 672 - protein, 6 aa, Artificial Sequence
  synthetic peptide
TSNDGL

SEQ ID NO: 673 - protein, 6 aa, Artificial Sequence
  synthetic peptide
TFHGYE

SEQ ID NO: 674 - protein, 6 aa, Artificial Sequence
  synthetic peptide
TFHSGE

SEQ ID NO: 675 - protein, 6 aa, Artificial Sequence
  synthetic peptide
TFGYHE

SEQ ID NO: 676 - protein, 6 aa, Artificial Sequence
  synthetic peptide
TFSGHE

SEQ ID NO: 677 - protein, 6 aa, Artificial Sequence
  synthetic peptide
TGYFHE

SEQ ID NO: 678 - protein, 6 aa, Artificial Sequence
  synthetic peptide
TSGFHE

SEQ ID NO: 679 - protein, 6 aa, Artificial Sequence
  synthetic peptide
TGFHYE

SEQ ID NO: 680 - protein, 6 aa, Artificial Sequence
  synthetic peptide
TSFHGE

SEQ ID NO: 681 - protein, 6 aa, Artificial Sequence
  synthetic peptide
THLGYK

SEQ ID NO: 682 - protein, 6 aa, Artificial Sequence
  synthetic peptide
THLSGK

SEQ ID NO: 683 - protein, 6 aa, Artificial Sequence
  synthetic peptide
THGYLK

SEQ ID NO: 684 - protein, 6 aa, Artificial Sequence
  synthetic peptide
THSGLK

SEQ ID NO: 685 - protein, 6 aa, Artificial Sequence
  synthetic peptide
TGYHLK

SEQ ID NO: 686 - protein, 6 aa, Artificial Sequence
  synthetic peptide
TSGHLK

SEQ ID NO: 687 - protein, 6 aa, Artificial Sequence
  synthetic peptide
TGHLYK

SEQ ID NO: 688 - protein, 6 aa, Artificial Sequence
  synthetic peptide
TSHLGK

SEQ ID NO: 689 - protein, 6 aa, Artificial Sequence
  synthetic peptide
TLNGYF

SEQ ID NO: 690 - protein, 6 aa, Artificial Sequence
  synthetic peptide
TLNSGF

SEQ ID NO: 691 - protein, 6 aa, Artificial Sequence
  synthetic peptide
TLGYNF

SEQ ID NO: 692 - protein, 6 aa, Artificial Sequence
  synthetic peptide
TLSGNF

SEQ ID NO: 693 - protein, 6 aa, Artificial Sequence
  synthetic peptide
TGYLNF

SEQ ID NO: 694 - protein, 6 aa, Artificial Sequence
  synthetic peptide
TSGLNF

SEQ ID NO: 695 - protein, 6 aa, Artificial Sequence
  synthetic peptide
TGLNYF

SEQ ID NO: 696 - protein, 6 aa, Artificial Sequence
  synthetic peptide
TSLNGF

SEQ ID NO: 697 - protein, 6 aa, Artificial Sequence
  synthetic peptide
TDEGYQ

SEQ ID NO: 698 - protein, 6 aa, Artificial Sequence
  synthetic peptide
TDESGQ

SEQ ID NO: 699 - protein, 6 aa, Artificial Sequence
  synthetic peptide
TDGYEQ

SEQ ID NO: 700 - protein, 6 aa, Artificial Sequence
  synthetic peptide
TDSGEQ

SEQ ID NO: 701 - protein, 6 aa, Artificial Sequence
  synthetic peptide
TGYDEQ

SEQ ID NO: 702 - protein, 6 aa, Artificial Sequence
  synthetic peptide
TSGDEQ

SEQ ID NO: 703 - protein, 6 aa, Artificial Sequence
  synthetic peptide
TGDEYQ

SEQ ID NO: 704 - protein, 6 aa, Artificial Sequence
  synthetic peptide
TSDEGQ

SEQ ID NO: 705 - protein, 6 aa, Artificial Sequence
  synthetic peptide
KEPGYH

SEQ ID NO: 706 - protein, 6 aa, Artificial Sequence
  synthetic peptide
KEPSGH

SEQ ID NO: 707 - protein, 6 aa, Artificial Sequence
  synthetic peptide
KEGYPH

SEQ ID NO: 708 - protein, 6 aa, Artificial Sequence
  synthetic peptide
KESGPH

SEQ ID NO: 709 - protein, 6 aa, Artificial Sequence
  synthetic peptide
KGYEPH

SEQ ID NO: 710 - protein, 6 aa, Artificial Sequence
  synthetic peptide
KSGEPH

SEQ ID NO: 711 - protein, 6 aa, Artificial Sequence
  synthetic peptide
KGEPYH

SEQ ID NO: 712 - protein, 6 aa, Artificial Sequence
  synthetic peptide
KSEPGH

SEQ ID NO: 713 - protein, 6 aa, Artificial Sequence
  synthetic peptide
KEDGYF

SEQ ID NO: 714 - protein, 6 aa, Artificial Sequence
  synthetic peptide
KEDSGF

SEQ ID NO: 715 - protein, 6 aa, Artificial Sequence
  synthetic peptide
KEGYDF

SEQ ID NO: 716 - protein, 6 aa, Artificial Sequence
  synthetic peptide
KESGDF

SEQ ID NO: 717 - protein, 6 aa, Artificial Sequence
  synthetic peptide
KGYEDF

SEQ ID NO: 718 - protein, 6 aa, Artificial Sequence
  synthetic peptide
KSGEDF

SEQ ID NO: 719 - protein, 6 aa, Artificial Sequence
  synthetic peptide
KGEDYF

SEQ ID NO: 720 - protein, 6 aa, Artificial Sequence
  synthetic peptide
KSEDGF

SEQ ID NO: 721 - protein, 6 aa, Artificial Sequence
  synthetic peptide
KPHGYN

SEQ ID NO: 722 - protein, 6 aa, Artificial Sequence
  synthetic peptide
KPHSGN

SEQ ID NO: 723 - protein, 6 aa, Artificial Sequence
  synthetic peptide
KPGYHN

SEQ ID NO: 724 - protein, 6 aa, Artificial Sequence
  synthetic peptide
KPSGHN

SEQ ID NO: 725 - protein, 6 aa, Artificial Sequence
  synthetic peptide
KGYPHN

SEQ ID NO: 726 - protein, 6 aa, Artificial Sequence
  synthetic peptide
KSGPHN

SEQ ID NO: 727 - protein, 6 aa, Artificial Sequence
  synthetic peptide
KGPHYN

SEQ ID NO: 728 - protein, 6 aa, Artificial Sequence
  synthetic peptide
KSPHGN

SEQ ID NO: 729 - protein, 6 aa, Artificial Sequence
  synthetic peptide
KQNGYT

SEQ ID NO: 730 - protein, 6 aa, Artificial Sequence
  synthetic peptide
KQNSGT

SEQ ID NO: 731 - protein, 6 aa, Artificial Sequence
  synthetic peptide
KQGYNT

SEQ ID NO: 732 - protein, 6 aa, Artificial Sequence
  synthetic peptide
KQSGNT

SEQ ID NO: 733 - protein, 6 aa, Artificial Sequence
  synthetic peptide
KGYQNT

SEQ ID NO: 734 - protein, 6 aa, Artificial Sequence
  synthetic peptide
KSGQNT

SEQ ID NO: 735 - protein, 6 aa, Artificial Sequence
  synthetic peptide
KGQNYT

SEQ ID NO: 736 - protein, 6 aa, Artificial Sequence
  synthetic peptide
KSQNGT

SEQ ID NO: 737 - protein, 6 aa, Artificial Sequence
  synthetic peptide
KNPGYL

SEQ ID NO: 738 - protein, 6 aa, Artificial Sequence
  synthetic peptide
KNPSGL

SEQ ID NO: 739 - protein, 6 aa, Artificial Sequence
  synthetic peptide
KNGYPL

SEQ ID NO: 740 - protein, 6 aa, Artificial Sequence
  synthetic peptide
KNSGPL

SEQ ID NO: 741 - protein, 6 aa, Artificial Sequence
  synthetic peptide
KGYNPL

SEQ ID NO: 742 - protein, 6 aa, Artificial Sequence
  synthetic peptide
KSGNPL

SEQ ID NO: 743 - protein, 6 aa, Artificial Sequence
  synthetic peptide
KGNPYL

SEQ ID NO: 744 - protein, 6 aa, Artificial Sequence
  synthetic peptide
KSNPGL

SEQ ID NO: 745 - protein, 6 aa, Artificial Sequence
  synthetic peptide
KNDGYQ

SEQ ID NO: 746 - protein, 6 aa, Artificial Sequence
  synthetic peptide
KNDSGQ

SEQ ID NO: 747 - protein, 6 aa, Artificial Sequence
  synthetic peptide
KNGYDQ

SEQ ID NO: 748 - protein, 6 aa, Artificial Sequence
  synthetic peptide
KNSGDQ

SEQ ID NO: 749 - protein, 6 aa, Artificial Sequence
  synthetic peptide
KGYNDQ

SEQ ID NO: 750 - protein, 6 aa, Artificial Sequence
  synthetic peptide
KSGNDQ

SEQ ID NO: 751 - protein, 6 aa, Artificial Sequence
  synthetic peptide
KGNDYQ

SEQ ID NO: 752 - protein, 6 aa, Artificial Sequence
  synthetic peptide
KSNDGQ

SEQ ID NO: 753 - protein, 6 aa, Artificial Sequence
  synthetic peptide
KFHGYP

SEQ ID NO: 754 - protein, 6 aa, Artificial Sequence
  synthetic peptide
KFHSGP

SEQ ID NO: 755 - protein, 6 aa, Artificial Sequence
  synthetic peptide
KFGYHP

SEQ ID NO: 756 - protein, 6 aa, Artificial Sequence
  synthetic peptide
KFSGHP

SEQ ID NO: 757 - protein, 6 aa, Artificial Sequence
  synthetic peptide
KGYFHP

SEQ ID NO: 758 - protein, 6 aa, Artificial Sequence
  synthetic peptide
KSGFHP

SEQ ID NO: 759 - protein, 6 aa, Artificial Sequence
  synthetic peptide
KGFHYP

SEQ ID NO: 760 - protein, 6 aa, Artificial Sequence
  synthetic peptide
KSFHGP

SEQ ID NO: 761 - protein, 6 aa, Artificial Sequence
  synthetic peptide
KFLGYH

SEQ ID NO: 762 - protein, 6 aa, Artificial Sequence
  synthetic peptide
KFLSGH

SEQ ID NO: 763 - protein, 6 aa, Artificial Sequence
  synthetic peptide
KFGYLH

SEQ ID NO: 764 - protein, 6 aa, Artificial Sequence
  synthetic peptide
KFSGLH

SEQ ID NO: 765 - protein, 6 aa, Artificial Sequence
  synthetic peptide
KGYFLH

SEQ ID NO: 766 - protein, 6 aa, Artificial Sequence
  synthetic peptide
KSGFLH

SEQ ID NO: 767 - protein, 6 aa, Artificial Sequence
  synthetic peptide
KGFLYH

SEQ ID NO: 768 - protein, 6 aa, Artificial Sequence
  synthetic peptide
KSFLGH

SEQ ID NO: 769 - protein, 6 aa, Artificial Sequence
  synthetic peptide
KHPGYD

SEQ ID NO: 770 - protein, 6 aa, Artificial Sequence
  synthetic peptide
KHPSGD

SEQ ID NO: 771 - protein, 6 aa, Artificial Sequence
  synthetic peptide
KHGYPD

SEQ ID NO: 772 - protein, 6 aa, Artificial Sequence
  synthetic peptide
KHSGPD

SEQ ID NO: 773 - protein, 6 aa, Artificial Sequence
  synthetic peptide
KGYHPD

SEQ ID NO: 774 - protein, 6 aa, Artificial Sequence
  synthetic peptide
KSGHPD

SEQ ID NO: 775 - protein, 6 aa, Artificial Sequence
  synthetic peptide
KGHPYD

SEQ ID NO: 776 - protein, 6 aa, Artificial Sequence
  synthetic peptide
KSHPGD

SEQ ID NO: 777 - protein, 6 aa, Artificial Sequence
  synthetic peptide
KTNGYD

SEQ ID NO: 778 - protein, 6 aa, Artificial Sequence
  synthetic peptide
KTNSGD

SEQ ID NO: 779 - protein, 6 aa, Artificial Sequence
  synthetic peptide
KTGYND

SEQ ID NO: 780 - protein, 6 aa, Artificial Sequence
  synthetic peptide
KTSGND

SEQ ID NO: 781 - protein, 6 aa, Artificial Sequence
  synthetic peptide
KGYTND

SEQ ID NO: 782 - protein, 6 aa, Artificial Sequence
  synthetic peptide
KSGTND

SEQ ID NO: 783 - protein, 6 aa, Artificial Sequence
  synthetic peptide
KGTNYD

SEQ ID NO: 784 - protein, 6 aa, Artificial Sequence
  synthetic peptide
KSTNGD

SEQ ID NO: 785 - protein, 6 aa, Artificial Sequence
  synthetic peptide
KDNGYL

SEQ ID NO: 786 - protein, 6 aa, Artificial Sequence
  synthetic peptide
KDNSGL

SEQ ID NO: 787 - protein, 6 aa, Artificial Sequence
  synthetic peptide
KDGYNL

SEQ ID NO: 788 - protein, 6 aa, Artificial Sequence
  synthetic peptide
KDSGNL

SEQ ID NO: 789 - protein, 6 aa, Artificial Sequence
  synthetic peptide
KGYDNL

SEQ ID NO: 790 - protein, 6 aa, Artificial Sequence
  synthetic peptide
KSGDNL

SEQ ID NO: 791 - protein, 6 aa, Artificial Sequence
  synthetic peptide
KGDNYL

SEQ ID NO: 792 - protein, 6 aa, Artificial Sequence
  synthetic peptide
KSDNGL

SEQ ID NO: 793 - protein, 6 aa, Artificial Sequence
  synthetic peptide
KDHGYE

SEQ ID NO: 794 - protein, 6 aa, Artificial Sequence
  synthetic peptide
KDHSGE

SEQ ID NO: 795 - protein, 6 aa, Artificial Sequence
  synthetic peptide
KDGYHE

SEQ ID NO: 796 - protein, 6 aa, Artificial Sequence
  synthetic peptide
KDSGHE

SEQ ID NO: 797 - protein, 6 aa, Artificial Sequence
  synthetic peptide
KGYDHE

SEQ ID NO: 798 - protein, 6 aa, Artificial Sequence
  synthetic peptide
KSGDHE

SEQ ID NO: 799 - protein, 6 aa, Artificial Sequence
  synthetic peptide
KGDHYE

SEQ ID NO: 800 - protein, 6 aa, Artificial Sequence
  synthetic peptide
KSDHGE

SEQ ID NO: 801 - protein, 6 aa, Artificial Sequence
  synthetic peptide
LEFGYK

SEQ ID NO: 802 - protein, 6 aa, Artificial Sequence
  synthetic peptide
LEFSGK

SEQ ID NO: 803 - protein, 6 aa, Artificial Sequence
  synthetic peptide
LEGYFK

SEQ ID NO: 804 - protein, 6 aa, Artificial Sequence
  synthetic peptide
LESGFK

SEQ ID NO: 805 - protein, 6 aa, Artificial Sequence
  synthetic peptide
LGYEFK

SEQ ID NO: 806 - protein, 6 aa, Artificial Sequence
  synthetic peptide
LSGEFK

SEQ ID NO: 807 - protein, 6 aa, Artificial Sequence
  synthetic peptide
LGEFYK

SEQ ID NO: 808 - protein, 6 aa, Artificial Sequence
  synthetic peptide
LSEFGK

SEQ ID NO: 809 - protein, 6 aa, Artificial Sequence
  synthetic peptide
LQEGYN

SEQ ID NO: 810 - protein, 6 aa, Artificial Sequence
  synthetic peptide
LQESGN

SEQ ID NO: 811 - protein, 6 aa, Artificial Sequence
  synthetic peptide
LQGYEN

SEQ ID NO: 812 - protein, 6 aa, Artificial Sequence
  synthetic peptide
LQSGEN

SEQ ID NO: 813 - protein, 6 aa, Artificial Sequence
  synthetic peptide
LGYQEN

SEQ ID NO: 814 - protein, 6 aa, Artificial Sequence
  synthetic peptide
LSGQEN

SEQ ID NO: 815 - protein, 6 aa, Artificial Sequence
  synthetic peptide
LGQEYN

SEQ ID NO: 816 - protein, 6 aa, Artificial Sequence
  synthetic peptide
LSQEGN

SEQ ID NO: 817 - protein, 6 aa, Artificial Sequence
  synthetic peptide
LNQGYT

SEQ ID NO: 818 - protein, 6 aa, Artificial Sequence
  synthetic peptide
LNQSGT

SEQ ID NO: 819 - protein, 6 aa, Artificial Sequence
  synthetic peptide
LNGYQT

SEQ ID NO: 820 - protein, 6 aa, Artificial Sequence
  synthetic peptide
LNSGQT

SEQ ID NO: 821 - protein, 6 aa, Artificial Sequence
  synthetic peptide
LGYNQT

SEQ ID NO: 822 - protein, 6 aa, Artificial Sequence
  synthetic peptide
LSGNQT

SEQ ID NO: 823 - protein, 6 aa, Artificial Sequence
  synthetic peptide
LGNQYT

SEQ ID NO: 824 - protein, 6 aa, Artificial Sequence
  synthetic peptide
LSNQGT

SEQ ID NO: 825 - protein, 6 aa, Artificial Sequence
  synthetic peptide
LFHGYK

SEQ ID NO: 826 - protein, 6 aa, Artificial Sequence
  synthetic peptide
LFHSGK

SEQ ID NO: 827 - protein, 6 aa, Artificial Sequence
  synthetic peptide
LFGYHK

SEQ ID NO: 828 - protein, 6 aa, Artificial Sequence
  synthetic peptide
LFSGHK

SEQ ID NO: 829 - protein, 6 aa, Artificial Sequence
  synthetic peptide
LGYFHK

SEQ ID NO: 830 - protein, 6 aa, Artificial Sequence
  synthetic peptide
LSGFHK

SEQ ID NO: 831 - protein, 6 aa, Artificial Sequence
  synthetic peptide
LGFHYK

SEQ ID NO: 832 - protein, 6 aa, Artificial Sequence
  synthetic peptide
LSFHGK

SEQ ID NO: 833 - protein, 6 aa, Artificial Sequence
  synthetic peptide
LFKGYD

SEQ ID NO: 834 - protein, 6 aa, Artificial Sequence
  synthetic peptide
LFKSGD

SEQ ID NO: 835 - protein, 6 aa, Artificial Sequence
  synthetic peptide
LFGYKD

SEQ ID NO: 836 - protein, 6 aa, Artificial Sequence
  synthetic peptide
LFSGKD

SEQ ID NO: 837 - protein, 6 aa, Artificial Sequence
  synthetic peptide
LGYFKD

SEQ ID NO: 838 - protein, 6 aa, Artificial Sequence
  synthetic peptide
LSGFKD

SEQ ID NO: 839 - protein, 6 aa, Artificial Sequence
  synthetic peptide
LGFKYD

SEQ ID NO: 840 - protein, 6 aa, Artificial Sequence
  synthetic peptide
LSFKGD

SEQ ID NO: 841 - protein, 6 aa, Artificial Sequence
  synthetic peptide
LHDGYF

SEQ ID NO: 842 - protein, 6 aa, Artificial Sequence
  synthetic peptide
LHDSGF

SEQ ID NO: 843 - protein, 6 aa, Artificial Sequence
  synthetic peptide
LHGYDF

SEQ ID NO: 844 - protein, 6 aa, Artificial Sequence
  synthetic peptide
LHSGDF

SEQ ID NO: 845 - protein, 6 aa, Artificial Sequence
  synthetic peptide
LGYHDF

SEQ ID NO: 846 - protein, 6 aa, Artificial Sequence
  synthetic peptide
LSGHDF

SEQ ID NO: 847 - protein, 6 aa, Artificial Sequence
  synthetic peptide
LGHDYF

SEQ ID NO: 848 - protein, 6 aa, Artificial Sequence
  synthetic peptide
LSHDGF

SEQ ID NO: 849 - protein, 6 aa, Artificial Sequence
  synthetic peptide
LTDGYK

SEQ ID NO: 850 - protein, 6 aa, Artificial Sequence
  synthetic peptide
LTDSGK

SEQ ID NO: 851 - protein, 6 aa, Artificial Sequence
  synthetic peptide
LTGYDK

SEQ ID NO: 852 - protein, 6 aa, Artificial Sequence
  synthetic peptide
LTSGDK

SEQ ID NO: 853 - protein, 6 aa, Artificial Sequence
  synthetic peptide
LGYTDK

SEQ ID NO: 854 - protein, 6 aa, Artificial Sequence
  synthetic peptide
LSGTDK

SEQ ID NO: 855 - protein, 6 aa, Artificial Sequence
  synthetic peptide
LGTDYK

SEQ ID NO: 856 - protein, 6 aa, Artificial Sequence
  synthetic peptide
LSTDGK

SEQ ID NO: 857 - protein, 6 aa, Artificial Sequence
  synthetic peptide
LDEGYH

SEQ ID NO: 858 - protein, 6 aa, Artificial Sequence
  synthetic peptide
LDESGH

SEQ ID NO: 859 - protein, 6 aa, Artificial Sequence
  synthetic peptide
LDGYEH

SEQ ID NO: 860 - protein, 6 aa, Artificial Sequence
  synthetic peptide
LDSGEH

SEQ ID NO: 861 - protein, 6 aa, Artificial Sequence
  synthetic peptide
LGYDEH

SEQ ID NO: 862 - protein, 6 aa, Artificial Sequence
  synthetic peptide
LSGDEH

SEQ ID NO: 863 - protein, 6 aa, Artificial Sequence
  synthetic peptide
LGDEYH

SEQ ID NO: 864 - protein, 6 aa, Artificial Sequence
  synthetic peptide
LSDEGH

SEQ ID NO: 865 - protein, 6 aa, Artificial Sequence
  synthetic peptide
DEPGYK

SEQ ID NO: 866 - protein, 6 aa, Artificial Sequence
  synthetic peptide
DEPSGK

SEQ ID NO: 867 - protein, 6 aa, Artificial Sequence
  synthetic peptide
DEGYPK

SEQ ID NO: 868 - protein, 6 aa, Artificial Sequence
  synthetic peptide
DESGPK

SEQ ID NO: 869 - protein, 6 aa, Artificial Sequence
  synthetic peptide
DGYEPK

SEQ ID NO: 870 - protein, 6 aa, Artificial Sequence
  synthetic peptide
DSGEPK

SEQ ID NO: 871 - protein, 6 aa, Artificial Sequence
  synthetic peptide
DGEPYK

SEQ ID NO: 872 - protein, 6 aa, Artificial Sequence
  synthetic peptide
DSEPGK

SEQ ID NO: 873 - protein, 6 aa, Artificial Sequence
  synthetic peptide
DELGYT

SEQ ID NO: 874 - protein, 6 aa, Artificial Sequence
  synthetic peptide
DELSGT

SEQ ID NO: 875 - protein, 6 aa, Artificial Sequence
  synthetic peptide
DEGYLT

SEQ ID NO: 876 - protein, 6 aa, Artificial Sequence
  synthetic peptide
DESGLT

SEQ ID NO: 877 - protein, 6 aa, Artificial Sequence
  synthetic peptide
DGYELT

SEQ ID NO: 878 - protein, 6 aa, Artificial Sequence
  synthetic peptide
DSGELT

SEQ ID NO: 879 - protein, 6 aa, Artificial Sequence
  synthetic peptide
DGELYT

SEQ ID NO: 880 - protein, 6 aa, Artificial Sequence
  synthetic peptide
DSELGT

SEQ ID NO: 881 - protein, 6 aa, Artificial Sequence
  synthetic peptide
DNKGYQ

SEQ ID NO: 882 - protein, 6 aa, Artificial Sequence
  synthetic peptide
DNKSGQ

SEQ ID NO: 883 - protein, 6 aa, Artificial Sequence
  synthetic peptide
DNGYKQ

SEQ ID NO: 884 - protein, 6 aa, Artificial Sequence
  synthetic peptide
DNSGKQ

SEQ ID NO: 885 - protein, 6 aa, Artificial Sequence
  synthetic peptide
DGYNKQ

SEQ ID NO: 886 - protein, 6 aa, Artificial Sequence
  synthetic peptide
DSGNKQ

SEQ ID NO: 887 - protein, 6 aa, Artificial Sequence
  synthetic peptide
DGNKYQ

SEQ ID NO: 888 - protein, 6 aa, Artificial Sequence
  synthetic peptide
DSNKGQ

SEQ ID NO: 889 - protein, 6 aa, Artificial Sequence
  synthetic peptide
DTEGYQ

SEQ ID NO: 890 - protein, 6 aa, Artificial Sequence
  synthetic peptide
DTESGQ

SEQ ID NO: 891 - protein, 6 aa, Artificial Sequence
  synthetic peptide
DTGYEQ

SEQ ID NO: 892 - protein, 6 aa, Artificial Sequence
  synthetic peptide
DTSGEQ

SEQ ID NO: 893 - protein, 6 aa, Artificial Sequence
  synthetic peptide
DGYTEQ

SEQ ID NO: 894 - protein, 6 aa, Artificial Sequence
  synthetic peptide
DSGTEQ

SEQ ID NO: 895 - protein, 6 aa, Artificial Sequence
  synthetic peptide
DGTEYQ

SEQ ID NO: 896 - protein, 6 aa, Artificial Sequence
  synthetic peptide
DSTEGQ

SEQ ID NO: 897 - protein, 6 aa, Artificial Sequence
  synthetic peptide
DKHGYP

SEQ ID NO: 898 - protein, 6 aa, Artificial Sequence
  synthetic peptide
DKHSGP

SEQ ID NO: 899 - protein, 6 aa, Artificial Sequence
  synthetic peptide
DKGYHP

SEQ ID NO: 900 - protein, 6 aa, Artificial Sequence
  synthetic peptide
DKSGHP

SEQ ID NO: 901 - protein, 6 aa, Artificial Sequence
  synthetic peptide
DGYKHP

SEQ ID NO: 902 - protein, 6 aa, Artificial Sequence
  synthetic peptide
DSGKHP

SEQ ID NO: 903 - protein, 6 aa, Artificial Sequence
  synthetic peptide
DGKHYP

SEQ ID NO: 904 - protein, 6 aa, Artificial Sequence
  synthetic peptide
DSKHGP

SEQ ID NO: 905 - protein, 6 aa, Artificial Sequence
  synthetic peptide
DLTGYF

SEQ ID NO: 906 - protein, 6 aa, Artificial Sequence
  synthetic peptide
DLTSGF

SEQ ID NO: 907 - protein, 6 aa, Artificial Sequence
  synthetic peptide
DLGYTF

SEQ ID NO: 908 - protein, 6 aa, Artificial Sequence
  synthetic peptide
DLSGTF

SEQ ID NO: 909 - protein, 6 aa, Artificial Sequence
  synthetic peptide
DGYLTF

SEQ ID NO: 910 - protein, 6 aa, Artificial Sequence
  synthetic peptide
DSGLTF

SEQ ID NO: 911 - protein, 6 aa, Artificial Sequence
  synthetic peptide
DGLTYF

SEQ ID NO: 912 - protein, 13 aa, Artificial Sequence
  E-tag Peptide
GAPVPYPDPLEPR

SEQ ID NO: 913 - protein, 8 aa, Artificial Sequence
  Flag Peptide
DYKDDDDK

SEQ ID NO: 914 - protein, 9 aa, Artificial Sequence
  Glu-Glu Peptide
EEEEYMPME

SEQ ID NO: 915 - protein, 9 aa, Artificial Sequence
  HA.11 Peptide
YPYDVPDYA

SEQ ID NO: 916 - protein, 11 aa, Artificial Sequence
  HSV Tag Peptide
QPELAPEDPED

SEQ ID NO: 917 - protein, 10 aa, Artificial Sequence
  c-myc Peptide
EQKLISEEDL

SEQ ID NO: 918 - protein, 11 aa, Artificial Sequence
  T7 Tag Peptide
MASMTGGQQMG

SEQ ID NO: 919 - protein, 11 aa, Artificial Sequence
  VSV-G Peptide
YTDIEMNRLGK

SEQ ID NO: 920 - protein, 14 aa, Artificial Sequence
  V5 Peptide
GKPIPNPLLGLDST

SEQ ID NO: 921 - protein, 12 aa, Artificial Sequence
  AB2 Peptide
LTPPMGPVIDQR

SEQ ID NO: 922 - protein, 12 aa, Artificial Sequence
  AB4 Peptide
QPQSKGFEPPPP

SEQ ID NO: 923 - protein, 12 aa, Artificial Sequence
  B34 Peptide
DLHDERTLQFKL

SEQ ID NO: 924 - protein, 12 aa, Artificial Sequence
  P5D4-A Peptide
HPNLPETRRYAL

SEQ ID NO: 925 - protein, 12 aa, Artificial Sequence
  P5D4-B Peptide
SYTGIEFDRLSN

SEQ ID NO: 926 - protein, 12 aa, Artificial Sequence
  4C10 Peptide
MVDPEAQDVPKW

SEQ ID NO: 927 - protein, 12 aa, Artificial Sequence
  AB3 Peptide
YEYAKGSEPPAL

SEQ ID NO: 928 - protein, 12 aa, Artificial Sequence
  AB6 Peptide
AGTQWCLTRPPC

SEQ ID NO: 929 - protein, 12 aa, Artificial Sequence
  KT3-A Peptide
KLMPNEFFGLLP

SEQ ID NO: 930 - protein, 12 aa, Artificial Sequence
  KT3-B Peptide
KLIPTQLYLLHP

SEQ ID NO: 931 - protein, 12 aa, Artificial Sequence
  KT3-C Peptide
SFMPIEFYARKL

SEQ ID NO: 932 - protein, 12 aa, Artificial Sequence
  723 Peptide
TNMEWMTSHRSA

SEQ ID NO: 933 - protein, 12 aa, Artificial Sequence
  HOPC1 Peptide
MPQQGDPDWVVP

SEQ ID NO: 934 - protein, 9 aa, Artificial Sequence
  S1 Peptide
NANNPDWDF

SEQ ID NO: 935 - protein, 10 aa, Artificial Sequence
  E2 Peptide
SSTSSDFRDR

SEQ ID NO: 936 - protein, 8 aa, Artificial Sequence
  His-tag Peptide
HHHHHHGS

SEQ ID NO: 937 - protein, 6 aa, Artificial Sequence
  AU1 Peptide
DTYRYI

SEQ ID NO: 938 - protein, 6 aa, Artificial Sequence
  AU5 Peptide
TDFYLK

SEQ ID NO: 939 - protein, 5 aa, Artificial Sequence
  IRS Peptide
RYIRS

SEQ ID NO: 940 - protein, 495 aa, Escherichia coli CFT073 [sequence omitted]

SEQ ID NO: 941 - protein, 396 aa, Escherichia coli
  ref <308>: DATABASE ACCESSION NUMBER: Genbank AAC43128
  ref <309>: DATABASE ENTRY DATE: 1993-09-03
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 942 - protein, 318 aa, Mus musculus
  ref <308>: DATABASE ACCESSION NUMBER: Genbank NP_037812
  ref <309>: DATABASE ENTRY DATE: 2002-01-07
MDQNNSLPPYAQGLASPQGAMTPGIPIFSPMMPYGTGLTPQPIQNTNSLSILEEQQRQQQQQQQQQQQQQQQAVATAAASVQQSTSQQPTQGASGQTPQLFHSQTLTTAPLPGTTPLYPSPMTPMTPITPATPASESSGIVPQLQNIVSTVNLGCKLDLKTIALRARNAEYNPKRFAAVIMRIREPRTTALIFSSGKMVCTGAKSEEQSRLAARKYARVVQKLGFPAKFLDFKIQNMVGSCDVKFPIRLEGLVLTHQQFSSYEPELFPGLIYRMIKPRIVLLIFVSGKVVLTGAKVRAEIYEAFENIYPILKGFRKTT

SEQ ID NO: 943 - protein, 105 aa, Mus musculus
  ref <308>: DATABASE ACCESSION NUMBER: Genbank BAA04881
  ref <309>: DATABASE ENTRY DATE: 2002-12-25
MVKLIESKEAFQEALAAAGDKLVVVDFSATWCGPCKMIKPFFHSLCDKYSNVVFLEVDVDDCQDVAADCEVKCMPTFQFYKKGQKVGEFSGANKEKLEASITEYA

SEQ ID NO: 944 - protein, 15 aa, Artificial Sequence
  S-tag Peptide
KETAAAKFERQHMDS

SEQ ID NO: 945 - protein, 11 aa, Artificial Sequence
  KT3 Peptide
KPPTPPPEPET

SEQ ID NO: 946 - protein, 43 aa, Homo sapiens
KISHFLKMESLNFIRAHTPYINIYNCEPANPSEKNSPSTQYCY

SEQ ID NO: 947 - DNA, 40 bp, Artificial Sequence
  Oligo JKF660
ctagaaaaaatttctcattttcttaaaatggaatctctta

SEQ ID NO: 948 - DNA, 40 bp, Artificial Sequence
  Oligo JKF661
attttattcgtgctcatactccttatattaatatttataa

SEQ ID NO: 949 - DNA, 40 bp, Artificial Sequence
  Oligo JKF662
ttgtgaacctgctaatccttctgaaaaaaattctccttct

SEQ ID NO: 950 - DNA, 16 bp, Artificial Sequence
  Oligo JKF663
actcaatattgttatc

SEQ ID NO: 951 - DNA, 40 bp, Artificial Sequence
  Oligo JKF664
tcgagataacaatattgagtagaaggagaatttttttcag

SEQ ID NO: 952 - DNA, 40 bp, Artificial Sequence
  Oligo JKF665
aaggattagcaggttcacaattataaatattaatataagg

SEQ ID NO: 953 - DNA, 40 bp, Artificial Sequence
  Oligo JKF666
agtatgagcacgaataaaattaagagattccattttaaga

SEQ ID NO: 954 - DNA, 16 bp, Artificial Sequence
  Oligo JKF667
aaatgagaaatttttt

SEQ ID NO: 955 - protein, 319 aa, Artificial Sequence
  pBAD S1C5 CD20 His Protein Sequence
MAEVQLQQSGAELVKPGASVKISCKASGYTFTDHVIHWVKQRPEQGLEWIGFISPGNGDIRYNEKFKDKATLTADKSSSTAYMQLNSLTSEDSAVYFCKRSFYYYDDNYGDYWGQGTTLTVSSSGAPGGGGSGGGGSGGGGSDIVLTQSPAIMSASLEERVTMTCTASSSVSSSYFHWYQQKPGSSPKLWIYTTSNLASGVPARFSGSGSGTSYSLTISSMEAEDAATYYCHQYHRSPLTFGAGTKLELKRADAAPTVSAAASFLKISHFLKMESLNFIRAHTPYINIYNCEPANPSEKNSPSTQYCYNSAVDHHHHHH

SEQ ID NO: 956 - DNA, 960 bp, Artificial Sequence
  pBAD S1C5 CD20 His DNA sequence
atggccgaggttcagcttcagcagtctggcgctgagttggtgaaacctggggcttcagtgaagatatcctgcaaggcttctggctacaccttcactgaccatgttattcactgggtgaagcagaggcctgaacagggcctggaatggattggatttatttctcccggaaatggtgatattagatataatgagaagttcaaggacaaggccacactgactgcagacaaatcctccagcactgcctacatgcagctcaatagtctgacatctgaggattctgcagtgtatttctgtaagagatccttttattactacgatgataactacggggactactggggccaaggcaccactctcacagtctcctcatcgggcgcgccaggtggaggcggttcaggcggaggtggctctggcggtggcggatcggacattgtgctcacccagtctccagcaatcatgtctgcatctctagaggaacgggtcactatgacctgcactgccagctcaagtgtaagttccagttacttccactggtaccagcagaagccaggatcctcccccaaactctggatttataccacatccaacctggcttctggagtcccagctcgcttcagtggcagtgggtctgggacctcttactctctcacaatcagcagcatggaggctgaagatgctgccacttattactgccaccagtatcatcgttccccgctcacgttcggtgctgggaccaagctggagctgaaacgggctgatgctgcaccaactgtatccgcggccgcaagctttctaaaaatttctcattttcttaaaatggaatctcttaattttattcgtgctcatactccttatattaatatttataattgtgaacctgctaatccttctgaaaaaaattctccttctactcaatattgttataatagcgccgtcgaccatcatcatcatcatcattga

SEQ ID NO: 957 - protein, 290 aa, Artificial Sequence
  pBAD S1C5 V5 His protein sequence
MAEVQLQQSGAELVKPGASVKISCKASGYTFTDHVIHWVKQRPEQGLEWIGFISPGNGDIRYNEKFKDKATLTADKSSSTAYMQLNSLTSEDSAVYFCKRSFYYYDDNYGDYWGQGTTLTVSSSGAPGGGGSGGGGSGGGGSDIVLTQSPAIMSASLEERVTMTCTASSSVSSSYFHWYQQKPGSSPKLWIYTTSNLASGVPARFSGSGSGTSYSLTISSMEAEDAATYYCHQYHRSPLTFGAGTKLELKRADAAPTVSAAASFLGKPIPNPLLGLDSTNSAVDHHHHHH

SEQ ID NO: 958 - DNA, 873 bp, Artificial Sequence
  pBAD S1C5 V5 His DNA sequence
atggccgaggttcagcttcagcagtctggcgctgagttggtgaaacctggggcttcagtgaagatatcctgcaaggcttctggctacaccttcactgaccatgttattcactgggtgaagcagaggcctgaacagggcctggaatggattggatttatttctcccggaaatggtgatattagatataatgagaagttcaaggacaaggccacactgactgcagacaaatcctccagcactgcctacatgcagctcaatagtctgacatctgaggattctgcagtgtatttctgtaagagatccttttattactacgatgataactacggggactactggggccaaggcaccactctcacagtctcctcatcgggcgcgccaggtggaggcggttcaggcggaggtggctctggcggtggcggatcggacattgtgctcacccagtctccagcaatcatgtctgcatctctagaggaacgggtcactatgacctgcactgccagctcaagtgtaagttccagttacttccactggtaccagcagaagccaggatcctcccccaaactctggatttataccacatccaacctggcttctggagtcccagctcgcttcagtggcagtgggtctgggacctcttactctctcacaatcagcagcatggaggctgaagatgctgccacttattactgccaccagtatcatcgttccccgctcacgttcggtgctgggaccaagctggagctgaaacgggctgatgctgcaccaactgtatccgcggccgcaagctttctaggtaagcctatccctaaccctctcctcggtctcgattctacgaatagcgccgtcgaccatcatcatcatcatcattga